(19) United States
(12) Patent Application Publication
(10) Pub. No.: US 2026/0337686 A1
(43) Pub. Date: Oct. 29, 2026

(54) METHODS AND COMPOSITIONS FOR QUANTIFYING IMMUNE CELL NUCLEIC ACIDS

(71) Applicant: GUARDANT HEALTH, INC., Palo Alto, CA (US)

(72) Inventors: Emily Katherine TSANG, Mountain View, CA (US); Noam VARDI, San Francisco, CA (US); Stefanie Ann Ward MORTIMER, Morgan Hill, CA (US); Andrew KENNEDY, San Diego, CA (US); Meromit SINGER, Menlo Park, CA (US)

(21) Appl. No.: 19/426,882

(22) Filed: Dec. 19, 2025

Related U.S. Application Data
(63) Continuation of application No. PCT/US2024/035306, filed on Jun. 24, 2024.
(60) Provisional application No. 63/510,026, filed on Jun. 23, 2023.

(51) Int. Cl. C12Q 1/6883

(52) U.S. Cl. CPC C12Q 1/6883; C12Q 2600/158

(57) ABSTRACT

Provided herein is an RNA analysis method for detecting and quantifying RNA (such as RNA from an immune cell or from a cancer cell) and/or for identifying and quantifying immune cell types from which the RNA originated. In some embodiments, expression levels of genes differentially expressed between healthy subjects and subjects having a disease or disorder are determined based on the RNA. In some embodiments, immune cell types from which the RNA originated are identified and quantified. Provided herein are also methods for determining the likelihood that a subject has the disease or disorder, such as a cancer.

### CROSS-REFERENCE TO RELATED APPLICATIONS

The present application is a Continuation of International Patent Application No. PCT/US2024/035306, filed Jun. 24, 2024, which claims the benefit of priority of U.S. Provisional Patent Application No. 63/510,026, filed Jun. 23, 2023, which is incorporated by reference herein in its entirety for all purposes.

### FIELD OF THE INVENTION

The present disclosure provides methods related to analyzing RNA, such as immune cell RNA or RNA from a cancer cell. In some embodiments, the RNA is from a subject having or suspected of having a disease or disorder, such as cancer. In some embodiments, expression levels of genes differentially expressed between healthy subjects and subjects having the disease or disorder are determined based on the RNA. In some embodiments, immune cell types from which the RNA originated are identified and quantified.

### SEQUENCE LISTING

The present application contains a sequence listing that has been submitted electronically in XML format. Said XML copy, created on Jun. 21, 2024, is named “01228-0036-00PCT.xml” and is 19,443 bytes in size. The information in the electronic format of the sequence listing is incorporated herein by reference in its entirety.

### INTRODUCTION AND SUMMARY

Invasive diagnostic procedures, including biopsies, are commonly used for detecting or diagnosing cancer, ulcers, liver diseases, infections, transplant rejections, and other diseases and disorders in which analysis of cells or tissue from a possible site of a malady are analyzed for relevant features. Detection of diseases and disorders based on analysis of body fluids (“liquid biopsies”), such as blood, is an intriguing alternative. A liquid biopsy is noninvasive, sometimes requiring only a blood draw. However, it has been challenging to develop accurate and sensitive methods for analyzing liquid biopsy material because the amount of nucleic acids released into body fluids is low and variable as is recovery of nucleic acids from such fluids in analyzable form.

An alternative or supplemental approach is to detect the signal linked to secondary effects of the presence of a disease such as cancer. One such secondary effect is the effect on RNA expression in other cells, including but not limited to cells involved in the immune response to tumorigenesis. In the presence of tumor cells, immune cells proliferate, differentiate, and potentially turn over at a higher rate than in a healthy subject. Such phenomena can result in changes in immune cell populations and the amount of RNA thereof in the blood. Therefore, a secondary RNA signal may be useful for detecting diseases or disorders, such as cancer, with improved sensitivity in at least some circumstances.

Furthermore, quantification of different blood cell types provides important information about a subject's overall health in addition to information about disease states. The ability to distinguish between different cell types, including closely related cell types, can be important for distinguishing between different types of diseases and disorders. In a disease state, RNA from certain immune cell types may be elevated, and can be used as an indicator of the disease. In a number of genomic regions, the DNA methylation signatures in different immune cell types are distinguishable from myeloid cells and other immune cell types. Existing methods, such as complete blood counts and microarray-based profiling of differentially methylated regions, allow detection of many cell types but do not discriminate between certain types of immune cells, e.g., naïve and activated lymphocytes, and therefore do not provide important information about the state of the subject's immune system.

The methods herein provide an approach to quantify the levels of RNA derived from different immune cell types based on RNA sequencing data, e.g., generated by sequencing RNA and determining expression levels for a target gene set comprising genes that are differentially expressed in one or more immune cell types and/or in samples from subjects with a disease or disorder (such as cancer) relative to samples from healthy subjects, thus facilitating determining the presence, absence, or likelihood of the disease or disorder in the subject. Applications of this approach include, e.g., cancer detection by detecting tumor-induced immune cell proliferation.

The present disclosure aims to meet the need for improved analysis of RNA originating from different immune cell types, including rare immune cell types, such as activated lymphocytes or regulatory T cells. Improved differentiation of immune cell types over existing methods, such as complete blood count (CBC) or DNA methylation-based methods that do not discriminate between immune cell types, allows for more accurate detection of diseases and disorders (diagnosis) and therefore improved treatments. Accordingly, the following exemplary embodiments are provided.

Embodiment 1 is a method of analyzing RNA in a sample from a subject, the method comprising:
a) sequencing the RNA and determining expression levels for a target gene set comprising a plurality of target genes that are differentially expressed in a plurality of immune cell types and/or in samples from subjects with a disease or disorder relative to samples from healthy subjects; and
b) determining
   (1) quantities of the immune cell types from which the RNA originated based on the expression levels; and/or
   (2) expression levels of the plurality of target genes; and
c) determining the presence, absence, or likelihood of the disease or disorder in the subject based on the quantities of the immune cell types and/or the expression levels of the plurality of target genes.

Embodiment 2 is the method of embodiment 1, wherein the plurality of immune cell types comprises one or more, or each, of neutrophils, lymphocytes, plasma cells, monocytes, macrophages, dendritic cells, mast cells, eosinophils, T cells, CD4+ T cells, B cells, NK cells, megakaryocytes, CD8+ central memory cells, CD8+ effector memory cells, CD4+ central memory cells, CD4+ effector memory cells, immature neutrophils, precursor B cells, plasma cells, memory-switched B cells, plasma cells, basophils, naïve B cells, memory B cells, CD8+ T cells, naïve CD4+ T cells, resting CD4+ memory T cells, activated CD4+ memory T cells, follicular helper T cells; regulatory T cells (Tregs), gamma delta T cells, resting NK cells, activated NK cells, M0 macrophages, M1 macrophages, M2 macrophages, resting dendritic cells, activated dendritic cells, resting mast cells, and activated mast cells.

Embodiment 2.1 is the method of embodiment 1 or embodiment 2, wherein the plurality of immune cell types comprises one or more, or each, of naïve B cells, naïve CD4+ T cells, CD8+ T cells, resting NK cells, Tregs, and monocytes.

Embodiment 3 is the method of embodiment 1 or embodiment 2, wherein the plurality of immune cell types comprises one or more, two or more, or three or more of CD8+ T cells, resting CD4+ memory T cells, Tregs, and naïve B cells.

Embodiment 4 is the method of embodiment 1 or embodiment 2, wherein the plurality of immune cell types comprises CD8+ T cells, resting CD4+ memory T cells, Tregs, and naïve B cells.

Embodiment 5 is the method of embodiment 1 or embodiment 2, wherein the plurality of immune cell types comprises CD8+ T cells, resting CD4+ memory T cells, and T regs.

Embodiment 6 is the method of embodiment 1 or embodiment 2, wherein the plurality of immune cell types comprises CD8+ T cells, Tregs, and naïve B cells.

Embodiment 7 is the method of embodiment 1 or embodiment 2, wherein the plurality of immune cell types comprises CD8+ T cells, resting CD4+ memory T cells, and naïve B cells.

Embodiment 8 is the method of embodiment 1 or embodiment 2, wherein the plurality of immune cell types comprises resting CD4+ memory T cells, Tregs, and naïve B cells.

Embodiment 9 is the method of embodiment 1 or embodiment 2, wherein the plurality of immune cell types comprises CD8+ T cells.

Embodiment 10 is the method of embodiment 1 or embodiment 2, wherein the plurality of immune cell types comprises resting CD4+ memory T cells.

Embodiment 11 is the method of embodiment 1 or embodiment 2, wherein the plurality of immune cell types comprises Tregs.

Embodiment 12 is the method of embodiment 1 or embodiment 2, wherein the plurality of immune cell types comprises naïve B cells.

Embodiment 13 is the method of embodiment 1 or embodiment 2, wherein the plurality of immune cell types comprises neutrophils.

Embodiment 13.1 is the method of embodiment 1 or embodiment 2, wherein the plurality of immune cell types comprises naïve CD4+ T cells

Embodiment 13.2 is the method of embodiment 1 or embodiment 2, wherein the plurality of immune cell types comprises resting NK cells.

Embodiment 13.3 is the method of embodiment 1 or embodiment 2, wherein the plurality of immune cell types comprises monocytes.

Embodiment 14 is the method of any one of the preceding embodiments, wherein the plurality of immune cell types comprises:
a) T cells, B cells, and NK cells;
b) neutrophils and lymphocytes;
c) neutrophils, T cells, B cells, and NK cells;
d) granulocytes and lymphocytes; or
e) granulocytes, T cells, B cells, and NK cells.

Embodiment 15 is the method of any one of the preceding embodiments, wherein determining the presence, absence, or likelihood of the disease or disorder in the subject based on the quantities of the immune cell types comprises determining the levels of one or more, or each, of the plurality of immune cell types relative to total blood cells.

Embodiment 16 is the method of any one of the preceding embodiments, wherein determining the presence, absence, or likelihood of the disease or disorder in the subject based on the quantities of the immune cell types comprises determining the levels of one or more, or each, of the plurality of immune cell types relative to total white blood cells.

Embodiment 17 is the method of any one of the preceding embodiments, wherein determining the presence, absence, or likelihood of the disease or disorder in the subject based on the quantities of the immune cell types comprises determining the levels of one or more, or each, of T, B, and NK cells relative to all lymphocytes.

Embodiment 18 is the method of any one of the preceding embodiments, wherein determining the presence, absence, or likelihood of the disease or disorder in the subject based on the quantities of the immune cell types comprises determining relative proportions of neutrophils and lymphocytes.

Embodiment 19 is the method of any one of the preceding embodiments, wherein determining the presence, absence, or likelihood of the disease or disorder in the subject based on the quantities of the immune cell types comprises determining relative proportions of neutrophils and one or more, or each, of T cells, B cells, and NK cells.

Embodiment 20 is the method of any one of the preceding embodiments, wherein the plurality of target genes comprises genes differentially expressed in an activated cell type relative to the same cell type that is not activated.

Embodiment 21 is the method of any one of the preceding embodiments, wherein the plurality of target genes comprises genes differentially expressed in at least (a) a first cell type that is activated relative to the same first cell type that is not activated, and (b) a second cell type that is activated relative to the same second cell type that is not activated.

Embodiment 22 is the method of embodiment 20 or embodiment 21, wherein the activated cell type is neutrophils, lymphocytes, plasma cells, monocytes, macrophages, dendritic cells, mast cells, or eosinophils.

Embodiment 23 is the method of any one of the preceding embodiments, wherein the plurality of target genes comprises genes differentially expressed in neutrophils relative to a non-neutrophil cell type.

Embodiment 24 is the method of the immediately preceding embodiment, wherein the non-neutrophil cell type is one or more, or each, of a non-immune cell type, a non-granulocyte cell type, a myeloid non-granulocyte cell type, a lymphoid cell type, lymphocytes, T cells, B cells, and NK cells.

Embodiment 25 is the method of any one of the preceding embodiments, wherein the plurality of target genes comprises genes differentially expressed in lymphocytes relative to a non-lymphocyte cell type.

Embodiment 26 is the method of any one of the preceding embodiments, wherein the plurality of target genes comprises genes differentially expressed in a first cell type relative to a second cell type different from the first cell type, and the first cell type is neutrophils, lymphocytes, plasma cells, monocytes, macrophages, dendritic cells, mast cells, eosinophils, T cells, CD4+ T cells, B cells, NK cells, megakaryocytes, CD8+ central memory cells, CD8+ effector memory cells, CD4+ central memory cells, CD4+ effector memory cells, immature neutrophils, precursor B cells, plasma cells, memory-switched B cells, plasma cells, basophils, naïve B cells, memory B cells, CD8+ T cells, naïve CD4+ T cells, resting CD4+ memory T cells, activated CD4+ memory T cells; follicular helper T cells; regulatory T cells (Tregs); gamma delta T cells; resting NK cells; activated NK cells, M0 macrophages, M1 macrophages, M2 macrophages, resting dendritic cells, activated dendritic cells, resting mast cells, or activated mast cells.

Embodiment 27 is the method of the immediately preceding embodiment, wherein the second cell type is neutrophils, lymphocytes, plasma cells, monocytes, macrophages, dendritic cells, mast cells, eosinophils, T cells, CD4+ T cells, B cells, NK cells, megakaryocytes, CD8+ central memory cells, CD8+ effector memory cells, CD4+ central memory cells, CD4+ effector memory cells, immature neutrophils, precursor B cells, plasma cells, memory-switched B cells, plasma cells, basophils, naïve B cells, memory B cells, CD8+ T cells, naïve CD4+ T cells, resting CD4+ memory T cells, activated CD4+ memory T cells; follicular helper T cells; regulatory T cells (Tregs); gamma delta T cells; resting NK cells; activated NK cells, M0 macrophages, M1 macrophages, M2 macrophages, resting dendritic cells, activated dendritic cells, resting mast cells, or activated mast cells.

Embodiment 28 is the method of any one of the preceding embodiments, wherein the plurality of target genes comprises genes differentially expressed in a first cell type relative to a second cell type different from the first cell type, and the first cell type is B cells, T cells, or NK cells.

Embodiment 29 is the method of any one of the preceding embodiments, wherein the plurality of target genes comprises genes differentially expressed when the disease or disorder is present relative to when the disease or disorder is not present.

Embodiment 30 is the method of any one of the preceding embodiments, wherein the plurality of target genes comprises genes differentially expressed in the disease or disorder cells relative to healthy cells of the same cell type as the disease or disorder cells.

Embodiment 31 is the method of any one of the preceding embodiments, wherein the plurality of target genes comprises genes differentially expressed in the disease or disorder cells relative to healthy colon epithelial cells.

Embodiment 32 is the method of any one of the preceding embodiments, wherein the plurality of target genes comprises genes differentially expressed in the disease or disorder cells relative to a myeloid cell type or an erythroid cell type.

Embodiment 33 is the method of any one of the preceding embodiments, wherein the plurality of target genes comprises genes differentially expressed in colon epithelial cells relative to a myeloid cell type or an erythroid cell type.

Embodiment 34 is the method of any one of the preceding embodiments, wherein the plurality of target genes comprises genes having above-average expression variance in a training set comprising gene expression data from samples from healthy subjects and from subjects with the disease or disorder.

Embodiment 35 is the method of embodiment 34, wherein the genes having above-average expression variance comprise genes having an expression variance in the top 25^{th}, top 20^{th}, top 15^{th}, top 10^{th}, top 9^{th}, top 8^{th}, top 7^{th}, top 6^{th}, top 5^{th}, top 4^{th}, top 3^{rd}, top 2^{nd}, or top 1^{st} percentile of the genes of the training set.

Embodiment 36 is the method of embodiment 35, wherein the genes having above-average expression variance are genes with an expression variance ranking in the top 1000, top 750, top 500, top 250, top 200, top 150, top 100, top 90, top 80, top 70, top 60, top 50, top 40, top 30, top 25, top 20, top 15, top 10, or top 5 genes in the training set.

Embodiment 37 is the method of any one of the preceding embodiments, wherein one or more, a majority, at least 75%, at least 80%, at least 85%, at least 90%, at least 95%, at least 96%, at least 97%, at least 98%, at least 99%, or all, of the target genes are protein-coding genes.

Embodiment 38 is the method of any one of the preceding embodiments, wherein the expression levels of the plurality of target genes are transcripts per million (TPM)-normalized, reads per kilobase million (RPKM)-normalized, or fragments per kilobase million (FPKM)-normalized.

Embodiment 39 is the method of any one of the preceding embodiments, wherein the expression levels of the plurality of target genes are mean-centered and/or are scaled to unit variance.

Embodiment 39.1 is the method of any one of the preceding embodiments, wherein the quantities of the immune cell types are mean-centered and/or are scaled to unit variance.

Embodiment 39.2 is method of any one of the preceding embodiments, wherein the quantities of the immune cell types are proportions of the immune cell types.

Embodiment 40 is the method of any one of the preceding embodiments, comprising:
a) determining the presence or absence of the disease or disorder; or
b) determining the likelihood of the disease or disorder.

Embodiment 41 is the method of any one of the preceding embodiments, wherein determining the presence, absence, or likelihood of the disease or disorder comprises compensating for effects of sex on gene expression.

Embodiment 42 is the method of the immediately preceding embodiment, wherein compensating for effects of sex on gene expression comprises regressing out the effects of sex on gene expression.

Embodiment 43 is the method of any one of the preceding embodiments, wherein determining the presence, absence, or likelihood of the disease or disorder comprises compensating for effects of sex on cell type quantity or cell type proportion.

Embodiment 44 is the method of the immediately preceding embodiment, wherein compensating for effects of sex on cell type quantity or cell type proportion comprises regressing out the effects of sex on cell type quantity or cell type proportion.

Embodiment 45 is the method of any one of the preceding embodiments, wherein the target genes comprise genes that are not differentially expressed according to sex.

Embodiment 46 is the method of the immediately preceding embodiment, wherein at least 50%, at least 60%, at least 70%, at least 75%, at least 80%, at least 85%, at least 90%, at least 95%, at least 96%, at least 97%, at least 98%, at least 99%, or 100% of the target genes are not differentially expressed according to sex.

Embodiment 47 is the method of any one of the preceding embodiments, wherein the target genes were identified using a training set comprising samples from individuals of the same sex as the subject.

Embodiment 47.1 is the method of any one of the preceding embodiments, wherein the quantities of the immune cell types were identified using a training set comprising samples from individuals of the same sex as the subject.

Embodiment 48 is the method of any one of the preceding embodiments, wherein the subject is female.

Embodiment 49 is the method of any one of the preceding embodiments, wherein the subject is male.

Embodiment 50 is the method of any one of the preceding embodiments, wherein determining the presence, absence, or likelihood of the disease or disorder comprises compensating for effects of age on gene expression.

Embodiment 51 is the method of the immediately preceding embodiment, wherein compensating for effects of age on gene expression comprises regressing out the effects of age on gene expression.

Embodiment 52 is the method of any one of the preceding embodiments, wherein determining the presence, absence, or likelihood of the disease or disorder comprises compensating for effects of age on cell type abundance or cell type proportion.

Embodiment 53 is the method of the immediately preceding embodiment, wherein compensating for effects of age on cell type abundance or cell type proportion comprises regressing out the effects of age on cell type abundance or cell type proportion.

Embodiment 54 is the method of any one of the preceding embodiments, wherein the target genes comprise genes that are not differentially expressed according to age.

Embodiment 54.1 is the method of any one of the preceding embodiments, wherein the quantities of the immune cell types do not differ according to age.

Embodiment 55 is the method of embodiment 54, wherein at least 50%, at least 60%, at least 70%, at least 75%, at least 80%, at least 85%, at least 90%, at least 95%, at least 96%, at least 97%, at least 98%, at least 99%, or 100% of the target genes are not differentially expressed according to age.

Embodiment 55.1 is the method of the embodiment 54.1, wherein at least 50%, at least 60%, at least 70%, at least 75%, at least 80%, at least 85%, at least 90%, at least 95%, at least 96%, at least 97%, at least 98%, at least 99%, or 100% of the immune cell types do not differ in quantity according to age.

Embodiment 56 is the method of any one of the preceding embodiments, wherein the target genes were identified using a training set comprising samples from individuals that, at the time of sample collection from each individual, were of an age that is within 1 year, within 2 years, within 3 years, within 4 years, within 5 years, within 6 years, within 7 years, within 8 years, within 9 years, within 10 years, within 11 years, within 12 years, within 13 years, within 14 years, or within 15 years of the age of the subject at the time of sample collection from the subject.

Embodiment 56.1 is the method of any one of the preceding embodiments, wherein the quantities of the immune cell types were identified using a training set comprising samples from individuals that, at the time of sample collection from each individual, were of an age that is within 1 year, within 2 years, within 3 years, within 4 years, within 5 years, within 6 years, within 7 years, within 8 years, within 9 years, within 10 years, within 11 years, within 12 years, within 13 years, within 14 years, or within 15 years of the age of the subject at the time of sample collection from the subject.

Embodiment 57 is the method of any one of the preceding embodiments, wherein the target genes comprise one or more, or each, of GZMH, PATL2, FCRL6, ZNF600, IL10RA, DTHD1, PYHIN1, HDAC11, XCL1, GZMA, RAB37, ID2, SLC9A3R1, MOSPD3, TES, EML4, CD99, ACSL6, YPEL1, H2AC17, FGFBP2, TBX21, F2R, PTGDS, DDX3Y, SYNGR1, ZNF683, SERPIN86, PRSS23, ID2, SRSF2, PRR5, CST3, SHISA4, PLAC8, BRD2, BMF, NIPAL2, UTS2, RARS2, ZNF320, RBL2, CREB3L2, RNF38, CCDC88A, MEX3C, SLC38A11, COL19A1, PNPLA7, AFF3, STEAP1B, CELSR1, GRAPL, CBX5, AKAP6, EVL, CRISP3, BPI, MMP8, OLFM4, MPO, MROH6, KDM5D, IDH2, ABI3, S100A4, H2AC17, GIMAP4, PPP1CB, XAF1, RPL37, PTPRO, CD177, RPS13, NAIP, RPS12, ID2, SH3BP1, RAB37, PLEKHG3, AFDN, SLC9A3R1, FLNA, SSBP3, RHOB, LIPA, ISCA1, PAG1, DDX3X, KDM5C, PIM2, IL4R, ZFX, FCGR2B, KDM6A, SARAF, TTC39C, NCOR2, ATP5MD, ARHGD1A, TRAF5, SCAMP2, RPS26, GSTM4, MICAL3, TOMM20, SSBP3, RALGDS, ITGB1, DYRK1B, ARRB1, TRIM11, LFNG, PAXX, ACCS, and SLC2A6.

Embodiment 57.1 is the method of any one of the preceding embodiments, wherein the target genes comprise one or more, or each, of GZMH, PATL2, FCRL6, ZNF600, IL10RA, DTHD1, PYHIN1, HDAC11, XCL1, GZMA, RAB37, ID2, SLC9A3R1, MOSPD3, TES, EML4, CD99, ACSL6, YPEL1, and H2AC17.

Embodiment 57.2 is the method of any one of the preceding embodiments, wherein the target genes comprise one or more (or each) of FGFBP2, TBX21, F2R, PTGDS, DDX3Y, SYNGR1, ZNF683, SERPIN86, PRSS23, ID2, SRSF2, PRR5, CST3, SHISA4, PLAC8, and BRD2.

Embodiment 57.3 is the method of any one of the preceding embodiments, wherein the target genes comprise one or more (or each) of BMF, NIPAL2, UTS2, RARS2, ZNF320, RBL2, CREB3L2, RNF38, CCDC88A, MEX3C, SLC38A11, COL19A1, PNPLA7, AFF3, STEAP1B, CELSR1, GRAPL, CBX5, AKAP6, and EVL.

Embodiment 57.4 is the method of any one of the preceding embodiments, wherein the target genes comprise one or more (or each) of CRISP3, BPI, MMP8, OLFM4, MPO, MROH6, KDM5D, IDH2, ABI3, S100A4, H2AC17, GIMAP4, PPP1CB, XAF1, RPL37, PTPRO, CD177, RPS13, NAIP, RPS12.

Embodiment 57.5 is the method of any one of the preceding embodiments, wherein the target genes comprise one or more (or each) of ID2, SH3BP1, RAB37, PLEKHG3, AFDN, SLC9A3R1, FLNA, SSBP3, RHOB, LIPA, ISCA1, PAG1, DDX3X, KDM5C, PIM2, IL4R, ZFX, FCGR2B, KDM6A, and SARAF.

Embodiment 57.6 is the method of any one of the preceding embodiments, wherein the target genes comprise one or more (or each) of TTC39C, NCOR2, ATP5MD, ARHGD1A, TRAF5, SCAMP2, RPS26, GSTM4, MICAL3, TOMM20, SSBP3, RALGDS, ITGB1, DYRK1B, ARRB1, TRIM11, LFNG, PAXX, ACCS, SLC2A6.

Embodiment 57.7 is the method of any one of the preceding embodiments, wherein the target genes comprise one or more (or each) of GZMH, PATL2, FCRL6, ZNF600, IL10RA, DTHD1, PYHIN1, HDAC11, XCL1, and GZMA.

Embodiment 57.8 is the method of any one of the preceding embodiments, wherein the target genes comprise one or more (or each) of RAB37, ID2, SLC9A3R1, MOSPD3, TES, EML4, CD99, ACSL6, YPEL1, and H2AC17.

Embodiment 57.9 is the method of any one of the preceding embodiments, wherein the target genes comprise one or more (or each) of FGFBP2, TBX21, F2R, PTGDS, DDX3Y, SYNGR1, ZNF683, SERPIN86, PRSS23, and ID2.

Embodiment 57.10 is the method of any one of the preceding embodiments, wherein the target genes comprise one or more (or each) of SRSF2, PRR5, CST3, SHISA4, PLAC8, and BRD2.

Embodiment 57.11 is the method of any one of the preceding embodiments, wherein the target genes comprise one or more (or each) of BMF, NIPAL2, UTS2, RARS2, ZNF320, RBL2, CREB3L2, RNF38, CCDC88A, and MEX3C.

Embodiment 57.12 is the method of any one of the preceding embodiments, wherein the target genes comprise one or more (or each) of SLC38A11, COL19A1, PNPLA7, AFF3, STEAP1B, CELSR1, GRAPL, CBX5, AKAP6, and EVL.

Embodiment 57.13 is the method of any one of the preceding embodiments, wherein the target genes comprise one or more (or each) of CRISP3, BPI, MMP8, OLFM4, MPO, MROH6, KDM5D, IDH2, ABI3, and S100A4.

Embodiment 57.14 is the method of any one of the preceding embodiments, wherein the target genes comprise one or more (or each) of H2AC17, GIMAP4, PPP1CB, XAF1, RPL37, PTPRO, CD177, RPS13, NAIP, and RPS12.

Embodiment 57.15 is the method of any one of the preceding embodiments, wherein the target genes comprise one or more (or each) of ID2, SH3BP1, RAB37, PLEKHG3, AFDN, SLC9A3R1, FLNA, SSBP3, RHOB, and LIPA.

Embodiment 57.16 is the method of any one of the preceding embodiments, wherein the target genes comprise one or more (or each) of ISCA1, PAG1, DDX3X, KDM5C, PIM2, IL4R, ZFX, FCGR2B, KDM6A, and SARAF.

Embodiment 57.17 is the method of any one of the preceding embodiments, wherein the target genes comprise one or more (or each) of TTC39C, NCOR2, ATP5MD, ARHGD1A, TRAF5, SCAMP2, RPS26, GSTM4, MICAL3, and TOMM20.

Embodiment 57.18 is the method of any one of the preceding embodiments, wherein the target genes comprise one or more (or each) of SSBP3, RALGDS, ITGB1, DYRK1B, ARRB1, TRIM11, LFNG, PAXX, ACCS, and SLC2A6.

Embodiment 58 is the method of any one of the preceding embodiments, wherein the target genes comprise one or more, or each, of ICA1, CD38, ABCB4, TNFRSF17, RRP12, CHI3L2, MAP3K13, IL4R, IL12RB2, ACHE, LAG3, CD209, GGT5, DEPDC5, UPK3A, GZMH, BPI, ACP5, CD37, MAST1, RASSF4, MS4A6A, PPFIBP1, MAK, IL18RAP, KYNU, FASLG, MYB, CCND2, TRPM6, FLVCR2, CD80, TMEM156, BHLHE41, NFE2, TREM1, TMEM255A, IL1B, PLEKHG3, VPREB3, TEP1, TRPM4, SMPDL3B, LILRB2, CHI3L1, IL2RA, TLR2, BMP2K, TNFRSF11A, PLA2G7, EPHA1, ABCB9, ZFP36L2, HHEX, SKA1, CLIC2, DUSP2, SLAMF8, CDA, ZNF222, ASGR2, PBXIP1, NIPSNAP3B, TSHR, NOD2, CD300A, NAALADL1, ADRB2, SCN9A, CEACAM3, COL8A2, CLEC7A, CTSW, HPSE, CD7, P2RY14, HIC1, P2RY13, GPR19, CCR3, MROH7, CSF1, FAM174B, TNFRSF4, PDCD1, RYR1, HLA-DQA1, NCR3, ZBTB10, APOBEC3G, LILRA2, TNFRSF13B, HLA-DOB, PGLYRP1, HGF, ATP9A, ATP2C2, JMJD6, DHRS9, SLC1A3, CEACAM1, DUSP13, CRISP3, ABLIM1, HSD3B7, OSM, UPB1, BIK, MMP9, SLCO4A1, BMX, KLF5, RETN, GRB10, PRUNE2, ERLIN1, TP5313, ILIR2, EPAS1, LRRC42, GADD45A, PHTF1, RCAN3, ARG1, CYSTM1, DACH1, FKBP9, GOS2, PFKFB2, CDH26, ARMC7, PPPIR3D, ECHDC3, RDH5, ACVR1B, CKAP4, MTHFS, IL10, MFSD13A, GPR84, MYLK3, ZNF787, MYO10, RAB19, OLAH, ANKRD22, RABGEF1, SAMSN1, CACNA1D, RGL4, TFF3, GYG1, ZDHHC19, SLC51A, POC1A, HPGD, FBN1, CLEC4D, SEMA6B, PFKFB3, PAQR8, SH3PXD2B, FOSL1, MSRA, LRRN1, KCNE5, B3GNT5, CD163, CRACR2B, OPLAH, EXOSC4, KCNE1, UPP1, ST6GALNAC3, SEPTIN5, FCAR, SPATC1, MAFG, IL27, ENTPD7, PLIN5, FAM228B, ORM1, STON1, CFH, HS3ST1, DBNDD1, CD22, SLC25A39, KCNG1, TGFBR3, ADD2, COL19A1, CD200, TCL1A, PROCR, CD40, NME4, TSPAN13, RGS9, FAM184A, KHDRBS2, ENPP5, MMP8, SATB2, GPR68, CEACAM8, MYO1B, LARGE1, NT5E, RAPGEF5, ABHD17C, ZNF365, GRTP1, IGFBP3, LCN2, GLB1L2, CNKSR2, PRSS23, RASGRP3, SCN3A, C16orf74, RETREG1, ERG, SNX22, CXCR5, BEND5, SLC1A7, LEXM, CAMK2N1, SPRY1, CDCA7L, SPIB, DLC1, DIPK1B, MTCL1, PARM1, MZB1, SLC23A1, PDGFD, NMUR1, NBEA, OLR1, PTPRM, PAWR, ABCA13, TAFA1, KCNH8, EFNA5, PRSS57, PTCH1, PRIN3, SULT1A1, ZNF667, SHISA4, L1CAM, RASGEFIA, DDR1, GPC2, PATL2, and IGLL5.

Embodiment 59 is the method of the immediately preceding embodiment, wherein the target genes comprise 2, 3, 4, 5, 6, 7, 8, 9, 10, 11, 12, 13, 14, 15, 16, 17, 18, 19, or 20 of ICA1, CD38, ABCB4, TNFRSF17, RRP12, CHI3L2, MAP3K13, IL4R, IL12RB2, ACHE, LAG3, CD209, GGT5, DEPDC5, UPK3A, GZMH, BPI, ACP5, CD37, MAST1, RASSF4, MS4A6A, PPFIBP1, MAK, IL18RAP, KYNU, FASLG, MYB, CCND2, TRPM6, FLVCR2, CD80, TMEM156, BHLHE41, NFE2, TREM1, TMEM255A, IL1B, PLEKHG3, VPREB3, TEP1, TRPM4, SMPDL3B, LILRB2, CHI3L1, IL2RA, TLR2, BMP2K, TNFRSF11A, PLA2G7, EPHA1, ABCB9, ZFP36L2, HHEX, SKA1, CLIC2, DUSP2, SLAMF8, CDA, ZNF222, ASGR2, PBXIP1, NIPSNAP3B, TSHR, NOD2, CD300A, NAALADL1, ADRB2, SCN9A, CEACAM3, COL8A2, CLEC7A, CTSW, HPSE, CD7, P2RY14, HIC1, P2RY13, GPR19, CCR3, MROH7, CSF1, FAM174B, TNFRSF4, PDCD1, RYR1, HLA-DQA1, NCR3, ZBTB10, APOBEC3G, LILRA2, TNFRSF13B, HLA-DOB, PGLYRP1, HGF, ATP9A, ATP2C2, JMJD6, DHRS9, SLC1A3, CEACAM1, DUSP13, CRISP3, ABLIM1, HSD3B7, OSM, UPB1, BIK, MMP9, SLCO4A1, BMX, KLF5, RETN, GRB10, PRUNE2, ERLIN1, TP5313, ILIR2, EPAS1, LRRC42, GADD45A, PHTF1, RCAN3, ARG1, CYSTM1, DACH1, FKBP9, GOS2, PFKFB2, CDH26, ARMC7, PPP1R3D, ECHDC3, RDH5, ACVR1B, CKAP4, MTHFS, IL10, MFSD13A, GPR84, MYLK3, ZNF787, MYO10, RAB19, OLAH, ANKRD22, RABGEF1, SAMSN1, CACNA1D, RGL4, TFF3, GYG1, ZDHHC19, SLC51A, POC1A, HPGD, FBN1, CLEC4D, SEMA6B, PFKFB3, PAQR8, SH3PXD2B, FOSL1, MSRA, LRRN1, KCNE5, B3GNT5, CD163, CRACR2B, OPLAH, EXOSC4, KCNE1, UPP1, ST6GALNAC3, SEPTIN5, FCAR, SPATC1, MAFG, IL27, ENTPD7, PLIN5, FAM228B, ORM1, STON1, CFH, HS3ST1, DBNDD1, CD22, SLC25A39, KCNG1, TGFBR3, ADD2, COL19A1, CD200, TCL1A, PROCR, CD40, NME4, TSPAN13, RGS9, FAM184A, KHDRBS2, ENPP5, MMP8, SATB2, GPR68, CEACAM8, MYO1B, LARGE1, NT5E, RAPGEF5, ABHD17C, ZNF365, GRTP1, IGFBP3, LCN2, GLB1L2, CNKSR2, PRSS23, RASGRP3, SCN3A, C16orf74, RETREG1, ERG, SNX22, CXCR5, BEND5, SLC1A7, LEXM, CAMK2N1, SPRY1, CDCA7L, SPIB, DLC1, DIPK1B, MTCL1, PARM1, MZB1, SLC23A1, PDGFD, NMUR1, NBEA, OLR1, PTPRM, PAWR, ABCA13, TAFA1, KCNH8, EFNA5, PRSS57, PTCH1, PRTN3, SULT1A1, ZNF667, SHISA4, LICAM, RASGEF1A, DDR1, GPC2, PATL2, and IGLL5.

Embodiment 60 is the method of any one of the preceding embodiments, wherein the target genes comprise one or more, or each, of PGLYRP1, HGF, ATP9A, ATP2C2, JMJD6, DHRS9, SLC1A3, CEACAM1, DUSP13, CRISP3, ABLIM1, HSD3B7, OSM, UPB1, BIK, MMP9, SLCO4A1, BMX, KLF5, RETN, GRB10, PRUNE2, ERLIN1, TP5313, IL1R2, EPAS1, LRRC42, GADD45A, PHTF1, RCAN3, ARG1, CYSTM1, DACH1, FKBP9, GOS2, PFKFB2, CDH26, ARMC7, PPPIR3D, ECHDC3, RDH5, ACVR1B, CKAP4, MTHFS, IL10, MFSD13A, GPR84, MYLK3, ZNF787, MYO10, RAB19, OLAH, ANKRD22, RABGEF1, SAMSN1, CACNA1D, RGL4, TFF3, GYG1, ZDHHC19, SLC51A, POC1A, HPGD, FBN1, CLEC4D, SEMA6B, PFKFB3, PAQR8, SH3PXD2B, FOSL1, MSRA, LRRN1, KCNE5, B3GNT5, CD163, CRACR2B, OPLAH, EXOSC4, KCNE1, UPP1, ST6GALNAC3, SEPTIN5, FCAR, SPATC1, MAFG, IL27, ENTPD7, PLIN5, FAM228B, ORM1, and STON1.

Embodiment 61 is the method of the immediately preceding embodiment, wherein the target genes comprise 2, 3, 4, 5, 6, 7, 8, 9, 10, 11, 12, 13, 14, 15, 16, 17, 18, 19, or 20 of PGLYRP1, HGF, ATP9A, ATP2C2, JMJD6, DHRS9, SLC1A3, CEACAM1, DUSP13, CRISP3, ABLIM1, HSD3B7, OSM, UPB1, BIK, MMP9, SLCO4A1, BMX, KLF5, RETN, GRB10, PRUNE2, ERLIN1, TP5313, ILIR2, EPAS1, LRRC42, GADD45A, PHTF1, RCAN3, ARG1, CYSTM1, DACH1, FKBP9, GOS2, PFKFB2, CDH26, ARMC7, PPPIR3D, ECHDC3, RDH5, ACVR1B, CKAP4, MTHFS, IL10, MFSD13A, GPR84, MYLK3, ZNF787, MYO10, RAB19, OLAH, ANKRD22, RABGEF1, SAMSN1, CACNA1D, RGL4, TFF3, GYG1, ZDHHC19, SLC51A, POC1A, HPGD, FBN1, CLEC4D, SEMA6B, PFKFB3, PAQR8, SH3PXD2B, FOSL1, MSRA, LRRN1, KCNE5, B3GNT5, CD163, CRACR2B, OPLAH, EXOSC4, KCNE1, UPP1, ST6GALNAC3, SEPTIN5, FCAR, SPATC1, MAFG, IL27, ENTPD7, PLIN5, FAM228B, ORM1, and STON1.

Embodiment 62 is the method of any one of the preceding embodiments, wherein the target genes comprise one or more, or each, of ICA1, CD38, ABCB4, TNFRSF17, RRP12, CHI3L2, MAP3K13, IL4R, IL12RB2, ACHE, LAG3, CD209, GGT5, DEPDC5, UPK3A, GZMH, BPI, ACP5, CD37, MAST1, RASSF4, MS4A6A, PPFIBP1, MAK, IL18RAP, KYNU, FASLG, MYB, CCND2, TRPM6, FLVCR2, CD80, TMEM156, BHLHE41, NFE2, TREM1, TMEM255A, IL1B, PLEKHG3, VPREB3, TEP1, TRPM4, SMPDL3B, LILRB2, CHI3L1, IL2RA, TLR2, BMP2K, TNFRSF11A, PLA2G7, EPHA1, ABCB9, ZFP36L2, HHEX, SKA1, CLIC2, DUSP2, SLAMF8, CDA, ZNF222, ASGR2, PBXIP1, NIPSNAP3B, TSHR, NOD2, CD300A, NAALADL1, ADRB2, SCN9A, CEACAM3, COL8A2, CLEC7A, CTSW, HPSE, CD7, P2RY14, HIC1, P2RY13, GPR19, CCR3, MROH7, CSF1, FAM174B, TNFRSF4, PDCD1, RYR1, HLA-DQA1, NCR3, ZBTB10, APOBEC3G, LILRA2, TNFRSF13B, and HLA-DOB.

Embodiment 63 is the method of the immediately preceding embodiment, wherein the target genes comprise 2, 3, 4, 5, 6, 7, 8, 9, 10, 11, 12, 13, 14, 15, 16, 17, 18, 19, or 20 of ICA1, CD38, ABCB4, TNFRSF17, RRP12, CHI3L2, MAP3K13, IL4R, IL12RB2, ACHE, LAG3, CD209, GGT5, DEPDC5, UPK3A, GZMH, BPI, ACP5, CD37, MAST1, RASSF4, MS4A6A, PPFIBP1, MAK, IL18RAP, KYNU, FASLG, MYB, CCND2, TRPM6, FLVCR2, CD80, TMEM156, BHLHE41, NFE2, TREM1, TMEM255A, IL1B, PLEKHG3, VPREB3, TEP1, TRPM4, SMPDL3B, LILRB2, CHI3L1, IL2RA, TLR2, BMP2K, TNFRSF11A, PLA2G7, EPHA1, ABCB9, ZFP36L2, HHEX, SKA1, CLIC2, DUSP2, SLAMF8, CDA, ZNF222, ASGR2, PBXIP1, NIPSNAP3B, TSHR, NOD2, CD300A, NAALADL1, ADRB2, SCN9A, CEACAM3, COL8A2, CLEC7A, CTSW, HPSE, CD7, P2RY14, HIC1, P2RY13, GPR19, CCR3, MROH7, CSF1, FAM174B, TNFRSF4, PDCD1, RYR1, HLA-DQA1, NCR3, ZBTB10, APOBEC3G, LILRA2, TNFRSF13B, and HLA-DOB.

Embodiment 64 is the method of any one of the preceding embodiments, wherein the target genes comprise one or more, or each, of CFH, HS3ST1, DBNDD1, CD22, SLC25A39, KCNG1, TGFBR3, ADD2, COL19A1, CD200, TCL1A, PROCR, CD40, NME4, TSPAN13, RGS9, FAM184A, KHDRBS2, ENPP5, MMP8, SATB2, GPR68, CEACAM8, MYO1B, LARGE1, NT5E, RAPGEF5, ABHD17C, ZNF365, GRTP1, IGFBP3, LCN2, GLB1L2, CNKSR2, PRSS23, RASGRP3, SCN3A, C16orf74, RETREG1, ERG, SNX22, CXCR5, BEND5, SLC1A7, LEXM, CAMK2N1, SPRY1, CDCA7L, SPIB, DLC1, DIPK1B, MTCL1, PARM1, MZB1, SLC23A1, PDGFD, NMUR1, NBEA, OLR1, PTPRM, PAWR, ABCA13, TAFA1, KCNH8, EFNA5, PRSS57, PTCH1, PRTN3, SULT1A1, ZNF667, SHISA4, L1CAM, RASGEF1A, DDR1, GPC2, PATL2, and IGLL5.

Embodiment 65 is the method of the immediately preceding embodiment, wherein the target genes comprise 2, 3, 4, 5, 6, 7, 8, 9, 10, 11, 12, 13, 14, 15, 16, 17, 18, 19, or 20 of CFH, HS3ST1, DBNDD1, CD22, SLC25A39, KCNG1, TGFBR3, ADD2, COL19A1, CD200, TCL1A, PROCR, CD40, NME4, TSPAN13, RGS9, FAM184A, KHDRBS2, ENPP5, MMP8, SATB2, GPR68, CEACAM8, MYO1B, LARGE1, NT5E, RAPGEF5, ABHD17C, ZNF365, GRTP1, IGFBP3, LCN2, GLB1L2, CNKSR2, PRSS23, RASGRP3, SCN3A, C16orf74, RETREG1, ERG, SNX22, CXCR5, BEND5, SLC1A7, LEXM, CAMK2N1, SPRY1, CDCA7L, SPIB, DLC1, DIPK1B, MTCL1, PARM1, MZB1, SLC23A1, PDGFD, NMUR1, NBEA, OLR1, PTPRM, PAWR, ABCA13, TAFA1, KCNH8, EFNA5, PRSS57, PTCH1, PRIN3, SULT1A1, ZNF667, SHISA4, L1CAM, RASGEF1A, DDR1, GPC2, PATL2, and IGLL5.

Embodiment 66 is the method of any one of the preceding embodiments, wherein the method comprises preparing cDNA from the RNA and ligating adapters to the cDNA, thereby producing adapter-ligated cDNA.

Embodiment 67 is the method of the immediately preceding embodiment, wherein the adapters comprise barcodes.

Embodiment 68 is the method of embodiment 66 or embodiment 67, wherein the adapter-ligated cDNA is amplified prior to the sequencing.

Embodiment 69 is the method of any one of the preceding embodiments, further comprising enriching for at least one target region set from the RNA, cDNA prepared from the RNA, or a subsample thereof, comprising contacting the RNA, cDNA prepared from the RNA, or a subsample thereof with target-specific probes specific for the at least one target region set.

Embodiment 70 is the method of any one of the preceding embodiments, further comprising a step of ribosomal RNA depletion.

Embodiment 71 is the method of any one of the preceding embodiments, further comprising a step of globin mRNA depletion.

Embodiment 72 is the method of any one of embodiments 1-69 or 71, further comprising a step of selection of polyadenylated RNA (poly (A)) transcripts.

Embodiment 73 is the method of any one of the preceding embodiments, further comprising a step of RNA fragmentation prior to the sequencing.

Embodiment 74 is the method of the immediately preceding embodiment, wherein the fragmenting provides RNA fragments of 25-400, 25-300, 25-200, 50-400, 50-300, 50-250, 50-200, 100-400, 100-300, 100-200, 125-400, 125-300, 125-200, 125-175, 150-400, 150-300, 200-400, 250-400, 300-400, 200-350, 200-300, 225-375, 250-350, or 275-325 base pairs in length.

Embodiment 75 is the method of any one of the preceding embodiments, wherein the sample is a whole blood sample.

Embodiment 76 is the method of any one of the preceding embodiments, wherein the RNA comprises RNA isolated from intact cells originally present in the sample.

Embodiment 77 is the method of any one of the preceding embodiments, wherein the determining quantities of each of the plurality of immune cell types or sequencing comprises generating a plurality of sequencing reads, and wherein the method further comprises mapping the plurality of sequence reads to one or more reference sequences to generate mapped sequence reads, and processing the mapped sequence reads to determine the presence or absence of the disease or disorder in the subject, or the likelihood that the subject has the disease or disorder.

Embodiment 77.1 is the method of any one of the preceding embodiments, further comprising dividing the sample from the subject into at least first and second subsamples, wherein the first subsample comprises RNA and the second subsample comprises DNA (optionally wherein the RNA is isolated from the first subsample and the DNA is isolated from the second subsample); or further comprising separately isolating DNA and RNA from the sample from the subject.

Embodiment 77.2 is the method of embodiment 77.1, further comprising capturing at least an epigenetic target region set from the DNA, optionally wherein the capturing comprises contacting the DNA with a plurality of target-specific probes specific for members of the epigenetic target region set, thereby providing captured DNA.

Embodiment 77.3 is the method of embodiment 77.2, further comprising determining a methylation level of the at least one of the plurality of epigenetic target regions.

Embodiment 77.4 is the method of any one of embodiments 77.1-77.3, further comprising capturing sequence-variable target regions of the DNA, optionally wherein the capturing comprises contacting the DNA with a plurality of target-specific probes specific for the sequence-variable target regions.

Embodiment 77.5 is the method of any one of embodiments 77.1-77.5, further comprising partitioning the DNA or a portion thereof into a plurality of further subsamples by contacting the DNA with an agent that recognizes methyl cytosine in the DNA, the plurality comprising a first subsample and a second subsample, wherein the first subsample comprises DNA with a methyl cytosine in a greater proportion than the second subsample.

Embodiment 77.6 is the method of the immediately preceding embodiment, wherein the agent that recognizes methyl cytosine is a methyl binding reagent.

Embodiment 77.7 is the method of the immediately preceding embodiment, wherein the methyl binding reagent is a methyl binding domain (MBD) protein or an antibody.

Embodiment 77.8 is the method of any one of embodiments 77.6 or 77.7, wherein the methyl binding reagent specifically recognizes 5-methylcytosine.

Embodiment 77.9 is the method of any one of embodiments 77.5-77.8, wherein the DNA of the first subsample and the DNA of the second subsample are differentially tagged.

Embodiment 77.10 is the method of any one of embodiments 77.1-77.9, further comprising subjecting the DNA or one or more subsamples thereof to a procedure that affects a first nucleobase in the DNA differently from a second nucleobase, wherein the first nucleobase is a modified or unmodified nucleobase, the second nucleobase is a modified or unmodified nucleobase different from the first nucleobase, and the first nucleobase and the second nucleobase have the same base pairing specificity.

Embodiment 77.11 is the method of the immediately preceding embodiment, wherein the first nucleobase is an unmodified cytosine and the second nucleobase is a modified cytosine, optionally wherein the modified cytosine is 5-methylcytosine or 5-hydroxymethylcytosine.

Embodiment 77.12 is the method of any one of embodiments 77.10 or 77.11, wherein the procedure that affects a first nucleobase of the DNA differently from a second nucleobase of the DNA is a methylation-sensitive conversion.

Embodiment 77.13 is the method of the immediately preceding embodiment, wherein the methylation-sensitive conversion is bisulfite conversion, oxidative bisulfite (Ox-BS) conversion, Tet-assisted bisulfite (TAB) conversion, APOBEC-coupled epigenetic (ACE) conversion, enzymatic methyl-seq (EM-seq) conversion, single-enzyme 5-methylcytosine sequencing (SEM-seq) conversion, or direct methylation sequencing (DM-seq).

Embodiment 77.14 is the method of any one of embodiments 77.1-77.13, further comprising sequencing the DNA or a portion thereof.

Embodiment 77.15 is the method of any one of embodiments 77.11-77.13, further comprising sequencing the DNA or a portion thereof in a manner that distinguishes the first nucleobase from the second nucleobase.

Embodiment 77.16 is the method of any one of embodiments 77.1-77.15, further comprising contacting the DNA or at least one subsample thereof with at least one nuclease, optionally prior to the capturing or prior to the sequencing, further optionally wherein the at least one nuclease is at least one restriction enzyme.

Embodiment 77.17 is the method of the immediately preceding embodiment, wherein the at least one restriction enzyme is at least one methylation-sensitive restriction enzyme (MSRE) and/or at least one methylation-dependent restriction enzyme (MDRE).

Embodiment 77.18 is the method of any one of embodiments 77.1-77.17, wherein the method comprises ligating one or more adapters to the DNA, thereby producing adapter-ligated DNA.

Embodiment 77.19 is the method of the immediately preceding embodiment, wherein the adapter-ligated DNA is amplified prior to the sequencing.

Embodiment 77.20 is the method of any one of embodiments 77.14-77.19, wherein the sequencing comprises generating a plurality of sequencing reads, and wherein the method further comprises mapping the plurality of sequence reads to one or more reference sequences to generate mapped sequence reads, and processing the mapped sequence reads to determine the likelihood that the subject has cancer or precancer.

Embodiment 77.21 is the method of any one of embodiments 77.1-77.20, wherein the DNA is cfDNA.

Embodiment 78 is the method of any one of the preceding embodiments, wherein the disease or disorder is a cancer or precancer.

Embodiment 79 is the method of the immediately preceding embodiment, wherein the cancer or precancer is advanced adenoma (AA) and/or colorectal cancer (CRC).

Embodiment 80 is the method of the immediately preceding embodiment, comprising:
a) determining the presence or absence of AA;
b) determining the likelihood of AA;
c) determining the presence or absence of CRC;
d) determining the likelihood of CRC;
e) determining the presence or absence of AA and CRC; or
f) determining the likelihood of AA and CRC.

Embodiment 81 is the method of any one of embodiments 78-80, wherein the sample is obtained from a subject who was previously diagnosed with the cancer and received one or more previous cancer treatments, optionally wherein the sample is obtained at one or more preselected time points following the one or more previous cancer treatments.

Embodiment 82 is the method of any one of embodiments 78-80, wherein the sample is obtained from a subject who was previously diagnosed with the cancer, and the sample is obtained from the subject before the subject receives a cancer treatment.

Embodiment 83 is the method of any one of embodiments 80-82, further comprising determining a cancer recurrence score, optionally wherein a cancer recurrence status of the subject is determined to be at risk for cancer recurrence when the cancer recurrence score is determined to be at or above a predetermined threshold or the cancer recurrence status of the subject is determined to be at lower risk for cancer recurrence when the cancer recurrence score is below the predetermined threshold.

Embodiment 84 is the method of the immediately preceding embodiment, further comprising comparing the cancer recurrence score of the subject with a predetermined cancer recurrence threshold, wherein the subject is classified as a candidate for a subsequent cancer treatment when the cancer recurrence score is above the cancer recurrence threshold or not a candidate for a subsequent cancer treatment when the cancer recurrence score is below the cancer recurrence threshold.

Embodiment 85 is the method of any one of the preceding embodiments, further comprising evaluating or monitoring a response to a treatment in the subject.

Embodiment 86 is the method of the immediately preceding embodiment, wherein the evaluating or monitoring the response to the treatment in the subject comprises comparing the expression levels for the target gene set comprising a plurality of target genes that are differentially expressed in a sample from the subject collected at at least a first time point and a sample from the subject collected at at least a second time point.

Embodiment 87 is the method of embodiment 85, wherein the evaluating or monitoring the response to the treatment in the subject comprises comparing the quantities of the immune cell types in a sample from the subject collected at at least a first time point and a sample from the subject collected at at least a second time point.

Embodiment 88 is the method of embodiment 86 or 87, wherein the first time point is a time point prior to administration of the treatment to the subject, and the second time point is a time point after the administration of the treatment to the subject.

Embodiment 89 is the method of embodiment 86 or 87, wherein the first time point is a time point after administration of the treatment to the subject, and the second time point is a time point after the administration of the treatment to the subject and after the first time point.

Embodiment 90 is the method of any one of the preceding embodiments, wherein the RNA comprises one or more of mRNA, lncRNA, or miRNA.

In some embodiments, including but not limited to any one of the embodiments described above, the results of the methods disclosed herein are used as an input to generate a report. The report may be in a paper or electronic format. For example, quantities of immune cell types and/or expression levels of a plurality of target genes, as obtained by the methods disclosed herein, or information derived therefrom, can be displayed directly in such a report. Alternatively or additionally, diagnostic information or therapeutic recommendations which are at least in part based on the methods disclosed herein can be included in the report.

The various steps of the methods disclosed herein may be carried out at the same or different times, in the same or different geographical locations, e.g. countries, and/or by the same or different people.

Additional advantages will be set forth in part in the description which follows or may be learned by practice. The advantages will be realized and attained by means of the elements and combinations particularly pointed out in the appended claims.

### BRIEF DESCRIPTION OF THE DRAWINGS

FIGS. 1A-1D show receiver operating characteristic (ROC) curves for four models that were used for training from the following gene sets: (1) a cell type deconvolution (CTD) gene panel (approximately 400 genes) (FIG. 1A); (2) the top 500 variable genes based on variance (FIG. 1B); (3) the top 100 differentially expressed genes for CRC (FIG. 1C); and (4) the top 100 differentially expressed genes for AA (FIG. 1D).

FIG. 2 is a schematic diagram of an example of a system suitable for use with some embodiments of the disclosure.

FIG. 3 shows a ROC curve for a model trained from a dataset comprising the compositions and relative proportions of certain cell types (naïve B cells, memory B cells, plasma cells, CD8+ T cells, CD4+ T cells, Treg cells, NK cells, monocytes, macrophages, and dendritic cells) in samples from subjects that had advanced adenoma and samples from healthy subjects. In the ROC curve, cohort A included training set samples, and cohort B included test set samples (See Table 4).

FIGS. 4A-4L show box and whisker plots illustrating within-sex differences in the proportions of particular cell types (FIG. 4A: Naïve B cells fraction, cohort A; FIG. 4B: Naïve B cells fraction, cohort B; FIG. 4C: Naïve CD4 T cells fraction, cohort A; FIG. 4D: Naïve CD4 T cells, cohort B; FIG. 4E: CD8 T cells fraction, cohort A; FIG. 4F: CD8 T cells fraction, cohort B; FIG. 4G: resting NK cells fraction, cohort A; FIG. 4H: resting NK cells fraction, cohort B; FIG. 4I: regulatory T cells fraction, cohort A; FIG. 4J: regulatory T cells fraction, cohort B; FIG. 4K: monocytes fraction, cohort A; FIG. 4L: monocytes fraction, cohort B) between samples from subjects that had AA and samples from healthy subjects in both cohorts A and B (See Table 4). Cell proportions were mean-centered and scaled to obtain Z-scores for plotting.

### DETAILED DESCRIPTION OF CERTAIN EMBODIMENTS

Reference will now be made in detail to certain embodiments of the invention. While the invention will be described in conjunction with such embodiments, it will be understood that they are not intended to limit the invention to those embodiments. On the contrary, the invention is intended to cover all alternatives, modifications, and equivalents, which may be included within the invention as defined by the appended claims.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a nucleic acid” includes a plurality of nucleic acids, reference to “a cell” includes a plurality of cells, and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The section headings used herein are for organizational purposes and are not to be construed as limiting the disclosed subject matter in any way. In the event that any document or other material incorporated by reference contradicts any explicit content of this specification, including definitions, this specification controls.

### I. Definitions

“Buffy coat” refers to the portion of a blood (such as whole blood) or bone marrow sample that contains all or most of the white blood cells and platelets of the sample. The buffy coat fraction of a sample can be prepared from the sample using centrifugation, which separates sample components by density. For example, following centrifugation of a whole blood sample, the buffy coat fraction is situated between the plasma and erythrocyte (red blood cell) layers. The buffy coat can contain both mononuclear (e.g., T cells, B cells, NK cells, dendritic cells, and monocytes) and polymorphonuclear (e.g., granulocytes such as neutrophils and eosinophils) white blood cells.

As used herein, “fragment” or “fragmenting” refers to the breaking or separation of a biological component, such as a nucleic acid molecule (such as RNA) into two or more pieces. Fragmentation, such as RNA fragmentation, can occur spontaneously or can be induced intentionally, such as using standard laboratory procedures, such as described herein. RNA fragmentation can be performed, for example, to prepare RNA (such as RNA isolated from a sample comprising cells) for sequencing (such as RNA-seq).

As used herein, “isolated” refers to a biological component (such as a nucleic acid molecule, protein, or cell) that has been substantially separated, produced apart from, or purified away from other components (for example, other components in a sample, cell, or organism in which the component naturally occurs). Nucleic acid molecules, proteins, or cells that have been “isolated” include those purified using standard purification methods. The term “isolated” or “purified” does not require absolute purity; rather, it is intended as a relative term. Thus, for example, an isolated biological component is one in which the biological component is more enriched in a preparation than the biological component is in its natural environment within a cell, organism, sample, or production vessel (for example, a cell culture system). For example, an isolated biological component can represent at least 50%, such as at least 70%, at least 80%, at least 90%, at least 95%, or greater, of the total biological component content of the preparation.

As used herein, “leukapheresis” refers to a procedure in which white blood cells (leukocytes) are isolated from a sample of blood collected from a subject. Leukapheresis may be performed, e.g., obtain cells for research, diagnostic, prognostic, or monitoring purposes, such as those described herein. Thus, as used herein, a “leukapheresis sample” refers to a sample comprising leukocytes collected from a subject using leukapheresis.

As used herein, “peripheral blood mononuclear cells” or “PBMCs” refers to immune cells having a single, round nucleus that originate in bone marrow and are found in the peripheral circulation. Such cells include, e.g., lymphocytes (T cells, B cells, and NK cells) as well as monocytes, and are isolated from blood samples (such as from a whole blood sample collected from a subject) using density gradient centrifugation.

As used herein, a “combination” comprising a plurality of members refers to either of a single composition comprising the members or a set of compositions in proximity, e.g., in separate containers or compartments within a larger container, such as a multiwell plate, tube rack, refrigerator, freezer, incubator, water bath, ice bucket, machine, or other form of storage.

The “capture yield” of a collection of probes for a given target set refers to the amount (e.g., amount relative to another target set or an absolute amount) of nucleic acid corresponding to the target set that the collection of probes captures under typical conditions. Exemplary typical capture conditions are an incubation of the sample nucleic acid and probes at 65° C. for 10-18 hours in a small reaction volume (about 20 μL) containing stringent hybridization buffer. The capture yield may be expressed in absolute terms or, for a plurality of collections of probes, relative terms. When capture yields for a plurality of sets of target regions are compared, they are normalized for the footprint size of the target region set (e.g., on a per-kilobase basis). Thus, for example, if the footprint sizes of first and second target regions are 50 kb and 500 kb, respectively (giving a normalization factor of 0.1), then the RNA or cDNA corresponding to the first target region set is captured with a higher yield than RNA or cDNA corresponding to the second target region set when the mass per volume concentration of the captured RNA or cDNA corresponding to the first target region set is more than 0.1 times the mass per volume concentration of the captured RNA or cDNA corresponding to the second target region set. As a further example, using the same footprint sizes, if the captured RNA or cDNA corresponding to the first target region set has a mass per volume concentration of 0.2 times the mass per volume concentration of the captured RNA or cDNA corresponding to the second target region set, then the RNA or cDNA corresponding to the first target region set was captured with a two-fold greater capture yield than the RNA or cDNA corresponding to the second target region set.

“Capturing” one or more target nucleic acids (such as RNA, or cDNA produced from the RNA) or one or more nucleic acids comprising at least one target region refers to preferentially isolating or separating the one or more target nucleic acids or one or more nucleic acids comprising at least one target region from non-target nucleic acids or from nucleic acids that do not comprise at least one target region.

A “captured set” of nucleic acids or “captured” nucleic acids refers to nucleic acids that have undergone capture.

As used herein, a “capture moiety” is a molecule that allows affinity separation of molecules, such as nucleic acids (such as RNA, or cDNA produced from the RNA), linked to the capture moiety from molecules lacking the capture moiety. Exemplary capture moieties include biotin, which allows affinity separation by binding to streptavidin linked or linkable to a solid phase or an oligonucleotide, which allows affinity separation through binding to a complementary oligonucleotide linked or linkable to a solid phase.

As used herein, a “cell type” is a set of cells having a shared characteristic. For example, immune cell types can include immune cells of different origins, differentiation types, different activation types, or any combination of different origins, different differentiation types, and different activation types. Indeed, differentiation status and activation status can overlap and often change together in a given immune cell. For example, activation of an immune cell may induce differentiation of the cell. Immune cells of different activation types can include activated cells (such as cells activated by inflammatory cytokines or antigens), suppressive cells (such as T regulatory cells (Tregs), M2 macrophages, and others, or their subsets), or suppressed cells, such as cells suppressed by Tregs. Exemplary immune cell types include, but are not limited to, macrophages (including M1 macrophages and M2 macrophages); activated B cells (including regulatory B cells, memory B cells, and plasma cells); T cell subsets, such as CD4 central memory T cells, CD8 central memory T cells, naïve-like T cells, naïve T cells, and activated T cells (including cytotoxic T cells, regulatory T cells (Tregs), CD4 effector memory T cells, and CD8 effector memory T cells); immature myeloid cells (including myeloid-derived suppressor cells (MDSCs), low-density neutrophils, immature neutrophils, and immature granulocytes); and natural killer (NK) cells. Additional exemplary immune cell types include neutrophils, lymphocytes, plasma cells, monocytes, macrophages, dendritic cells, mast cells, eosinophils, T cells, CD4+ T cells, B cells, megakaryocytes, CD8+ central memory cells, CD4+ central memory cells, precursor B cells, plasma cells, memory-switched B cells, plasma cells, basophils, naïve B cells, memory B cells, CD8+ T cells, naïve CD4+ T cells, resting CD4+ memory T cells, activated CD4+ memory T cells, follicular helper T cells, gamma delta T cells, resting NK cells; activated NK cells, M0 macrophages, resting dendritic cells, activated dendritic cells, resting mast cells, and activated mast cells. In some embodiments, cell types may be distinguished based on characteristics such as one or more cell surface markers, a genetic signature (such as expression (or expression level) of a particular gene or set of genes). As used herein, the term “based on,” such as in the context of determining quantities of immune cell types from which RNA originated “based on” expression levels of a target gene set comprising a plurality of target genes” does not require exclusivity. In other words, a determination of quantities of immune cell types from which RNA originated may be further based on one or more additional measures or features, such as one or more additional target regions.

As used herein, a “cell cluster” or “cluster” is a plurality of related cell types, e.g., immune cell types. In some embodiments, the cell types within a cluster have similar RNA expression profiles, such as within a set of target genes.

A “target region” refers to a nucleic acid targeted for identification and/or capture, for example, by using probes (e.g., through sequence complementarity). A “target region set” or “set of target regions” refers to a plurality of loci targeted for identification and/or capture, for example, by using a set of probes (e.g., through sequence complementarity).

“Specifically binds” in the context of a primer, a probe, or other oligonucleotide and a target sequence means that under appropriate hybridization conditions, the primer, oligonucleotide, or probe hybridizes to its target sequence, or replicates thereof, to form a stable hybrid, while at the same time formation of stable non-target hybrids is minimized. Thus, a primer or probe hybridizes to a target sequence or replicate thereof to a sufficiently greater extent than to a non-target sequence, to ultimately enable capture or detection of the target sequence. Appropriate hybridization conditions are well-known in the art, may be predicted based on sequence composition, or can be determined by using routine testing methods (see, e.g., Sambrook et al., Molecular Cloning, A Laboratory Manual, 2^{nd} ed. (Cold Spring Harbor Laboratory Press, Cold Spring Harbor, NY, 1989, incorporated by reference herein).

A nucleic acid (such as RNA) is “produced by a tumor” if it originated from a tumor cell. Tumor cells are neoplastic cells that originated from a tumor, regardless of whether they remain in the tumor or become separated from the tumor (as in the cases, e.g., of metastatic cancer cells and circulating tumor cells).

As used herein, “precancer” or a “precancerous condition” is an abnormality that has the potential to become cancer, wherein the potential to become cancer is greater than the potential if the abnormality was not present, i.e., was normal. Examples of precancer include but are not limited to adenomas, hyperplasias, metaplasias, dysplasias, benign neoplasias (benign tumors), premalignant carcinoma in situ, and polyps. It should be noted that certain types of carcinoma in situ are recognized in the field as cancerous, e.g., Stage 0 cancer, as opposed to premalignant.

The terms “or a combination thereof” and “or combinations thereof”′ as used herein refers to any and all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

A nucleic acid molecule is “differentially expressed” when the amount of one or more of its expression products (e.g., transcript, such as mRNA, and/or protein) is higher or lower in one sample (such as a sample from a subject that has a disease or disorder, such as a cancer) as compared to another sample (such as a sample from a subject that does not have the disease or disorder). Detecting differential expression can include measuring a change in gene (such as by measuring mRNA) or protein expression. An exemplary gene expression measurement method is RNA sequencing.

As used herein, the term “regressing out” refers to use of a statistical procedure intended to remove the correlative influence of one variable on another, e.g., intended to remove the effects of a variable from an analysis. For example, the effect of sex on expression levels or quantities of immune cell types can be regressed out in certain embodiments disclosed herein.

“Or” is used in the inclusive sense, i.e., equivalent to “and/or,” unless the context requires otherwise.

### II. Exemplary Methods

### A. Overview

In some embodiments, methods disclosed herein comprise sequencing RNA (such as mRNA, lncRNA, and/or miRNA) isolated from a blood sample and determining expression levels for a target gene set comprising a plurality of target genes that are differentially expressed in a plurality of immune cell types and/or in samples from subjects with a disease or disorder relative to samples from healthy subjects. In some embodiments, the RNA to be sequenced is isolated from a blood sample, such as a buffy coat sample, a whole blood sample, a leukapheresis sample, or a PBMC sample. In some embodiments, the RNA to be sequenced is isolated from cells of a blood sample, such as a buffy coat sample, a whole blood sample, a leukapheresis sample, or a PBMC sample. In any of the embodiments of this disclosure, the RNA isolated from any type of sample comprising cells, including but not limited to a blood sample (e.g., a buffy coat sample, a whole blood sample, a leukapheresis sample, or a PBMC sample) may be RNA isolated from the cells of that sample. In some embodiments, methods disclosed herein comprise enriching for (e.g., using hybrid capture) at least one target region set from the RNA, cDNA prepared from the RNA (e.g., cDNA prepared from mRNA), or a subsample thereof, comprising contacting the RNA, cDNA prepared from the RNA, or a subsample thereof with target-specific probes specific for the at least one target region set.

The expression levels of the target gene set (e.g., as determined by RNA sequencing (RNA-seq) of RNA isolated from a blood sample and optionally additional RNA, e.g., including analysis of data obtained from RNA-seq) can be used to determine quantities of each of a plurality of immune cell types from which the RNA originated. This can be useful, e.g., to detect the presence of cancer or precancer, or other conditions (e.g., infection, transplant rejection), in that the state of the immune system as reflected in the distribution of cell types that contribute to RNA isolated from a blood sample and optionally additional RNA can change as a result of such conditions. In some embodiments, the RNA isolated from a blood sample and optionally additional RNA originated from a tumor cell, and the cancer is advanced adenoma (AA) or colorectal cancer (CRC). In some embodiments, the RNA isolated from a blood sample and optionally additional RNA did not originate from a tumor cell, and instead originated from an immune cell.

In some embodiments, the disease or disorder is a cancer or precancer. In some embodiments, the cancer is a solid tumor cancer, e.g., colorectal cancer, or a hematological cancer. In some embodiments, the cancer is a carcinoma or sarcoma. Without wishing to be bound by theory, cancers, including solid tumor cancers such as carcinomas and sarcomas, may cause changes to immune cell type distribution, including with respect to differentiated immune cell types and immune cell activation states, relative to the immune cell distribution in a healthy subject or subject that does not have cancer. Such changes may be detected in the methods herein and can be useful in detecting cancer as well as determining cancer prognosis and/or treatment options.

Some embodiments of the present disclosure comprise steps of isolating RNA from a sample, e.g., a blood sample. Other sample types that include immune and/or cancer-derived cells (e.g., a whole blood sample, a buffy coat sample, a leukapheresis sample, or a peripheral blood PBMC sample) may also be used in embodiments of the disclosed methods. The RNA may be isolated from the cells of any such sample, such as using a method described herein.

The disclosed methods can be combined with analysis of one or more additional biomarkers. In some embodiments, the disclosed methods are combined with one or more methods, such as but not limited to, methods for assessing DNA methylation patterns, DNA mutations (such as somatic mutations), nucleic acid fragmentation patterns, non-coding RNA (such as micro RNAs (miRNAs), ribosomal RNAs, transfer RNAs, small nucleolar RNAs (snow RNAs), and/or small nuclear RNAs (snRNAs)) levels, and/or levels, cellular locations, and/or structural modifications of one or more proteins (such as in a sample from a subject). In some embodiments, the disclosed methods are combined with one or more analyses of genetic variations including mutations, rare mutations, indels, rearrangements, copy number variations, transversions, translocations, recombinations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and/or abnormal changes in nucleic acid 5-methylcytosine.

Some embodiments of the disclosed methods further comprise dividing a sample from a subject into at least first and second subsamples, wherein the first subsample comprises RNA and the second subsample comprises DNA (optionally wherein the RNA is isolated from the first subsample and the DNA is isolated from the second subsample). Some embodiments of the disclosed methods further comprise separately isolating DNA and RNA from the sample from the subject. In some embodiments, the methods further comprise capturing at least an epigenetic target region set from the DNA, as described elsewhere herein. In particular embodiments, the capturing comprises contacting the DNA with a plurality of target-specific probes specific for members of the epigenetic target region set, thereby providing captured DNA. Some embodiments of the disclosed methods further comprise determining a methylation level of the at least one of the plurality of epigenetic target regions. In some embodiments, sequence-variable target regions of the DNA are also (or alternatively) captured. In some such embodiments, the capturing comprises contacting the DNA with a plurality of target-specific probes specific for the sequence-variable target regions.

In some embodiments, the DNA or a portion thereof is partitioned into a plurality of further subsamples by contacting the DNA with an agent that recognizes methyl cytosine in the DNA. In particular embodiments, the plurality comprises a first subsample and a second subsample, wherein the first subsample comprises DNA with a methyl cytosine in a greater proportion than the second subsample. In particular embodiments, the agent that recognizes methyl cytosine is a methyl binding reagent In particular embodiments, the methyl binding reagent is a methyl binding domain (MBD) protein or an antibody. In some embodiments, the methyl binding reagent specifically recognizes 5-methylcytosine. In some embodiments, DNA of a first subsample and DNA of a second subsample are differentially tagged.

In some embodiments of the disclosed methods, the DNA or one or more subsamples thereof is subjected to a procedure that affects a first nucleobase in the DNA differently from a second nucleobase. In particular embodiments, the first nucleobase is a modified or unmodified nucleobase, the second nucleobase is a modified or unmodified nucleobase different from the first nucleobase, and the first nucleobase and the second nucleobase have the same base pairing specificity. In particular embodiments, the first nucleobase is an unmodified cytosine and the second nucleobase is a modified cytosine, optionally wherein the modified cytosine is 5-methylcytosine or 5-hydroxymethylcytosine.

In some embodiments, the procedure that affects a first nucleobase of the DNA differently from a second nucleobase of the DNA is a methylation-sensitive conversion. In particular embodiments, the methylation-sensitive conversion is bisulfite conversion, oxidative bisulfite (Ox-BS) conversion, Tet-assisted bisulfite (TAB) conversion, APOBEC-coupled epigenetic (ACE) conversion, enzymatic methyl-seq (EM-seq) conversion, single-enzyme 5-methylcytosine sequencing (SEM-seq) conversion, or direct methylation sequencing (DM-seq).

In some embodiments, the DNA, or a portion thereof, is sequenced. In particular embodiments, the DNA or a portion thereof is sequenced in a manner that distinguishes the first nucleobase from the second nucleobase.

Some embodiments of the disclosed methods further comprise contacting the DNA or at least one subsample thereof with at least one nuclease, such as prior to the capturing or prior to the sequencing. In some embodiments, the at least one nuclease comprises at least one restriction enzyme. In some embodiments, the at least one nuclease comprises at least one methylation-sensitive restriction enzyme (MSRE) and/or at least one methylation-dependent restriction enzyme (MDRE).

In some embodiments, the method further comprises ligating one or more adapters to the DNA, thereby producing adapter-ligated DNA. In particular embodiments, the adapter-ligated DNA is amplified prior to the sequencing.

In some embodiments, the DNA sequencing comprises generating a plurality of sequencing reads, and wherein the method further comprises mapping the plurality of sequence reads to one or more reference sequences to generate mapped sequence reads, and processing the mapped sequence reads to determine the likelihood that the subject has cancer or precancer.

In particular embodiments, the DNA is cfDNA.

### B. Target Gene Lists

Embodiments of the present disclosure comprise sequencing RNA (such as RNA isolated from a blood sample) and determining expression levels for a target gene set. A target gene set comprises a plurality of target genes that are differentially expressed in a plurality of immune cell types and/or in samples from subjects with a disease or disorder (such as a cancer, such as CRC and/or AA) relative to samples from subjects that do not have the disease or disorder, e.g., healthy subjects (or relative to another control, such as a historical control or standard reference value or range of values (e.g., a group of subjects that represent baseline or normal values not associated with the disease or disorder or values that are associated with not having the disease or disorder)). Expression levels of the genes of the target gene set are determined. Additionally or alternatively, quantities of the immune cell types from which the RNA originated can be determined based on the expression levels of the genes of the target gene set, or on inferred levels of genes within each of a plurality of inferred cell types.

In some embodiments, determining quantities of each of the plurality of immune cell types or sequencing comprises generating a plurality of sequencing reads, mapping the sequence reads to one or more reference sequences to generate mapped sequence reads. The mapped sequence reads can be processed to determine the presence or absence of a disease or disorder (such as a cancer, such as CRC and/or AA) in the subject, or the likelihood that the subject has the disease or disorder.

### 1. RNA Extraction and Isolation

RNA for use in the methods disclosed herein may be isolated from a blood sample or the cells of a sample comprising cells (such as a sample that includes immune and/or cancer-derived cells (e.g., a blood sample such as a whole blood sample, a buffy coat sample, a leukapheresis sample, or a peripheral blood PBMC sample)). General methods for RNA extraction and isolation (such as mRNA extraction and isolation) are known in the art and are disclosed in standard textbooks of molecular biology, including Ausubel et al., Current Protocols of Molecular Biology, John Wiley and Sons (1997). Methods for RNA extraction from paraffin embedded tissues are disclosed, for example, in Rupp and Locker, Lab Invest. 56: A67 (1987), and De Andrés et al., BioTechniques 18:42044 (1995). In particular, RNA isolation can be performed using a purification kit, buffer set, and protease(s) from commercial manufacturers, such as PreAnalytix GmbH or Qiagen, according to the manufacturer's instructions. For example, RNA can be extracted from whole blood samples using the PAXgene® Blood RNA Kit (PreAnalytix GmbH). Other commercially available RNA isolation kits include MasterPure™ Complete DNA and RNA Purification Kit (EPICENTRE®, Madison, WI), and Paraffin Block RNA Isolation Kit (Ambion, Inc.). Total RNA from tissue samples can be isolated using RNA Stat-60 (Tel-Test). RNA prepared from tumor tissue can be isolated, for example, by cesium chloride density gradient centrifugation.

### 2. Library Preparation

Following RNA extraction from a sample (such as a blood sample), a cDNA library is typically prepared in preparation for sequencing, e.g., as in RNA-Seq. In some embodiments, the cDNAs in a library, such as an RNA-Seq library, can comprise a cDNA insert flanked by adapter sequences, such as adapter sequences used for amplification and sequencing on a particular platform. Exemplary cDNA library preparation methods are discussed below; however, cDNA library preparation methods can vary depending on the RNA species under investigation, which can differ in size, sequence, structural features and abundance. One of ordinary skill in the art will be able to select cDNA library preparation methods suitable for cDNA library preparation using an RNA species of interest.

a. RNA Depletion; Poly (A) Selection

Ribosomal RNAs (rRNAs) are the most abundant RNA species in most cells. Globin mRNA is also abundant in certain cell types found in the blood. Thus, some embodiments of the present disclosure comprise a step of ribosomal RNA (rRNA) depletion and/or a step of globin mRNA depletion. Such steps can be performed, e.g., following RNA extraction from a sample, and prior to a step of RNA fragmentation or cDNA fragmentation, prior to a step preparing cDNA from the RNA, prior to a step of ligating adapters to the cDNA, and prior to a sequencing step. In some embodiments, the methods include a step of rRNA depletion. In other embodiments, the methods include a step of globin mRNA depletion. In yet other embodiments, the methods disclosed herein include both a step of rRNA depletion and a step of globin mRNA depletion.

Any suitable rRNA depletion and/or globin mRNA depletion methods are of use in the present disclosure. One approach to eliminate rRNAs uses sequence-specific probes that can hybridize to rRNAs (Hrdlickova et al., Wiley Interdiscip Rev RNA. 2017; 8(1): 10.1002/wrna.1364). Unwanted rRNAs or their cDNAs are hybridized with biotinylated DNA or locked nucleic acid (LNA) probes, followed by depletion with streptavidin beads. Alternatively, rRNAs can be targeted by anti-sense DNA oligos and digested by RNase H, a method also known as probe-directed degradation (PDD). Another approach for rRNA reduction uses specific, not-so-random (NSR) primers that bind to the RNA molecules of interest during reverse transcription, thus avoiding reverse transcription of the rRNAs. For example, a method known as Ovation RNA-Seq (NuGen) uses hexamer or heptamer primers whose sequences are not present in rRNAs. In addition to sequence-based approaches, some methods take advantage of certain features of rRNAs for their elimination. The C₀T-hybridization method is based on heat denaturation, re-annealing, and selective degradation by a duplex-specific nuclease (DSN). Double-stranded cDNAs from abundant sequences are preferentially degraded because of their more rapid annealing kinetics compared to less abundant ones. Selective degradation has also been achieved using the enzyme terminator 5′-phosphate-dependent exonuclease (TEX), which recognizes RNA molecules with 5′-monophosphate, as with rRNAs and tRNAs. Further, commercial kits are available for rRNA and globin mRNA depletion, including, e.g., the Watchmaker Genomics RNA Library Prep Kit with Polaris Depletion.

Other embodiments of the present disclosure comprise a step of poly (A) selection. Such a step can be performed, e.g., following RNA extraction from a sample, and prior to a step of RNA fragmentation or cDNA fragmentation, prior to a step preparing cDNA from the RNA, prior to a step of ligating adapters to the cDNA, and prior to a sequencing step. In eukaryotic organisms, most protein coding RNAs (mRNAs) and many long noncoding RNAs (lncRNAs) (>200 nt) comprise a poly (A) tail (“polyadenylated RNAs”). The poly (A) tail may be used to enrich for polyadenylated RNAs from total cellular RNA, in which polyadenylated RNAs may account for approximately 1-5% of total cellular RNA (Hrdlickova et al., Wiley Interdiscip Rev RNA. 2017; 8(1): 10.1002/wrna.1364). Exemplary poly (A) selection methods include, but are not limited to, use of magnetic or cellulose beads coated with oligo-dT molecules. Alternatively, polyadenylated RNAs can be selected using oligo-dT priming for reverse transcription (RT). Poly (A) selection may be combined with globin mRNA depletion.

a. Fragmentation

In some embodiments, methods disclosed herein comprise fragmenting RNA isolated from a sample (such as RNA isolated from a sample comprising cells, such as a whole blood sample, a buffy coat sample, a leukapheresis sample, or a PBMC sample), such as following poly (A) selection or rRNA and/or globin mRNA depletion. RNA fragmentation methods can include physical fragmentation, chemical fragmentation, and/or enzymatic fragmentation. Physical fragmentation methods include, but are not limited to, acoustic shearing, hydrodynamic shearing (such as sonication or point-sink shearing), needle shearing, and nebulization. Enzymatic fragmentation methods can include use of a ribonuclease (such as RNase III). RNA may also be fragmented using chemical shearing methods. Chemical fragmentation methods can include, but are not limited to, heat digestion of RNA in the presence of a divalent metal cation (such as magnesium or zinc). In some embodiments, the fragmenting provides RNA (such as mRNA) fragments of 25-400, 25-300, 25-200, 50-400, 50-300, 50-250, 50-200, 100-400, 100-300, 100-200, 125-400, 125-300, 125-200, 125-175, 150-400, 150-300, 200-400, 250-400, 300-400, 200-350, 200-300, 225-375, 250-350, or 275-325 base pairs in length.

Alternatively, non-fragmented RNAs can be reverse transcribed, and the resultant cDNA can be fragmented. cDNA fragmentation methods can include physical fragmentation, chemical fragmentation, and/or enzymatic fragmentation. Physical fragmentation methods include, but are not limited to, acoustic shearing, hydrodynamic shearing (such as sonication or point-sink shearing), needle shearing, and nebulization. Enzymatic fragmentation methods can include use of a restriction endonuclease (such as a 4-cutter or 5-cutter restriction endonuclease, e.g., AluI, DpnI, Eco47I, HaeIII, HpaII, Mbo I, Msel, MspI, PspGI, RsaI, Sse9I, or TaqI), a non-specific nuclease (e.g., micrococcal nuclease), or a transposase (for example, when insertion of an adapter into a fragmented double-stranded cDNA molecule is desired). cDNA may also be fragmented using chemical shearing methods. Chemical fragmentation methods can include, but are not limited to, heat digestion of cDNA in the presence of a divalent metal cation (such as magnesium or zinc). In some embodiments, the fragmenting provides cDNA fragments of 25-400, 25-300, 25-200, 50-400, 50-300, 50-250, 50-200, 100-400, 100-300, 100-200, 125-400, 125-300, 125-200, 125-175, 150-400, 150-300, 200-400, 250-400, 300-400, 200-350, 200-300, 225-375, 250-350, or 275-325 base pairs in length.

b. cDNA Preparation

Some embodiments of the disclosed methods comprise preparing cDNA from RNA (such as RNA extracted from a blood sample), such as by reverse transcription of the RNA template into cDNA. Reverse transcription is generally followed by exponential amplification of the cDNA, e.g., in a PCR reaction. Two commonly used reverse transcriptases are avian myeloblastosis vims reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse transcribed using a Gene Amp RNA PCR kit (Perkin Elmer, Calif., USA), following the manufacturer's instructions. The derived cDNA can then be used as a template in the subsequent amplification (e.g., PCR) reaction. In some embodiments, RNA is converted to cDNA using random priming, followed by second strand synthesis, end repair, and optional A-tailing. Adapters comprising barcodes can then be ligated to the cDNA, which is then amplified.

Amplification is typically primed by primers that anneal or bind to primer binding sites in adapters flanking a cDNA molecule to be amplified. Amplification methods can involve cycles of denaturation, annealing and extension, resulting from thermocycling or can be isothermal as in transcription-mediated amplification. Other amplification methods include the ligase chain reaction, strand displacement amplification, nucleic acid sequence-based amplification, and self-sustained sequence-based replication.

Although a PCR step can use a variety of thermostable DNA-dependent DNA polymerases, it typically employs the Taq DNA polymerase. TaqMan® PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

The primers used for the amplification are selected so as to amplify a unique segment of the gene of interest, such as RNA (such as mRNA) encoding a gene of a target gene set described herein. In some embodiments, expression of other genes is also detected, such as other known disease markers (such as known cancer markers) or housekeeping genes. Primers that can be used to amplify disease-related molecules are commercially available or can be designed and synthesized. In some examples, the primers specifically hybridize to a promoter or promoter region of a disease-related molecule. An alternative quantitative nucleic acid amplification procedure is described in U.S. Pat. No. 5,219,727. In this procedure, the amount of a target sequence in a sample is determined by simultaneously amplifying the target sequence and an internal standard nucleic acid segment. The amount of amplified cDNA from each segment is determined and compared to a standard curve to determine the amount of the target nucleic acid segment that was present in the sample prior to amplification. In some embodiments, the expression of a “housekeeping” gene or “internal control” can also be evaluated. These terms include any constitutively or globally expressed gene whose presence enables an assessment of mRNA levels provided herein. Such an assessment includes a determination of the overall constitutive level of gene transcription and a control for variations in RNA recovery. Exemplary housekeeping genes include tubulin, glyceraldehyde-3-phosphate-dehydrogenase (GAPDH), beta-actin, and 18S ribosomal RNA.

c. Adapter Ligation or Addition

In some embodiments, adapters are added to RNA or to cDNA prepared from the RNA. This may be done concurrently with an amplification procedure, e.g., by providing the adapters in a 5′ portion of a primer (where PCR is used, this can be referred to as library prep-PCR or LP-PCR). In some embodiments, adapters are added by other approaches, such as ligation. In some such methods, prior to capturing, first adapters are added to the nucleic acids by ligation to the 3′ ends thereof, which may include ligation to single-stranded cDNA. The adapter can be used as a priming site for second-strand synthesis, e.g., using a universal primer and a DNA polymerase. A second adapter can then be ligated to at least the 3′ end of the second strand of the now double-stranded molecule. In some embodiments, the first adapter comprises an affinity tag, such as biotin, and nucleic acid ligated to the first adapter is bound to a solid support (e.g., bead), which may comprise a binding partner for the affinity tag such as streptavidin. For further discussion of a related procedure, see Gansauge et al., Nature Protocols 8:737-748 (2013). In some embodiments, after adapter ligation, nucleic acids are amplified.

Preferably, the adapters include different tags of sufficient numbers that the number of combinations of tags results in a low probability e.g., less than or equal to 5%, such as 5%, 4%, 3%, 2%, 1%, 0.5%, 0.1%, or less than 0.1% of two nucleic acids with the same start and stop points receiving the same combination of tags. Adapters, whether bearing the same or different tags, can include the same or different primer binding sites, but preferably adapters include the same primer binding site.

In some embodiments, following attachment of adapters, the nucleic acids are subject to amplification. The amplification can use, e.g., universal primers that recognize primer binding sites in the adapters.

In some embodiments, the nucleic acids are linked at both ends to Y-shaped adapters including primer binding sites and tags. The molecules are amplified.

In some embodiments, e.g., where cDNA has been subjected to a treatment that renders some or all of the cDNA single-stranded, a library preparation procedure appropriate for samples comprising single-stranded cDNA may be used. See, e.g., Gansauge & Meyer, Nature Protocols 8, 737-748 (2013). For example, biotinylated adapters can be ligated to 3′ ends of cDNA, followed by immobilization on streptavidin-coated beads. Complementary strands can be synthesized using a primer that anneals to the adapter and a DNA polymerase. A second adapter can then be attached to the now-double stranded molecule, e.g., by blunt-ended ligation. The biotinylated adapters may comprise any embodiments of tags and/or barcodes as described elsewhere herein. The molecules can then be amplified, e.g., via PCR, and the amplification products can be sequenced.

d. Tagging

“Tagging” RNA or cDNA molecules is a procedure in which a tag is attached to or associated with the RNA or cDNA molecules. Tags can be molecules, such as nucleic acids, containing information that indicates a feature of the molecule with which the tag is associated. For example, molecules can bear a sample tag (which distinguishes molecules in one sample from those in a different sample) or a molecular tag/molecular barcode/barcode (which distinguishes different molecules from one another (in both unique and non-unique tagging scenarios). For methods that involve a partitioning step, a partition tag (which distinguishes molecules in one partition from those in a different partition) may be included. In some embodiments, adapters comprising tags are added to cDNA molecules, such as cDNA prepared from RNA extracted from a blood sample (such as a buffy coat sample, a whole blood sample, a leukapheresis sample, a PBMC sample) and/or additional cDNA. In certain embodiments, a tag can comprise one or a combination of barcodes. As used herein, the term “barcode” refers to a nucleic acid molecule having a particular nucleotide sequence, or to the nucleotide sequence, itself, depending on context. A barcode can have, for example, between 10 and 100 nucleotides. A collection of barcodes can have degenerate sequences or can have sequences having a certain hamming distance, as desired for the specific purpose. So, for example, a molecular barcode can be comprised of one barcode or a combination of two barcodes, each attached to different ends of a molecule. Additionally or alternatively, for different partitions and/or samples, different sets of molecular barcodes, or molecular tags can be used such that the barcodes serve as a molecular tag through their individual sequences and also serve to identify the partition and/or sample to which they correspond based the set of which they are a member.

In some embodiments, two or more partitions, e.g., each partition, is/are differentially tagged. In some embodiments, the partitions comprise cDNA prepared from RNA extracted from a sample comprising cells or a blood sample (such as a buffy coat sample, a whole blood sample, a leukapheresis sample, or a PBMC sample). In some embodiments, the partitions comprise additional cDNA, such as cDNA prepared from RNA extracted from a tumor sample. Tags can be used to label the individual polynucleotide population partitions so as to correlate the tag (or tags) with a specific partition. Alternatively, tags can be used in embodiments that do not employ a partitioning step. In some embodiments, a single tag can be used to label a specific partition. In some embodiments, multiple different tags can be used to label a specific partition. In embodiments employing multiple different tags to label a specific partition, the set of tags used to label one partition can be readily differentiated for the set of tags used to label other partitions. In some embodiments, the tags may have additional functions, for example the tags can be used to index sample sources or used as unique molecular identifiers (which can be used to improve the quality of sequencing data by differentiating sequencing errors from mutations, for example as in Kinde et al., Proc Nat'l Acad Sci USA 108:9530-9535 (2011), Kou et al., PloS ONE, 11: e0146638 (2016)) or used as non-unique molecule identifiers, for example as described in U.S. Pat. No. 9,598,731. Similarly, in some embodiments, the tags may have additional functions, for example the tags can be used to index sample sources or used as non-unique molecular identifiers (which can be used to improve the quality of sequencing data by differentiating sequencing errors from mutations).

In some embodiments, partition tagging comprises tagging molecules in each partition with a partition tag. After re-combining partitions (e.g., to reduce the number of sequencing runs needed and avoid unnecessary cost) and sequencing molecules, the partition tags identify the source partition. In some embodiments, the partition tags can serve as identifiers of the source partition and the molecule, i.e., different partitions are tagged with different sets of molecular tags, e.g., comprised of a pair of barcodes. In this way, the one or more molecular barcodes attached to the molecule indicates the source partition as well as being useful to distinguish molecules within a partition. For example, a first set of 35 barcodes can be used to tag molecules in a first partition, while a second set of 35 barcodes can be used tag molecules in a second partition.

In some embodiments, after partitioning and tagging with partition tags, the molecules may be pooled for sequencing in a single run. In some embodiments, a sample tag is added to the molecules, e.g., in a step subsequent to addition of partition tags and pooling. Sample tags can facilitate pooling material generated from multiple samples for sequencing in a single sequencing run.

Alternatively, in some embodiments, partition tags may be correlated to the sample as well as the partition. As a simple example, a first tag can indicate a first partition of a first sample; a second tag can indicate a second partition of the first sample; a third tag can indicate a first partition of a second sample; and a fourth tag can indicate a second partition of the second sample.

While tags may be attached to molecules already partitioned based on one or more characteristics, the final tagged molecules in the library may no longer possess that characteristic. For example, while single stranded cDNA molecules may be partitioned and tagged, the final tagged molecules in the library are likely to be double stranded. Accordingly, the tag attached to molecule in the library typically indicates the characteristic of the “parent molecule” from which the ultimate tagged molecule is derived, not necessarily to characteristic of the tagged molecule, itself.

As an example, barcodes 1, 2, 3, 4, etc. are used to tag and label molecules in the first partition; barcodes A, B, C, D, etc. are used to tag and label molecules in the second partition; and barcodes a, b, c, d, etc. are used to tag and label molecules in the third partition. Differentially tagged partitions can be pooled prior to sequencing. Differentially tagged partitions can be separately sequenced or sequenced together concurrently, e.g., in the same flow cell of an Illumina sequencer.

Tags comprising barcodes can be incorporated into or otherwise joined to adapters. Tags can be incorporated by ligation, overlap extension PCR among other methods.

i. Molecular Tagging Strategies

Molecular tagging refers to a tagging practice that allows one to differentiate among cDNA molecules from which sequence reads originated. Tagging strategies can be divided into unique tagging and non-unique tagging strategies. In unique tagging, all or substantially all of the molecules in a sample bear a different tag, so that reads can be assigned to original molecules based on tag information alone. Tags used in such methods are sometimes referred to as “unique tags”. In non-unique tagging, different molecules in the same sample can bear the same tag, so that other information in addition to tag information is used to assign a sequence read to an original molecule. Such information may include start and stop coordinate, coordinate to which the molecule maps, start or stop coordinate alone, etc. Tags used in such methods are sometimes referred to as “non-unique tags”. Accordingly, it is not necessary to uniquely tag every molecule in a sample. It suffices to uniquely tag molecules falling within an identifiable class within a sample. Thus, molecules in different identifiable families can bear the same tag without loss of information about the identity of the tagged molecule.

In certain embodiments of non-unique tagging, the number of different tags used can be sufficient that there is a very high likelihood (e.g., at least 99%, at least 99.9%, at least 99.99% or at least 99.999% that all cDNA molecules of a particular group bear a different tag. It is to be noted that when barcodes are used as tags, and when barcodes are attached, e.g., randomly, to both ends of a molecule, the combination of barcodes, together, can constitute a tag.

This Number, in Term, is a Function of the Number of Molecules Falling into the Calls. For example, the class may be all molecules mapping to the same start-stop position on a reference genome. The class may be all molecules mapping across a particular genetic locus, e.g., a particular base or a particular region (e.g., up to 100 bases or a gene or an exon of a gene). In certain embodiments, the number of different tags used to uniquely identify a number of molecules, z, in a class can be between any of 2*z, 3*z, 4*z, 5*z, 6*z, 7*z, 8*z, 9*z, 10*z, 11 *z, 12*z, 13*z, 14*z, 15*z, 16*z, 17*z, 18*z, 19*z, 20*z or 100*z (e.g., lower limit) and any of 100,000*z, 10,000*z, 1000*z or 100*z (e.g., upper limit).

For example, in a sample of about 5 ng to 30 ng of cDNA, one expects around 3000 molecules to map to a particular nucleotide coordinate, and between about 3 and 10 molecules having any start coordinate to share the same stop coordinate. Accordingly, about 50 to about 50,000 different tags (e.g., between about 6 and 220 barcode combinations) can suffice to uniquely tag all such molecules. To uniquely tag all 3000 molecules mapping across a nucleotide coordinate, about 1 million to about 20 million different tags would be required.

Generally, assignment of unique or non-unique tags barcodes in reactions follows methods and systems described by U.S. patent applications Ser. No. 20/010,053519, 20030152490, 20110160078, and U.S. Pat. Nos. 6,582,908 and 7,537,898 and 9,598,731. Tags can be linked to sample nucleic acids randomly or non-randomly.

The unique tags may be loaded so that more than about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags are loaded per sample. In some cases, the unique tags may be loaded so that less than about 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags are loaded per sample. In some cases, the average number of unique tags loaded per sample is less than, or greater than, about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags per sample.

A preferred format uses 20-50 different tags (e.g., barcodes) ligated to both ends of target nucleic acids. For example, 35 different tags (e.g., barcodes) ligated to both ends of target molecules creating 35×35 permutations, which equals 1225 for 35 tags. Such numbers of tags are sufficient so that different molecules having the same start and stop points have a high probability (e.g., at least 94%, 99.5%, 99.99%, 99.999%) of receiving different combinations of tags. Other barcode combinations include any number between 10 and 500, e.g., about 15×15, about 35×35, about 75×75, about 100×100, about 250×250, about 500×500.

In some cases, unique tags may be predetermined or random or semi-random sequence oligonucleotides. In other cases, a plurality of barcodes may be used such that barcodes are not necessarily unique to one another in the plurality. In this example, barcodes may be ligated to individual molecules such that the combination of the barcode and the sequence it may be ligated to creates a unique sequence that may be individually tracked. As described herein, detection of non-unique barcodes in combination with sequence data of beginning (start) and end (stop) portions of sequence reads may allow assignment of a unique identity to a particular molecule. The length or number of base pairs, of an individual sequence read may also be used to assign a unique identity to such a molecule. As described herein, fragments from a single strand of nucleic acid having been assigned a unique identity, may thereby permit subsequent identification of fragments from the parent strand.

e. Enriching/Capturing; Amplification

Methods disclosed herein can comprise capturing RNA or cDNA, such as target regions, e.g., of RNA (or of cDNA prepared from the RNA) extracted from a sample comprising cells or a blood sample (e.g., a buffy coat sample, a whole blood sample, a leukapheresis sample, or a PBMC sample). In some embodiments, target regions of additional RNA (or of cDNA prepared from the RNA), such as RNA (or cDNA prepared from the RNA) from tumor cells, are also captured. Capturing of nucleic acids from a sample comprising cells or a blood sample (e.g., a buffy coat sample, a whole blood sample, a leukapheresis sample, or a PBMC sample) and/or from additional nucleic acids may be performed in parallel (e.g., on pooled and/or differentially tagged nucleic acids from a sample comprising cells or a blood sample (e.g., a buffy coat sample, a whole blood sample, a leukapheresis sample, or a PBMC sample) and/or additional nucleic acids) or separately. Any of the embodiments described herein relating to enrichment or capture may be performed on nucleic acids from a sample comprising cells or a blood sample (e.g., a buffy coat sample, a whole blood sample, a leukapheresis sample, or a PBMC sample) and/or additional nucleic acids.

In some embodiments, the capturing comprises contacting the nucleic acids with probes (e.g., oligonucleotides) specific for the target regions. Enrichment or capture may be performed on any sample or subsample described herein using any suitable approach known in the art.

In some embodiments, enrichment or capture is performed after attachment of adapters to sample molecules. In some embodiments, enrichment or capture is performed after a partitioning step. In some embodiments, enrichment or capture is performed after an amplification step. In some embodiments, sample molecules are partitioned, then adapters are attached, then sample molecules are amplified, and then the amplified molecules are subjected to enrichment or capture. The enriched or captured molecules may then be subjected to another amplification and then sequenced.

In some embodiments, the probes specific for the target regions comprise a capture moiety that facilitates the enrichment or capture of the nucleic acids hybridized to the probes. In some embodiments, the capture moiety is biotin. In some such embodiments, streptavidin attached to a solid support, such as magnetic beads, is used to bind to the biotin. Nonspecifically bound nucleic acids that do not comprise a target region are washed away from the captured nucleic acids. In some embodiments, the nucleic acid is then dissociated from the probes and eluted from the solid support using salt washes or buffers comprising another nucleic acid denaturing agent. In some embodiments, the probes are also eluted from the solid support by, e.g., disrupting the biotin-streptavidin interaction. In some embodiments, captured nucleic acid is amplified following elution from the solid support. In some such embodiments, nucleic acids comprising adapters are amplified using PCR primers that anneal to the adapters. In some embodiments, captured nucleic acids are amplified while attached to the solid support. In some such embodiments, the amplification comprises use of a PCR primer that anneals to a sequence within an adapter and a PCR primer that anneals to a sequence within a probe annealed to the target region of the nucleic acid.

The capturing step may be performed using conditions suitable for specific nucleic acid hybridization, which generally depend to some extent on features of the probes such as length, base composition, etc. Those skilled in the art will be familiar with appropriate conditions given general knowledge in the art regarding nucleic acid hybridization. In some embodiments, complexes of target-specific probes and nucleic acids are formed.

In some embodiments, cDNA is amplified. In some embodiments, amplification is performed before the capturing step. In some embodiments, amplification is performed after the capturing step. In some embodiments, amplification is performed before and after the capturing step. In various embodiments, the methods further comprise sequencing the captured cDNA.

In some embodiments, adapters are included in the cDNA as described herein. In some embodiments, tags, which may be or include barcodes, are included in the cDNA. In some embodiments, such tags are included in adapters. Tags can facilitate identification of the origin of a nucleic acid. For example, barcodes can be used to allow the origin (e.g., subject) whence the RNA (or cDNA prepared from the RNA) came to be identified following pooling of a plurality of samples for parallel sequencing. This may be done concurrently with an amplification procedure, e.g., by providing the barcodes in a 5′ portion of a primer, e.g., as described herein. In some embodiments, adapters and tags/barcodes are provided by the same primer or primer set. For example, the barcode may be located 3′ of the adapter and 5′ of the target-hybridizing portion of the primer. Alternatively, barcodes can be added by other approaches, such as ligation, optionally together with adapters in the same ligation substrate.

Additional details regarding amplification, tags, and barcodes are discussed herein, which can be combined to the extent practicable with any of these embodiments.

i. Captured Set; Target Regions

In some embodiments, nucleic acids captured or enriched using a method described herein from RNA (or of cDNA prepared from the RNA) from a sample comprising cells or a blood sample (e.g., a buffy coat sample, a whole blood sample, a leukapheresis sample, or a PBMC sample) and/or from additional nucleic acids, comprise captured RNA (or of cDNA prepared from the RNA), such as one or more captured sets of RNA (or of cDNA prepared from the RNA). In some embodiments, the captured nucleic acids comprise target regions (or a target region set, such as a target region set comprising one or more genes of a target gene list disclosed herein, or comprising one or more genes of a target gene list generated using the methods disclosed herein) that are differentially expressed in different immune cell types or in a sample from a subject having a disease or disorder as compared to a sample from a subject that does not have the disease or disorder. In some embodiments, the immune cell types comprise rare or closely related immune cell types, such as activated and naïve lymphocytes or myeloid cells at different stages of differentiation.

Without wishing to be bound by any particular theory, in an individual with cancer, proliferating or activated immune cells (and potentially also cancer cells) may be present in different (e.g., greater or fewer) quantities in the bloodstream and/or may shed more nucleic acids into the bloodstream than immune cells in a healthy individual (and healthy cells of the same tissue type, respectively). As such, the distribution of cell type and/or tissue of origin of RNA (or of cDNA prepared from the RNA) and/or additional nucleic acids may change upon carcinogenesis. For example, the distribution of immune cell type of origin may change in a subject having cancer, precancer, infection, transplant rejection, or other disease or disorder directly or indirectly affecting the immune system. Thus, variations in such distributions can be an indicator of disease.

### 3. Sequencing; Generating Target Gene Lists

In the disclosed methods, nucleic acids, including nucleic acids flanked by adapters, with or without prior amplification are subject to sequencing, such as RNA sequencing (RNA-seq) (see Stark, et al., Nat Rev Genet. 2019; 20, 631-656; Haque, et al, Genome Med. 2017; 9 (75)). RNA-seq is most frequently used for analyzing differential gene expression between samples. In traditional RNA-seq analyses, the process of analyzing differential gene expression via RNA-seq begins with RNA extraction (such as from a blood sample as described herein), followed by polyadenylated (e.g., mRNA) enrichment or ribosomal RNA depletion and/or globin mRNA depletion. cDNA is then synthesized, and an adaptor-ligated sequencing library is prepared. The library is sequenced to a read depth of, for example, 10-60 million reads per sample (such as 50 million reads, with deduplication) on a high-throughput platform (such as an Illumina platform). The sequencing reads (most often in the form of FASTQ files) can be computationally aligned and/or assembled to a transcriptome. The reads are most often mapped to a known transcriptome or annotated genome, matching each read to one or more genomic coordinates. This process is often accomplished using alignment tools such as STAR, TopHat, or

HISAT, which each rely on a reference genome. If no genome annotation containing known exon boundaries is available (such as if a reference genome annotation is missing or is incomplete), or if reads are to be associated with transcripts rather than genes, aligned reads can be used in a transcriptome assembly step using tools such as StringTie or SOAPdenovo-Trans. Tools such as Sailfish, Kallisto, and Salmon can associate sequencing reads directly with transcripts, without the need for a separate quantification step.

Reads that have been mapped to transcriptomic or genomic locations can be quantified using tools such as RSEM, Cufflinks, MMSeq, or HTSeq, or the alignment-free direct quantification tools Sailfish, Kallisto, or Salmon. Quantification results are often combined into an expression matrix, with one row for each expression feature (gene or transcript) and one column for each sample, with values being read counts or estimated abundances. Samples are then filtered and normalized to account for differences in expression patterns, read depth, and/or technical biases. In some embodiments, the expression levels of a plurality of genes (such as target genes) are transcripts per million (TPM)-normalized, reads per kilobase million (RPKM)-normalized, or fragments per kilobase million (FPKM)-normalized. In some embodiments, the expression levels of the genes (such as the target genes) are mean-centered and/or are scaled to unit variance. As described elsewhere herein, short sequences or barcodes may be added during library preparation or by direct RNA ligation, before amplification, to mark a sequence read as coming from a specific starting molecule. In some embodiments, quantities of immune cell types (e.g., quantities of immune cell types from which the RNA originated based on expression levels of the RNAs) are mean-centered and/or are scaled to unit variance. In some embodiments, the quantities of the immune cell types are proportions of the immune cell types.

Changes in expression of individual genes and or transcripts between sample groups can be statistically modeled using one or more of various tools and computational methods. For example, a logistic regression model can be used to determine whether the presence, absence, or likelihood of a disease or disorder (such as a cancer, such as AA and/or CRC) in s subject can be determined based on the quantities (e.g., proportions) of the expression levels of genes differentially expressed between sample groups (such as between a cohort comprising individuals having a disease or condition (such as a cancer) and a cohort comprising healthy individuals (e.g., individuals who do not have the disease or condition), or on the quantities (e.g., proportions) of the immune cell types from which the RNA originated.

For example, in some embodiments of the present disclosure, a list of target genes (genes differentially expressed between sample groups (such as between a cohort comprising individuals having a disease or condition (such as a cancer) and a cohort comprising healthy individuals (e.g., individuals who do not have the disease or condition)), and/or genes that are differentially expressed in a plurality of immune cell types may be generated as described in Examples 1 and 2. Expression counts can be de-duplicated, restricted to protein coding genes only, and transcripts per million (TPM)-normalized. The TPM-normalized values can be further mean-centered and scaled to unit variance for each gene. In some embodiments, modeling features can include the estimated proportions of one or more cell types described herein, such as a quantity of one or more cell types relative to a quantity of a different one or more cell types (such as in the same sample or in different samples), or a quantity of one or more cell types in a first sample (such as a sample from a subject) relative to a quantity of the same one or more cell types in a second sample (such as a second sample from the same subject or a sample from a different subject). Some cell types may be difficult to quantify at low values; thus, in some embodiments, a pseudocount can be used, e.g., by adding the pseudocount to each feature (e.g., cell type or gene expression value), or as a minimum value that replaces any observed values of zero, or of zero or a value lower than the pseudocount value. For example, pseudocounts can be used for genes for which no transcript is detected or for which the expression value would otherwise be zero or substantially zero, e.g., before transforming (e.g., logit-transforming) the features, such as to minimize the effects of noise. In another example, pseudocounts can be used for cell types for which the detected value is zero, or a value lower than the pseudocount value. In some embodiments, pseudocount values are applied to both gene expression values and to cell type values. Use of such pseudocounts may improve model training (e.g., model performance using training data and/or subsequent test data after training), and/or may produce a more balanced overall distribution of output model probabilities than, for example, a model trained without using pseudocounts (e.g., without using pseudocounts for genes for which no transcript is detected or an expression value would otherwise be zero or substantially zero), or a model trained using an overly small pseudocount, such as 0.00001 (as a proportion of 1). In some embodiments, the pseudocount is approximately equal to (e.g., within 50%, 40%, 30%, 20%, 15%, 10%, or 5% of) a limit of detection, such as a limit of detection for cell types generally, for a particular cell type, for transcripts generally, or for a particular transcript. In some embodiments, the pseudocount (e.g., a constant value, or expressed as a proportion of total cells or transcripts as the case may be) is greater than 0.00001. In some embodiments, the pseudocount is 0.00002-0.1, such as 0.0001-0.1, 0.0001-0.01, 0.0005-0.01, 0.0006-0.01, 0.0007-0.01, 0.0008-0.01, 0.0009-0.01, 0.001-0.01 or 0.0005-0.005. In some embodiments, the pseudocount is 0.0001-0.1, 0.0005-0.01, or 0.0003-0.002. In some embodiments, the pseudocount is 0.0005, 0.0006, 0.0007, 0.0008, 0.0009, 0.001, 0.0015, 0.002, 0.005, or 0.01. In some embodiments, the pseudocount is 0.00075. In some embodiments, the pseudocount is 0.001. In some embodiments, the pseudocount is 0.0011. In some embodiments, the pseudocount is 0.0012.

Model training can be performed, e.g., using the sklearn (scikit-learn) package in python. Three folds of cross validation may be randomly generated, and the area under the curve (AUC) calculated for each test fold. The L2 and L1 penalty corresponding with the best average AUC across all five test folds is taken, and a final refit is performed using the entire training dataset with the optimal penalty. Genes differentially expressed between sample groups can be ranked, and the top, e.g., 10, 20, 30, 40, 50, 60, 70, 80, 90, 100, 150, 200, 250, 300, 400, 450, or 500 or more differentially expressed genes identified. Further, cell type deconvolution using the sequencing data (such as RNA-seq data) returns the cell type composition (quantities of cell types) of each sample.

In some embodiments, the plurality of target genes comprises genes having above-average expression variance in a training set comprising gene expression data from samples from healthy subjects and from subjects with a disease or disorder (such as a cancer, such as AA and/or CRC). In some embodiments, the plurality of target genes having above-average expression variance comprise genes having an expression variance in the top 25^{th}, top 20^{th}, top 15^{th}, top 10^{th}, top 9^{th}, top 8^{th}, top 7^{th}, top 6^{th}, top 5^{th}, top 4^{th}, top 3^{rd}, top 2^{nd}, or top 1^{st} percentile of the genes of the training set. In some embodiments, the plurality of target genes having above-average expression variance are genes with an expression variance ranking in the top 1000, top 750, top 500, top 250, top 200, top 150, top 100, top 90, top 80, top 70, top 60, top 50, top 40, top 30, top 25, top 20, top 15, top 10, or top 5 genes in the training set. In particular embodiments, the majority, at least 75%, at least 80%, at least 85%, at least 90%, at least 95%, at least 96%, at least 97%, at least 98%, at least 99%, or all, of the target genes are protein-coding genes.

In some embodiments, the target genes comprise one or more, or each, of ICA1, CD38, ABCB4, TNFRSF17, RRP12, CHI3L2, MAP3K13, IL4R, IL12RB2, ACHE, LAG3, CD209, GGT5, DEPDC5, UPK3A, GZMH, BPI, ACP5, CD37, MAST1, RASSF4, MS4A6A, PPFIBP1, MAK, IL18RAP, KYNU, FASLG, MYB, CCND2, TRPM6, FLVCR2, CD80, TMEM156, BHLHE41, NFE2, TREM1, TMEM255A, IL1B, PLEKHG3, VPREB3, TEP1, TRPM4, SMPDL3B, LILRB2, CHI3L1, IL2RA, TLR2, BMP2K, TNFRSF11A, PLA2G7, EPHA1, ABCB9, ZFP36L2, HHEX, SKA1, CLIC2, DUSP2, SLAMF8, CDA, ZNF222, ASGR2, PBXIP1, NIPSNAP3B, TSHR, NOD2, CD300A, NAALADL1, ADRB2, SCN9A, CEACAM3, COL8A2, CLEC7A, CTSW, HPSE, CD7, P2RY14, HIC1, P2RY13, GPR19, CCR3, MROH7, CSF1, FAM174B, TNFRSF4, PDCD1, RYR1, HLA-DQA1, NCR3, ZBTB10, APOBEC3G, LILRA2, TNFRSF13B, HLA-DOB, PGLYRP1, HGF, ATP9A, ATP2C2, JMJD6, DHRS9, SLC1A3, CEACAM1, DUSP13, CRISP3, ABLIM1, HSD3B7, OSM, UPB1, BIK, MMP9, SLCO4A1, BMX, KLF5, RETN, GRB10, PRUNE2, ERLIN1, TP5313, IL1R2, EPAS1, LRRC42, GADD45A, PHTF1, RCAN3, ARG1, CYSTM1, DACH1, FKBP9, GOS2, PFKFB2, CDH26, ARMC7, PPPIR3D, ECHDC3, RDH5, ACVR1B, CKAP4, MTHFS, IL10, MFSD13A, GPR84, MYLK3, ZNF787, MYO10, RAB19, OLAH, ANKRD22, RABGEF1, SAMSN1, CACNA1D, RGL4, TFF3, GYG1, ZDHHC19, SLC51A, POC1A, HPGD, FBN1, CLEC4D, SEMA6B, PFKFB3, PAQR8, SH3PXD2B, FOSL1, MSRA, LRRN1, KCNE5, B3GNT5, CD163, CRACR2B, OPLAH, EXOSC4, KCNE1, UPP1, ST6GALNAC3, SEPTIN5, FCAR, SPATC1, MAFG, IL27, ENTPD7, PLIN5, FAM228B, ORM1, STON1, CFH, HS3ST1, DBNDD1, CD22, SLC25A39, KCNG1, TGFBR3, ADD2, COL19A1, CD200, TCL1A, PROCR, CD40, NME4, TSPAN13, RGS9, FAM184A, KHDRBS2, ENPP5, MMP8, SATB2, GPR68, CEACAM8, MYO1B, LARGE1, NT5E, RAPGEF5, ABHD17C, ZNF365, GRTP1, IGFBP3, LCN2, GLB1L2, CNKSR2, PRSS23, RASGRP3, SCN3A, C16orf74, RETREG1, ERG, SNX22, CXCR5, BEND5, SLC1A7, LEXM, CAMK2N1, SPRY1, CDCA7L, SPIB, DLC1, DIPK1B, MTCL1, PARM1, MZB1, SLC23A1, PDGFD, NMUR1, NBEA, OLR1, PTPRM, PAWR, ABCA13, TAFA1, KCNH8, EFNA5, PRSS57, PTCH1, PRTN3, SULT1A1, ZNF667, SHISA4, LICAM, RASGEF1A, DDR1, GPC2, PATL2, and IGLL5.PDCD1, ZNK222, CCR3, TRPM4, GPR19, LAG3, TMEM156, MAST1, TSHR, TNFRSF17, ASGR2, TRPM6, TNFRSF11A, CRISP3, RYR1, PLEKHG3, CD209, ACP5, OSM, and CSF1.

In other embodiments, the target genes comprise 2, 3, 4, 5, 6, 7, 8, 9, 10, 11, 12, 13, 14, 15, 16, 17, 18, 19, or 20 of ICA1, CD38, ABCB4, TNFRSF17, RRP12, CHI3L2, MAP3K13, IL4R, IL12RB2, ACHE, LAG3, CD209, GGT5, DEPDC5, UPK3A, GZMH, BPI, ACP5, CD37, MAST1, RASSF4, MS4A6A, PPFIBP1, MAK, IL18RAP, KYNU, FASLG, MYB, CCND2, TRPM6, FLVCR2, CD80, TMEM156, BHLHE41, NFE2, TREM1, TMEM255A, IL1B, PLEKHG3, VPREB3, TEP1, TRPM4, SMPDL3B, LILRB2, CHI3L1, IL2RA, TLR2, BMP2K, TNFRSF11A, PLA2G7, EPHA1, ABCB9, ZFP36L2, HHEX, SKA1, CLIC2, DUSP2, SLAMF8, CDA, ZNF222, ASGR2, PBXIP1, NIPSNAP3B, TSHR, NOD2, CD300A, NAALADL1, ADRB2, SCN9A, CEACAM3, COL8A2, CLEC7A, CTSW, HPSE, CD7, P2RY14, HIC1, P2RY13, GPR19, CCR3, MROH7, CSF1, FAM174B, TNFRSF4, PDCD1, RYR1, HLA-DQA1, NCR3, ZBTB10, APOBEC3G, LILRA2, TNFRSF13B, HLA-DOB, PGLYRP1, HGF, ATP9A, ATP2C2, JMJD6, DHRS9, SLC1A3, CEACAM1, DUSP13, CRISP3, ABLIM1, HSD3B7, OSM, UPB1, BIK, MMP9, SLCO4A1, BMX, KLF5, RETN, GRB10, PRUNE2, ERLIN1, TP5313, IL1R2, EPAS1, LRRC42, GADD45A, PHTF1, RCAN3, ARG1, CYSTM1, DACH1, FKBP9, GOS2, PFKFB2, CDH26, ARMC7, PPP1R3D, ECHDC3, RDH5, ACVR1B, CKAP4, MTHFS, IL10, MFSD13A, GPR84, MYLK3, ZNF787, MYO10, RAB19, OLAH, ANKRD22, RABGEF1, SAMSN1, CACNA1D, RGL4, TFF3, GYG1, ZDHHC19, SLC51A, POC1A, HPGD, FBN1, CLEC4D, SEMA6B, PFKFB3, PAQR8, SH3PXD2B, FOSL1, MSRA, LRRN1, KCNE5, B3GNT5, CD163, CRACR2B, OPLAH, EXOSC4, KCNE1, UPP1, ST6GALNAC3, SEPTIN5, FCAR, SPATC1, MAFG, IL27, ENTPD7, PLIN5, FAM228B, ORM1, STON1, CFH, HS3ST1, DBNDD1, CD22, SLC25A39, KCNG1, TGFBR3, ADD2, COL19A1, CD200, TCLIA, PROCR, CD40, NME4, TSPAN13, RGS9, FAM184A, KHDRBS2, ENPP5, MMP8, SATB2, GPR68, CEACAM8, MYO1B, LARGE1, NT5E, RAPGEF5, ABHD17C, ZNF365, GRTP1, IGFBP3, LCN2, GLB1L2, CNKSR2, PRSS23, RASGRP3, SCN3A, C16orf74, RETREG1, ERG, SNX22, CXCR5, BEND5, SLC1A7, LEXM, CAMK2N1, SPRY1, CDCA7L, SPIB, DLC1, DIPK1B, MTCL1, PARM1, MZB1, SLC23A1, PDGFD, NMUR1, NBEA, OLR1, PTPRM, PAWR, ABCA13, TAFA1, KCNH8, EFNA5, PRSS57, PTCH1, PRIN3, SULTIA1, ZNF667, SHISA4, L1CAM, RASGEF1A, DDR1, GPC2, PATL2, and IGLL5.PDCD1, ZNK222, CCR3, TRPM4, GPR19, LAG3, TMEM156, MAST1, TSHR, TNFRSF17, ASGR2, TRPM6, TNFRSF11A, CRISP3, RYR1, PLEKHG3, CD209, ACP5, OSM, and CSF1.

In some embodiments, the target genes comprise one or more, or each, of PGLYRP1, HGF, ATP9A, ATP2C2, JMJD6, DHRS9, SLC1A3, CEACAM1, DUSP13, CRISP3, ABLIM1, HSD3B7, OSM, UPB1, BIK, MMP9, SLCO4A1, BMX, KLF5, RETN, GRB10, PRUNE2, ERLIN1, TP5313, ILIR2, EPAS1, LRRC42, GADD45A, PHTF1, RCAN3, ARG1, CYSTM1, DACH1, FKBP9, GOS2, PFKFB2, CDH26, ARMC7, PPPIR3D, ECHDC3, RDH5, ACVRIB, CKAP4, MTHFS, IL10, MFSD13A, GPR84, MYLK3, ZNF787, MYO10, RAB19, OLAH, ANKRD22, RABGEF1, SAMSN1, CACNA1D, RGL4, TFF3, GYG1, ZDHHC19, SLC51A, POCIA, HPGD, FBN1, CLEC4D, SEMA6B, PFKFB3, PAQR8, SH3PXD2B, FOSL1, MSRA, LRRN1, KCNE5, B3GNT5, CD163, CRACR2B, OPLAH, EXOSC4, KCNE1, UPP1, ST6GALNAC3, SEPTIN5, FCAR, SPATC1, MAFG, IL27, ENTPD7, PLIN5, FAM228B, ORM1, and STON1. In other embodiments, the target genes comprise 2, 3, 4, 5, 6, 7, 8, 9, 10, 11, 12, 13, 14, 15, 16, 17, 18, 19, or 20 of PGLYRP1, HGF, ATP9A, ATP2C2, JMJD6, DHRS9, SLC1A3, CEACAM1, DUSP13, CRISP3, ABLIM1, HSD3B7, OSM, UPB1, BIK, MMP9, SLCO4A1, BMX, KLF5, RETN, GRB10, PRUNE2, ERLIN1, TP5313, IL1R2, EPAS1, LRRC42, GADD45A, PHTF1, RCAN3, ARG1, CYSTM1, DACH1, FKBP9, GOS2, PFKFB2, CDH26, ARMC7, PPP1R3D, ECHDC3, RDH5, ACVR1B, CKAP4, MTHFS, IL10, MFSD13A, GPR84, MYLK3, ZNF787, MYO10, RAB19, OLAH, ANKRD22, RABGEF1, SAMSN1, CACNA1D, RGL4, TFF3, GYG1, ZDHHC19, SLC51A, POC1A, HPGD, FBN1, CLEC4D, SEMA6B, PFKFB3, PAQR8, SH3PXD2B, FOSL1, MSRA, LRRN1, KCNE5, B3GNT5, CD163, CRACR2B, OPLAH, EXOSC4, KCNE1, UPP1, ST6GALNAC3, SEPTIN5, FCAR, SPATC1, MAFG, IL27, ENTPD7, PLIN5, FAM228B, ORM1, and STON1.

In some embodiments, the target genes comprise one or more, or each, of ICA1, CD38, ABCB4, TNFRSF17, RRP12, CHI3L2, MAP3K13, IL4R, IL12RB2, ACHE, LAG3, CD209, GGT5, DEPDC5, UPK3A, GZMH, BPI, ACP5, CD37, MAST1, RASSF4, MS4A6A, PPFIBP1, MAK, IL18RAP, KYNU, FASLG, MYB, CCND2, TRPM6, FLVCR2, CD80, TMEM156, BHLHE41, NFE2, TREM1, TMEM255A, IL1B, PLEKHG3, VPREB3, TEP1, TRPM4, SMPDL3B, LILRB2, CHI3L1, IL2RA, TLR2, BMP2K, TNFRSF11A, PLA2G7, EPHA1, ABCB9, ZFP36L2, HHEX, SKA1, CLIC2, DUSP2, SLAMF8, CDA, ZNF222, ASGR2, PBXIP1, NIPSNAP3B, TSHR, NOD2, CD300A, NAALADL1, ADRB2, SCN9A, CEACAM3, COL8A2, CLEC7A, CTSW, HPSE, CD7, P2RY14, HIC1, P2RY13, GPR19, CCR3, MROH7, CSF1, FAM174B, TNFRSF4, PDCD1, RYR1, HLA-DQA1, NCR3, ZBTB10, APOBEC3G, LILRA2, TNFRSF13B, and HLA-DOB. In other embodiments, the target genes comprise 2, 3, 4, 5, 6, 7, 8, 9, 10, 11, 12, 13, 14, 15, 16, 17, 18, 19, or 20 of ICA1, CD38, ABCB4, TNFRSF17, RRP12, CHI3L2, MAP3K13, IL4R, IL12RB2, ACHE, LAG3, CD209, GGT5, DEPDC5, UPK3A, GZMH, BPI, ACP5, CD37, MAST1, RASSF4, MS4A6A, PPFIBP1, MAK, IL18RAP, KYNU, FASLG, MYB, CCND2, TRPM6, FLVCR2, CD80, TMEM156, BHLHE41, NFE2, TREM1, TMEM255A, IL1B, PLEKHG3, VPREB3, TEP1, TRPM4, SMPDL3B, LILRB2, CHI3L1, IL2RA, TLR2, BMP2K, TNFRSF11A, PLA2G7, EPHA1, ABCB9, ZFP36L2, HHEX, SKA1, CLIC2, DUSP2, SLAMF8, CDA, ZNF222, ASGR2, PBXIP1, NIPSNAP3B, TSHR, NOD2, CD300A, NAALADL1, ADRB2, SCN9A, CEACAM3, COL8A2, CLEC7A, CTSW, HPSE, CD7, P2RY14, HIC1, P2RY13, GPR19, CCR3, MROH7, CSF1, FAM174B, TNFRSF4, PDCD1, RYR1, HLA-DQA1, NCR3, ZBTB10, APOBEC3G, LILRA2, TNFRSF13B, and HLA-DOB.

In some embodiments, the target genes comprise one or more, or each, of CFH, HS3ST1, DBNDD1, CD22, SLC25A39, KCNG1, TGFBR3, ADD2, COL19A1, CD200, TCL1A, PROCR, CD40, NME4, TSPAN13, RGS9, FAM184A, KHDRBS2, ENPP5, MMP8, SATB2, GPR68, CEACAM8, MYO1B, LARGE1, NT5E, RAPGEF5, ABHD17C, ZNF365, GRTP1, IGFBP3, LCN2, GLB1L2, CNKSR2, PRSS23, RASGRP3, SCN3A, C16orf74, RETREG1, ERG, SNX22, CXCR5, BEND5, SLC1A7, LEXM, CAMK2N1, SPRY1, CDCA7L, SPIB, DLC1, DIPK1B, MTCL1, PARM1, MZB1, SLC23A1, PDGFD, NMUR1, NBEA, OLR1, PTPRM, PAWR, ABCA13, TAFA1, KCNH8, EFNA5, PRSS57, PTCH1, PRTN3, SULT1A1, ZNF667, SHISA4, L1CAM, RASGEF1A, DDR1, GPC2, PATL2, and IGLL5.

In other embodiments, the target genes comprise 2, 3, 4, 5, 6, 7, 8, 9, 10, 11, 12, 13, 14, 15, 16, 17, 18, 19, or 20 of CFH, HS3ST1, DBNDD1, CD22, SLC25A39, KCNG1, TGFBR3, ADD2, COL19A1, CD200, TCL1A, PROCR, CD40, NME4, TSPAN13, RGS9, FAM184A, KHDRBS2, ENPP5, MMP8, SATB2, GPR68, CEACAM8, MYO1B, LARGE1, NT5E, RAPGEF5, ABHD17C, ZNF365, GRTP1, IGFBP3, LCN2, GLB1L2, CNKSR2, PRSS23, RASGRP3, SCN3A, C16orf74, RETREG1, ERG, SNX22, CXCR5, BEND5, SLC1A7, LEXM, CAMK2N1, SPRY1, CDCA7L, SPIB, DLC1, DIPK1B, MTCL1, PARM1, MZB1, SLC23A1, PDGFD, NMUR1, NBEA, OLR1, PTPRM, PAWR, ABCA13, TAFA1, KCNH8, EFNA5, PRSS57, PTCH1, PRTN3, SULT1A1, ZNF667, SHISA4, L1CAM, RASGEF1A, DDR1, GPC2, PATL2, and IGLL5.

In some embodiments, the target genes comprise one or more (such as 1, 2, 3, 4, 5, 6, 7, 8, 9, 10, 11, 12, 13, 14, 15, 16, 17, 18, 19, 20, 30, 40, 50, 60, 70, 80, more than 80, 1-10, 1-20, 1-30, 1-40, 1-50, 1-60, 1-70, 1-80, 5-80, 10-80, 20-80, 30-80, or each) of the genes listed in Table 1. In some embodiments, the target genes comprise one or more (such as 1, 2, 3, 4, 5, 6, 7, 8, 9, 10, 11, 12, 13, 14, 15, 16, 17, 18, 19, 20, 30, 40, 50, 60, 70, 80, more than 80, 1-10, 1-20, 1-30, 1-40, 1-50, 1-60, 1-70, 1-80, 5-80, 10-80, 20-80, 30-80, or each) of GZMH, PATL2, FCRL6, ZNF600, IL10RA, DTHD1, PYHIN1, HDAC11, XCL1, GZMA, RAB37, ID2, SLC9A3R1, MOSPD3, TES, EML4, CD99, ACSL6, YPEL1, H2AC17, FGFBP2, TBX21, F2R, PTGDS, DDX3Y, SYNGR1, ZNF683, SERPIN86, PRSS23, ID2, SRSF2, PRR5, CST3, SHISA4, PLAC8, BRD2, BMF, NIPAL2, UTS2, RARS2, ZNF320, RBL2, CREB3L2, RNF38, CCDC88A, MEX3C, SLC38A11, COL19A1, PNPLA7, AFF3, STEAP1B, CELSR1, GRAPL, CBX5, AKAP6, EVL, CRISP3, BPI, MMP8, OLFM4, MPO, MROH6, KDM5D, IDH2, ABI3, S100A4, H2AC17, GIMAP4, PPP1CB, XAF1, RPL37, PTPRO, CD177, RPS13, NAIP, RPS12, ID2, SH3BP1, RAB37, PLEKHG3, AFDN, SLC9A3R1, FLNA, SSBP3, RHOB, LIPA, ISCA1, PAG1, DDX3X, KDM5C, PIM2, IL4R, ZFX, FCGR2B, KDM6A, SARAF, TTC39C, NCOR2, ATP5MD, ARHGD1A, TRAF5, SCAMP2, RPS26, GSTM4, MICAL3, TOMM20, SSBP3, RALGDS, ITGB1, DYRK1B, ARRB1, TRIM11, LFNG, PAXX, ACCS, and SLC2A6.

In some embodiments, the target genes comprise one or more (or each) of GZMH, PATL2, FCRL6, ZNF600, IL10RA, DTHD1, PYHIN1, HDAC11, XCL1, GZMA, RAB37, ID2, SLC9A3R1, MOSPD3, TES, EML4, CD99, ACSL6, YPEL1, and H2AC17. In some embodiments, the target genes comprise one or more (or each) of FGFBP2, TBX21, F2R, PTGDS, DDX3Y, SYNGR1, ZNF683, SERPIN86, PRSS23, ID2, SRSF2, PRR5, CST3, SHISA4, PLAC8, and BRD2. In some embodiments, the target genes comprise one or more (or each) of BMF, NIPAL2, UTS2, RARS2, ZNF320, RBL2, CREB3L2, RNF38, CCDC88A, MEX3C, SLC38A11, COL19A1, PNPLA7, AFF3, STEAP1B, CELSR1, GRAPL, CBX5, AKAP6, and EVL. In some embodiments, the target genes comprise one or more (or each) of CRISP3, BPI, MMP8, OLFM4, MPO, MROH6, KDM5D, IDH2, ABI3, S100A4, H2AC17, GIMAP4, PPP1CB, XAF1, RPL37, PTPRO, CD177, RPS13, NAIP, RPS12. In some embodiments, the target genes comprise one or more (or each) of ID2, SH3BP1, RAB37, PLEKHG3, AFDN, SLC9A3R1, FLNA, SSBP3, RHOB, LIPA, ISCA1, PAG1, DDX3X, KDM5C, PIM2, IL4R, ZFX, FCGR2B, KDM6A, and SARAF. In some embodiments, the target genes comprise one or more (or each) of TTC39C, NCOR2, ATP5MD, ARHGD1A, TRAF5, SCAMP2, RPS26, GSTM4, MICAL3, TOMM20, SSBP3, RALGDS, ITGB1, DYRK1B, ARRB1, TRIM11, LFNG, PAXX, ACCS, SLC2A6.

In some embodiments, the target genes comprise one or more (or each) of GZMH, PATL2, FCRL6, ZNF600, IL10RA, DTHD1, PYHIN1, HDAC11, XCL1, and GZMA. In some embodiments, the target genes comprise one or more (or each) of RAB37, ID2, SLC9A3R1, MOSPD3, TES, EML4, CD99, ACSL6, YPEL1, and H2AC17. In some embodiments, the target genes comprise one or more (or each) of FGFBP2, TBX21, F2R, PTGDS, DDX3Y, SYNGR1, ZNF683, SERPIN86, PRSS23, and ID2. In some embodiments, the target genes comprise one or more (or each) of SRSF2, PRR5, CST3, SHISA4, PLAC8, and BRD2. In some embodiments, the target genes comprise one or more (or each) of BMF, NIPAL2, UTS2, RARS2, ZNF320, RBL2, CREB3L2, RNF38, CCDC88A, and MEX3C. In some embodiments, the target genes comprise one or more (or each) of SLC38A11, COL19A1, PNPLA7, AFF3, STEAP1B, CELSR1, GRAPL, CBX5, AKAP6, and EVL. In some embodiments, the target genes comprise one or more (or each) of CRISP3, BPI, MMP8, OLFM4, MPO, MROH6, KDM5D, IDH2, ABI3, and S100A4. In some embodiments, the target genes comprise one or more (or each) of H2AC17, GIMAP4, PPP1CB, XAF1, RPL37, PTPRO, CD177, RPS13, NAIP, and RPS12. In some embodiments, the target genes comprise one or more (or each) of ID2, SH3BP1, RAB37, PLEKHG3, AFDN, SLC9A3R1, FLNA, SSBP3, RHOB, and LIPA. In some embodiments, the target genes comprise one or more (or each) of ISCA1, PAG1, DDX3X, KDM5C, PIM2, IL4R, ZFX, FCGR2B, KDM6A, and SARAF. In some embodiments, the target genes comprise one or more (or each) of TTC39C, NCOR2, ATP5MD, ARHGD1A, TRAF5, SCAMP2, RPS26, GSTM4, MICAL3, and TOMM20. In some embodiments, the target genes comprise one or more (or each) of SSBP3, RALGDS, ITGB1, DYRK1B, ARRB1, TRIM11, LFNG, PAXX, ACCS, and SLC2A6.

In some cases, a sample group may exhibit a sample imbalance, such as a sex imbalance or an age imbalance. For example, if a healthy subject group is mostly females, a model could potentially learn genes associated with sex differences rather than genes that are associated with a disease or condition (such as a cancer). In such instances, female healthy samples can be down-weighted, and male disease state samples, female disease state samples, and male healthy samples can each be up-weighted. Alternatively, in such examples, the number of female healthy samples can be downsampled, such that the numbers of male and female samples and/or the number of healthy and diseased samples are the same in each sample group.

Thus, in some embodiments, determining the presence, absence, or likelihood of the disease or disorder comprises compensating for effects of sex on gene expression. In some embodiments, compensating for effects of sex on gene expression comprises regressing out the effects of sex on gene expression. In some embodiments, determining the presence, absence, or likelihood of the disease or disorder comprises compensating for effects of sex on cell type quantity or cell type proportion. In some embodiments, compensating for effects of sex on cell type quantity or cell type proportion comprises regressing out the effects of sex on cell type quantity or cell type proportion. The effect of sex can be learned for a particular sample group on a per-gene basis, such as using males and females from a healthy donor population, after which sex-specific differences in gene expression can be corrected for across all samples in the group (e.g., in a training set). In particular embodiments, the target genes comprise genes that are not differentially expressed according to sex. For example, a population of healthy and disease-state matched males and healthy females can be subjected to the methods described herein, genes that are not differentially expressed between males and females can be identified. Genes identified as differentially expressed between males and females (i.e., genes that are differentially expressed (such as above a specified threshold) according to sex, as opposed to, e.g., according to a disease state) are optionally removed from a target gene list. In particular embodiments, at least 50%, at least 60%, at least 70%, at least 75%, at least 80%, at least 85%, at least 90%, at least 95%, at least 96%, at least 97%, at least 98%, at least 99%, or 100% of the target genes are not differentially expressed (e.g., are not differentially expressed above a specified threshold) according to sex. In some embodiments, the target genes are identified using a training set comprising samples from individuals of the same sex as a subject. In some embodiments, quantities of the immune cell types (e.g., quantities of immune cell types from which RNA originated based on the expression levels of the RNAs) are identified using a training set comprising samples from individuals of the same sex as the subject. In some embodiments, the subject is female. In some embodiments, the subject is male.

In some embodiments, determining the presence, absence, or likelihood of a disease or disorder comprises compensating for effects of age on gene expression. In such embodiments, compensating for effects of age on gene expression can comprise regressing out the effects of age on gene expression, such as described herein for the effects of sex on gene expression. In some embodiments, determining the presence, absence, or likelihood of the disease or disorder comprises compensating for effects of age on cell type quantity or cell type proportion. In some embodiments, compensating for effects of age on cell type quantity or cell type proportion comprises regressing out the effects of age on cell type quantity or cell type proportion. In particular examples, target genes comprise genes that are not differentially expressed (such as above a specified threshold) according to age. In specific, non-limiting examples, at least 50%, at least 60%, at least 70%, at least 75%, at least 80%, at least 85%, at least 90%, at least 95%, at least 96%, at least 97%, at least 98%, at least 99%, or 100% of the target genes are not differentially expressed (e.g., are not differentially expressed above a specified threshold) according to age. In some examples, the target genes were identified using a training set comprising samples from individuals that, at the time of sample collection from each individual, were of an age that is within 1 year, within 2 years, within 3 years, within 4 years, within 5 years, within 6 years, within 7 years, within 8 years, within 9 years, within 10 years, within 11 years, within 12 years, within 13 years, within 14 years, or within 15 years of the age of the subject at the time of sample collection from the subject.

In other particular examples, quantities of immune cell types do not differ according to age. In some embodiments, at least 50%, at least 60%, at least 70%, at least 75%, at least 80%, at least 85%, at least 90%, at least 95%, at least 96%, at least 97%, at least 98%, at least 99%, or 100% of the immune cell types do not differ in quantity according to age. In some embodiments, the quantities of the immune cell types were identified using a training set comprising samples from individuals that, at the time of sample collection from each individual, were of an age that is within 1 year, within 2 years, within 3 years, within 4 years, within 5 years, within 6 years, within 7 years, within 8 years, within 9 years, within 10 years, within 11 years, within 12 years, within 13 years, within 14 years, or within 15 years of the age of the subject at the time of sample collection from the subject.

### C. Immune Cell Type Identification and Quantification

In some embodiments, methods disclosed herein comprise steps of sequencing RNA (e.g., RNA from a sample comprising cells, such as a buffy coat sample, a whole blood sample, a leukapheresis sample, a PBMC sample, and/or additional RNA), and determining levels of each of a plurality of immune cell types from which the RNA originated. The levels of immune cell types may be expressed, e.g., as relative amounts or percentages for each cell type being quantified.

The methods herein thus allow for detection and/or identification of immune cell-specific differentially expressed genes (such as CTD genes of the target gene list of Example 2, or the genes listed in Table 1 below) that can be used to identify and quantify different immune cell types from which RNA in a sample originated. The immune cell types may comprise immune cells of different origins, different differentiation types, different activation types, or any combination of different origins, different differentiation types, and different activation types. Indeed, differentiation status and activation status significantly overlap and often change together in a given immune cell. For example, activation of an immune cell may induce differentiation of the cell. Immune cells of different activation types include activated cells, such as cells activated by inflammatory cytokines or antigens, and suppressed cells, such as cells suppressed by Tregs. The immune cell types include neutrophils, lymphocytes, plasma cells, monocytes, macrophages, dendritic cells, mast cells, eosinophils, T cells, CD4+ T cells, B cells, NK cells, megakaryocytes, CD8+ central memory cells, CD8+ effector memory cells, CD4+ central memory cells, CD4+ effector memory cells, immature neutrophils, precursor B cells, plasma cells, memory-switched B cells, plasma cells, basophils, naïve B cells, memory B cells, CD8+ T cells, naïve CD4+ T cells, resting CD4+ memory T cells, activated CD4+ memory T cells; follicular helper T cells; regulatory T cells (Tregs); gamma delta T cells; resting NK cells; activated NK cells, M0 macrophages, M1 macrophages, M2 macrophages, resting dendritic cells, activated dendritic cells, resting mast cells, and activated mast cells. In some embodiments, genes from such cell types may exhibit increased expression in samples, such as RNA from a buffy coat sample, a whole blood sample, a leukapheresis sample, a PBMC sample, and/or additional RNA samples from healthy individuals, but decreased expression in such samples from individuals with a disease or disorder such as cancer or a precancerous condition. In other embodiments, genes from such cell types may exhibit decreased expression in samples, such as RNA from a buffy coat sample, a whole blood sample, a leukapheresis sample, a PBMC sample, and/or additional RNA samples from healthy individuals, but increased expression in such samples from individuals with a disease or disorder such as cancer or a precancerous condition. In some embodiments, to distinguish RNA from closely related cell types, such as naïve and activated B cells, naïve and activated T cells, or different stages of myeloid lineages, differentially expressed genes (such as one or more genes of a plurality of target genes identified using the methods disclosed herein) may be detected. In some embodiments, at least some of the differentially expressed genes are exclusively expressed in only one cell type or in only one cell type within a cluster. In some embodiments, at least 2, at least 3, at least 4, at least 5, at least 6, at least 7, at least 8, at least 9, or at least 10 differentially expressed genes are exclusively expressed in only one cell type that is being identified or quantified within a cluster.

TABLE 1
Genes observed to be differentially expressed in particular
cell types, inferred as a component of bulk expression (See Example 3).

| | Genes with | Genes with |
|---|---|---|
| | increased expression | decreased expression |
| | in AA samples relative to | in AA samples relative to |
| Cell Type | healthy samples | healthy samples |
| CD8+ T | GZMH, PATL2, | RAB37, ID2, SLC9A3R1, |
| cells | FCRL6, ZNF600, | MOSPD3, TES, |
| | IL10RA, DTHD1, PYHIN1, | EML4, CD99, |
| | HDAC11, XCL1, GZMA | ACSL6, YPEL1, H2AC17 |
| NK cells | FGFBP2, TBX21, | SRSF2, PRR5, |
| | F2R, PTGDS, DDX3Y, | CST3, SHISA4, |
| | SYNGR1, ZNF683, | PLAC8, BRD2 |
| | SERPIN86, PRSS23, ID2 | |
| B cells | BMF, NIPAL2, | SLC38A11, COL19A1, |
| | UTS2, RARS2, | PNPLA7, AFF3, |
| | ZNF320, RBL2, | STEAPIB, CELSR1, |
| | CREB3L2, RNF38, | GRAPL, CBX5, |
| | CCDC88A, MEX3C | AKAP6, EVL |
| Monocytes | CRISP3, BPI, MMP8, | H2AC17, GIMAP4, |
| and | OLFM4, MPO, MROH6, | PPP1CB, XAF1, RPL37, |
| Macrophages | KDM5D, IDH2, | PTPRO, CD177, |
| | ABI3, S100A4 | RPS13, NAIP, RPS12 |
| Neutrophils | ID2, SH3BP1, RAB37, | ISCA1, PAG1, |
| | PLEKHG3, AFDN, | DDX3X, KDM5C, |
| | SLC9A3R1, | PIM2, IL4R, |
| | FLNA, SSBP3, | ZFX, FCGR2B, |
| | RHOB, LIPA | KDM6A, SARAF |
| CD4+ T cells | TTC39C, NCOR2, | SSBP3, RALGDS, |
| | ATP5MD, ARHGD1A, | ITGB1, DYRK1B, |
| | TRAF5, SCAMP2, | ARRB1, TRIM11, |
| | RPS26, GSTM4, MICAL3, | LFNG, PAXX, |
| | TOMM20 | ACCS, SLC2A6 |

In some embodiments, determining the levels of different immune cell types from which RNA in a sample originated facilitates disease diagnosis or identification of appropriate treatments. In some embodiments, a change in the levels of one or more immune cell types is indicative of the presence of a disease or disorder in a subject, such as cancer, precancer, an infection, transplant rejection, or other disorder that causes changes in the relative amounts of certain immune cell types relative to the amounts present in a healthy subject. In some embodiments, changes in both the levels of one or more immune cell types in combination with sequence-independent changes, such as epigenetic changes in DNA from the subject, are indicative of the presence of a disease or disorder in a subject, such as cancer, precancer, an infection, transplant rejection, or other disorder that causes changes in the relative amounts of certain immune cell types and epigenetic changes relative to a healthy subject. In some embodiments, the methods facilitate identification of appropriate treatments based on the likelihood that a subject will respond to the treatment. In some such embodiments, determining the levels of RNA from one or more immune cell types in a sample from a subject having a certain cancer type facilitates prediction of the clinical outcome for immunotherapy in the subject. Where determining the levels of different immune cell types facilitates disease diagnosis, identification of appropriate treatments and association with therapeutic response, the thresholds for disease diagnosis and for identification of appropriate treatments may be the same or different. The levels can be determined based on a count of molecules corresponding to different immune cell types, or the relative frequency of such molecules or any value or ratio based on a count of molecules corresponding to one or more different immune cell types.

In some embodiments, expression levels are determined (such as using the methods described herein) for a target gene set comprising a plurality of target genes that are differentially expressed in a plurality of immune cell types, wherein the plurality of immune cell types comprises one or more, or each, of neutrophils, lymphocytes, plasma cells, monocytes, macrophages, dendritic cells, mast cells, eosinophils, T cells, CD4+ T cells, B cells, NK cells, megakaryocytes, CD8+ central memory cells, CD8+ effector memory cells, CD4+ central memory cells, CD4+ effector memory cells, immature neutrophils, precursor B cells, plasma cells, memory-switched B cells, plasma cells, basophils, naïve B cells, memory B cells, CD8+ T cells, naïve CD4+ T cells, resting CD4+ memory T cells, activated CD4+ memory T cells; follicular helper T cells; regulatory T cells (Tregs); gamma delta T cells; resting NK cells; activated NK cells, M0 macrophages, M1 macrophages, M2 macrophages, resting dendritic cells, activated dendritic cells, resting mast cells, and activated mast cells. In some embodiments, the plurality of immune cell types comprises one or more of naïve B cells, naïve CD4+ T cells, CD8+ T cells, resting NK cells, Tregs, and monocytes. In some embodiments, the plurality of immune cell types comprises two or more, three or more, four or more, or five or more of naïve B cells, naïve CD4+ T cells, CD8+ T cells, resting NK cells, Tregs, and monocytes. In some embodiments, the plurality of immune cell types comprises naïve B cells, naïve CD4+ T cells, CD8+ T cells, resting NK cells, Tregs, and monocytes.

In particular embodiments, the plurality of immune cell types comprises one or more, or each, of CD8+ T cells, resting CD4+ memory T cells, Tregs, and naïve B cells. In other particular embodiments, the plurality of immune cell types comprises CD8+ T cells, resting CD4+ memory T cells, Tregs, and naïve B cells. In another particular embodiment, the plurality of immune cell types comprises CD8+ T cells, resting CD4+ memory T cells, and T regs. In another particular embodiment, the plurality of immune cell types comprises CD8+ T cells, Tregs, and naïve B cells. In another particular embodiment, the plurality of immune cell types comprises CD8+ T cells, resting CD4+ memory T cells, and naïve B cells. In other particular embodiments, the plurality of immune cell types comprises resting CD4+ memory T cells, Tregs, and naïve B cells. In another particular embodiment, the plurality of immune cell types comprises CD8+ T cells. In another particular embodiment, the plurality of immune cell types comprises resting CD4+ memory T cells. In another particular embodiment, the plurality of immune cell types comprises Tregs. In another particular embodiment, the plurality of immune cell types comprises naïve B cells. In yet other particular embodiments, the plurality of immune cell types comprises neutrophils. In another particular embodiment, the plurality of immune cell types comprises naïve CD4+ T cells. In another particular embodiment, the plurality of immune cell types comprises resting NK cells. In another particular embodiment, the plurality of immune cell types comprises monocytes. In some embodiments, the plurality of immune cell types comprises In other particular embodiments, the plurality of immune cell types comprises T cells, B cells, and NK cells; neutrophils and lymphocytes; neutrophils, T cells, B cells, and NK cells; granulocytes and lymphocytes; or granulocytes, T cells, B cells, and NK cells.

In some embodiments, determining the presence, absence, or likelihood of a disease or disorder (such as a cancer, such as AA and/or CRC) in the subject based on the quantities of the immune cell types comprises determining the levels of one or more, or each, of the plurality of immune cell types relative to total blood cells. In some embodiments, determining the presence, absence, or likelihood of a disease or disorder (such as a cancer, e.g., any type of cancer mentioned elsewhere herein, such as AA and/or CRC) in the subject based on the quantities of the immune cell types comprises determining the levels of one or more, or each, of the plurality of immune cell types relative to total white blood cells. In certain embodiments, determining the presence, absence, or likelihood of the disease or disorder (such as the cancer, e.g., any type of cancer mentioned elsewhere herein, such as AA and/or CRC) in the subject based on the quantities of the immune cell types comprises determining the levels of one or more, or each, of T, B, and NK cells relative to all lymphocytes. In certain embodiments, determining the presence, absence, or likelihood of the disease or disorder (such as the cancer, e.g., any type of cancer mentioned elsewhere herein, such as AA and/or CRC) in the subject based on the quantities of the immune cell types comprises determining relative proportions of neutrophils and lymphocytes, or of neutrophils and one or more, or each, of T cells, B cells, and NK cells.

In some embodiments, the plurality of target genes comprises genes differentially expressed in an activated cell type relative to the same cell type that is not activated. In some embodiments, the plurality of target genes comprises genes differentially expressed in at least (a) a first cell type that is activated relative to the same first cell type that is not activated, and (b) a second cell type that is activated relative to the same second cell type that is not activated. In some embodiments, the activated cell type is neutrophils, lymphocytes, plasma cells, monocytes, macrophages, dendritic cells, mast cells, or eosinophils. In particular embodiments, the plurality of target genes comprises genes differentially expressed in neutrophils relative to a non-neutrophil cell type. In some embodiments, the non-neutrophil cell type is one or more, or each, of a non-immune cell type, a non-granulocyte cell type, a myeloid non-granulocyte cell type, a lymphoid cell type, lymphocytes, T cells, B cells, and NK cells.

In some embodiments, the plurality of target genes comprises genes differentially expressed in lymphocytes relative to a non-lymphocyte cell type. In some embodiments, the plurality of target genes comprises genes differentially expressed in a first cell type relative to a second cell type different from the first cell type, and the first cell type is neutrophils, lymphocytes, plasma cells, monocytes, macrophages, dendritic cells, mast cells, eosinophils, T cells, CD4+ T cells, B cells, NK cells, megakaryocytes, CD8+ central memory cells, CD8+ effector memory cells, CD4+ central memory cells, CD4+ effector memory cells, immature neutrophils, precursor B cells, plasma cells, memory-switched B cells, plasma cells, basophils, naïve B cells, memory B cells, CD8+ T cells, naïve CD4+ T cells, resting CD4+ memory T cells, activated CD4+ memory T cells; follicular helper T cells; regulatory T cells (Tregs); gamma delta T cells; resting NK cells; activated NK cells, M0 macrophages, M1 macrophages, M2 macrophages, resting dendritic cells, activated dendritic cells, resting mast cells, or activated mast cells. In some embodiments, the plurality of target genes comprises genes differentially expressed in a first cell type relative to a second cell type different from the first cell type, and the first cell type is B cells, T cells, or NK cells. In some embodiments, the second cell type is neutrophils, lymphocytes, plasma cells, monocytes, macrophages, dendritic cells, mast cells, eosinophils, T cells, CD4+ T cells, B cells, NK cells, megakaryocytes, CD8+ central memory cells, CD8+ effector memory cells, CD4+ central memory cells, CD4+ effector memory cells, immature neutrophils, precursor B cells, plasma cells, memory-switched B cells, plasma cells, basophils, naïve B cells, memory B cells, CD8+ T cells, naïve CD4+ T cells, resting CD4+ memory T cells, activated CD4+ memory T cells; follicular helper T cells; regulatory T cells (Tregs); gamma delta T cells; resting NK cells; activated NK cells, M0 macrophages, M1 macrophages, M2 macrophages, resting dendritic cells, activated dendritic cells, resting mast cells, or activated mast cells.

In some embodiments, the plurality of target genes comprises genes differentially expressed when the disease or disorder is present relative to when the disease or disorder is not present. In some embodiments, the plurality of target genes comprises genes differentially expressed in the disease or disorder cells relative to healthy cells of the same cell type as the disease or disorder cells. In some embodiments, the disease or disorder is a cancer or precancer. In particular embodiments, the cancer or precancer is advanced adenoma (AA) and/or colorectal cancer (CRC). In other particular embodiments, the cancer or precancer is a blood cancer, brain cancer, lung cancer, skin cancer, nose cancer, throat cancer, liver cancer, bone cancer, lymphoma, pancreatic cancer, skin cancer, bowel cancer, rectal cancer, thyroid cancer, bladder cancer, kidney cancer, mouth cancer, stomach cancer, solid state tumor, heterogeneous tumor, homogenous tumor, or the like. Specific examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic leukemia (CLL), chronic myeloid leukemia (CML), chronic myelomonocytic leukemia (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas, prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma. In some embodiments, the cancer is a hematological cancer. In other embodiments, the cancer is a type of cancer that is not a hematological cancer, e.g., a solid tumor cancer such as a carcinoma, adenocarcinoma, or sarcoma.

In certain embodiments, the plurality of target genes comprises genes differentially expressed in the disease or disorder cells relative to healthy colon epithelial cells. In some embodiments, the plurality of target genes comprises genes differentially expressed in the disease or disorder cells relative to a myeloid cell type or an erythroid cell type. In some embodiments, the plurality of target genes comprises genes differentially expressed in colon epithelial cells relative to a myeloid cell type or an erythroid cell type.

### D. Analysis

In some embodiments, a method described herein comprises sequencing RNA to determine the levels of particular immune cell types from which RNA originated, and/or expression levels of a plurality of target genes. The immune cell types may comprise naïve and activated lymphocytes, myeloid cells at different points of differentiation, and/or other types described elsewhere herein. In some methods, the determination of levels of immune cell types and/or the determination of expression levels of a plurality of target genes facilitates determination of the likelihood that the subject from which the RNA was obtained has a disease or disorder related to the immune system, such as an infection, transplant rejection, or cancer or precancer.

In some embodiments, a method described herein comprises identifying the presence of RNA produced by a tumor (or neoplastic cells, or cancer cells) or by precancer cells. In some embodiments, a method described herein comprises identifying the presence of RNA produced by immune cells that are not tumor cells, cancer cells, or precancer cells. In some such embodiments, determination of immune cell distribution facilitates detection or diagnosis or cancer or precancer, or determination of cancer prognosis or cancer treatment options. For example, determining the ratios of different immune cell types may facilitate such detection or determination. In some embodiments, the ratio numerator is the number or relative number of neutrophils, monocytes, or both, and the ratio denominator is the number or relative number of T cells, B cells, NK cells, or all lymphocytes. In some embodiments, the ratio numerator is the number or relative number of neutrophils, and the ratio denominator is the number or relative number of T cells, B cells, NK cells, or all lymphocytes. In some embodiments, the ratio numerator is the number or relative number of monocytes, and the ratio denominator is the number or relative number of T cells, B cells, NK cells, or all lymphocytes. In some embodiments, the ratio numerator is the number or relative number of neutrophils and monocytes, and the ratio denominator is the number or relative number of T cells, B cells, NK cells, or all lymphocytes. In some embodiments, the ratio is a neutrophil to lymphocyte ratio. In some embodiments, the ratio is a monocyte to T cell ratio. In some embodiments, elevations in such ratios are associated with cancer. In other embodiments, reductions in such ratios are associated with cancer.

The present methods can be used to determine (diagnose) the presence or absence of a disease or disorder, or determine the likelihood of the disease or disorder, particularly cancer or precancer, in a subject, to characterize conditions (e.g., staging cancer or determining heterogeneity of a cancer), monitor response to treatment of a condition, effect prognosis risk of developing a condition or subsequent course of a condition. The present disclosure can also be useful in determining the efficacy of a particular treatment option. Successful treatment options may increase the amount or change the distribution (type) of immune cells present in a subject's blood. In other examples, this may not occur. In another example, perhaps certain treatment options may be correlated with genetic profiles of cancers over time. This correlation may be useful in selecting a therapy.

Additionally, if a cancer is observed to be in remission after treatment, the present methods can be used to monitor residual disease or recurrence of disease.

The types and number of cancers that may be detected may include blood cancers, brain cancers, lung cancers, skin cancers, nose cancers, throat cancers, liver cancers, bone cancers, lymphomas, pancreatic cancers, skin cancers, bowel cancers, rectal cancers, thyroid cancers, bladder cancers, kidney cancers, mouth cancers, stomach cancers, solid state tumors, heterogeneous tumors, homogenous tumors and the like. Type and/or stage of cancer can be detected from genetic variations including mutations, rare mutations, indels, copy number variations, transversions, translocations, recombination, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and abnormal changes in nucleic acid 5-methylcytosine.

In particular embodiments, the disease or disorder is a cancer. In specific, non-limiting embodiments, the cancer is advanced adenoma (AA) and/or colorectal cancer (CRC). In some examples, the method comprises determining the presence or absence of AA. In some examples, the method comprises determining the likelihood of AA. In some examples, the method comprises determining the presence or absence of CRC. In some examples, the method comprises determining the likelihood of CRC. In some examples, the method comprises determining the presence or absence of AA and CRC. In some examples, the method comprises determining the likelihood of AA and CRC.

Genetic data can also be used for characterizing a specific form of cancer. Cancers are often heterogeneous in both composition and staging. Genetic profile data may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

Further, the methods of the disclosure may be used to characterize the heterogeneity of an abnormal condition in a subject. Such methods can include, e.g., generating a genetic profile of extracellular polynucleotides derived from the subject, wherein the genetic profile comprises a plurality of data resulting from copy number variation and rare mutation analyses. In some embodiments, an abnormal condition is cancer or precancer. In some embodiments, the abnormal condition may be one resulting in a heterogeneous genomic population. In the example of cancer, some tumors are known to comprise tumor cells in different stages of the cancer. In other examples, heterogeneity may comprise multiple foci of disease. Again, in the example of cancer, there may be multiple tumor foci, perhaps where one or more foci are the result of metastases that have spread from a primary site.

The present methods can be used to generate a profile, fingerprint, or set of data that is a summation of genetic information derived from different cells in a heterogeneous disease. Such a set of data may comprise differential expression of one or more target genes identified using the methods disclosed herein, copy number variation, or other mutation analyses alone or in combination.

The present methods can be used to diagnose, prognose, monitor or observe cancers, or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose RNA and other polynucleotides may co-circulate with maternal molecules.

In general, after sequencing, analysis of reads can be performed on a partition-by-partition level, as well as a whole RNA population level. Tags can be used to sort reads from, e.g., different partitions and/or from different samples.

An exemplary method for determining quantities of the immune cell types from which RNA originated based on the expression levels, and/or determining expression levels of a plurality of target genes comprises the following steps:

1. Preparing extracted RNA samples (e.g., RNA isolated from a sample comprising cells or a blood sample (e.g., a whole blood sample, a buffy coat sample, a leukapheresis sample, or a PBMC sample) and/or additional RNA) from samples of a training set comprising samples from healthy subjects and from subjects having a disease or disorder (such as a cancer, such as AA and/or CRC). In some embodiments, poly (A) selection or rRNA depletion and/or globin mRNA depletion are performed using the extracted RNA.
2. Preparing an RNA-seq library from the RNA sample, including ligating adapters comprising molecular tags to the cDNA, and amplifying the cDNA. In some embodiments, such as embodiments wherein RNA isolated from a sample comprising cells or a blood sample (e.g., a whole blood sample, a buffy coat sample, a leukapheresis sample, or a PBMC sample) is used, RNA is fragmented prior to adapter ligation, e.g., by sonication or enzymatic digestion.
3. Sequencing the adapter-ligated, amplified cDNA using RNA-seq.
4. Analyzing the resulting expression dataset to identify genes differentially expressed between samples from the healthy subjects and from the subjects having the disease or disorder (such as a cancer, such as AA and/or CRC), and ranking the differentially expressed genes by expression variance between the two sample groups. Cell type deconvolution using the expression data returns the cell type composition (quantities of cell types) of each sample in the training set. In some embodiments, the 50-500 top-ranked differentially expressed genes are selected as the target gene list for use in determining the presence, absence, or likelihood of the disease or disorder in a subject based on the quantities of the immune cell types and/or the expression levels of the plurality of target genes.
5. Repeating steps 1-2 using at least one sample (e.g., RNA isolated from a sample comprising cells or a blood sample (e.g., a whole blood sample, a buffy coat sample, a leukapheresis sample, or a PBMC sample) and/or additional RNA) from a subject that may have or is at risk of having a disease or condition (such as a cancer, such as AA and/or CRC).
6. Determining expression levels of one or more of the plurality genes of the target gene list (generated in step 4) in the sample from the subject, and/or determining quantities of the immune cell types in the sample from the subject, and determining, based on the quantities of the immune cell types and/or the expression levels of the one or more of the plurality of target genes, the presence, absence, or likelihood of the disease or disorder (such as a cancer, such as AA and/or CRC) in the subject.

An exemplary workflow is described in Examples 1 and 2.

An exemplary method for training a model for determining quantities of the immune cell types from which RNA originated based on the expression levels comprises the following steps:
1. Preparing extracted RNA samples (e.g., RNA isolated from a sample comprising cells or a blood sample (e.g., a whole blood sample, a buffy coat sample, a leukapheresis sample, or a PBMC sample) and/or additional RNA) from samples of a training set comprising samples from healthy subjects and from subjects having a disease or disorder (such as a cancer, such as AA and/or CRC). In some embodiments, poly (A) selection or rRNA depletion and/or globin mRNA depletion are performed using the extracted RNA.
2. Preparing an RNA-seq library from the RNA sample, including ligating adapters comprising molecular tags to the cDNA, and amplifying the cDNA. In some embodiments, such as embodiments wherein RNA isolated from a sample comprising cells or a blood sample (e.g., a whole blood sample, a buffy coat sample, a leukapheresis sample, or a PBMC sample) is used, RNA is fragmented prior to adapter ligation, e.g., by sonication or enzymatic digestion.
3. Sequencing the adapter-ligated, amplified cDNA using RNA-seq.
4. Analyzing the resulting expression dataset to identify genes differentially expressed between samples from the healthy subjects and from the subjects having the disease or disorder (such as a cancer, such as AA and/or CRC), and ranking the differentially expressed genes by expression variance between the two sample groups. Cell type deconvolution using the expression data returns the cell type composition (quantities of cell types) of each sample in the training set. In some embodiments, genes differentially expressed in particular cell types of interest are selected as the target gene list(s) for use in determining the presence, absence, or likelihood of the disease or disorder in a subject.

An exemplary method for determining quantities of the immune cell types from which RNA originated based on the expression levels comprises the following steps:
1. Preparing extracted RNA from at least one sample (e.g., RNA isolated from a sample comprising cells or a blood sample (e.g., a whole blood sample, a buffy coat sample, a leukapheresis sample, or a PBMC sample) and/or additional RNA) from a subject that may have or is at risk of having a disease or condition (such as a cancer, such as AA and/or CRC). In some embodiments, poly (A) selection or rRNA depletion and/or globin mRNA depletion are performed using the extracted RNA.
2. Preparing an RNA-seq library from the at least one RNA sample, including ligating adapters comprising molecular tags to the cDNA, and amplifying the cDNA. In some embodiments, such as embodiments wherein RNA isolated from a sample comprising cells or a blood sample (e.g., a whole blood sample, a buffy coat sample, a leukapheresis sample, or a PBMC sample) is used, RNA is fragmented prior to adapter ligation, e.g., by sonication or enzymatic digestion.
3. Sequencing the adapter-ligated, amplified cDNA using RNA-seq.
4. Determining quantities and/or relative proportions of immune cell types in the at least one sample from the subject based on expression levels of one or more of a plurality genes of a target gene list (e.g., a target gene list generated by training a model, e.g., using the above “exemplary method for training a model for determining quantities of the immune cell types from which RNA originated based on the expression levels”) in the sample from the subject, and determining, based on the quantities and/or relative proportions of the immune cell types, the presence, absence, or likelihood of the disease or disorder (such as a cancer, such as AA and/or CRC) in the subject.

An exemplary workflow is described in Example 3.

### E. Partitioning

In some instances, a sample from a subject (e.g., heterogeneous sample) is divided into two or more subsamples. A sample can be any biological sample isolated from a subject. A sample can be a bodily sample. Samples can include body tissues, such as known or suspected solid tumors, whole blood, buffy coat, PBMCs, platelets, serum, plasma, stool, red blood cells, white blood cells or leukocytes, endothelial cells, tissue biopsies, cerebrospinal fluid synovial fluid, lymphatic fluid, ascites fluid, interstitial or extracellular fluid, the fluid in spaces between cells, including gingival crevicular fluid, bone marrow, pleural effusions, cerebrospinal fluid, saliva, mucous, sputum, semen, sweat, urine. Samples are preferably body fluids, particularly blood and fractions thereof, and urine. A sample can be in the form originally isolated from a subject or can have been subjected to further processing to remove or add components, such as cells, or enrich for one component relative to another. Thus, preferred body fluids for analysis include body fluids comprising cells, such as whole blood, buffy coat separated from whole blood, PBMCs separated from whole blood, a leukapheresis sample, and/or plasma or serum.

In some embodiments, RNA is isolated from at least a first subsample, and DNA is isolated from at least a second subsample. Alternatively, RNA and DNA can be separately isolated from the sample. RNA isolated from the sample or at least first subsample can be used in any of the methods disclosed herein. In particular embodiments, isolated DNA or a subsample comprising DNA is further partitioned, and each partition of the DNA subsample is differentially tagged. Tagged partitions can then be pooled together for collective sample prep and/or sequencing. The partitioning-tagging-pooling steps can occur more than once, with each round of partitioning occurring based on a different characteristics and tagged using differential tags that are distinguished from other partitions and partitioning means. In some embodiments, the separating comprises partitioning the DNA in the sample into a plurality of partitioned subsamples. In some embodiments, the plurality of partitioned subsamples comprises a first partitioned subsample and a second partitioned subsample. In some embodiments, the first partitioned subsample comprises methylated DNA (e.g., methyl cytosine) in a greater proportion than the second partitioned subsample. In some embodiments, the partitioning the DNA into a plurality of subsamples comprises contacting the DNA with an agent that recognizes methyl cytosine in the DNA. The partitioning step can occur prior to or after capturing an epigenetic target region set of DNA or a sequence-variable target region of the DNA. The partitioning step can occur prior to capturing an epigenetic target region set of DNA or a sequence-variable target region of the DNA. The partitioning step can occur prior to or after capturing an epigenetic target region set of DNA or a sequence-variable target region of the DNA and prior to or after sequencing the DNA. The partitioning step can occur after capturing an epigenetic target region set of DNA or a sequence-variable target regions of the DNA and prior to sequencing the DNA.

Disclosed methods herein comprise analyzing DNA in a sample or subsample, or isolated from a sample or subsample. In some embodiments described herein, the disclosed methods comprise partitioning DNA. In such methods, different forms of DNA (e.g., hypermethylated and hypomethylated DNA) can be physically partitioned based on one or more characteristics of the DNA. This approach can be used to determine, for example, whether certain sequences are hypermethylated or hypomethylated. In some embodiments, a first subsample or aliquot of a sample is subjected to steps for making capture probes as described elsewhere herein and a second subsample or aliquot of a sample is subjected to partitioning. In some embodiments, a sample or subsample or aliquot thereof is subjected to partitioning and differential tagging, followed by a capture step using capture probes for rearranged sequences and optionally additional capture probes, e.g., for sequence-variable and/or epigenetic target regions.

Methylation profiling can involve determining methylation patterns across different regions of the genome. For example, after partitioning molecules based on extent of methylation (e.g., relative number of methylated nucleobases per molecule) and sequencing, the sequences of molecules in the different partitions can be mapped to a reference genome. This can show regions of the genome that, compared with other regions, are more highly methylated or are less highly methylated. In this way, genomic regions, in contrast to individual molecules, may differ in their extent of methylation.

In some embodiments, the partitioning comprises contacting a subsample comprising DNA with an agent that recognizes a modification associated with (e.g., in) the DNA. In some embodiments, the agent that recognizes the modification is an antibody or a methyl binding domain (MBD) protein. In some embodiments, the agent is immobilized on a solid support. In some embodiments, the solid support comprises a bead. In some embodiments, the partitioning comprises immunoprecipitation, e.g., using the agent that recognizes the modification, such as an antibody or an MBD protein, immobilized on solid support.

In some embodiments, the partitioning comprises precipitating methylated DNA. In some embodiments, the partitioning comprises precipitating the methylated DNA to separate it from the unmethylated DNA. In some embodiments, the precipitating the methylated DNA can be performed using any pair of binding partners. In some embodiments, one of the binding partners may be linked to the MBD protein or antibody, and the other binding partner may be linked to a solid support. In some embodiments, the binding partner comprises biotin and streptavidin. In some embodiments, the biotin may be linked to the MBD protein, and the streptavidin may be linked to a solid support. In some embodiments, the MBD protein is linked to a solid support, optionally using any pair of binding partners. In some embodiments, the partitioning comprises immunoprecipitating the methylated DNA. In some embodiments, the partitioning comprises immunoprecipitating the methylated DNA separately from the unmethylated DNA.

In some embodiments, the modification is methylation, and in some such embodiments, the partitioning comprises partitioning on the basis of methylation level. In some such embodiments, the agent is a methyl binding reagent. In some embodiments, the methyl binding reagent specifically recognizes 5-methylcytosine. In some such embodiments, the agent is a hydroxymethyl binding reagent. In some embodiments, the methyl binding reagent specifically recognizes 5-hydroxymethylcytosine, biotinylated 5-hydroxymethylcytosine, glucosylated 5-hydroxymethylcytosine, or sulfonylated 5-hydroxymethylcytosine. In some embodiments, the partitioning comprises partitioning on the basis of binding to a protein comprising contacting the sample comprising the DNA with a binding reagent specific for the protein. In some such embodiments, binding reagent specifically binds a methylated protein or an acetylated protein, such as a methylated or acetylated histone, or an unmethylated protein or an unacetylated protein such as an unmethylated or unacetylated histone. In some embodiments, the binding reagent specifically binds an unmethylated or unacetylated protein epitope.

In some embodiments, the modification is hydroxymethylation, and in some such embodiments, the partitioning comprises partitioning on the basis of hydroxymethylation level. In some such embodiments, the agent is a hydroxymethyl binding reagent, such as an antibody. In some embodiments, the hydroxymethyl binding reagent (e.g., antibody) specifically recognizes 5-hydroxymethylcytosine (5-hmC). In some embodiments, a modification such as hydroxymethylation is labeled (e.g., biotinylated, glucosylated, or sulfonated) before being contacted with an agent that recognizes the labeled form of the modification. For example, 5-hmC can be enzymatically glucosylated and then partitioned based on binding to J-binding protein 1. Exemplary methods of labeling and/or partitioning 5-hmC are provided, e.g., in Song et al., Nat. Biotech. 29:68-72 (2010); Ko et al., Nature 468:839-843 (2010); and Robertson et al., Nucleic Acids Res. 39: e55 (2011).

Where immunoprecipitation is used and involves an antibody that recognizes single-stranded DNA, the DNA may be converted to double-stranded form by complementary strand synthesis before a subsequent step. Such synthesis may use an adapter as a primer binding site, or can use random priming.

Partitioning nucleic acid molecules in a sample can increase a rare signal, e.g., by enriching rare nucleic acid molecules that are more prevalent in one partition of the sample. For example, a genetic variation present in hypermethylated DNA but less (or not) present in hypomethylated DNA can be more easily detected by partitioning a sample into hypermethylated and hypomethylated nucleic acid molecules. By analyzing multiple partitions of a sample, a multi-dimensional analysis of a single molecule can be performed and hence, greater sensitivity can be achieved. Partitioning may include physically partitioning nucleic acid molecules into partitions or subsamples based on the presence or absence of one or more methylated nucleobases. A sample may be partitioned into subsamples (and/or further partitioned as described elsewhere herein, such as further partitioning of DNA isolated from a subsample) based on a characteristic that is indicative of differential gene expression or a disease state. A sample may be partitioned based on a characteristic, or combination thereof that provides a difference in signal between a normal and diseased state during analysis of nucleic acids, e.g., cell free DNA (cfDNA), non-cfDNA, tumor DNA, circulating tumor DNA (ctDNA) and cell free nucleic acids (cfNA).

In some embodiments, hypermethylation and/or hypomethylation variable epigenetic target regions are analyzed to determine whether they show differential methylation characteristic of tumor cells or cells of a type that does not normally contribute to the DNA sample being analyzed (such as cfDNA), and/or particular immune cell types.

In some instances, heterogeneous DNA in a sample is partitioned into two or more partitions (e.g., at least 3, 4, 5, 6 or 7 partitions). In some embodiments, each partition is differentially tagged. Tagged partitions can then be pooled together for collective sample prep and/or sequencing. The partitioning-tagging-pooling steps can occur more than once, with each round of partitioning occurring based on a different characteristic (examples provided herein), and tagged using differential tags that are distinguished from other partitions and partitioning means. In other instances, the differentially tagged partitions are separately sequenced.

In some embodiments, sequence reads from differentially tagged and pooled DNA are obtained and analyzed in silico. After sequencing, analysis of reads can be performed on a partition-by-partition level, as well as a whole DNA population level. Tags are used to sort reads from different partitions. Analysis to detect genetic variants can be performed on a partition-by-partition level, as well as whole nucleic acid population level. For example, analysis can include in silico analysis to determine genetic variants, such as copy number variations (CNVs), single nucleotide variations (SNVs), insertions/deletions (indels), and/or fusions in nucleic acids in each partition. In some instances, in silico analysis can include analysis to determine epigenetic variation (one or more of methylation chromatin structure, etc.). Analysis can include in silico analysis using sequence information, genomic coordinates length, coverage, and/or copy number. For example, coverage of sequence reads can be used to determine nucleosome positioning in chromatin. Tags can be used to sort reads from different partitions. Higher coverage can correlate with higher nucleosome occupancy in genomic region while lower coverage can correlate with lower nucleosome occupancy or nucleosome depleted region (NDR).

Examples of characteristics that can be used for partitioning isolated DNA include sequence length, methylation level, nucleosome binding, sequence mismatch, immunoprecipitation, and/or proteins that bind to DNA. Resulting partitions can include one or more of the following nucleic acid forms: single-stranded DNA (ssDNA), double-stranded DNA (dsDNA), shorter DNA fragments and longer DNA fragments. In some embodiments, partitioning based on a cytosine modification (e.g., cytosine methylation) or methylation generally is performed and is optionally combined with at least one additional partitioning step, which may be based on any of the foregoing characteristics or forms of DNA. In some embodiments, a heterogeneous population of nucleic acids is partitioned into nucleic acids with one or more epigenetic modifications and without the one or more epigenetic modifications. Examples of epigenetic modifications include presence or absence of methylation; level of methylation; type of methylation (e.g., 5-methylcytosine versus other types of methylation, such as adenine methylation and/or cytosine hydroxymethylation); and association and level of association with one or more proteins, such as histones. Alternatively or additionally, a heterogeneous population of nucleic acids can be partitioned into nucleic acid molecules associated with nucleosomes and nucleic acid molecules devoid of nucleosomes. Alternatively or additionally, a heterogeneous population of nucleic acids may be partitioned into single-stranded DNA (ssDNA) and double-stranded DNA (dsDNA). Alternatively, or additionally, a heterogeneous population of nucleic acids may be partitioned based on nucleic acid length (e.g., molecules of up to 160 bp and molecules having a length of greater than 160 bp).

In some cases, different procedures are applied to different partitions to determine different characteristics of the initial sample. In some embodiments, the DNA of at least one partition is subjected to end repair and sequencing. In some embodiments at least one partition is not subjected to the end repair and sequencing procedure. In cases where the method comprises a conversion procedure, corresponding sequences from the converted and non-converted partitions can be compared to identify single nucleotides that have undergone conversion and therefore identify corresponding modified nucleosides in the initial sample.

In some embodiments, partition tagging comprises tagging molecules in each partition with a partition tag. After re-combining partitions (e.g., to reduce the number of sequencing runs needed and avoid unnecessary cost) and sequencing molecules, the partition tags identify the source partition. In another embodiment, different partitions are tagged with different sets of molecular tags, e.g., comprised of a pair of barcodes. In this way, each molecular barcode indicates the source partition as well as being useful to distinguish molecules within a partition. For example, a first set of 35 barcodes can be used to tag molecules in a first partition, while a second set of 35 barcodes can be used tag molecules in a second partition.

In some embodiments, after partitioning and tagging with partition tags, the molecules may be pooled for sequencing in a single run. In some embodiments, a sample tag is added to the molecules, e.g., in a step subsequent to addition of partition tags and pooling. Sample tags can facilitate pooling material generated from multiple samples for sequencing in a single sequencing run.

Alternatively, in some embodiments, partition tags may be correlated to the sample as well as the partition. As a simple example, a first tag can indicate a first partition of a first sample; a second tag can indicate a second partition of the first sample; a third tag can indicate a first partition of a second sample; and a fourth tag can indicate a second partition of the second sample.

While tags may be attached to molecules already partitioned based on one or more characteristics, the final tagged molecules in the library may no longer possess that characteristic. For example, while single-stranded DNA molecules may be partitioned and tagged, the final tagged molecules in the library are likely to be double stranded. Similarly, while DNA may be subject to partition based on different levels of methylation, in the final library, tagged molecules derived from these molecules are likely to be unmethylated. Accordingly, the tag attached to a molecule in the library typically indicates the characteristic of the “parent molecule” from which the ultimate tagged molecule is derived, not necessarily to characteristic of the tagged molecule, itself.

As an example, barcodes 1, 2, 3, 4, etc. are used to tag and label molecules in the first partition; barcodes A, B, C, D, etc. are used to tag and label molecules in the second partition; and barcodes a, b, c, d, etc. are used to tag and label molecules in the third partition. Differentially tagged partitions can be pooled prior to sequencing. Differentially tagged partitions can be separately sequenced or sequenced together concurrently, e.g., in the same flow cell of an Illumina sequencer.

After sequencing, analysis of reads can be performed on a partition-by-partition level, as well as a whole DNA population level. Tags are used to sort reads from different partitions. Analysis can include in silico analysis to determine genetic and epigenetic variation (one or more of methylation, chromatin structure, etc.) using sequence information, genomic coordinates length, coverage, and/or copy number. In some embodiments, higher coverage can correlate with higher nucleosome occupancy in a genomic region, while lower coverage can correlate with lower nucleosome occupancy or a nucleosome depleted region (NDR).

The agents used to partition populations of nucleic acids within a sample can be affinity agents, such as antibodies with the desired specificity, natural binding partners or variants thereof (Bock et al., Nat Biotech 28:1106-1114 (2010); Song et al., Nat Biotech 29:68-72 (2011)), or artificial peptides selected e.g., by phage display to have specificity to a given target. In some embodiments, the agent used in the partitioning is an agent that recognizes a modified nucleobase. In some embodiments, the modified nucleobase recognized by the agent is a modified cytosine, such as a methylcytosine (e.g., 5-methylcytosine). In some embodiments, the modified nucleobase recognized by the agent is a product of a procedure that affects the first nucleobase in the DNA differently from the second nucleobase in the DNA of the sample. In some embodiments, the modified nucleobase may be a “converted nucleobase,” meaning that its base pairing specificity was changed by a procedure. For example, certain procedures convert unmethylated or unmodified cytosine to dihydrouracil, or more generally, at least one modified or unmodified form of cytosine undergoes deamination, resulting in uracil (considered a modified nucleobase in the context of DNA) or a further modified form of uracil. Examples of partitioning agents include antibodies, such as antibodies that recognize a modified nucleobase, which may be a modified cytosine, such as a methylcytosine (e.g., 5-methylcytosine). In some embodiments, the partitioning agent is an antibody that recognizes a modified cytosine other than 5-methylcytosine, such as 5-carboxylcytosine (5-caC). Alternative partitioning agents include methyl binding domain (MBDs) and methyl binding proteins (MBPs) as described herein, including proteins such as MeCP2, MBD2, and antibodies preferentially binding to 5-methylcytosine. Where an antibody is used to immunoprecipitate methylated DNA, the methylated DNA may be recovered in single-stranded form. In such embodiments, a second strand can be synthesized. Hypermethylated (and optionally intermediately methylated) subsamples may then be contacted with a methylation sensitive nuclease that does not cleave hemi-methylated DNA, such as HpaII, BstUI, or Hin6i. Alternatively or in addition, hypomethylated (and optionally intermediately methylated) subsamples may then be contacted with a methylation dependent nuclease that cleaves hemi-methylated DNA.

Additional, non-limiting examples of partitioning agents are histone binding proteins which can separate nucleic acids bound to histones from free or unbound nucleic acids. Examples of histone binding proteins that can be used in the methods disclosed herein include RBBP4, RbAp48 and SANT domain peptides.

In some embodiments, partitioning can comprise both binary partitioning and partitioning based on degree/level of modifications. For example, methylated fragments in a DNA sample can be partitioned by methylated DNA immunoprecipitation (MeDIP), or all methylated fragments can be partitioned from unmethylated fragments using methyl binding domain proteins (e.g., MethylMinder Methylated DNA Enrichment Kit (ThermoFisher Scientific). Subsequently, additional partitioning may involve eluting fragments having different levels of methylation by adjusting the salt concentration in a solution with the methyl binding domain and bound fragments. As salt concentration increases, fragments having greater methylation levels are eluted.

Analyzing DNA may comprise detecting or quantifying DNA of interest. Analyzing DNA can comprise detecting genetic variants and/or epigenetic features (e.g., DNA methylation and/or DNA fragmentation). In some embodiments, the DNA of interest is one or more differentially methylated regions of the DNA. In some embodiments, the detecting or quantifying the DNA of interest comprises quantifying and/or detecting a level of methylation at one or more differentially methylated regions of the DNA. In some embodiments, quantifying and/or detecting the level of methylation at one or more differentially methylated regions of the DNA comprises sequencing at least a portion of the amplified DNA or quantitative PCR (qPCR). In some embodiments, the DNA of interest is a copy number variant. In some embodiments, the detecting or quantifying the DNA of interest comprises quantifying and/or detecting a level of a copy number variant of the DNA. In some embodiments, quantifying and/or detecting the level of a copy number variant of the DNA comprises quantitative PCR (qPCR).

In some embodiments, methylation levels can be determined using partitioning, modification-sensitive conversion such as bisulfite conversion, direct detection during sequencing, methylation-sensitive restriction enzyme digestion, methylation-dependent restriction enzyme digestion, or any other suitable approach. For example, different forms of DNA (e.g., hypermethylated and hypomethylated DNA) can be physically partitioned based on one or more characteristics of the DNA. For example, a methylated DNA binding protein (e.g., an MBD such as MBD2, MBD4, or MeCP2) or an antibody specific for 5-methylcytosine (as in MeDIP) can be used to partition the DNA. This approach can be used to determine, for example, whether certain sequences are hypermethylated or hypomethylated. In some embodiments, a DNA fragmentation pattern can be determined based on endpoints and/or centerpoints of DNA molecules, such as cfDNA molecules.

In some instances, the final partitions are enriched in nucleic acids having different extents of modifications (overrepresentative or underrepresentative of modifications). Overrepresentation and underrepresentation can be defined by the number of modifications born by a nucleic acid relative to the median number of modifications per strand in a population. For example, if the median number of 5-methylcytosine residues in nucleic acid in a sample is 2, a nucleic acid including more than two 5-methylcytosine residues is overrepresented in this modification and a nucleic acid with 1 or zero 5-methylcytosine residues is underrepresented. The effect of affinity separation is to enrich for nucleic acids overrepresented in a modification in a bound phase and for nucleic acids underrepresented in a modification in an unbound phase (i.e. in solution). The nucleic acids in the bound phase can be eluted before subsequent processing.

When using MeDIP or MethylMiner®Methylated DNA Enrichment Kit (ThermoFisher Scientific) various levels of methylation can be partitioned using sequential elutions. For example, a hypomethylated partition (no methylation) can be separated from a methylated partition by contacting the nucleic acid population with the MBD from the kit, which is attached to magnetic beads. The beads are used to separate out the methylated nucleic acids from the non-methylated nucleic acids. Subsequently, one or more elution steps are performed sequentially to elute nucleic acids having different levels of methylation. For example, a first set of methylated nucleic acids can be eluted at a salt concentration of 160 mM or higher, e.g., at least 150 mM, at least 200 mM, 300 mM, 400 mM, 500 mM, 600 mM, 700 mM, 800 mM, 900 mM, 1000 mM, or 2000 mM. After such methylated nucleic acids are eluted, magnetic separation is once again used to separate higher level of methylated nucleic acids from those with lower level of methylation. The elution and magnetic separation steps can be repeated to create various partitions such as a hypomethylated partition (enriched in nucleic acids comprising no methylation), a methylated partition (enriched in nucleic acids comprising low levels of methylation), and a hyper methylated partition (enriched in nucleic acids comprising high levels of methylation).

In some methods, nucleic acids bound to an agent used for affinity separation-based partitioning are subjected to a wash step. The wash step washes off nucleic acids weakly bound to the affinity agent. Such nucleic acids can be enriched in nucleic acids having the modification to an extent close to the mean or median (i.e., intermediate between nucleic acids remaining bound to the solid phase and nucleic acids not binding to the solid phase on initial contacting of the sample with the agent).

The affinity separation results in at least two, and sometimes three or more partitions of nucleic acids with different extents of a modification. While the partitions are still separate, the nucleic acids of at least one partition, and usually two or three (or more) partitions are linked to nucleic acid tags, usually provided as components of adapters, with the nucleic acids in different partitions receiving different tags that distinguish members of one partition from another. The tags linked to nucleic acid molecules of the same partition can be the same or different from one another. But if different from one another, the tags may have part of their code in common so as to identify the molecules to which they are attached as being of a particular partition.

For further details regarding portioning nucleic acid samples based on characteristics such as methylation, see WO2018/119452, which is incorporated herein by reference.

In some embodiments, the nucleic acid molecules can be partitioned into different partitions based on the nucleic acid molecules that are bound to a specific protein or a fragment thereof and those that are not bound to that specific protein or fragment thereof.

Nucleic acid molecules can be partitioned based on DNA-protein binding. Protein-DNA complexes can be partitioned based on a specific property of a protein. Examples of such properties include various epitopes, modifications (e.g., histone methylation or acetylation) or enzymatic activity. Examples of proteins which may bind to DNA and serve as a basis for fractionation may include, but are not limited to, protein A and protein G. Any suitable method can be used to partition the nucleic acid molecules based on protein bound regions. Examples of methods used to partition nucleic acid molecules based on protein bound regions include, but are not limited to, SDS-PAGE, chromatin-immuno-precipitation (ChIP), heparin chromatography, and asymmetrical field flow fractionation (AF4).

In some embodiments, the partitioning comprises contacting the DNA with a methylation sensitive restriction enzyme (MSRE) and/or a methylation dependent restriction enzyme (MDRE). Following the treatment of the DNA with a MSRE or a MDRE, the DNA may be partitioned based on size to generate hypermethylated (longest DNA molecules following MSRE treatment and shortest DNA fragments following MDRE treatment), intermediate (intermediate length DNA molecules following MSRE or MDRE treatment), and hypomethylated (shortest DNA molecules following MSRE treatment and longest DNA fragments following MDRE treatment) subsamples.

In some embodiments, the partitioning is performed by contacting the nucleic acids with a methyl binding domain (“MBD”) of a methyl binding protein (“MBP”). In some such embodiments, the nucleic acids are contacted with an entire MBP. In some embodiments, an MBD binds to 5-methylcytosine (5mC), and an MBP comprises an MBD and is referred to interchangeably herein as a methyl binding protein or a methyl binding domain protein. In some embodiments, MBD is coupled to paramagnetic beads, such as Dynabeads® M-280 Streptavidin via a biotin linker. Partitioning into fractions with different extents of methylation can be performed by eluting fractions by increasing the NaCl concentration.

In some embodiments, bound DNA is eluted by contacting the antibody or MBD with a protease, such as proteinase K. This may be performed instead of or in addition to elution steps using NaCl as discussed above.

Examples of agents that recognize a modified nucleobase contemplated herein include, but are not limited to:
(a) MeCP2 is a protein that preferentially binds to 5-methyl-cytosine over unmodified cytosine.
(b) RPL26, PRP8 and the DNA mismatch repair protein MHS6 preferentially bind to 5-hydroxymethyl-cytosine over unmodified cytosine.
(c) FOXK1, FOXK2, FOXP1, FOXP4 and FOXI3 preferably bind to 5-formyl-cytosine over unmodified cytosine (Iurlaro et al., Genome Biol. 14: R119 (2013)).
(d) Antibodies specific to one or more methylated or modified nucleobases or conversion products thereof, such as 5mC, 5-caC, or DHU.

In general, elution is a function of the number of modifications, such as the number of methylated sites per molecule, with molecules having more methylation eluting under increased salt concentrations. To elute the DNA into distinct populations based on the extent of methylation, one can use a series of elution buffers of increasing NaCl concentration. Salt concentration can range from about 100 nm to about 2500 mM NaCl. In one embodiment, the process results in three (3) partitions. Molecules are contacted with a solution at a first salt concentration and comprising a molecule comprising an agent that recognizes a modified nucleobase, which molecule can be attached to a capture moiety, such as streptavidin. At the first salt concentration a population of molecules will bind to the agent and a population will remain unbound. The unbound population can be separated as a “hypomethylated” population. For example, a first partition enriched in hypomethylated form of DNA is that which remains unbound at a low salt concentration, e.g., 100 mM or 160 mM. A second partition enriched in intermediate methylated DNA is eluted using an intermediate salt concentration, e.g., between 100 mM and 2000 mM concentration. This is also separated from the sample. A third partition enriched in hypermethylated form of DNA is eluted using a high salt concentration, e.g., at least about 2000 mM.

In some embodiments, a monoclonal antibody raised against 5-methylcytidine (5mC) is used to purify methylated DNA. DNA is denatured, e.g., at 95° C. in order to yield single-stranded DNA fragments. Protein G coupled to standard or magnetic beads as well as washes following incubation with the anti-5mC antibody are used to immunoprecipitate DNA bound to the antibody. Such DNA may then be eluted. Partitions may comprise unprecipitated DNA and one or more partitions eluted from the beads.

In some embodiments, the partitions of DNA are desalted and concentrated in preparation for enzymatic steps of library preparation.

Sequences that comprise aberrantly high copy numbers may tend to be hypermethylated. Accordingly, in some embodiments, the DNA contacted with target-specific probes specific for members of an epigenetic target region set comprising a plurality of target regions that are both type-specific differentially methylated regions and copy number variants comprises at least a portion of a hypermethylated partition. The DNA from or comprising at least a portion of the hypermethylated partition may or may not be combined with DNA from or comprising at least a portion of one or more other partitions, such as an intermediate partition or a hypomethylated partition.

In some cases, different procedures are applied to different partitions to determine different characteristics of the initial sample. In some embodiments, the DNA of at least one partition is subjected to an end repair and sequencing procedure. In some embodiments at least one partition is not subjected to the end repair and sequencing procedure. In cases where the sequencing procedure comprises a conversion procedure, corresponding sequences from the converted and non-converted partitions can be compared to identify single nucleotides that have undergone conversion and therefore identify corresponding modified nucleosides in the initial sample.

Disclosed methods herein can comprise analyzing DNA in a sample. In some embodiments described herein, the disclosed methods comprise partitioning DNA. In such methods, different forms of DNA (e.g., hypermethylated and hypomethylated DNA) can be physically partitioned based on one or more characteristics of the DNA. This approach can be used to determine, for example, whether certain sequences are hypermethylated or hypomethylated and whether certain hypermethylated regions overlap with regions with copy number variants. In some embodiments, a first subsample or aliquot of a sample is subjected to steps for making capture probes as described elsewhere herein and a second subsample or aliquot of a sample is subjected to partitioning. In some embodiments, a sample or subsample or aliquot thereof is subjected to partitioning and differential tagging, followed by a capture step using capture probes for rearranged sequences and optionally additional capture probes, e.g., for sequence-variable and/or epigenetic target regions.

Methylation profiling can involve determining methylation patterns across different regions of the genome. For example, after partitioning molecules based on extent of methylation (e.g., relative number of methylated nucleobases per molecule) and sequencing, the sequences of molecules in the different partitions can be mapped to a reference genome. This can show regions of the genome that, compared with other regions, are more highly methylated or are less highly methylated. In this way, genomic regions, in contrast to individual molecules, may differ in their extent of methylation.

F. Conversion, Contacting DNA with a Deaminase

The methods disclosed herein can comprise subjecting DNA, such as DNA isolated from a sample or one or more subsamples or DNA contained in one or more subsamples, to a procedure that affects a first nucleobase in the DNA differently from a second nucleobase. In some embodiments, the first nucleobase is a modified or an unmodified nucleobase, and the second nucleobase is a modified or an unmodified nucleobase different from the first nucleobase. In some embodiments, the first nucleobase and the second nucleobase have the same base pairing specificity. In some embodiments, the procedure that affects a first nucleobase in the DNA differently from a second nucleobase comprises a conversion procedure that changes the base pairing specificity of the base or does not change the base pairing specificity of the base, depending on the modification status of the base. In some embodiments, the first nucleobase is an unmodified cytosine and the second nucleobase is a modified cytosine (e.g., 5-methylcytosine or 5-hydroxymethylcytosine).

In some embodiments, the procedure that affects a first nucleobase of the DNA differently from a second nucleobase of the DNA is conversion. In some embodiments, the procedure that affects a first nucleobase of the DNA differently from a second nucleobase of the DNA is methylation-sensitive conversion. The methods disclosed herein can comprise contacting DNA in a sample with a deaminase, thereby providing a converted sample. In some embodiments, the deaminase is a methyl-sensitive deaminase or a methyl-insensitive deaminase. In some embodiments, the deaminase is a dsDNA deaminase and/or a ssDNA deaminase. This step of contacting the DNA in the sample with a deaminase can be referred to as, or be included in, a conversion procedure, such as any of the conversion procedures described elsewhere herein. For an exemplary description of conversion using a deaminase, see, e.g., Schutsky et al., Nature Biotechnology 2018; 36:1083-1090. In some embodiments, the DNA in the converted sample is then sequenced, and a level or methylation at one or more differentially methylated regions of the DNA is quantified, or a variation of the copy number at one or more regions of the DNA is quantified.

Table 1 summarizes exemplary methods of deamination with the type of modified bases detectable with these methods. These are described in more detail below. A gene is considered to comprise a DMR when the DMR is located within an untranslated region (UTR), intron, or exon of the gene, or within 500 nucleotides of either the 5′ end of the sense strand of the 5′ UTR or the 3′ end of the sense strand of the 3′ UTR.

| Conversion method | Base modification detectable |
|---|---|
| Bisulfite conversion | 5 mC and 5hmC distinguishable from C |
| Oxidative bisulfite conversion | 5 mC distinguishable from 5 hmC and C |
| Tet-assisted bisulfite | 5 hmC distinguishable from 5 mC and C |
| (TAB) conversion | |
| APOBEC-coupled epigenetic | 5 hmC distinguishable from 5 mC and C |
| (ACE) conversion | |
| Enzymatic methyl-seq | 5 mC and 5 hmC distinguishable from C |
| (EM-seq). | |
| Single-enzyme | 5 mC and 5 hmC distinguishable from C |
| 5-methylctyosine | |
| sequencing (SEM-seq) | |
| Tet-assisted pyridine borane | 5 mC and 5 hmC distinguishable from C |
| sequencing (TAPS) | |

As outlined below, there are various methods of detecting and/or identifying modified nucleosides that rely on a conversion procedure that changes the base-pairing specificity of a nucleoside, based on the modification status of the nucleosides. These changes of base-pairing specificity can then be detected, and thus the modification status of the nucleoside inferred, by sequencing.

In some embodiments, the conversion procedure used in the methods of the disclosure is one that changes the base pairing specificity of a modified nucleoside (e.g. methylated cytosine), but does not change the base pairing specificity of the corresponding unmodified nucleoside (e.g. cytosine) or does not change the base pairing specificity of any un-modified nucleoside (e.g. cytosine, adenosine, guanosine and thymidine (or uracil)). Advantages of methods that do not convert the base-pairing specificity of unmodified nucleosides include reduced loss of sequence complexity, higher sequencing efficiency and reduced alignment losses. Additionally, methods such as TAPS may in some cases be preferred over methods such as bisulfite sequencing and EM-seq because they are less destructive (especially important for low yield samples such as cfDNA or FFPE samples) and do not require denaturation, meaning that non-conversion errors are theoretically more likely to be random. In methods that require denaturation for conversion, failure to denature a DNA molecule will result in non-conversion of all bases in the DNA molecule. As biological changes in methylation are predominantly concerted to a localized regions of interest, these non-random (localized) non-conversion events can appear as false negatives (non-methylated regions). Random non-conversion methods can maximally affect a low percent of bases within a region, and thus the specificity of methylation change detection can be maximized (reduce false positives) by placing a threshold on percentage of bases within a region that are methylated/non-methylated. Hence, in some cases, a conversion procedure that does not involve denaturation is preferred.

In other cases, the conversion procedure used in the methods of the disclosure is one that changes the base pairing specificity of an unmodified nucleoside (e.g. cytosine), but does not change the base pairing specificity of the corresponding modified nucleoside (e.g. methylated cytosine such as 5hmC and/or 5mC). Such methods include, for example, bisulfite sequencing.

The skilled person can select a suitable method according to their needs, including which nucleoside modifications are to be detected and/or identified and which type of modified base is used in an end repair reaction.

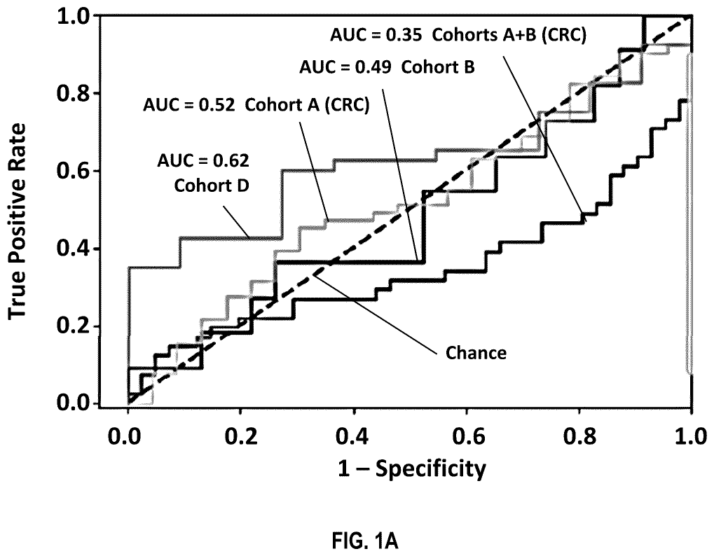

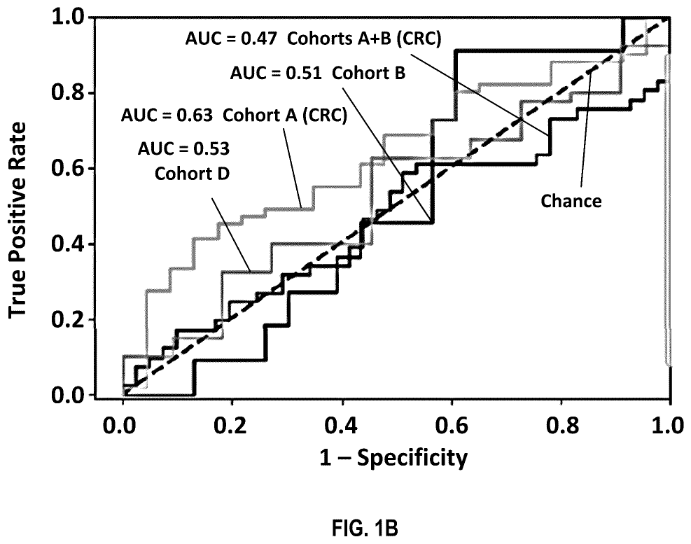

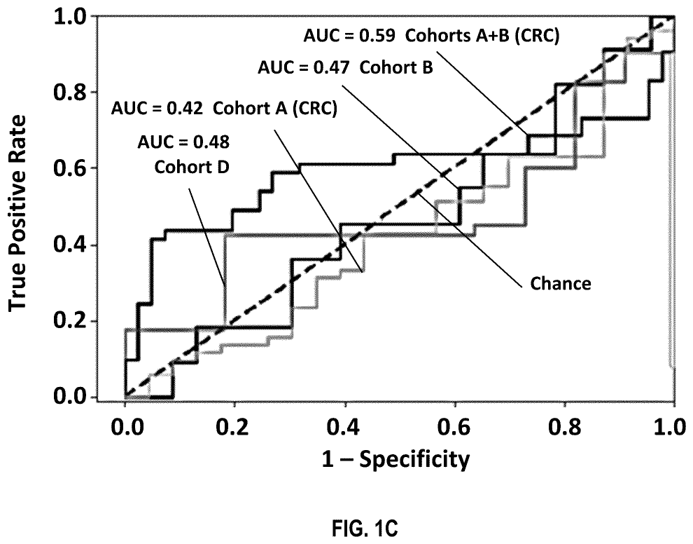

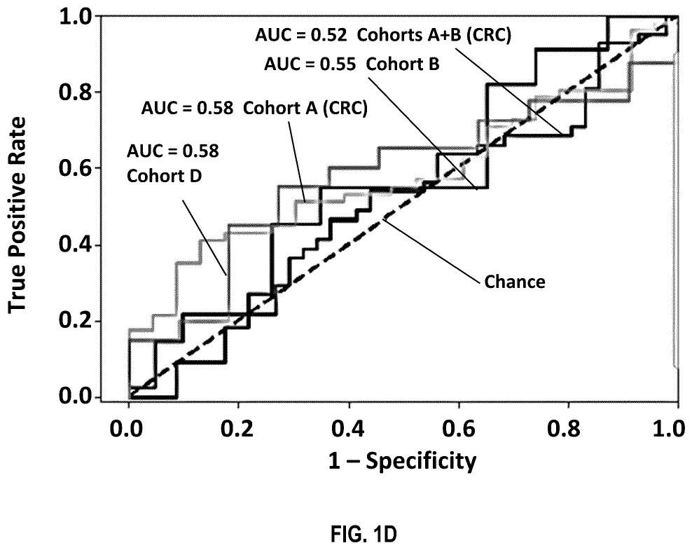

In some embodiments, the conversion procedure converts modified nucleosides. In some embodiments, the conversion procedure which converts modified nucleosides comprises Tet-assisted conversion with a substituted borane reducing agent, optionally wherein the substituted borane reducing agent is 2-picoline borane, borane pyridine, tert-butylamine borane, ammonia borane or pyridine borane. In Tet-assisted pic-borane conversion with a substituted borane reducing agent conversion, a TET protein is used to convert 5mC and 5hmC to 5caC, without affecting unmodified C. 5caC, and 5fC if present, are then converted to dihydrouracil (DHU) by treatment with 2-picoline borane (pic-borane) or another substituted borane reducing agent such as borane pyridine, tert-butylamine borane, or ammonia borane, also without affecting unmodified C. See, e.g., Liu et al., Nature Biotechnology 2019; 37:424-429 (e.g., at Supplementary FIG. 1 and Supplementary Note 7). Thus, when this type of conversion is used, the first nucleobase comprises one or more of 5mC, 5fC, 5caC, or 5hmC, and the second nucleobase comprises unmodified cytosine. DHU is read as a T in sequencing. Sequencing of the converted DNA identifies positions that are read as cytosine as being unmodified C positions. Meanwhile, positions that are read as T are identified as being T, 5mC, 5fC, 5caC, or 5hmC. Performing TAP conversion, such as on a DNA sample as described herein, thus facilitates identifying positions containing unmodified C using the sequence reads obtained.

Hence, in these embodiments, an end repair reaction can be performed with dNTPs, wherein the at least one type of dNTP comprises a 5mC or 5hmC, and regions synthesized during an end repair reaction can be identified as those regions comprising 5mC or 5hmC (via T being called at positions which are C in the reference) at non-CpG positions. This procedure encompasses Tet-assisted pyridine borane sequencing (TAPS), described in further detail in Liu et al. 2019, supra. In this method Tet enzyme is used to progressively oxidize 5mC and 5hmC to 5fC or 5caC, then pyridine borane deaminates 5fC, 5CaC to DHU, amplified as T.

Alternatively, protection of 5hmC (e.g., using βGT or 5-hydroxymethylcytosine carbamoyltransferase) can be combined with Tet-assisted conversion with a substituted borane reducing agent, e.g. as described above. In this method (TAPS-B), 5hmC can be protected from conversion, for example through glucosylation using β-glucosyl transferase (BGT), forming (forming 5-glucosylhydroxymethylcytosine) 5ghmC, or through carbamoylation using 5-hydroxymethylcytosine carbamoyltransferase, forming 5cmC. This is described in Yu et al., Cell 2012; 149:1368-80. Treatment with a TET protein such as mTet1 then converts 5mC to 5caC but does not convert C, 5ghmC, or 5cmC. 5caC is then converted to DHU by treatment with pic-borane or another substituted borane reducing agent such as borane pyridine, tert-butylamine borane, or ammonia borane, also without affecting ghmC, 5cmC, or unmodified C. Thus, when Tet-assisted conversion with a substituted borane reducing agent is used, the first nucleobase comprises mC, and the second nucleobase comprises one or more of unmodified cytosine or hmC, such as unmodified cytosine and optionally hmC, fC, and/or caC. Sequencing of the converted DNA identifies positions that are read as cytosine as being either 5hmC or unmodified C positions. Meanwhile, positions that are read as T are identified as being T, 5fC, 5caC, or 5mC. Performing TAPSβ conversion on a sample as described herein thus facilitates distinguishing positions containing unmodified C or 5hmC on the one hand from positions containing 5mC using the sequence reads obtained. Hence, in these embodiments, an end repair reaction can be performed with dNTPs, wherein the at least one type of dNTP comprises a 5mC, and regions synthesized during an end repair reaction can be identified as those regions comprising 5mC (via T being called at positions which are C in the reference) at non-CpG positions. For an exemplary description of this type of conversion, see, e.g., Liu et al., Nature Biotechnology 2019; 37:424-429. 5-hydroxymethylcytosine carbamoyltransferase is described in Yang et al., Bio-protocol, 2023; 12 (17): e4496.

In some embodiments, the conversion procedure converts modified nucleosides. In some embodiments, the conversion procedure which converts modified nucleosides comprises chemical-assisted conversion with a substituted borane reducing agent, optionally wherein the substituted borane reducing agent is 2-picoline borane, borane pyridine, tert-butylamine borane, borane pyridine or ammonia borane. In chemical-assisted conversion with a substituted borane reducing agent, an oxidizing agent such as potassium perruthenate (KRuO₄) (also suitable for use in ox-BS conversion) is used to specifically oxidize 5hmC to 5fC. Treatment with pic-borane or another substituted borane reducing agent such as borane pyridine, tert-butylamine borane, or ammonia borane converts 5fC and 5caC to DHU but does not affect 5mC or unmodified C. Thus, when this type of conversion is used, the first nucleobase comprises one or more of hmC, fC, and caC, and the second nucleobase comprises one or more of unmodified cytosine or mC, such as unmodified cytosine and optionally mC. Sequencing of the converted DNA identifies positions that are read as cytosine as being either 5mC or unmodified C positions. Meanwhile, positions that are read as T are identified as being T, 5fC, 5caC, or 5hmC. Performing this type of conversion as described herein thus facilitates distinguishing positions containing unmodified C or 5mC on the one hand from positions containing 5hmC using the sequence reads obtained. Hence, in these embodiments, an end repair reaction can be performed with dNTPs, wherein at least one type of dNTP comprises a 5hmC, and regions synthesized during an end repair reaction can be identified as those regions comprising 5hmC (via T being called at positions which are C in the reference) at non-CpG positions. For an exemplary description of this type of conversion, see, e.g., Liu et al., Nature Biotechnology 2019; 37:424-429.

Exemplary conversion procedures that change the base-pairing specificity of modified cytosines have been described. However, the methods described herein could in principle use any modified nucleoside and suitable conversion procedure (i.e. single-base epigenetic conversion assay) that changes the base-pairing specificity of the modified nucleoside and thereby allows the modified base to be distinguished from the corresponding unmodified nucleoside and/or other types of modification when sequenced. For example, any conversion procedure could be used allowing any one of N⁶-methyladenine (6 mA), N⁶-hydroxymethyladenine (6hmA), or N⁶-formyladenine (6fA) to be distinguished from unmodified adenosine.

In some embodiments, the conversion procedure converts unmodified nucleosides. In some embodiments, the conversion procedure which converts unmodified nucleosides comprises bisulfite conversion. Treatment with bisulfite converts unmodified cytosine and certain modified cytosine nucleotides (e.g. 5-formyl cytosine (5fC) or 5-carboxylcytosine (5caC)) to uracil whereas other modified cytosines (e.g., 5mC and 5hmC) are not converted. Thus, where bisulfite conversion is used, the first nucleobase comprises one or more of unmodified cytosine, 5fC, 5caC, or other cytosine forms affected by bisulfite, and the second nucleobase may comprise one or more of 5mC and 5hmC, such as 5mC and optionally 5hmC. Sequencing of bisulfite-treated DNA identifies positions that are read as cytosine as being 5mC or 5hmC positions. Meanwhile, positions that are read as T are identified as being T or a bisulfite-susceptible form of C, such as unmodified cytosine, 5fC, or 5caC. Thus, performing bisulfite conversion, such as on a DNA sample as described herein facilitates identifying positions containing 5mC or 5hmC. Hence, in these embodiments, an end repair reaction can be performed with dNTPs, wherein at least one type of dNTP comprises a 5mC and/or a 5hmC, and regions synthesized during an end repair reaction can be identified as those regions comprising 5mC or a 5hmC (via C being called at these positions) at non-CpG positions. For an exemplary description of bisulfite conversion, see, e.g., Moss et al., Nat Commun. 2018; 9:5068.

In some embodiments, the procedure which converts unmodified nucleosides comprises oxidative bisulfite (Ox-BS) conversion. This procedure first converts 5hmC to 5fC, which is bisulfite susceptible, followed by bisulfite conversion. Thus, when oxidative bisulfite conversion is used, the first nucleobase comprises one or more of unmodified cytosine, 5fC, 5caC, 5hmC, or other cytosine forms affected by bisulfite, and the second nucleobase comprises 5mC. Sequencing of Ox-BS converted DNA identifies positions that are read as cytosine as being 5mC positions. Meanwhile, positions that are read as T are identified as being T or a bisulfite-susceptible form of C, such as unmodified cytosine, 5fC, or 5hmC. Hence, in these embodiments, an end repair reaction can be performed with dNTPs, wherein at least one type of dNTP comprises a 5mC, and regions synthesized during an end repair reaction can be identified as those regions comprising 5mC (via C being called at these positions) at non-CpG positions. Performing Ox-BS conversion thus facilitates identifying positions containing mC. For an exemplary description of oxidative bisulfite conversion, see, e.g., Booth et al., Science 2012; 336:934-937.

In some embodiments, the procedure which converts unmodified nucleosides comprises Tet-assisted bisulfite (TAB) conversion. In TAB conversion, 5hmC is protected from conversion and 5mC is oxidized in advance of bisulfite treatment, so that positions originally occupied by 5mC are converted to U while positions originally occupied by 5hmC remain as a protected form of cytosine. For example, as described in Yu et al., Cell 2012; 149:1368-80, β-glucosyl transferase can be used to protect 5hmC (forming 5-glucosylhydroxymethylcytosine (5ghmC)), then a TET protein such as mTet1 can be used to convert 5mC to 5caC, and then bisulfite treatment can be used to convert C and 5caC to U while 5ghmC remains unaffected.

Alternatively, a carbamoyltransferase enzyme, such as 5-hydroxymethylcytosine carbamoyltransferase as described in Yang et al., Bio-protocol, 2023; 12 (17): e4496, can be used to protect hmC (by converting hmC to 5-carbamoyloxymethylcytosine (5cmC)), then a TET protein such as mTet1 can be used to convert mC to caC, and then bisulfite treatment can be used to convert C and caC to U while 5cmC remains unaffected. Thus, when TAB conversion is used, the first nucleobase comprises one or more of unmodified cytosine, 5fC, 5caC, 5mC, or other cytosine forms affected by bisulfite, and the second nucleobase comprises 5hmC. Sequencing of TAB-converted DNA identifies positions that are read as cytosine as being 5hmC positions. Meanwhile, positions that are read as T are identified as being T, or a bisulfite-susceptible form of C, such as unmodified cytosine, 5mC, 5fC, or 5caC. Performing TAB conversion on a first subsample as described herein thus facilitates identifying positions containing 5hmC. Hence, in these embodiments, an end repair reaction can be performed with dNTPs, wherein at least one type of dNTP comprises a 5hmC, and regions synthesized during an end repair reaction can be identified as those regions comprising 5hmC (via C being called at these positions) at non-CpG positions.

In some embodiments, the conversion procedure which converts unmodified cytosines comprises APOBEC-coupled epigenetic (ACE) conversion. In ACE conversion, an AID/APOBEC family DNA deaminase enzyme such as APOBEC3A (A3A) is used to deaminate an unmodified cytosine and 5mC without deaminating 5hmC, 5fC, or 5-caC. Thus, when ACE conversion is used, the first nucleobase comprises unmodified C and/or mC (e.g., unmodified C and optionally mC), and the second nucleobase comprises hmC. Sequencing of ACE-converted DNA identifies positions that are read as cytosine as being 5hmC, 5fC, or 5-caC positions. Meanwhile, positions that are read as T are identified as being T, unmodified C, or 5mC. Performing ACE conversion as described herein thus facilitates distinguishing positions containing 5hmC from positions containing 5mC or unmodified C using the sequence reads obtained from the first subsample. In some embodiments, an end repair reaction can be performed with dNTPs, wherein at least one type of dNTP comprises a 5hmC, and regions synthesized during an end repair reaction can be identified as those regions comprising 5hmC (via C being called at these positions) at non-CpG positions. For an exemplary description of ACE conversion, see, e.g., Schutsky et al., Nature Biotechnology 2018; 36:1083-1090.

In some embodiments, the procedure that affects a first nucleobase in the DNA differently from a second nucleobase in the DNA of the first subsample comprises enzymatic conversion of the first nucleobase, e.g., as in EM-Seq. See, e.g., Vaisvila R, et al. (2019) EM-seq: Detection of DNA methylation at single base resolution from picograms of DNA. bioRxiv; DOI: 10.1101/2019.12.20.884692, available at www.biorxiv.org/content/10.1101/2019.12.20.884692v1. For example, TET2 and T4-βGT or 5-hydroxymethylcytosine carbamoyltransferase (described in Yang et al., Bio-protocol, 2023; 12(17): e4496) can be used to convert 5mC and 5hmC into substrates that cannot be deaminated by a deaminase (e.g., APOBEC3A), and then a deaminase (e.g., APOBEC3A) can be used to deaminate unmodified cytosines, converting them to uracils.

In some embodiments, the procedure that affects a first nucleobase in the DNA differently from a second nucleobase in the DNA comprises enzymatic conversion of the first nucleobase using a non-specific, modification-sensitive double-stranded DNA deaminase, e.g., as in SEM-seq. See, e.g., Vaisvila et al. (2023) Discovery of novel DNA cytosine deaminase activities enables a nondestructive single-enzyme methylation sequencing method for base resolution high-coverage methylome mapping of cell-free and ultra-low input DNA. bioRxiv; DOI: 10.1101/2023.06.29.547047, available at https://www.biorxiv.org/content/10.1101/2023.06.29.547047v1. SEM-Seq employs a non-specific, modification-sensitive double-stranded DNA deaminase (MsddA) in a nondestructive single-enzyme 5-methylctyosine sequencing (SEM-seq) method that deaminates unmodified cytosines. Accordingly, SEM-seq does not require the TET2 and T4-βGT or 5-hydroxymethylcytosine carbamoyltransferase protection and denaturing steps that are of use, e.g., in APOEC3A-based protocols. Additionally, MsddA does not deaminate 5-formylated cytosines (5fC) or 5-carboxylated cytosines (5-caC). In SEM-seq, unmodified cytosines in the DNA are deaminated to uracil and is read as “T” during sequencing. Modified cytosines (e.g., 5mC) are not converted and are read as “C” during sequencing. Cytosines that are read as thymines are identified as unmodified (e.g., unmethylated) cytosines or as thymines in the DNA. Performing SEM-seq conversion thus facilitates identifying positions containing 5mC using the sequence reads obtained. In some embodiments, the procedure that affects a first nucleobase in the DNA differently from a second nucleobase in the DNA comprises enzymatic conversion of unmodified cytosine using MsddA.

In some embodiments, the conversion procedure converts modified nucleosides. In some embodiments, the conversion procedure which converts modified nucleosides comprises enzymatic conversion, such as DM-seq, for example, as described in WO2023/288222A1. In DM-seq, unmodified cytosines in the DNA are enzymatically protected from a subsequent deamination step wherein 5mC in 5mCpG is converted to T. The enzymatically protected unmodified (e.g., unmethylated) cytosines are not converted and are read as “C” during sequencing. Cytosines that are read as thymines (in a CpG context) are identified as methylated cytosines in the DNA.

Thus, when this type of conversion is used, the first nucleobase comprises unmodified (such as unmethylated) cytosine, and the second nucleobase comprises modified (such as methylated) cytosine. Sequencing of the converted DNA identifies positions that are read as cytosine as being unmodified C positions. Meanwhile, positions that are read as T are identified as being T or 5mC. Performing DM-seq conversion thus facilitates identifying positions containing 5mC using the sequence reads obtained.

Exemplary cytosine deaminases for use herein include APOBEC enzymes, for example, APOBEC3A. Generally, AID/APOBEC family DNA deaminase enzymes such as APOBEC3A (A3A) are used to deaminate (unprotected) unmodified cytosine and 5mC. For an exemplary description of APOBEC conversion, see, e.g., Schutsky et al., Nature Biotechnology 2018; 36:1083-1090.

The enzymatic protection of unmodified cytosines in the DNA comprises addition of a protective group to the unmodified cytosines. Such protective groups can comprise an alkyl group, an alkyne group, a carboxyl group, a carboxyalkyl group, an amino group, a hydroxymethyl group, a glucosyl group, a glucosylhydroxymethyl group, an isopropyl group, or a dye. For example, DNA can be treated with a methyltransferase, such as a CpG-specific methyltransferase, which adds the protective group to unmodified cytosines. The term methyltransferase is used broadly herein to refer to enzymes capable of transferring a methyl or substituted methyl (e.g., carboxymethyl) to a substrate (e.g., a cytosine in a nucleic acid). In some embodiments, the DNA is contacted with a CpG-specific DNA methyltransferase (MTase), such as a CpG-specific carboxymethyltransferase (CxMTase), and a substituted methyl donor, such as a carboxymethyl donor (e.g., carboxymethyl-S-adenosyl-L-methionine). See, e.g., WO2021/236778A2. In particular embodiments, the CxMTase can facilitate the addition of a protective carboxymethyl group to an unmethylated cytosine. In some embodiments, the unmethylated cytosine is unmodified cytosine. The carboxymethyl group can prevent deamination of the cytosine during a deamination step (such as a deamination step using an APOBEC enzyme, such as A3A). Substituted methyl or carboxymethyl donors useful in the disclosed methods include but are not limited to, S-adenosyl-L-methionine (SAM) analogs, optionally wherein the SAM analog is carboxy-S-adenosyl-L-methionine (CxSAM). SAM analogs are described, for example, in WO2022/197593A1. The MTase may be, for example, a CpG methyltransferase from Spiroplasma sp. strain MQ1 (M.SssI), DNA-methyltransferase 1 (DNMT1), DNA-methyltransferase 3 alpha (DNMT3A), DNA-methyltransferase 3 beta (DNMT3B), or DNA adenine methyltransferase (Dam). The CxMTase may be a CpG methyltransferase from Mycoplasma penetrans (M.MpeI). In a particular embodiment, the methyltransferase enzyme is a variant of M.MpeI having SEQ ID NO: 1 or SEQ ID NO: 2, or a sequence at least 90%, at least 92%, at least 94%, at least 96%, at least 97%, at least 98%, or at least 99% identical thereto, optionally wherein the amino acid corresponding to position 374 is R or K.

In one embodiment, the methyltransferase enzyme is a variant of M.MpeI having an N374R substitution or an N374K substitution. The methyltransferase of SEQ ID NO: 1 or SEQ ID NO: 2 can further comprise one or more amino acid substitutions selected from a) substitution of one or both residues T300 and E305 with S, A, G, Q, D, or N; b) substitution of one or more residues A323, N306, and Y299 with a positively charged amino acid selected from K, R or H; and/or c) substitution of S323 with A, G, K, R or H, which may enhance the activity of the enzyme.

Optionally, the conversion procedure further includes enzymatic protection of 5ahmCs, such as by glucosylation of the 5hmCs (e.g., using BGT) or by carbamoylation of the 5hmCs (e.g., using 5-hydroxymethylcytosine carbamoyltransferase), in the DNA prior to the deamination of unprotected modified cytosines. In this method, 5hmC can be protected from conversion, for example through glucosylation using β-glucosyl transferase (BGT), forming (5-glucosylhydroxymethylcytosine) 5ghmC, or through carbamoylation using 5-hydroxymethylcytosine carbamoyltransferase, forming 5cmC. This is described, for example, in Yu et al., Cell 2012; 149:1368-80, and in Yang et al., Bio-protocol, 2023; 12 (17): e4496. Glucosylation or carbamoylation of 5hmC can reduce or eliminate deamination of 5hmC by a deaminase such as APOBEC3A. Treatment with an MTase or CxMTase then adds a protecting group to unmodified (unmethylated) cytosines in the DNA. 5mC (but not protected, unmodified cytosine and not 5ghmC or 5cmC) is then deaminated (converted to T in the case of 5mC) by treatment with a deaminase, for example, an APOBEC enzyme (such as APOBEC3A). Sequencing of the converted DNA identifies positions that are read as cytosine as being either 5hmC or unmodified C positions. Meanwhile, positions that are read as T are identified as being T or 5mC. Performing DM-seq conversion with glucosylation of 5hmC on a sample as described herein thus facilitates distinguishing positions containing unmodified C or 5hmC on the one hand from positions containing 5mC using the sequence reads obtained.

Also provided herein are methods in which alternative base conversion schemes are used. For example, unmethylated cytosines can be left intact while methylated cytosines and hydroxymethylcytosines are converted to a base read as a thymine (e.g., uracil, thymine, or dihydrouracil).

In some embodiments, methylating a cytosine in at least one first complementary strand or second complementary strand comprises contacting the cytosine with a methyltransferase such as DNMT1 or DNMT5. In such embodiments, the step of oxidizing a 5-hydroxymethylated cytosine to 5-formylcytosine (such as by contacting the 5-hydroxymethyl cytosine in a first strand and a second strand with KRuO₄) can be optional.

In some embodiments, converting the modified cytosine in at least one first or second strand to a thymine or a base read as thymine comprises oxidizing a hydroxymethyl cytosine, e.g., the hydroxymethyl cytosine is oxidized to formylcytosine. In some embodiments, oxidizing the hydroxymethyl cytosine to formylcytosine comprises contacting the hydroxymethyl cytosine with a ruthenate, such as potassium ruthenate (KRuO₄).

In some embodiments, the modified cytosine is converted to thymine, uracil, or dihydrouracil. In any such embodiments, amplification methods may comprise uracil-and/or dihydrouracil-tolerant amplification methods, such as PCR using a uracil-and/or dihydrouracil-tolerant DNA polymerase.

In some embodiments, the method comprises converting a formylcytosine and/or a methylcytosine to carboxylcytosine as part of converting the modified cytosine in at least one first or second strand to a thymine or a base read as thymine. For example, converting the formylcytosine and/or the methylcytosine to carboxylcytosine can comprise contacting the formylcytosine and/or the methylcytosine with a TET enzyme, such as TET1, TET2, or TET3. In some embodiments, the method comprises reducing the carboxylcytosine as part of converting the modified cytosine in at least one first or second strand to a thymine or a base read as thymine, and/or the carboxylcytosine is reduced to dihydrouracil. In some embodiments, reducing the carboxylcytosine comprises contacting the carboxylcytosine with a borane or borohydride reducing agent.

In some embodiments, the borane or borohydride reducing agent comprises pyridine borane, 2-picoline borane, borane, tert-butylamine borane, ammonia borane, sodium borohydride, sodium cyanoborohydride (NaBH3CN), lithium borohydride (LiBH4), ethylenediamine borane, dimethylamine borane, sodium triacetoxyborohydride, morpholine borane, 4-methylmorpholine borane, trimethylamine borane, dicyclohexylamine borane, or a salt thereof. In other embodiments, the reducing agent comprises lithium aluminum hydride, sodium amalgam, amalgam, sulfur dioxide, dithionate, thiosulfate, iodide, hydrogen peroxide, hydrazine, diisobutylaluminum hydride, oxalic acid, carbon monoxide, cyanide, ascorbic acid, formic acid, dithiothreitol, beta-mercaptoethanol, or any combination thereof.

As discussed above, in some embodiments, a TET protein can be used to convert 5mC and optionally 5hmC (but not unmodified C) into substrates (e.g., 5caC) that cannot be deaminated by a deaminase, and then a deaminase (e.g., APOBEC3A) can be used to deaminate unmodified cytosines, converting them to uracils. Various TET enzymes may be used in the disclosed methods as appropriate. In some embodiments, the one or more TET enzymes comprise TETv. TETv is described in U.S. Pat. No. 10,260,088 and its sequence is SEQ ID NO: 1 therein (SEQ ID NO: 3 in the present application). In some embodiments, the one or more TET enzymes comprise TETcd. TETcd is described in U.S. Pat. No. 10,260,088 and its sequence is SEQ ID NO: 3 therein (SEQ ID NO: 4 in the present application). In some embodiments, the one or more TET enzymes comprise TET1. In some embodiments, the one or more TET enzymes comprise TET2. TET2 may be expressed and used as a fragment comprising TET2 residues 1129-1480 joined to TET2 residues 1844-1936 by a linker (SEQ ID NO: 5 of the present application) as described, e.g., in U.S. Pat. No. 10,961,525. In some embodiments, the one or more TET enzymes comprise TET1 and TET2. In some embodiments, the one or more TET enzymes comprise a T1372 TET mutant, such as T1372S. In some embodiments, the one or more TET enzymes comprise a V1900 TET mutant, such as a V1900A, V1900C, V1900G, V1900I, or V1900P TET mutant. In some embodiments, the one or more TET enzymes comprise a V1900 TET2 mutant, such as a V1900A, V1900C, V1900G, V1900I, or V1900P TET2 mutant. Examples of V1900A, V1900C, V1900G, V1900I, and V1900P TET2 mutants are provided as SEQ ID NOs: 6-10. In some embodiments, the V1900 TET mutant has at least 80%, 85%, 90%, 95%, 96%, 97%, 98%, or 99% sequence identity to SEQ ID NO: 6, 7, 8, 9, or 10. Position 1900 of the wild-type TET2 sequence corresponds to position 438 in each of SEQ ID NOs: 5-10. It can be beneficial to use a TET enzyme that maximizes formation of 5-carboxylcytosine (5-caC) relative to less oxidized modified cytosines, particularly 5-formylcytosine, because 5-caC is not a substrate for enzymatic deamination, e.g., by APOBEC enzymes such as APOBEC3A. Maximizing formation of 5-caC thus reduces the risk of false calls in which a base is identified as unmethylated because it underwent deamination even though it was methylated (or hydroxymethylated) in the original sample. Accordingly, in some embodiments, the TET enzyme comprises a mutation that increases formation of 5-caC. Exemplary mutations are set forth above. “A mutation that increases formation of 5-caC” means that the TET enzyme having the mutation produces more 5-caC than a TET enzyme that lacks the mutation but is otherwise identical. 5-caC production can be measured as described, e.g., in Liu et al., Nat Chem Biol 13:181-187 (2017) (see Online Methods section, TET reactions in vitro subsection, “driving” conditions). Any variants and/or mutants described in Liu et al. (2017) can be used in the disclosed methods as appropriate.

In some embodiments, the one or more TET enzymes comprise a TET2 enzyme comprising a T1372S mutation, such as TET2-CS-T1372S and TET2-CD-T1372S. Examples of TET2-CS-T1372S and TET2-CD-T1372S are provided as SEQ ID NOs: 11 and 12. A TET2 comprising a T1372S mutation is described in U.S. Pat. No. 10,961,525 and may be expressed and used as a fragment comprising TET2 residues 1129-1480 joined to TET2 residues 1844-1936 by a linker. Position 1372 of TET2 corresponds to position 258 of SEQ ID NO: 21 (wild type TET2 catalytic domain) of U.S. Pat. No. 10,961,525. Thus, the sequence of a T1372S TET2 catalytic domain may be obtained by changing the threonine at position 258 of SEQ ID NO: 21 of U.S. Pat. No. 10,961,525 to serine. TET2 comprising a T1372S mutation is also described in Liu et al., Nat Chem Biol. 2017 February; 13 (2): 181-187. As demonstrated in Liu et al., TET2 comprising a T1372S mutation can more efficiently oxidize 5mC to produce 5-carboxylcytosine (5-caC) than other versions of TET2 such as TET2 lacking a T1372S mutation. In some embodiments, the TET2 enzyme comprises SEQ ID NO: 14 or optionally a variant of SEQ ID NO: 14 in which at least 5, 6, 7, or 8 positions match SEQ ID NO: 14 including position 5 of SEQ ID NO: 14. In some embodiments, the TET2 enzyme is a human TET2 enzyme comprising a T1372S mutation. In some embodiments, the TET2 enzyme comprises the sequence of SEQ ID NO: 11. In some embodiments, the TET2 enzyme comprises a sequence having at least 80%, 85%, 90%, 95%, 96%, 97%, 98%, or 99% identity to SEQ ID NO: 11. In some embodiments, the TET2 enzyme comprises a sequence having at least 80%, 85%, 90%, 95%, 96%, 97%, 98%, or 99% identity to SEQ ID NO: 12. In some embodiments, the TET2 enzyme comprises the sequence of SEQ ID NO: 12. The sequences of SEQ ID NOs: 11 and 12 are shown below.

In some embodiments, the deaminase is thermally inactivated after contacting DNA with the deaminase. In some embodiments, the thermal inactivation comprises heating or cooling of the deaminase to a temperature at which the deaminase has reduced or inhibited activity relative to a deaminase that has not been subjected to heating or cooling. In some embodiments, the thermal inactivation completely inhibits the activity of the deaminase or reduces the activity of the deaminase by at least about 5%, about 10%, about 15%, about 20%, about 25%, about 50%, about 75%, about 90%, about 95%, about 98%, about 99%, or 100% relative to a deaminase that has not been subjected to heating or cooling.

G. Contacting DNA with a Methylation-Sensitive or Methylation-Dependent Nuclease

In some embodiments, DNA or a subsample thereof (e.g., a first, second, or third subsample prepared by partitioning a DNA sample as described herein, such as on the basis of a level of a cytosine modification, such as methylation, e.g., 5-methylation, such as of cytosine) or DNA contained in a sample is contacted with a methylation-dependent nuclease or methylation-sensitive nuclease. The contacting can be performed using a sample that has been divided into a plurality of subsamples as disclosed herein, and/or using a sample that has been partitioned into a plurality of subsamples as disclosed herein. Unless otherwise indicated, where partitioning is performed on the basis of a cytosine modification, the first subsample of DNA is the subsample with a higher level of the modification; the second subsample is the subsample with a lower level of the modification; and, when present, the third subsample has a level of the modification intermediate between the first and second subsamples has a level of the modification intermediate between the first and second subsamples.

In some embodiments, methods herein comprise contacting DNA with a methylation-sensitive nuclease, thereby degrading DNA comprising unmethylated sequences or sequences having low levels of methylation. In some such embodiments, the methylation-sensitive nuclease is a methylation-sensitive restriction enzyme (MSRE), thereby degrading DNA comprising an unmethylated recognition site of the MSRE. Methylation-sensitive nucleases can thus be used in methods herein comprising one or more steps that deplete unmodified or unmethylated sequences, such as those that are prevalent in cfDNA from a subject.

In some embodiments, methods herein comprise contacting DNA with a methylation-dependent nuclease, thereby degrading DNA comprising methylated sequences or sequences having high levels of methylation. In some such embodiments, the methylation-dependent nuclease is a methylation-dependent restriction enzyme (MDRE), thereby degrading DNA comprising a methylated recognition site of the MDRE. Methylation-dependent nucleases can thus be used in methods herein comprising one or more steps that deplete modified or methylated sequences, such as those that are prevalent in cfDNA from a subject.

As discussed above, partitioning procedures may result in imperfect sorting of DNA molecules among the subsamples. The choice of a methylation-dependent nuclease or methylation-sensitive nuclease can be made so as to degrade nonspecifically partitioned DNA. For example, the second subsample can be contacted with a methylation-dependent nuclease, such as a methylation-dependent restriction enzyme. This can degrade nonspecifically partitioned DNA in the second subsample (e.g., methylated DNA) to produce a treated second subsample. Alternatively or in addition, the first subsample can be contacted with a methylation-sensitive endonuclease, such as a methylation-sensitive restriction enzyme, thereby degrading nonspecifically partitioned DNA in the first subsample to produce a treated first subsample. Degradation of nonspecifically partitioned DNA in either or both of the first or second subsamples is proposed as an improvement to the performance of methods that rely on accurate partitioning of DNA on the basis of a cytosine modification, e.g., to detect the presence of aberrantly modified DNA in a sample, to determine the tissue of origin of DNA, and/or to determine whether a subject has cancer. For example, such degradation may provide improved sensitivity and/or simplify downstream analyses. In general, where nonspecifically partitioned DNA would be hypermethylated, such as in a hypomethylated partition, a methylation-dependent nuclease, such as a methylation-dependent restriction enzyme, should be used. Conversely, where nonspecifically partitioned DNA would be hypomethylated, such as in a hypermethylated partition, a methylation-sensitive nuclease, such as a methylation-sensitive restriction enzyme, should be used. Methylation-dependent nucleases, such as methylation-dependent restriction enzymes, preferentially cut methylated DNA relative to unmethylated DNA, while methylation-sensitive nucleases, such as methylation-sensitive restriction enzymes, preferentially cut unmethylated DNA relative to methylated DNA.

In contacting a DNA sample or subsample with a nuclease, one or more nucleases can be used. In some embodiments, a subsample is contacted with a plurality of nucleases. The subsample may be contacted with the nucleases sequentially or simultaneously. Simultaneous use of nucleases may be advantageous when the nucleases are active under similar conditions (e.g., buffer composition) to avoid unnecessary sample manipulation. Contacting the second subsample with more than one methylation-dependent restriction enzyme can more completely degrade nonspecifically partitioned hypermethylated DNA. Similarly, contacting the first subsample with more than one methylation-sensitive restriction enzyme can more completely degrade nonspecifically partitioned hypomethylated and/or unmethylated DNA.

In some embodiments, a methylation-dependent nuclease comprises one or more of MspJI, LpnPI, FspEI, or McrBC. In some embodiments, at least two methylation-dependent nucleases are used. In some embodiments, at least three methylation-dependent nucleases are used. In some embodiments, the methylation-dependent nuclease comprises FspEI. In some embodiments, the methylation-dependent nuclease comprises FspEI and MspJI, e.g., used sequentially.

In some embodiments, a methylation-sensitive nuclease comprises one or more of AatII, AccII, AciI, Aor13HI, Aor15HI, BspT104I, BssHII, BstUI, Cfr10I, ClaI, Cpol, Eco52I, HaeII, HapII, HhaI, Hin6I, HpaII, HpyCH4IV, MluI, MspI, Nael, NotI, Nrul, NsbI, PmaCI, Psp1406I, Pvul, SacII, Sall, Smal, and SnaBI. In some embodiments, at least two methylation-sensitive nucleases are used. In some embodiments, at least three methylation-sensitive nucleases are used. In some embodiments, the methylation-sensitive nucleases comprise BstUI and HpaII. In some embodiments, the two methylation-sensitive nucleases comprise HhaI and AccII. In some embodiments, the methylation-sensitive nucleases comprise BstUI, Hpall and Hin6I.

In some embodiments, FspEI is used for digesting the nucleic acid molecules in at least one subsample (e.g., a hypomethylated partition). In some embodiments, BstUI, Hpall and Hin6I are used for digesting the nucleic acid molecules in at least one subsample (e.g., a hypermethylated partition) and FspEI is used for digesting the nucleic acid molecules in at least one other subsample (e.g., a hypomethylated partition). In embodiments involving an intermediately methylated partition, the nucleic acid molecules therein may be digested with a methylation-sensitive nuclease or a methylation-dependent nuclease. In some embodiments, the nucleic acid molecules in an intermediately methylated partition are digested with the same nuclease(s) as the hypermethylated partition. For example, the intermediately methylated partition may be pooled with the hypermethylated partition and then the pooled partitions may be subjected to digestion. In some embodiments, the nucleic acid molecules in an intermediately methylated partition are digested with the same nuclease(s) as the hypomethylated partition. For example, the intermediately methylated partition may be pooled with the hypomethylated partition and then the pooled partitions may be subjected to digestion.

In some embodiments, a subsample is contacted with a nuclease as described above after a step of tagging or attaching adapters to both ends of the DNA. The tags or adapters can be resistant to cleavage by the nuclease using any of the approaches described above. In this approach, cleavage can prevent the nonspecifically partitioned molecule from being carried through the analysis because the cleavage products lack tags or adapters at both ends.

Alternatively, a step of tagging or attaching adapters can be performed after cleavage with a nuclease as described above. Cleaved molecules can be then identified in sequence reads based on having an end (point of attachment to tag or adapter) corresponding to a nuclease recognition site. Processing the molecules in this way can also allow the acquisition of information from the cleaved molecule, e.g., observation of somatic mutations. When tagging or attaching adapters after contacting the subsample with a nuclease, and low molecular weight DNA such as cfDNA is being analyzed, it may be desirable to remove high molecular weight DNA (such as contaminating genomic DNA) from the sample before the contacting step. It may also be desirable to use nucleases that can be heat-inactivated at a relatively low temperature (e.g., 65° C. or less, or 60° C. or less) to avoid denaturing DNA, in that denaturation may interfere with subsequent ligation steps.

Where a DNA sample is partitioned into three subsamples, including a third subsample containing intermediately methylated molecules, the third subsample is in some embodiments contacted with a methylation-sensitive nuclease. Such a step may have any of the features described elsewhere herein with respect to contacting steps, and may be performed before or after a step of tagging or attaching adapters as discussed above. In some embodiments, the first and third subsamples are combined before being contacted with a methylation-sensitive nuclease. Such a step may have any of the features described elsewhere herein with respect to contacting steps, and may be performed before or after a step of tagging or attaching adapters as discussed above. In some embodiments, the first and third subsamples are differentially tagged before being combined.

Alternatively, where a DNA sample is partitioned into three subsamples, including a third subsample containing intermediately methylated molecules, the third subsample is in some embodiments contacted with a methylation-dependent nuclease. Such a step may have any of the features described elsewhere herein with respect to contacting steps, and may be performed before or after a step of tagging or attaching adapters as discussed above. In some embodiments, the second and third subsamples are combined before being contacted with a methylation-dependent nuclease. Such a step may have any of the features described elsewhere herein with respect to contacting steps, and may be performed before or after a step of tagging or attaching adapters as discussed above. In some embodiments, the second and third subsamples are differentially tagged before being combined.

In some embodiments, the DNA is purified after being contacted with the nuclease, e.g., using SPRI beads. Such purification may occur after heat inactivation of the nuclease. Alternatively, purification can be omitted; thus, for example, a subsequent step such as amplification can be performed on the subsample containing heat-inactivated nuclease. In another embodiment, the contacting step can occur in the presence of a purification reagent such as SPRI beads, e.g., to minimize losses associated with tube transfers. After cleavage and heat inactivation, the SPRI beads can be re-used for cleanup by adding molecular crowding reagents (e.g., PEG) and salt.

In some embodiments, DNA fragmentation is detected by determining the endpoints and/or midpoints of sequenced fragments of DNA (e.g., cfDNA). For example, differences in fragmentation patterns may occur depending on whether the fragments originated from a tumor or from healthy cells. To detect tumor-cell derived DNA of cfDNA based on fragmentation, the presence or absence of an increased level of abnormal fragments can be determined at regions with copy-number amplifications, (e.g., proportional to the degree of amplification), e.g., where the increase and abnormality are relative to control or healthy samples.

In some embodiments, where a modification sensitive conversion is performed on a DNA sample or subsample, the subsequent capturing of one or more target region sets (e.g., at least an epigenetic target region set) from that sample or subsample uses target-specific probes that comprise probes specific for a modification state (e.g., of at least one base in the sequence to which the probe hybridizes), e.g., complementary to target sequences that have undergone conversion (e.g., conversion of modified or unmodified cytosines to uracils or analogs thereof, such as DHU, that preferentially pair with adenine) or that have not undergone conversion, as desired. As such, the probes can be specific for sequences in which a modification of interest, such as methylation, was or was not present. In some embodiments, where a modification sensitive conversion is performed on a sample or subsample, the subsequent capturing of one or more target region sets (e.g., at least an epigenetic target region set) from that sample or subsample uses target-specific probes that comprise probes that can hybridize to target sequences regardless of modification state (e.g., comprise a promiscuously pairing nucleobase at a position that may or may not have undergone conversion of modified or unmodified cytosines to uracils or analogs thereof, such as DHU, that preferentially pair with adenine; for example, inosine can pair with C or U).

In some embodiments, the methods comprise preparing a pool comprising at least a portion of the DNA of the second subsample (also referred to as the hypomethylated partition) and at least a portion of the DNA of the first subsample (also referred to as the hypermethylated partition). Target regions, e.g., including epigenetic target regions and/or sequence-variable target regions, may be captured from the pool. The steps of capturing a target region set from at least a portion of a subsample described elsewhere herein encompass capture steps performed on a pool comprising DNA from the first and second subsamples. A step of amplifying DNA in the pool may be performed before capturing target regions from the pool. The capturing step may have any of the features described elsewhere herein.

The epigenetic target regions may show differences in methylation levels and/or fragmentation patterns depending on whether they originated from a tumor or from healthy cells, or what type of tissue they originated from, as discussed elsewhere herein. The sequence-variable target regions may show differences in sequence depending on whether they originated from a tumor or from healthy cells.

Analysis of epigenetic target regions from the hypomethylated partition may be less informative in some applications than analysis of sequence-variable target-regions from the hypermethylated and hypomethylated partitions and epigenetic target regions from the hypermethylated partition. As such, in methods where sequence-variable target-regions and epigenetic target regions are being captured, the latter may be captured to a lesser extent than one or more of the sequence-variable target-regions from the hypermethylated and hypomethylated partitions and epigenetic target regions from the hypermethylated partition. For example, sequence-variable target regions can be captured from the portion of the hypomethylated partition not pooled with the hypermethylated partition, and the pool can be prepared with some (e.g., a majority, substantially all, or all) of the DNA from the hypermethylated partition and none or some (e.g., a minority) of the DNA from the hypomethylated partition. Such approaches can reduce or eliminate sequencing of epigenetic target regions from the hypomethylated partition, thereby reducing the amount of sequencing data that suffices for further analysis.

In some embodiments, including a minority of the DNA of the hypomethylated partition in the pool facilitates quantification of one or more epigenetic features (e.g., methylation or other epigenetic feature(s) discussed in detail elsewhere herein), e.g., on a relative basis.

In some embodiments, the pool comprises a minority of the DNA of the hypomethylated partition, e.g., less than about 50% of the DNA of the hypomethylated partition, such as less than or equal to about 45%, 40%, 35%, 30%, 25%, 20%, 15%, 10%, or 5% of the DNA of the hypomethylated partition. In some embodiments, the pool comprises about 5%-25% of the DNA of the hypomethylated partition. In some embodiments, the pool comprises about 10%-20% of the DNA of the hypomethylated partition. In some embodiments, the pool comprises about 10% of the DNA of the hypomethylated partition. In some embodiments, the pool comprises about 15% of the DNA of the hypomethylated partition. In some embodiments, the pool comprises about 20% of the DNA of the hypomethylated partition.

In some embodiments, the pool comprises a portion of the hypermethylated partition, which may be at least about 50% of the DNA of the hypermethylated partition. For example, the pool may comprise at least about 55%, 60%, 65%, 70%, 75%, 80%, 85%, 90%, or 95% of the DNA of the hypermethylated partition. In some embodiments, the pool comprises 50-55%, 55-60%, 60-65%, 65-70%, 70-75%, 75-80%, 80-85%, 85-90%, 90-95%, or 95-100% of the DNA of the hypermethylated partition. In some embodiments, the second pool comprises all or substantially all of the hypermethylated partition.

In some embodiments, the methods comprise preparing a first pool comprising at least a portion of the DNA of the hypomethylated partition. In some embodiments, the methods comprise preparing a second pool comprising at least a portion of the DNA of the hypermethylated partition. In some embodiments, the first pool further comprises a portion of the DNA of the hypermethylated partition. In some embodiments, the second pool further comprises a portion of the DNA of the hypomethylated partition. In some embodiments, the first pool comprises a majority of the DNA of the hypomethylated partition, and optionally and a minority of the DNA of the hypermethylated partition. In some embodiments, the second pool comprises a majority of the DNA of the hypermethylated partition and a minority of the DNA of the hypomethylated partition. In some embodiments involving an intermediately methylated partition, the second pool comprises at least a portion of the DNA of the intermediately methylated partition, e.g., a majority of the DNA of the intermediately methylated partition. In some embodiments, the first pool comprises a majority of the DNA of the hypomethylated partition, and the second pool comprises a majority of the DNA of the hypermethylated partition and a majority of the DNA of the intermediately methylated partition.

In some embodiments, the methods comprise capturing at least a first set of target regions from the first pool, e.g., wherein the first pool is as set forth in any of the embodiments above. In some embodiments, the first set comprises sequence-variable target regions. In some embodiments, the first set comprises hypomethylation variable target regions and/or fragmentation variable target regions. In some embodiments, the first set comprises sequence-variable target regions and fragmentation variable target regions. In some embodiments, the first set comprises sequence-variable target regions, hypomethylation variable target regions and fragmentation variable target regions. A step of amplifying DNA in the first pool may be performed before this capture step. In some embodiments, capturing the first set of target regions from the first pool comprises contacting the DNA of the first pool with a first set of target-specific probes. In some embodiments, the first set of target-specific probes comprises target-binding probes specific for the sequence-variable target regions. In some embodiments, the first set of target-specific probes comprises target-binding probes specific for the sequence-variable target regions, hypomethylation variable target regions and/or fragmentation variable target regions.

In some embodiments, the methods comprise capturing a second set of target regions or plurality of sets of target regions from the second pool, e.g., wherein the first pool is as set forth in any of the embodiments above. In some embodiments, the second plurality comprises epigenetic target regions, such as hypermethylation variable target regions and/or fragmentation variable target regions. In some embodiments, the second plurality comprises sequence-variable target regions and epigenetic target regions, such as hypermethylation variable target regions and/or fragmentation variable target regions. A step of amplifying DNA in the second pool may be performed before this capture step. In some embodiments, capturing the second plurality of sets of target regions from the second pool comprises contacting the DNA of the first pool with a second set of target-specific probes, wherein the second set of target-specific probes comprises target-binding probes specific for the sequence-variable target regions and target-binding probes specific for the epigenetic target regions. In some embodiments, the first set of target regions and the second set of target regions are not identical. For example, the first set of target regions may comprise one or more target regions not present in the second set of target regions. Alternatively or in addition, the second set of target regions may comprise one or more target regions not present in the first set of target regions. In some embodiments, at least one hypermethylation variable target region is captured from the second pool but not from the first pool. In some embodiments, a plurality of hypermethylation variable target regions are captured from the second pool but not from the first pool. In some embodiments, the first set of target regions comprises sequence-variable target regions and/or the second set of target regions comprises epigenetic target regions. In some embodiments, the first set of target regions comprises sequence-variable target regions, and fragmentation variable target regions; and the second set of target regions comprises epigenetic target regions, such as hypermethylation variable target regions and fragmentation variable target regions. In some embodiments, the first set of target regions comprises sequence-variable target regions, fragmentation variable target regions, and comprises hypomethylation variable target regions; and the second set of target regions comprises epigenetic target regions, such as hypermethylation variable target regions and fragmentation variable target regions.

In some embodiments, the first pool comprises a majority of the DNA of the hypomethylated partition and a portion of the DNA of the hypermethylated partition (e.g., about half), and the second pool comprises a portion of the DNA of the hypermethylated partition (e.g., about half). In some such embodiments, the first set of target regions comprises sequence-variable target regions and/or the second set of target regions comprises epigenetic target regions. The sequence-variable target regions and/or the epigenetic target regions may be as set forth in any of the embodiments described elsewhere herein.

### H. Ligation to Adapters

In some embodiments, the methods comprise ligating adapters to DNA. In some embodiments, the ligating adapters to DNA produces adapter-ligated DNA. In some embodiments, DNA molecules can be subjected to blunt-end ligation with blunt-ended adapters. In some embodiments, DNA molecules can be subjected to sticky-end ligation with sticky-ended adapters. DNA molecules can be ligated to adapters at either one end or both ends. DNA molecules can be ligated with at least partially double stranded adapter (e.g., a Y shaped or bell-shaped adapter).

In some embodiments, the ligation step can take place prior to or after sequencing the DNA. In some embodiments, the ligation step can take place prior to sequencing the DNA. In some embodiments, the ligation step can take place prior to or after capturing the DNA. In some embodiments, the ligation step can take place prior to capturing the DNA. In some embodiments, the ligation step can take place prior to or after capturing the DNA and prior to or after sequencing the DNA. In some embodiments, the ligation step can take place after capturing the DNA and prior to sequencing the DNA. In some embodiments, the ligation step can take place prior to or after partitioning the DNA into a plurality of subsamples. In some embodiments, the ligation step can take place prior to partitioning the DNA into a plurality of subsamples. In some embodiments, the ligation step can take place prior to or after partitioning the DNA into a plurality of subsamples and prior to or after sequencing the DNA. In some embodiments, the ligation step can take place after partitioning the DNA into a plurality of subsamples and prior to the sequencing the DNA. In some embodiments, the ligation step can take place prior to or after subjecting the sample or one or more subsamples to a procedure that affects a first nucleobase in the DNA differently from a second nucleobase. In some embodiments, the ligation step can take place prior to subjecting the sample or one or more subsamples to a procedure that affects a first nucleobase in the DNA differently from a second nucleobase. In some embodiments, the ligation step can take place prior to or after the subjecting the sample or one or more subsamples to a procedure that affects a first nucleobase in the DNA differently from a second nucleobase and prior to or after the sequencing the DNA. In some embodiments, the ligation step can take place after subjecting the sample or one or more subsamples to a procedure that affects a first nucleobase in the DNA differently from a second nucleobase and prior to the sequencing the DNA.

In some embodiments, the procedure that affects a first nucleobase in the DNA differently from a second nucleobase is a conversion step. In some embodiments, the ligation step can take place before or after the conversion step. In general, “conversion step” or “conversion procedure” refers to any step or procedure that changes the base pairing specificity of one or more nucleotides. In some embodiments, the conversion step comprises contacting DNA with a deaminase.

DNA ligase and adapters are added to ligate DNA molecules in the sample with an adapter on one or both ends, i.e. to form adapted DNA. As used herein, “adapter” refers to short nucleic acids (e.g., less than about 500, less than about 100 or less than about 50 nucleotides in length, or be 20-30, 20-40, 30-50, 30-60, 40-60, 40-70, 50-60, 50-70, 20-500, or 30-100 bases from end to end) that are typically at least partially double-stranded and can be ligated to the end of a given sample DNA molecule. In some instances, two adapters can be ligated to a single sample DNA molecule, with one adapter ligated to each end of the sample nucleic acid molecule.

In some embodiments, the ligase used in ligation reactions can act on both single strand DNA nicks and double stranded DNA ends. In some cases, the ligase is T4 DNA ligase or T3 DNA ligase. Adapters can include nucleic acid primer binding sites to permit amplification of a sample DNA molecule flanked by adapters at both ends, and/or a sequencing primer binding site, including primer binding sites for sequencing applications, such as various next generation sequencing (NGS) applications. Adapters can include a sequence for hybridizing to a solid support, e.g., a flow cell sequence. Adapters can also include binding sites for capture probes, such as an oligonucleotide attached to a flow cell support or the like. Adapters can also include sample indexes and/or molecular barcodes. These are typically positioned relative to amplification primer and sequencing primer binding sites, such that the sample index and/or molecular barcode is included in amplicons and sequencing reads of a given DNA molecule. Adapters of the same or different sequence can be linked to the respective ends of a sample DNA molecule. In some cases, adapters of the same or different sequence are linked to the respective ends of the DNA molecule except that the sample index and/or molecular barcode differs in its sequence. In some embodiments, the adapter is a Y-shaped adapter in which one end is blunt ended or tailed as described herein, for joining to a nucleic acid molecule, which is also blunt ended or tailed with one or more complementary nucleotides to those in the tail of the adapter. In another exemplary embodiment, an adapter is a bell-shaped adapter that includes a blunt or tailed end for joining to a DNA molecule to be analyzed. Other exemplary adapters include T-tailed, C-tailed or hairpin shaped adapters. For example, a hairpin shaped adapter can comprise a complementary double stranded portion and a loop portion, where the double stranded portion can be attached (e.g. ligated) to a double-stranded polynucleotide. Hairpin shaped sequencing adapters can be attached to both ends of a polynucleotide fragment to generate a circular molecule, which can be sequenced multiple times. The adapters used in the methods of the present disclosure comprise one or more known modified nucleosides, such as methylated nucleosides. In some embodiments, the modified nucleosides comprise modification resistant cytosines. In some embodiments, each cytosine in each adapter is a modification resistant cytosine. In some embodiments, the modification resistant cytosine is a deamination resistant cytosine. In some embodiments, the deamination resistant cytosine comprises 5-propynylC (5pyC), 5-pyrrolo-dC (5pyrC), 5-hydroxymethylcytosine (5hmC), glucosylated5-hydroxymethylcytosine (5ghmC), cytosine 5-methylenesulfonate (CMS), or N4-modified cytosine. In some embodiments, the adapters are resistant to digestion by a (methylation resistant restriction enzyme) MSRE. In some embodiments, the MSRE digestion-resistant adapters comprise one or more methylated nucleotides (e.g., 5-methylcytosine, 5-hydroxymethylcytosine, or a combination thereof), comprise one or more nucleotide analogs resistant to methylation sensitive restriction enzymes, or do not comprise a nucleotide sequence recognized by the MSRE. In some embodiments, the one or more methylated nucleotides in the MSRE digestion-resistant adapters comprise 5-methylcytosine and/or 5-hydroxymethylcytosine. In some embodiments, the adapters are resistant to digestion by a methylation dependent restriction enzyme (MDRE). In some embodiments, the MDRE digestion-resistant adapters comprise one or more unmethylated nucleotides, comprise one or more nucleotide analogs resistant to methylation dependent restriction enzymes, or do not comprise a nucleotide sequence recognized by the MDRE.

In instances where two adapters are ligated to a sample nucleic acid (one at each end), either or both of the adapters may comprise one or more known modified nucleosides. Typically, the primer binding site(s), sequencing primer binding site(s), sample index(es) and/or molecular barcode(s), if present, do not comprise the known modified nucleosides that change base pairing specificity as a result of the conversion procedure.

In some embodiments, adapters may be added to the DNA or a subsample thereof. Adapters can be ligated to DNA at any point in the methods herein. In some embodiments, adapters are ligated to the DNA in a sample. In some embodiments, adapters are ligated to the DNA of a sample or subsample thereof prior to annealing primers to the DNA for capture probe generation. In some such embodiments, the adapter-ligated DNA is amplified prior to annealing primers to the DNA for capture probe generation. In some embodiments, adapters are ligated to the DNA of a sample or subsample thereof before the DNA is contacted with the capture probes. In some embodiments, the DNA to which the adapters are ligated is in the same sample or subsample as the DNA used as a template to generate capture probes. In some embodiments, the DNA to which the adapters are ligated is in a different sample or subsample, e.g., a second sample or a second subsample of a first sample, than the DNA used as a template to generate capture probes. In some embodiments, the adapters ligated to DNA captured by the capture probes.

In some embodiments, the primers used to generate capture probes are not complementary to adapters, and the resulting capture probes therefore do not comprise adapters. Adapter-ligated DNA can therefore be selectively amplified in the presence of capture probes that do not comprise adapters. Similarly, adapter-ligated DNA can be separated from DNA that does not comprise adapters.

In some embodiments, the disclosed methods comprise analyzing DNA in a sample. In such methods, adapters may be added to the DNA. This may be done concurrently with an amplification procedure, e.g., by providing the adapters in a 5′ portion of a primer (where PCR is used, this can be referred to as library prep-PCR or LP-PCR), before, or after an amplification step. In some embodiments, adapters are added by other approaches, such as ligation. In some such methods, first adapters are added to the 3′ ends of the nucleic acids by ligation, which may include ligation to single-stranded DNA. In some embodiments, prior to any partitioning or capturing steps, first adapters are added to the nucleic acids by ligation, which may include ligation to single-stranded DNA (e.g., to the 3′ ends thereof). In some embodiments, the capture probes can be isolated after partitioning and ligation. For example, the hypomethylated partition can be ligated with adapters and a portion of the ligated hypomethylated partition can then be used to generate the capture probes for rearrangements. The adapter can be used as a priming site for second-strand synthesis, e.g., using a universal primer and a DNA polymerase. A second adapter can then be ligated to at least the 3′ end of the second strand of the now double-stranded molecule. In some embodiments, the first adapter comprises an affinity tag, such as biotin, and nucleic acid ligated to the first adapter is bound to a solid support (e.g., bead), which may comprise a binding partner for the affinity tag such as streptavidin. For further discussion of a related procedure, see Gansauge et al., Nature Protocols 8:737-748 (2013). Commercial kits for sequencing library preparation compatible with single-stranded nucleic acids are available, e.g., the Accel-NGS® Methyl-Seq DNA Library Kit from Swift Biosciences. In some embodiments, after adapter ligation, nucleic acids are amplified.

In some embodiments, the single-stranded DNA library preparation is performed in a one-step combined phosphorylation/ligation reaction, e.g., as described in Troll et al., BMC Genomics, 20:1023 (2019), available at https://doi.org/10.1186/s12864-019-6355-0. This method, called Single Reaction Single-stranded LibrarY (“SRSLY,”) can be performed without end-polishing. SRSLY may be useful for converting short and fragmented DNA molecules, e.g., cfDNA fragments, into sequencing libraries while retaining native lengths and ends. The SRSLY method can create sequencing libraries (e.g., Illumina sequencing libraries) from fragmented or degraded template (input) DNA. In particular embodiments, template DNA is first heat denatured and then immediately cold shocked to render the template DNA molecules single-stranded. The DNA can be maintained as single-stranded throughout the ligation reaction by the inclusion of a thermostable single-stranded binding protein (SSB). Next, the template DNA, which at this point can be single-stranded and coated with SSB, is placed in a phosphorylation/ligation dual reaction with directional dsDNA NGS adapters that contain single-stranded overhangs. Both the forward and reverse sequencing adapters can share similar structures but differ in which termini is unblocked in order to facilitate proper ligations. Both sequencing adapters can comprise a dsDNA portion and a single-stranded splint overhang of random nucleotides that occurs on the 3-prime terminus of the bottom strand of the forward adapter and the 5-prime terminus of the bottom strand of the reverse adapter. In this way, the forward adapter (e.g., (P5) Illumina adapter) can be delivered to the 5-prime end of template molecules and the reverse adapter (e.g., (P7) Illumina adapter) is delivered to the 3-prime end of template molecules. Thus, the native polarity of input DNA molecules can be retained.

During the dual phosphorylation/ligation reaction, T4 Polynucleotide Kinase (PNK) can be used to prepare template DNA termini for ligation by phosphorylating 5-prime termini and dephosphorylating 3-prime termini. T4 PNK works on both ssDNA and dsDNA molecules and has no activity on the phosphorylation state of proteins. Simultaneously, the random nucleotides of the splint adapter can be annealed to the single-stranded template molecule. This creates a short, localized dsDNA molecule, enabling ligation of template to adapter with a ligase such as T4 DNA ligase, which has high ligation efficiency on dsDNA templates but low efficiency on ssDNA. After the single phosphorylation/ligation reaction is complete, the library DNA can be, e.g., purified and placed directly into standard NGS indexing PCR, compatible with both traditional single or dual index primers.

In some embodiments, the adapters include different tags of sufficient numbers that the number of combinations of tags results in a low probability e.g., 95, 99 or 99.9% of two nucleic acids with the same start and stop points receiving the same combination of tags. Adapters, whether bearing the same or different tags, can include the same or different primer binding sites, but preferably adapters include the same primer binding site.

In some embodiments, following attachment of adapters, the nucleic acids are subject to amplification. The amplification can use, e.g., universal primers that recognize primer binding sites in the adapters.

In some embodiments, following attachment of adapters, the DNA or a subsample or portion of the DNA is partitioned, comprising contacting the DNA with an agent that preferentially binds to nucleic acids bearing an epigenetic modification. The nucleic acids are partitioned into at least two partitioned subsamples differing in the extent to which the nucleic acids bear the modification from binding to the agents. For example, if the agent has affinity for nucleic acids bearing the modification, nucleic acids overrepresented in the modification (compared with median representation in the population) preferentially bind to the agent, whereas nucleic acids underrepresented for the modification do not bind or are more easily eluted from the agent. The nucleic acids can then be amplified from primers binding to the primer binding sites within the adapters. Partitioning may be performed instead before adapter attachment, in which case the adapters may comprise differential tags that include a component that identifies which partition a molecule occurred in.

In some embodiments, the nucleic acids are linked at both ends to Y-shaped adapters including primer binding sites and tags. The molecules are amplified.

### I. Molecular Tagging

In some embodiments, the DNA molecules may be tagged with sample indexes and/or molecular barcodes (referred to generally as “tags”). In some embodiments, the DNA molecules of the sample comprise barcodes. Tags can be molecules, such as nucleic acids, containing information that indicates a feature of the molecule with which the tag is associated. For example, DNA molecules can bear a sample tag or sample index (which distinguishes molecules in one sample from those in a different sample), a partition tag (which distinguishes molecules in one partition from those in a different partition) and/or a molecular tag/molecular barcode (which distinguishes different molecules from one another (in both unique and non-unique tagging scenarios).

Tagging strategies can be divided into unique tagging and non-unique tagging strategies. In unique tagging, all or substantially all of the molecules in a sample bear a different tag, so that reads can be assigned to original molecules based on tag information alone. Tags used in such methods are sometimes referred to as “unique tags”. In non-unique tagging, different molecules in the same sample can bear the same tag, so that other information in addition to tag information is used to assign a sequence read to an original molecule. Such information may include start and stop coordinate, coordinate to which the molecule maps, start or stop coordinate alone, etc. Tags used in such methods are sometimes referred to as “non-unique tags”. Accordingly, it is not necessary to uniquely tag every molecule in a sample. It suffices to uniquely tag molecules falling within an identifiable class within a sample. Thus, molecules in different identifiable families can bear the same tag without loss of information about the identity of the tagged molecule.

In certain embodiments, a tag can comprise one or a combination of barcodes. As used herein, the term “barcode” refers to a nucleic acid molecule having a particular nucleotide sequence, or to the nucleotide sequence, itself, depending on context. A barcode can have, for example, between 10 and 100 nucleotides. A collection of barcodes can have degenerate sequences or can have sequences having a certain Hamming distance, as desired for the specific purpose. So, for example, a molecular barcode can be comprised of one barcode or a combination of two barcodes, each attached to different ends of a molecule. Additionally or alternatively, for different partitions and/or samples, different sets of molecular barcodes, molecular tags, or molecular indexes can be used such that the barcodes serve as a molecular tag through their individual sequences and also serve to identify the partition and/or sample to which they correspond based the set of which they are a member.

For example, barcodes can be used to allow the origin of the DNA (e.g., the subject, biological sample (e.g., samples collected at various time points), enriched DNA sample (e.g., enriched DNA comprising an epigenetic target region set or enriched DNA comprising a sequence-variable target region set), partition, or similar) to be identified, e.g., following pooling of a plurality of samples for parallel sequencing. Tags comprising barcodes can be incorporated into or otherwise joined to adapters. Tags can be incorporated by ligation, overlap extension PCR among other methods. Tags can be used to label the individual polynucleotide population partitions so as to correlate the tag (or tags) with a specific partition. Alternatively, tags can be used in embodiments of the disclosure that do not employ a partitioning step. In some embodiments, a single tag can be used to label a specific partition. In some embodiments, multiple different tags can be used to label a specific partition. In embodiments employing multiple different tags to label a specific partition, the set of tags used to label one partition can be readily differentiated for the set of tags used to label other partitions. In some embodiments, the tags may have additional functions, for example the tags can be used to index sample sources or used as unique molecular identifiers (which can be used to improve the quality of sequencing data by differentiating sequencing errors from mutations, for example as in Kinde et al., Proc Nat'l Acad Sci USA 108:9530-9535 (2011), Kou et al., PLOS ONE, 11: e0146638 (2016)) or used as non-unique molecule identifiers, for example as described in U.S. Pat. No. 9,598,731. Similarly, in some embodiments, the tags may have additional functions, for example the tags can be used to index sample sources or used as non-unique molecular identifiers (which can be used to improve the quality of sequencing data by differentiating sequencing errors from mutations).

Tags may be incorporated into or otherwise joined to adapters by chemical synthesis, ligation (e.g., as described above, e.g. by blunt-end ligation or sticky-end ligation), or overlap extension polymerase chain reaction (PCR), among other methods. Such adapters are ultimately joined to the sample DNA molecule. In other embodiments, one or more rounds of amplification cycles (e.g., PCR amplification) may be applied to introduce sample indexes to a nucleic acid molecule using conventional nucleic acid amplification methods. The amplifications may be conducted in one or more reaction mixtures (e.g., a plurality of microwells in an array). Molecular barcodes and/or sample indexes may be introduced simultaneously, or in any sequential order. In some embodiments, molecular barcodes and/or sample indexes are introduced prior to and/or after any conversion procedure. In the case of molecular barcodes and/or sample indexes being introduced through amplification processes, the conversion step will occur before the molecular barcodes and/or sample indexes are introduced. In some embodiments, molecular barcodes and/or sample indexes are introduced prior to and/or after sequence capturing steps, if present, are performed. In some embodiments, only the molecular barcodes are introduced prior to probe capturing and the sample indexes are introduced after sequence capturing steps are performed. In some embodiments, both the molecular barcodes and the sample indexes are introduced prior to performing probe-based capturing steps, if present. In some embodiments, the sample indexes are introduced after sequence capturing steps are performed, if present. In some embodiments, sample indexes are incorporated through overlap extension polymerase chain reaction (PCR).

In some embodiments, the tags may be located at one end or at both ends of the sample DNA molecule. In some embodiments, tags are predetermined or random or semi-random sequence oligonucleotides. In some embodiments, the tag(s) may together be less than about 500, 200, 100, 50, 20, 10, 9, 8, 7, 6, 5, 4, 3, 2, or 1 nucleotides in length. Typically tags are about 5 to 20 or 6 to 15 nucleotides in length. The tags may be linked to sample DNA molecules randomly or non-randomly.

In some embodiments, each sample or partition (discussed below) is uniquely tagged with a sample index or a combination of sample indexes. In some embodiments, each nucleic acid molecule of a sample or sub-sample is uniquely tagged with a molecular barcode or a combination of molecular barcodes. In other embodiments, a plurality of molecular barcodes may be used such that molecular barcodes are not necessarily unique to one another in the plurality (e.g., non-unique molecular barcodes). In these embodiments, molecular barcodes are generally attached (e.g., by ligation as part of an adapter) to individual molecules such that the combination of the molecular barcode and the sequence it may be attached to creates a unique sequence that may be individually tracked. Detection of non-unique molecular barcodes in combination with endogenous sequence information (e.g., the beginning (start) and/or end (stop) genomic location/position corresponding to the sequence of the original DNA molecule in the sample, start and stop genomic positions corresponding to the sequence of the original DNA molecule in the sample, the beginning (start) and/or end (stop) genomic location/position of the sequence read that is mapped to the reference sequence, start and stop genomic positions of the sequence read that is mapped to the reference sequence, sub-sequences of sequence reads at one or both ends, length of sequence reads, and/or length of the original DNA molecule in the sample) typically allows for the assignment of a unique identity to a particular molecule. In some embodiments, beginning region comprises the first 1, first 2, the first 5, the first 10, the first 15, the first 20, the first 25, the first 30 or at least the first 30 base positions at the 5′ end of the sequencing read that align to the reference sequence. In some embodiments, the end region comprises the last 1, last 2, the last 5, the last 10, the last 15, the last 20, the last 25, the last 30 or at least the last 30 base positions at the 3′ end of the sequencing read that align to the reference sequence. The length, or number of base pairs, of an individual sequence read are also optionally used to assign a unique identity to a given molecule. As described herein, fragments from a single strand of nucleic acid having been assigned a unique identity, may thereby permit subsequent identification of fragments from the parent strand, and/or a complementary strand.

In certain embodiments of non-unique tagging, the number of different tags used can be sufficient that there is a very high likelihood (e.g., at least 99%, at least 99.9%, at least 99.99% or at least 99.999% that all DNA molecules of a particular group bear a different tag. It is to be noted that when barcodes are used as tags, and when barcodes are attached, e.g., randomly, to both ends of a molecule, the combination of barcodes, together, can constitute a tag. This number, in term, is a function of the number of molecules falling into the calls. For example, the class may be all molecules mapping to the same start-stop position on a reference genome. The class may be all molecules mapping across a particular genetic locus, e.g., a particular base or a particular region (e.g., up to 100 bases or a gene or an exon of a gene).

In certain embodiments, the number of different tags used to uniquely identify a number of molecules, z, in a class can be between any of 2*z, 3*z, 4*z, 5*z, 6*z, 7*z, 8*z, 9*z, 10*z, 11*z, 12*z, 13*z, 14*z, 15*z, 16*z, 17*z, 18*z, 19*z, 20*z or 100*z (e.g., lower limit) and any of 100,000*z, 10,000*z, 1000*z or 100*z (e.g., upper limit).

In some embodiments, molecular barcodes are introduced at an expected ratio of a set of identifiers (e.g., a combination of unique or non-unique molecular barcodes) to molecules in a sample. One example format uses from about 2 to about 1,000,000 different molecular barcode sequences, or from about 5 to about 150 different molecular barcode sequences, or from about 20 to about 50 different molecular barcode sequences, ligated to both ends of a target molecule. Alternatively, from about 25 to about 1,000,000 different molecular barcode sequences may be used. For example, 20-50×20-50 molecular barcode sequences (i.e., one of the 20-50 different molecular barcode sequences can be attached to each end of the target molecule) can be used. Such numbers of identifiers are typically sufficient for different molecules having the same start and stop points to have a high probability (e.g., at least 94%, 99.5%, 99.99%, or 99.999%) of receiving different combinations of identifiers. In some embodiments, about 80%, about 90%, about 95%, or about 99% of molecules have the same combinations of molecular barcodes. For example, in a sample of about 5 ng to 30 ng of cell free DNA, one expects around 3000 molecules to map to a particular nucleotide coordinate, and between about 3 and 10 molecules having any start coordinate to share the same stop coordinate. Accordingly, about 50 to about 50,000 different tags (e.g., between about 6 and 220 barcode combinations) can suffice to uniquely tag all such molecules. To uniquely tag all 3000 molecules mapping across a nucleotide coordinate, about 1 million to about 20 million different tags would be required.

In some embodiments, the assignment of unique or non-unique molecular barcodes in reactions is performed using methods and systems described in, for example, U.S. patents application Nos. 20010053519, 20030152490, and 20110160078, and U.S. Pat. Nos. 6,582,908, 7,537,898, 9,598,731, and 9,902,992, each of which is hereby incorporated by reference in its entirety. Alternatively, in some embodiments, different nucleic acid molecules of a sample may be identified using only endogenous sequence information (e.g., start and/or stop positions, sub-sequences of one or both ends of a sequence, and/or lengths). Tags can be linked to sample nucleic acids randomly or non-randomly.

In some embodiments, the tagged nucleic acids are sequenced after loading into a microwell plate. The microwell plate can have 96, 384, or 1536 microwells. In some cases, they are introduced at an expected ratio of unique tags to microwells. For example, the unique tags may be loaded so that more than about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags are loaded per genome sample. In some cases, the unique tags may be loaded so that less than about 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags are loaded per genome sample. In some cases, the average number of unique tags loaded per sample genome is less than, or greater than, about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags per genome sample.

In some embodiments, a format uses 20-50 different tags (e.g., barcodes) ligated to both ends of target nucleic acids. For example, 35 different tags (e.g., barcodes) ligated to both ends of target molecules creating 35×35 permutations, which equals 1225 for 35 tags. Such numbers of tags are sufficient so that different molecules having the same start and stop points have a high probability (e.g., at least 94%, 99.5%, 99.99%, 99.999%) of receiving different combinations of tags. Other barcode combinations include any number between 10 and 500, e.g., about 15×15, about 35×35, about 75×75, about 100×100, about 250×250, about 500×500.

In some cases, unique tags may be predetermined or random or semi-random sequence oligonucleotides. In other cases, a plurality of barcodes may be used such that barcodes are not necessarily unique to one another in the plurality. In this example, barcodes may be ligated to individual molecules such that the combination of the barcode and the sequence it may be ligated to creates a unique sequence that may be individually tracked. As described herein, detection of non-unique barcodes in combination with sequence data of beginning (start) and end (stop) portions of sequence reads may allow assignment of a unique identity to a particular molecule. The length or number of base pairs, of an individual sequence read may also be used to assign a unique identity to such a molecule. As described herein, fragments from a single strand of nucleic acid having been assigned a unique identity, may thereby permit subsequent identification of fragments from the parent strand.

In some embodiments, the method includes adding one or more internal control DNAs and forward and reverse primers for amplifying the internal control DNAs. The internal control DNAs may be added before amplification using the primers that anneal upstream and downstream of the rearrangement breakpoints. The forward and reverse primers for amplifying the internal control DNAs may be included with, or added at the same time as, the primers that anneal upstream and downstream of the rearrangement breakpoints. The internal control DNAs may comprise or consist of sequences that do not occur in the genome of the subject, or that do not occur in the genome of the species of which the subject is a member (e.g., the human genome). The forward and/or reverse primers for amplifying the internal control DNAs may comprise sequences that are not complementary to any sequence in the genome of the subject, e.g., the human genome. The internal control DNAs may be used to ensure that the amplification process proceeded as designed. As such, the method may comprise detecting (e.g., sequencing) molecules amplified from and/or captured by the one or more internal control DNAs. The method can comprise comparing an amount of internal control DNAs (e.g., number of molecules or reads detected that correspond to an internal control DNA sequence) to a predetermined threshold, and either rejecting sequencing results if the predetermined threshold is not met or accepting sequencing results if the predetermined threshold is met. The predetermined threshold may be established, e.g., based on historical data or by testing the method on samples of DNA from test subjects, such as healthy volunteers. For example, amplification and detection of the one or more internal control DNAs provides confirmation that the amplification process proceeded properly, thus reducing the likelihood of a false negative.

### J. DNA Sequencing

In some embodiments, the method comprises sequencing at least a portion of the DNA in a sample, such as the DNA in one or more subsamples or the DNA contained in a sample. In some embodiments, sequencing comprises sequencing the DNA in a manner that distinguishes the first nucleobase from the second nucleobase. In some embodiments, subsamples are pooled prior to the sequencing. In some embodiments, subsamples are produced using a partitioning step. In general, sample nucleic acids, including nucleic acids flanked by adapters, with or without prior amplification can be subject to sequencing. Sequencing methods include, for example, Sanger sequencing, high-throughput sequencing, pyrosequencing, sequencing-by-synthesis, long-read sequencing (also known as single-molecule sequencing or third generation sequencing), nanopore sequencing (a type of long-read sequencing), 5-letter sequencing or 6-letter sequencing, semiconductor sequencing, sequencing-by-ligation, sequencing-by-hybridization, Digital Gene Expression (Helicos), Next generation sequencing (NGS), Single Molecule Sequencing by Synthesis (SMSS) (Helicos), massively-parallel sequencing, Clonal Single Molecule Array (Solexa), shotgun sequencing, Ion Torrent, Oxford Nanopore, Roche Genia, Maxim-Gilbert sequencing, primer walking, and sequencing using PacBio, SOLID, Ion Torrent, or Nanopore platforms. Sequencing reactions can be performed in a variety of sample processing units, which may include multiple lanes, multiple channels, multiple wells, or other means of processing multiple sample sets substantially simultaneously. Sample processing unit can also include multiple sample chambers to enable processing of multiple runs simultaneously.

In some embodiments, sequencing comprises detecting and/or distinguishing unmodified and modified nucleobases. For example, long-read sequencing (also referred to herein as third generation sequencing) methods include those that can generate longer sequencing reads, such as reads in excess of 10 kilobases, as compared to short-read sequencing methods, which generally produce reads of up to about 600 bases in length. Compared to short reads, long reads can improve de novo assembly, transcript isoform identification, and detection and/or mapping of structural variants. Furthermore, long-read sequencing of native DNA or RNA molecules reduces amplification bias and preserves base modifications, such as methylation status. Long-read sequencing technologies useful herein can include any suitable long-read sequencing methods, including, but not limited to, Pacific Biosciences (PacBio) single-molecule real-time (SMRT) sequencing, Oxford Nanopore Technologies (ONT) nanopore sequencing, and synthetic long-read sequencing approaches, such as linked reads, proximity ligation strategies, and optical mapping. Synthetic long-read approaches comprise assembly of short reads from the same DNA molecule to generate synthetic long reads, and may be used in conjunction with “true” long-read sequencing technologies, such as SMRT and nanopore sequencing methods.

Single-molecule real-time (SMRT) sequencing can facilitate direct detection of, e.g., 5-methylcytosine and 5-hydroxymethylcytosine as well as unmodified cytosine (Weirather J L, et al., “Comprehensive comparison of Pacific Biosciences and Oxford Nanopore Technologies and their applications to transcriptome analysis,” F1000Research, 6:100, 2017). Whereas next-generation sequencing methods detect augmented signals from a clonal population of amplified DNA fragments, SMRT sequencing captures a single DNA molecule, maintaining base modification during sequencing. The error rate of raw PacBio SMRT sequencing-generated data is about 13-15%, as the signal-to-noise ratio from single DNA molecules not high. To increase accuracy, this platform uses a circular DNA template by ligating hairpin adapters to both ends of target double-stranded DNA. As the polymerase repeatedly traverses and replicates the circular molecule, the DNA template is sequenced multiple times to generate a continuous long read (CLR). The CLR can be split into multiple reads (“subreads”) by removing adapter sequences, and multiple subreads generate circular consensus sequence (“CCS”) reads with higher accuracy. The average length of a CLR is >10 kb and up to 60 kb, with length depending on the polymerase lifetime. Thus, the length and accuracy of CCS reads depends on the fragment sizes. PacBio sequencing has been utilized for genome (e.g., de novo assembly, detection of structural variants and haplotyping) and transcriptome (e.g., gene isoform reconstruction and novel gene/isoform discovery) studies.

SMRT sequencing relies on sequencing-by-synthesis, where the sequence of a circular DNA template is determined from the succession of fluorescence pulses, each resulting from the addition of one labelled nucleotide by a polymerase fixed to the bottom of a well. Base modifications do not affect the base-called sequence, but they affect the kinetics of the polymerase. By considering the inter-pulse duration (IPD), base modifications can be inferred from the comparison of a modified template to an in silico model or an unmodified template. Such methods can therefore use the pulse width of a signal from sequencing bases, the interpulse duration (IPD) of bases, and the identity of the bases in order to detect a modification in a base or in a neighboring base. (See e.g., Weirather et al., F1000Research, 6:100, 2017.) SMRT sequencing can thus be used to detect base modifications such as 5-caC, 4mC, 5mC, 5hmC, 6 mA, and 80×0G (Gouil & Keniry Essays in Biochemistry (2019) 63 639-648). Accordingly, in some embodiments, the sequencing comprises SMRT sequencing.

Some sequencing reactions involve use of an enzyme to control passage of a nucleic acid through a nanopore, and in such cases reaction data can include both kinetics and other behavior of the enzyme and fluctuations in current through the nanopore. For example, ratchet proteins, helicases, or motor proteins can be used to push or pull a nucleic acid molecule through a hole in a biological or synthetic membrane. The kinetics of these proteins can vary depending on the sequence context of a nucleic acid on which they are acting. For example, they may slow down or pause at a modified base, and this behavior, captured as a part of the reaction data, is indicative of the presence of the modified base even where the modified base is not within the sensing portion of the nanopore.

One example of a nanopore-based single molecule sequencing system is that commercialized by Oxford Nanopore Technologies (ONT). (Weirather J L, et al., F1000Research, 6:100, 2017). ONT directly sequences a native single-stranded DNA (ssDNA) molecule by measuring characteristic current changes as the bases are threaded through the nanopore by a molecular motor protein. ONT uses a hairpin library structure similar to the PacBio circular DNA template: the DNA template and its complement are bound by a hairpin adapter. Therefore, the DNA template passes through the nanopore, followed by a hairpin and finally the complement. The raw read can be split into two “ID” reads (“template” and “complement”) by removing the adapter. The consensus sequence of two “ID” reads is a “2D” read with a higher accuracy.

Nanopore sequencing can be used to detect base modifications including 5-caC, 5mC, 5hmC, 6 mA, BrdU, FldU, IdU, and EdU (see e.g., Gouil & Keniry Essays in Biochemistry (2019) 63 639-648; Kutyavin, Biochemistry (2008), 47, 51, 13666-1367; Müller et al., Nature Methods (2019), volume 16, pages 429-436; Hennion et al., Genome Biology (2020), volume 21, Article number: 125). Accordingly, in some embodiments, the sequencing comprises nanopore sequencing.

5-letter and 6-letter sequencing methods include whole genome sequencing methods capable of sequencing A, C, T, and G in addition to 5mC and 5hmC to provide a 5-letter (A, C, T, G, and either 5mC or 5hmC) or 6-letter (A, C, T, G, 5mC, and 5hmC) digital readout in a single workflow. The processing of the DNA sample is entirely enzymatic and avoids the DNA degradation and genome coverage biases of bisulfite treatment. In an exemplary 5-letter sequencing method developed by Cambridge Epigenetix, the sample DNA is first fragmented via sonication and then ligated to short, synthetic DNA hairpin adapters at both ends (Füllgrabe, et al. 2022, bioRxiv doi: https://doi.org/10.1101/2022.07.08.499285). The construct is then split to separate the sense and antisense sample strands. For each original sample strand a complementary copy strand is synthesized by DNA polymerase extension of the 3′-end to generate a hairpin construct with the original sample DNA strand connected to its complementary strand, lacking epigenetic modifications, via a synthetic loop. Sequencing adapters are then ligated to the end. Modified cytosines are enzymatically protected. The unprotected Cs are then deaminated to uracil, which is subsequently read as thymine. In any such embodiments, amplification methods may comprise uracil-and/or dihydrouracil-tolerant amplification methods, such as PCR using a uracil-and/or dihydrouracil-tolerant DNA polymerase (i.e., a DNA polymerase that can read and amplify templates comprising uracil and/or dihydrouracil bases). The deaminated constructs are no longer fully complementary and have substantially reduced duplex stability, thus the hairpins can be readily opened and amplified by PCR. The constructs can be sequenced in paired-end format whereby read 1 (P1 primed) is the original stand and read 2 (P2 primed) is the copy stand. The read data is pairwise aligned so read 1 is aligned to its complementary read 2. Cognate residues from both reads are computationally resolved to produce a single genetic or epigenetic letter. Pairings of cognate bases that differ from the permissible five are the result of incomplete fidelity at some stage(s) comprising sample preparation, amplification, or erroneous base calling during sequencing. As these errors occur independently to cognate bases on each strand, substitutions result in a non-permissible pair. Non-permissible pairs are masked (marked as N) within the resolved read and the read itself is retained, leading to minimal information loss and high accuracy at read-level. The resolved read is aligned to the reference genome. Genetic variants and methylation counts are produced by read-counting at base-level.

5hmC has been shown to have value as a marker of biological states and disease which includes early cancer detection from cell-free DNA. In adapting 5-letter to 6-letter sequencing, 5mC is disambiguated from 5hmC without compromising genetic base calling within the same sample fragment. The first three steps of the workflow are identical to 5-letter sequencing described above, to generate the adapter ligated sample fragment with the synthetic copy strand. Methylation at 5mC is enzymatically copied across the CpG unit to the C on the copy strand, whilst 5hmC is enzymatically protected from such a copy. Thus, unmodified C, 5mC and 5hmC in each of the original CpG units are distinguished by unique 2-base combinations. The unmodified cytosines are then deaminated to uracil, which is subsequently read as thymine. The DNA is subjected to PCR amplification and sequencing as described earlier. The reads are pairwise aligned and resolved using a 2-base code. Each of unmodified C, 5mC, and 5hmC can be resolved as the three CpG units are distinct sequencing environments of the 2-base code.

In some embodiments, sequence coverage of the genome may be, for example, less than 5%, 10%, 15%, 20%, 25%, 30%, 40%, 50%, 60%, 70%, 80%, 90%, 95%, 99%, 99.9% or 100%. In some embodiments, the sequence reactions may provide for sequence coverage of at least 5%, 10%, 15%, 20%, 25%, 30%, 40%, 50%, 60%, 70%, or 80% of the genome. Sequence coverage can be performed on at least 5, 10, 20, 70, 100, 200 or 500 different genes, or up to, for example, 5000, 2500, 1000, 500 or 100 different genes.

Simultaneous sequencing reactions may be performed using multiplex sequencing. In some cases, cell-free nucleic acids may be sequenced with at least, for example, 1000, 2000, 3000, 4000, 5000, 6000, 7000, 8000, 9000, 10000, 50000, 100,000 sequencing reactions. In other embodiments, cell-free nucleic acids may be sequenced with less than, for example, 1000, 2000, 3000, 4000, 5000, 6000, 7000, 8000, 9000, 10000, 50000, 100,000 sequencing reactions. Sequencing reactions may be performed sequentially or simultaneously. Subsequent data analysis may be performed on all or part of the sequencing reactions. In some cases, data analysis may be performed on at least, for example, 1000, 2000, 3000, 4000, 5000, 6000, 7000, 8000, 9000, 10000, 50000, 100,000 sequencing reactions. In other cases, data analysis may be performed on less than, for example, 1000, 2000, 3000, 4000, 5000, 6000, 7000, 8000, 9000, 10000, 50000, 100,000 sequencing reactions. An exemplary read depth is 1000-50000,1000-10000, or 1000-20000 reads per locus (base).

In general, sequencing of epigenetic target regions, e.g. to analyze a modified nucleoside profile of DNA, requires a lesser depth of sequencing than sequencing of a sequence-variable target region, e.g. for analysis of mutations. Hence, lesser sequencing depths, as described herein, may in some cases be adequate for the methods described herein.

### 1. Differential Depth of Sequencing

In some embodiments, nucleic acids corresponding to the sequence-variable target region set are sequenced to a greater depth of sequencing than nucleic acids corresponding to the epigenetic target region set. In some embodiments, nucleic acids corresponding to the hydroxymethylation-variable target region set are sequenced to a greater depth of sequencing than nucleic acids corresponding to at least one other target region set. For example, the depth of sequencing for nucleic acids corresponding to the sequence-variable and/or hydroxymethylation-variable target region sets may be at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold greater, or 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, 14- to 15-fold, or 15- to 100-fold greater, than the depth of sequencing for nucleic acids corresponding to the epigenetic target region set or to at least one other target region set. In some embodiments, said depth of sequencing is at least 2-fold greater. In some embodiments, said depth of sequencing is at least 5-fold greater. In some embodiments, said depth of sequencing is at least 10-fold greater. In some embodiments, said depth of sequencing is 4- to 10-fold greater. In some embodiments, said depth of sequencing is 4- to 100-fold greater. Each of these embodiments refer to the extent to which nucleic acids corresponding to the sequence-variable target region set are sequenced to a greater depth of sequencing than nucleic acids corresponding to the epigenetic target region set.

In some embodiments, the captured cfDNA corresponding to the sequence-variable target region set and the captured cfDNA corresponding to the epigenetic target region set are sequenced concurrently, e.g., in the same sequencing cell (such as the flow cell of an Illumina sequencer) and/or in the same composition, which may be a pooled composition resulting from recombining separately captured sets or a composition obtained by capturing the cfDNA corresponding to the sequence-variable target region set and the captured cfDNA corresponding to the epigenetic target region set in the same vessel.

In some embodiments, the captured cfDNA corresponding to the hydroxymethylation variable target region set and the captured cfDNA corresponding to the at least one other target region set are sequenced concurrently, e.g., in the same sequencing cell (such as the flow cell of an Illumina sequencer) and/or in the same composition, which may be a pooled composition resulting from recombining separately captured sets or a composition obtained by capturing the cfDNA corresponding to the hydroxymethylation variable target region set and the captured cfDNA corresponding to the at least one other target region set in the same vessel.

### K. DNA Amplification

In some embodiments, DNA is amplified. In some embodiments, the DNA can be subjected to a plurality of distinct amplification reactions. Adapted DNA can be amplified (e.g. by PCR) prior to, or as part of, sequencing. For example, in sequencing procedures which comprise a conversion step, the adapted DNA may be amplified after the conversion step. In sequencing procedures which involve single molecule sequencing (such a nanopore-based sequencing or SMRT sequencing), there may be no amplification step. In some embodiments, the DNA amplification step is performed prior to a step of subjecting adapter-ligated DNA to sequencing.

Amplification is typically primed by primers binding to primer binding sites in adapters flanking a DNA molecule to be amplified. Amplification methods can involve cycles of denaturation, annealing and extension, resulting from thermocycling or can be isothermal as in transcription-mediated amplification. For example, sample nucleic acids flanked by adapters can be amplified by PCR and other amplification methods. Amplification methods of use herein can include any suitable methods, such as known to those of ordinary skill in the art. In some embodiments, amplification is primed by primers binding to primer binding sites in adapters flanking a DNA molecule to be amplified. Amplification methods can involve cycles of denaturation, annealing and extension, resulting from thermocycling, such as polymerase chain reaction (PCR), or can be isothermal, such as in linear amplification methods, transcription-mediated amplification, recombinase polymerase amplification (RPA), helices dependent amplification (HDA), loop-mediated isothermal amplification (LAMP) (Notomi et al., Nuc. Acids Res., 28, e63, 2000), rolling-circle amplification (RCA) (Blanco et al., J. Biol. Chem., 264, 8935-8940, 1989), or hyperbranched rolling circle amplification (Lizard et al., Nat. Genetics, 19, 225-232, 1998). Other amplification methods include the ligase chain reaction, strand displacement amplification, nucleic acid sequence based amplification, and self-sustained sequence based replication.

In some embodiments, the present methods perform dsDNA ligations with T-tailed and C-tailed adapters. The addition of C-tailed adapters can increase ligation efficiency because the A-tailing reaction can also add G-tails to a small portion of the DNA molecules, when the A tailing is performed in the presence of dGTP, such as when the A-tailing is performed in the same reaction as the end repair. The use of T-tailed and C-tailed adapters can result in amplification of at least 50, 60, 70 or 80% of double stranded nucleic acids before. The present methods can increase the amount or number of amplified molecules relative to control methods performed with T-tailed adapters alone by at least 10, 15 or 20%.

In some embodiments, adapted DNA is amplified before sequencing. Amplification may in some cases be before one or more capture steps. In some embodiments, the ligation step occurs after the conversion step. In some embodiments, the ligation occurs before or simultaneously with amplification.

In some embodiments, the amplification of the DNA (e.g., adapter ligated DNA) comprises using a DNA polymerase. In some embodiments, the DNA polymerase may be Q5® High-Fidelity DNA Polymerase, Q5U® Hot Start High-Fidelity DNA Polymerase, Phusion® High-Fidelity DNA Polymerase, OneTaq® DNA Polymerase, Taq DNA Polymerase, LongAmp® Taq DNA Polymerase, Hemo KlenTaq, Epimark® Hot Start Taq DNA Polymerase, Bst DNA Polymerase, Full Length, Bst DNA Polymerase, Large Fragment, Bst 2.0 DNA Polymerase, Bst 3.0 DNA Polymerase, Bsu DNA Polymerase, Large Fragment, phi29 DNA Polymerase, phi29-XT DNA Polymerase, Sulfolobus DNA Polymerase IV, Therminator™ DNA Polymerase, T7 DNA Polymerase, DNA Polymerase I (E. coli), DNA Polymerase I, Large (Klenow) Fragment (“Klenow fragment”), Klenow Fragment (3′→5′ exo-), T4 DNA Polymerase, Vent® DNA Polymerase, Vent® (exo-) DNA Polymerase, Deep Vent® DNA Polymerase, Deep Vent® (exo-) DNA Polymerase, or any combination thereof.

In some embodiments, DNA can be amplified by methylation-preserving amplification. In some embodiments, the methylation-preserving amplification can occur before the contacting the DNA in a sample with an mCpG-binding protein.

Amplification, including methylation-preserving amplification, is typically primed by primers binding to primer binding sites in adapters flanking a DNA molecule to be amplified. Amplification methods can involve cycles of denaturation, annealing and extension, resulting from thermocycling or can be isothermal as in transcription-mediated amplification. For example, DNA flanked by adapters added to the DNA as described herein can be amplified by PCR or other amplification methods. Amplification methods of use herein, including methylation-preserving amplification, can include any suitable methods, such as known to those of ordinary skill in the art. In some embodiments, amplification is primed by primers binding to primer binding sites in adapters flanking a DNA molecule to be amplified. Amplification methods can involve cycles of denaturation, annealing and extension, resulting from thermocycling, such as polymerase chain reaction (PCR), or can be isothermal, such as in linear amplification methods, transcription-mediated amplification, recombinase polymerase amplification (RPA), helices dependent amplification (HDA), loop-mediated isothermal amplification (LAMP) (Notomi et al., Nuc. Acids Res., 28, e63, 2000), rolling-circle amplification (RCA) (Blanco et al., J. Biol. Chem., 264, 8935-8940, 1989), or hyperbranched rolling circle amplification (Lizard et al., Nat. Genetics, 19, 225-232, 1998). Other amplification methods include the ligase chain reaction, strand displacement amplification, nucleic acid sequence-based amplification, and self-sustained sequence based replication. In some embodiments, the methylation-preserving amplification comprises linear amplification with thermocycling.

In some embodiments, methylation-preserving amplification comprises amplification performed in the presence of a methyltransferase. Methylating agents of use in methylation-preserving amplification methods described herein are known to those of ordinary skill in the art, and can include, for example, any suitable methyltransferase. In some embodiments, the methylating agent is DNMT1. DNMT1 is the most abundant DNA methyltransferase in mammalian cells and predominantly methylates hemimethylated CpG di-nucleotides in the mammalian genome. For example, DNA molecules replicated using PCR amplification with DNMT1 incubation will maintain their methylation status post-amplification, for use in further analyses, such as those described herein (such as an epigenetic base conversion step and/or an enrichment step).

Additional methylating agents useful herein include the mammalian methyltransferases, DNMT3a and DNMT3b, the plant methyltransferases, MET1, and CMT3. In some embodiments, DNMT1 or another suitable methyltransferase is used with a methyl donor and may be used with or without cofactors known to those of ordinary skill in the art. DNMT1 works in vitro at 95% efficiency without a cofactor; however, DNMT1 may be used with a cofactor such as NP95 (Uhrfl), such as described in Bashtrykov P I, et al. “The UHRF1 protein stimulates the activity and specificity of the maintenance DNA methyltransferase DNMT1 by an allosteric mechanism.” J Biol Chem. 2014. In some embodiments, DNMT1 is used at a concentration of about 50-10000 U/mL, such as about 50-2000, about 50-5000, about 2500-7500, or about 5000-10000 U/mL. In some embodiments, DNMT1 is used at a concentration of about 100-500, about 500-1000, about 100-1000, about 1000-1500, about 500-1500, about 600-1400, about 700-1300, about 800-1200, about 900-1100, or about 950-1050 U/mL. In some embodiments, DNMT1 is used at a concentration of about 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, or about 2000 U/mL. In some embodiments, DNMT1 is used at a concentration of about 1,000 U/ml.

In some embodiments, enriching methylated DNA in a sample comprises amplification, such as embodiments comprising quantitative PCR (qPCR) or digital PCR. Some such embodiments comprising targeted detection of DNA sequences using qPCR or digital PCR do not comprise standard DNA library preparation steps, such as adapter ligation or tagging.

In some embodiments, the present methods perform dsDNA ligations with T-tailed and C-tailed adapters. The addition of C-tailed adapters can increase ligation efficiency because the A-tailing reaction can also add G-tails to a small portion of the DNA molecules, when the A tailing is performed in the presence of dGTP, such as when the A-tailing is performed in the same reaction as the end repair. The use of T-tailed and C-tailed adapters can result in amplification of at least 50, 60, 70 or 80% of double stranded nucleic acids. The present methods can increase the amount or number of amplified molecules relative to control methods performed with T-tailed adapters alone by at least 10, 15, or 20%.

In some embodiments, adapted DNA is amplified before sequencing. Amplification may in some cases be before one or more capture steps. In some embodiments, the ligation step occurs after the conversion step. In some embodiments, the ligation occurs before or simultaneously with amplification.

### III. Additional Features of Certain Disclosed Methods

### A. Samples

A sample can be any biological sample isolated from a subject. A sample can be a bodily sample. Samples can include body tissues, such as known or suspected solid tumors, whole blood, buffy coat, PBMCs, platelets, serum, plasma, stool, red blood cells, white blood cells or leukocytes, endothelial cells, tissue biopsies, cerebrospinal fluid synovial fluid, lymphatic fluid, ascites fluid, interstitial or extracellular fluid, the fluid in spaces between cells, including gingival crevicular fluid, bone marrow, pleural effusions, cerebrospinal fluid, saliva, mucous, sputum, semen, sweat, urine. Samples are preferably body fluids, particularly blood and fractions thereof, and urine. A sample can be in the form originally isolated from a subject or can have been subjected to further processing to remove or add components, such as cells, or enrich for one component relative to another. Thus, preferred body fluids for analysis include body fluids comprising cells, such as whole blood, buffy coat separated from whole blood, PBMCs separated from whole blood, a leukapheresis sample, and/or plasma or serum.

In some embodiments, a population of nucleic acids is obtained from a serum, plasma, or blood sample (such as a buffy coat sample or any other sample comprising cells or a blood sample (e.g., a whole blood sample, a leukapheresis sample, or a PBMC sample)) from a subject suspected of having neoplasia, a tumor, precancer, or cancer or previously diagnosed with neoplasia, a tumor, precancer, or cancer. The population includes nucleic acids having varying levels of sequence variation, epigenetic variation, and/or post-replication or transcriptional modifications.

A sample can be isolated or obtained from a subject and transported to a site of sample analysis. The sample may be preserved and shipped at a desirable temperature, e.g., room temperature, 4° C., −20° C., and/or −80° C. A sample can be isolated or obtained from a subject at the site of the sample analysis. The subject can be a human, a mammal, an animal, a companion animal, a service animal, or a pet. The subject may have a cancer, precancer, infection, transplant rejection, or other disease or disorder related to changes in the immune system. The subject may not have cancer or a detectable cancer symptom. The subject may have been treated with one or more cancer therapy, e.g., any one or more of chemotherapies, antibodies, vaccines, or biologics. The subject may be in remission. The subject may or may not be diagnosed of being susceptible to cancer or any cancer-associated genetic mutations/disorders.

In some embodiments, the sample comprises plasma. The volume of plasma obtained can depend on the desired read depth for sequenced regions. Exemplary volumes are 0.4-40 mL, 5-20 mL, 10-20 mL, and 3-5 mL. For example, the volume can be 0.5 mL, 1 mL, 2 mL, 3 mL, 4 mL, 5 mL, 6 mL, 7 mL, 8 mL, 9 mL, 10 mL, 20 mL, 30 mL, or 40 mL. A volume of sampled plasma may be 5 to 20 mL. In some embodiments, the sample volume is 3-5 mL of plasma, such as 4 mL of plasma, per 10 mL whole blood.

In some embodiments, the sample comprises whole blood. Exemplary volumes of sampled whole blood are 0.4-40 mL, 5-20 mL, 10-20 mL, 1-6 mL, 1-3 mL, and 3-5 mL. For example, the volume can be 0.5 mL, 1 mL, 2 mL, 3 mL, 4 mL, 5 mL, 6 mL, 7 mL, 8 mL, 9 mL, 10 mL, 20 mL, 30 mL, or 40 mL. A volume of sampled whole blood may be 5 to 20 mL. In some embodiments, the sample volume is 1-5 mL of whole blood, such as 2.5 mL of whole blood.

In some embodiments, the sample comprises buffy coat separated from whole blood. Exemplary volumes of sampled buffy coat are 0.1-20 mL, 1-10 mL, 1-5 mL, 0.2-0.6 mL, and 0.3-0.5 mL. For example, the volume can be 0.1 mL, 0.2 mL, 0.3 mL, 0.4 mL, 0.5 mL, 0.6 mL, 0.7 mL, 0.8 mL, 0.9 mL, 1 mL, 2 mL, 3 mL, 4 mL, 5 mL 10 mL, or 20 mL. A volume of sampled buffy coat may be 1 to 10 mL. In some embodiments, the sample volume is 0.1-0.5 mL of buffy coat, such as 0.3 mL of buffy coat, per 10 mL whole blood.

In some embodiments, the sample comprises PBMCs separated from whole blood. Exemplary volumes of sampled PBMCs are 0.1-20 mL, 1-10 mL, 1-5 mL, 0.2-0.6 mL, and 0.3-0.5 mL. For example, the volume can be 0.1 mL, 0.2 mL, 0.3 mL, 0.4 mL, 0.5 mL, 0.6 mL, 0.7 mL, 0.8 mL, 0.9 mL, 1 mL, 2 mL, 3 mL, 4 mL, 5 mL 10 mL, or 20 mL. A volume of sampled PBMCs may be 1 to 10 mL. In some embodiments, the sample volume is 0.1-0.5 mL of PBMCs, such as 0.3 mL of PBMCs, per 10 mL whole blood.

In some embodiments, the sample comprises leukocytes separated from subject blood using leukapheresis. Exemplary volumes of sampled leukocytes from leukapheresis are 0.1-20 mL, 1-10 mL, 1-5 mL, 0.2-0.6 mL, and 0.3-0.5 mL. For example, the volume can be 0.1 mL, 0.2 mL, 0.3 mL, 0.4 mL, 0.5 mL, 0.6 mL, 0.7 mL, 0.8 mL, 0.9 mL, 1 mL, 2 mL, 3 mL, 4 mL, 5 mL, 10 mL, or 20 mL. A volume of sampled leukocytes from leukapheresis may be 1 to 10 mL. In some embodiments, the sample volume is 0.1-0.6 mL of leukocytes from leukapheresis, such as 0.4 mL of leukocytes, per 10 mL whole blood.

A sample can comprise various amount of nucleic acid that contains genome equivalents. For example, a sample of about 30 ng cDNA can contain about 10,000 (104) haploid human genome equivalents. Similarly, a sample of about 100 ng of cDNA can contain about 30,000 haploid human genome equivalents.

A sample can comprise nucleic acids from different sources, e.g., from cells of the same subject or from cells of different subjects. A sample can comprise nucleic acids carrying mutations. For example, a sample can comprise cDNA carrying germline mutations and/or somatic mutations. Germline mutations refer to mutations existing in germline nucleic acids of a subject. Somatic mutations refer to mutations originating in somatic cells of a subject, e.g., precancer cells or cancer cells. A sample can comprise cDNA carrying cancer-associated mutations (e.g., cancer-associated somatic mutations). A sample can comprise an epigenetic variant (i.e., a chemical or protein modification), wherein the epigenetic variant associated with the presence of a genetic variant such as a cancer-associated mutation. In some embodiments, the sample comprises an epigenetic variant associated with the presence of a genetic variant, wherein the sample does not comprise the genetic variant.

Exemplary amounts of nucleic acids (e.g., cDNA prepared from RNA from a sample comprising cells or a blood sample (e.g., a whole blood sample, a buffy coat sample, a leukapheresis sample, or a PBMC sample) in a sample before amplification range from about 1 fg to about 1 μg, e.g., 1 μg to 200 ng, 1 ng to 100 ng, 10 ng to 1000 ng. For example, the amount can be up to about 600 ng, up to about 500 ng, up to about 400 ng, up to about 300 ng, up to about 200 ng, up to about 100 ng, up to about 50 ng, or up to about 20 ng of nucleic acid molecules. The amount can be at least 1 fg, at least 10 fg, at least 100 fg, at least 1 μg, at least 10 pg, at least 100 μg, at least 1 ng, at least 10 ng, at least 100 ng, at least 150 ng, or at least 200 ng of nucleic acid molecules. The amount can be up to 1 femtogram (fg), 10 fg, 100 fg, 1 picogram (pg), 10 μg, 100 μg, 1 ng, 10 ng, 100 ng, 150 ng, or 200 ng of nucleic acid molecules. The method can comprise obtaining 1 femtogram (fg) to 200 ng.

Nucleic acids can be isolated from cells or bodily fluids, which may comprise cells. Cells can be lysed and cellular nucleic acids processed. Generally, after addition of buffers and wash steps, nucleic acids can be precipitated with an alcohol. Further clean up steps may be used such as silica-based columns to remove contaminants or salts. Non-specific bulk carrier nucleic acids, such as C 1 DNA, or DNA or protein for hybridization and/or ligation, may be added throughout the reaction to optimize certain aspects of the procedure such as yield.

After such processing, samples can include various forms of nucleic acid including double stranded cDNA, single stranded cDNA, and single stranded RNA. In some embodiments, single stranded cDNA and RNA can be converted to double stranded forms so they are included in subsequent processing and analysis steps.

cDNA molecules can be linked to adapters at either one end or both ends. Typically, double-stranded molecules are blunt ended by treatment with a polymerase with a 5′-3′ polymerase and a 3′-5′ exonuclease (or proof-reading function), in the presence of all four standard nucleotides. Klenow large fragment and T4 polymerase are examples of suitable polymerase. The blunt ended cDNA molecules can be ligated with at least partially double stranded adapter (e.g., a Y shaped or bell-shaped adapter). Alternatively, complementary nucleotides can be added to blunt ends of sample nucleic acids and adapters to facilitate ligation. Contemplated herein are both blunt end ligation and sticky end ligation. In blunt end ligation, both the nucleic acid molecules and the adapter tags have blunt ends. In sticky-end ligation, typically, the nucleic acid molecules bear an “A” overhang and the adapters bear a “T” overhang.

### B. Subjects

In some embodiments, the RNA (e.g., RNA from a sample comprising cells or a blood sample (e.g., a whole blood sample, a buffy coat sample, a leukapheresis sample, or a PBMC sample) and/or additional RNA) is obtained from a subject having a cancer or a precancer (such as any type of cancer mentioned elsewhere herein, e.g., advanced adenoma and/or colorectal cancer), an infection, transplant rejection, or other disease directly or indirectly affecting the immune system. In some embodiments, the RNA (e.g., RNA from a sample comprising cells or a blood sample (e.g., a whole blood sample, a buffy coat sample, a leukapheresis sample, or a PBMC sample) and/or additional RNA) is obtained from a subject suspected of having a cancer or a precancer, an infection, transplant rejection, or other disease directly or indirectly affecting the immune system. In some embodiments, the RNA (e.g., RNA from a sample comprising cells or a blood sample (e.g., a whole blood sample, a buffy coat sample, a leukapheresis sample, or a PBMC sample) and/or additional RNA) is obtained from a subject having a tumor. In some embodiments, the RNA (e.g., RNA from a sample comprising cells or a blood sample (e.g., a whole blood sample, a buffy coat sample, a leukapheresis sample, or a PBMC sample) and/or additional RNA) is obtained from a subject suspected of having a tumor. In some embodiments, the RNA (e.g., RNA from a sample comprising cells or a blood sample (e.g., a whole blood sample, a buffy coat sample, a leukapheresis sample, or a PBMC sample) and/or additional RNA) is obtained from a subject having neoplasia. In some embodiments, the RNA (e.g., RNA from a sample comprising cells or a blood sample (e.g., a whole blood sample, a buffy coat sample, a leukapheresis sample, or a PBMC sample) and/or additional RNA) is obtained from a subject suspected of having neoplasia. In some embodiments, the RNA (e.g., RNA from a sample comprising cells or a blood sample (e.g., a whole blood sample, a buffy coat sample, a leukapheresis sample, or a PBMC sample) and/or additional RNA) is obtained from a subject in remission from a tumor, cancer, or neoplasia (e.g., following chemotherapy, surgical resection, radiation, or a combination thereof). In some embodiments, the RNA (e.g., RNA from a sample comprising cells or a blood sample (e.g., a whole blood sample, a buffy coat sample, a leukapheresis sample, or a PBMC sample) and/or additional RNA) is obtained from a subject that does not have a cancer or a precancer, an infection, transplant rejection, or other disease directly or indirectly affecting the immune system. In some embodiments, the subject that does not have a cancer or a precancer, an infection, transplant rejection, or other disease directly or indirectly affecting the immune system has self-reported not having the cancer or the precancer, the infection, transplant rejection, or other disease directly or indirectly affecting the immune system. In some embodiments, the RNA (e.g., RNA from a sample comprising cells or a blood sample (e.g., a whole blood sample, a buffy coat sample, a leukapheresis sample, or a PBMC sample) and/or additional RNA) is obtained from a subject that has self-reported not having a cancer or a precancer, an infection, transplant rejection, or other disease directly or indirectly affecting the immune system. In some embodiments, the RNA (e.g., RNA from a sample comprising cells or a blood sample (e.g., a whole blood sample, a buffy coat sample, a leukapheresis sample, or a PBMC sample) and/or additional RNA) is obtained from a subject that has been confirmed to not have the cancer or the precancer, the infection, transplant rejection, or other disease directly or indirectly affecting the immune system, such as by one or more medical tests (such as by an analysis of one or more blood and/or tissue samples from the subject), such as one or more medical tests performed by a medical professional (such as a clinician, such as a nurse or a medical doctor).

In any of the foregoing embodiments, the precancer, cancer, tumor, or neoplasia or suspected precancer, cancer, tumor, or neoplasia may be of the skin, bladder, head or neck, lung, colon, rectum, kidney, breast, prostate, or liver. In some embodiments, the precancer, cancer, tumor, or neoplasia or suspected precancer, cancer, tumor, or neoplasia is of the lung. In some embodiments, the precancer, cancer, tumor, or neoplasia or suspected precancer, cancer, tumor, or neoplasia is of the colon or rectum. In some embodiments, the precancer, cancer, tumor, or neoplasia or suspected precancer, cancer, tumor, or neoplasia is of the breast. In some embodiments, the precancer, cancer, tumor, or neoplasia or suspected precancer, cancer, tumor, or neoplasia is of the prostate. In any of the foregoing embodiments, the subject may be a human subject.

### C. Capture Moieties

As discussed above, nucleic acids in a sample (such as RNA (such as RNA of a first subsample or RNA isolated from a sample) or DNA (such as DNA of a second subsample or isolated from a sample, or cDNA prepared from RNA)) can be subject to a capture step, in which molecules having target regions are captured for subsequent analysis. Target capture can involve use of probes (e.g., oligonucleotides) labeled with a capture moiety, such as biotin, and a second moiety or binding partner that binds to the capture moiety, such as streptavidin. Methods comprising capture moieties are further described in, for example, U.S. Pat. No. 9,850,523, issuing Dec. 26, 2017, which is incorporated herein by reference.

Capture moieties include, without limitation, biotin, avidin, streptavidin, a nucleic acid comprising a particular nucleotide sequence, a hapten recognized by an antibody, and magnetically attractable particles. The extraction moiety can be a member of a binding pair, such as biotin/streptavidin or hapten/antibody. In some embodiments, a capture moiety that is attached to an analyte is captured by its binding pair which is attached to an isolatable moiety, such as a magnetically attractable particle or a large particle that can be sedimented through centrifugation. The capture moiety can be any type of molecule that allows affinity separation of nucleic acids bearing the capture moiety from nucleic acids lacking the capture moiety. Exemplary capture moieties are biotin which allows affinity separation by binding to streptavidin linked or linkable to a solid phase or an oligonucleotide, which allows affinity separation through binding to a complementary oligonucleotide linked or linkable to a solid phase.

In some embodiments, methods disclosed herein can comprise a step of capturing (e.g., enriching) DNA, such as type-specific cfDNA target regions that are also copy number variants. In some embodiments, the capturing step comprises contacting the DNA with probes specific for the target regions. Enrichment or capture may be performed on any sample or subsample described herein using any suitable approach known in the art.

As discussed above, nucleic acids in a sample can be subject to a capture step, in which molecules having certain characteristics are captured and analyzed. Target capture can involve use of a bait set comprising oligonucleotide baits labeled with a capture moiety, such as biotin or the other examples noted below. The probes can have sequences selected to tile across a panel of regions, such as genes. In some embodiments, a bait set can have higher and lower capture yields for sets of target regions such as those of the sequence-variable target region set and the epigenetic target region set, respectively, as discussed elsewhere herein. Such bait sets are combined with a sample under conditions that allow hybridization of the target molecules with the baits. Then, captured molecules are isolated using the capture moiety. DNA capture can involve use of oligonucleotides labeled with a capture moiety, such as target-specific probes labeled with biotin, and a second moiety or binding partner that binds to the capture moiety, such as streptavidin. In some embodiments, a capture moiety and binding partner can have higher and lower capture yields for different sets of probes, such as those used to capture a sequence-variable target region set and an epigenetic target region set, respectively, as discussed elsewhere herein.

Capture may be performed using any suitable approach known in the art. Target capture can involve use of a bait set comprising oligonucleotide baits (a type of probe useful herein) labeled with a capture moiety, such as biotin or the other examples noted below. The probes can have sequences selected to tile across a panel of regions, such as genes. Such bait sets are combined with a sample under conditions that allow hybridization of the target molecules with the baits. Then, captured molecules are isolated using the capture moiety. For example, a biotin capture moiety by bead-based streptavidin. Such methods are further described in, for example, U.S. Pat. No. 9,850,523, issuing Dec. 26, 2017, which is incorporated herein by reference.

Capture moieties include, without limitation, biotin, avidin, streptavidin, a nucleic acid comprising a particular nucleotide sequence, digoxygenin, a histidine tag, an affinity tag, an immunoglobulin constant domain, a hapten recognized by an antibody, and magnetically attractable particles. In some embodiments, the immunoglobulin constant domain may be bound using protein A, protein G, or a secondary antibody. In some embodiments, the secondary antibody comprises an anti-mouse secondary antibody. In some embodiments, the anti-mouse secondary antibody is a goat anti-mouse secondary antibody, rabbit anti-mouse secondary antibody, or a donkey anti-mouse secondary antibody. The extraction moiety can be a member of a binding pair, such as biotin/streptavidin or hapten/antibody. In some embodiments, a capture moiety that is attached to an analyte is captured by its binding pair which is attached to an isolatable moiety, such as a magnetically attractable particle or a large particle that can be sedimented through centrifugation. The capture moiety can be any type of molecule that allows affinity separation of nucleic acids bearing the capture moiety from nucleic acids lacking the capture moiety. Exemplary capture moieties are biotin that allows affinity separation by binding to streptavidin linked or linkable to a solid phase or an oligonucleotide, which allows affinity separation through binding to a complementary oligonucleotide linked or linkable to a solid phase.

In some embodiments, the probes specific for the target regions (i.e., target-specific probes) comprise a capture moiety that facilitates the enrichment or capture of the DNA hybridized to the probes. In some embodiments, the capture moiety is biotin. In some such embodiments, streptavidin attached to a solid support, such as magnetic beads, is used to bind to the biotin. Nonspecifically bound DNA that does not comprise a target region is washed away from the captured DNA. In some embodiments, DNA is then dissociated from the probes and eluted from the solid support using salt washes or buffers comprising another DNA denaturing agent. In some embodiments, the probes are also eluted from the solid support by, e.g., disrupting the biotin-streptavidin interaction. In some embodiments, captured DNA is amplified following elution from the solid support. In some such embodiments, DNA comprising adapters is amplified using PCR primers that anneal to the adapters. In some embodiments, captured DNA is amplified while attached to the solid support. In some such embodiments, the amplification comprises use of a PCR primer that anneals to a sequence within an adapter and a PCR primer that anneals to a sequence within a probe annealed to the target region of the DNA.

A panel of regions targeted for enrichment can be selected such that they do not contain regions known to include the base modification used in an end repair reaction. When end repair is performed with dNTPs comprising 5mC or 5hmC, a panel of regions targeted for enrichment may be selected such that they do not contain CpH dinucleotides which are known to be naturally methylated in the subject (e.g. humans). Such CpH dinucleotides can be identified through the use of publicly available resources (e.g. MethBank3.0: a database of DNA methylomes across a variety of species Nucleic Acids Res 2018). Such an approach has the advantage that any detected methylated CpH dinucleotides can unambiguously be attributed to regions synthesized in end repair.

In some embodiments, capturing comprises contacting the DNA to be captured with a set of target-specific probes. The set of target-specific probes may have any of the features described herein for sets of target-specific probes, including but not limited to in the embodiments set forth above and the sections relating to probes below. Capturing may be performed on one or more subsamples prepared during methods disclosed herein. In some embodiments, DNA is captured from at least the first subsample or the second subsample, e.g., at least the first subsample and the second subsample. In some embodiments, the subsamples are differentially tagged (e.g., as described herein) and then pooled before undergoing capture.

The capturing step may be performed using conditions suitable for specific nucleic acid hybridization, which generally depend to some extent on features of the probes such as length, base composition, etc. Those skilled in the art will be familiar with appropriate conditions given general knowledge in the art regarding nucleic acid hybridization. In some embodiments, complexes of target-specific probes and DNA are formed.

In some embodiments, a method described herein comprises capturing DNA (such as cfDNA) obtained from a subject for a plurality of sets of target regions. The target regions comprise epigenetic target regions, which may show differences in methylation levels and/or fragmentation patterns depending on whether they originated from a tumor or from healthy cells. The target regions also comprise sequence-variable target regions, which may show differences in sequence depending on whether they originated from a tumor or from healthy cells. The capturing step produces a captured set of cfDNA molecules and the cfDNA molecules corresponding to the sequence-variable target region set are captured at a greater capture yield in the captured set of cfDNA molecules than cfDNA molecules corresponding to the epigenetic target region set. For additional discussion of capturing steps, capture yields, and related aspects, see WO2020/160414, which is incorporated herein by reference for all purposes.

In some embodiments, a method described herein comprises contacting cfDNA obtained from a subject with a set of target-specific probes, wherein the set of target-specific probes is configured to capture cfDNA corresponding to the sequence-variable target region set at a greater capture yield than cfDNA corresponding to the epigenetic target region set.

It can be beneficial to capture cfDNA corresponding to the sequence-variable target region set at a greater capture yield than cfDNA corresponding to the epigenetic target region set because a greater depth of sequencing may be necessary to analyze the sequence-variable target regions with sufficient confidence or accuracy than may be necessary to analyze the epigenetic target regions. The volume of data needed to determine fragmentation patterns (e.g., to test for perturbation of transcription start sites or CTCF binding sites) or fragment abundance (e.g., in hypermethylated and hypomethylated partitions) is generally less than the volume of data needed to determine the presence or absence of cancer-related sequence mutations. Capturing the target region sets at different yields can facilitate sequencing the target regions to different depths of sequencing in the same sequencing run (e.g., using a pooled mixture and/or in the same sequencing cell). Although copy number variations such as focal amplifications are somatic mutations, they can be detected by sequencing based on read frequency in a manner analogous to approaches for detecting certain epigenetic changes such as changes in methylation. Thus, they can be considered epigenetic target regions for functional reasons. Additionally, regions showing copy number variation that are also hypermethylation-variable or fragmentation-variable target regions are considered epigenetic target regions because they may show epigenetic variation.

In various embodiments, the methods further comprise sequencing the captured DNA, e.g., to different degrees of sequencing depth for the epigenetic and sequence-variable target region sets, consistent with the discussion herein.

In some embodiments, complexes of target-specific probes and DNA are separated from DNA not bound to target-specific probes. For example, where target-specific probes are bound covalently or noncovalently to a solid support, a washing or aspiration step can be used to separate unbound material. Alternatively, where the complexes have chromatographic properties distinct from unbound material (e.g., where the probes comprise a ligand that binds a chromatographic resin), chromatography can be used.

As discussed in detail elsewhere herein, the set of target-specific probes may comprise a plurality of sets such as probes for a sequence-variable target region set and probes for an epigenetic target region set. In some embodiments, a capturing step is performed with the probes for a sequence-variable target region set and the probes for an epigenetic target region set in the same vessel at the same time, e.g., the probes for the sequence-variable and epigenetic target region sets and capture probes are in the same composition. This approach provides a relatively streamlined workflow. In some embodiments, the concentration of the probes for the sequence-variable target region set is greater than the concentration of the probes for the epigenetic target region set.

Alternatively, the capturing step is performed with the sequence-variable target region probe set in a first vessel and with the epigenetic target region probe set in a second vessel, or the contacting step is performed with the sequence-variable target region probe set at a first time and a first vessel and the epigenetic target region probe set at a second time before or after the first time. This approach allows for preparation of separate first and second compositions comprising captured DNA corresponding to the sequence-variable target region set and captured DNA corresponding to the epigenetic target region set. The compositions can be processed separately as desired (e.g., to fractionate based on methylation as described elsewhere herein) and recombined in appropriate proportions to provide material for further processing and analysis such as sequencing.

In some embodiments, a captured set of DNA (e.g., cfDNA) is provided. With respect to the disclosed methods, the captured set of DNA may be provided, e.g., by performing a capturing step prior to a sequencing step as described herein. The captured set may comprise DNA corresponding to a sequence-variable target region set, an epigenetic target region set, or a combination thereof. In some embodiments, a capture step is performed prior to a conversion step or after a conversion step.

In some embodiments, a first target region set is captured (e.g., from a sample or a first subsample), comprising at least epigenetic target regions. The epigenetic target regions captured from the first subsample may comprise hypermethylation variable target regions. In some embodiments, the hypermethylation variable target regions are CpG-containing regions that are unmethylated or have low methylation in cfDNA from healthy subjects (e.g., below-average methylation relative to bulk cfDNA). In some embodiments, the hypermethylation variable target regions are regions that are regions that show lower methylation in healthy cfDNA than in at least one other tissue type. Without wishing to be bound by any particular theory, cancer cells may shed more DNA into the bloodstream that healthy cells of the same tissue type. As such, the distribution of tissue of origin of cfDNA may change upon carcinogenesis. Thus, an increase in the level of hypermethylation variable target regions in the first subsample can be an indicator of the presence (or recurrence, depending on the history of the subject) of cancer.

In some embodiments, a second target region set is captured from the second subsample comprising at least epigenetic target regions. The epigenetic target regions may comprise hypomethylation variable target regions. In some embodiments, the hypomethylation variable target regions are CpG-containing regions that are methylated or have high methylation in cfDNA from healthy subjects (e.g., above-average methylation relative to bulk cfDNA). In some embodiments, the hypomethylation variable target regions are regions that show higher methylation in healthy cfDNA than in at least one other tissue type. Without wishing to be bound by any particular theory, cancer cells may shed more DNA into the bloodstream than healthy cells of the same tissue type. As such, the distribution of tissue of origin of cfDNA may change upon carcinogenesis. Thus, an increase in the level of hypomethylation variable target regions in the second subsample can be an indicator of the presence (or recurrence, depending on the history of the subject) of cancer.

In some embodiments the quantity of captured sequence-variable target region DNA is greater than the quantity of the captured epigenetic target region DNA, when normalized for the difference in the size of the targeted regions (footprint size).

Alternatively, first and second captured sets may be provided, comprising, respectively, DNA corresponding to a sequence-variable target region set and DNA corresponding to an epigenetic target region set. The captured sets may be combined to provide a combined captured set.

In some embodiments in which a captured set comprising DNA corresponding to the sequence-variable target region set and the epigenetic target region set includes a combined captured set as discussed above, the DNA corresponding to the sequence-variable target region set may be present at a greater concentration than the DNA corresponding to the epigenetic target region set, e.g., a 1.1 to 1.2-fold greater concentration, a 1.2- to 1.4-fold greater concentration, a 1.4- to 1.6-fold greater concentration, a 1.6- to 1.8-fold greater concentration, a 1.8- to 2.0-fold greater concentration, a 2.0- to 2.2-fold greater concentration, a 2.2- to 2.4-fold greater concentration a 2.4- to 2.6-fold greater concentration, a 2.6- to 2.8-fold greater concentration, a 2.8- to 3.0-fold greater concentration, a 3.0- to 3.5-fold greater concentration, a 3.5- to 4.0, a 4.0- to 4.5-fold greater concentration, a 4.5- to 5.0-fold greater concentration, a 5.0 to 5.5-fold greater concentration, a 5.5- to 6.0-fold greater concentration, a 6.0- to 6.5-fold greater concentration, a 6.5- to 7.0-fold greater, a 7.0- to 7.5-fold greater concentration, a 7.5- to 8.0-fold greater concentration, an 8.0- to 8.5-fold greater concentration, an 8.5- to 9.0-fold greater concentration, a 9.0- to 9.5-fold greater concentration, 9.5- to 10.0-fold greater concentration, a 10- to 11-fold greater concentration, an 11- to 12-fold greater concentration a 12- to 13-fold greater concentration, a 13- to 14-fold greater concentration, a 14- to 15-fold greater concentration, a 15- to 16-fold greater concentration, a 16- to 17-fold greater concentration, a 17- to 18-fold greater concentration, an 18- to 19-fold greater concentration, a 19- to 20-fold greater concentration, a 20- to 30-fold greater concentration, a 30- to 40-fold greater concentration, a 40- to 50-fold greater concentration, a 50- to 60-fold greater concentration, a 60- to 70-fold greater concentration, a 70- to 80-fold greater concentration, a 80 to 90-fold greater concentration, or a 90- to 100-fold greater concentration. The degree of difference in concentrations accounts for normalization for the footprint sizes of the target regions, as discussed in the definition section.

In some embodiments, the DNA that is captured comprises intronic regions. In some embodiments, the intronic regions comprise one or more introns likely to differentiate DNA from neoplastic (e.g., tumor or cancer) cells and from healthy cells, e.g., non-neoplastic circulating cells. For example, an intron comprising a rearrangement known to be present in some neoplastic cells and absent from healthy cells can be used to differentiate DNA from neoplastic (e.g., tumor or cancer) cells and from healthy cells. In some embodiments, the rearrangement is a translocation.

In some embodiments, captured intronic regions have a footprint of at least 30 bp, e.g., at least 100 bp, at least 200 bp, at least 500 bp, at least 1 kb, at least 2 kb, at least 5 kb, at least 10 kb, at least 20 kb, at least 50 kb, at least 200 kb, at least 300 kb, or at least 400 kb. In some embodiments, the intronic target region set has a footprint in the range of 30 bp-1000 kb, e.g., 30 bp-100 bp, 100 bp-200 bp, 200 bp-500 bp, 500 bp-1 kb, 1 kb-2 kb, 2 kb-5 kb, 5 kb-10 kb, 10 kb-20 kb, 20 kb-50 kb, 50 kb-100 kb, 100-200 kb, 200-300 kb, 300-400 kb, 400-500 kb, 500-600 kb, 600-700 kb, 700-800 kb, 800-900 kb, and 900-1,000 kb.

Exemplary rearrangements, such as intronic translocations that can be detected using the methods described herein include but are not limited to translocations wherein at least one of the two genes involved in the translocation is a receptor tyrosine kinase. Exemplary translocation products are the BCR-ABL fusion, and fusions comprising any of ALK, FGFR2, FGFR3, NTRK1, RET, or ROS1.

In some embodiments, the DNA that is captured comprises target regions having a type-specific epigenetic variation and/or a copy number variation. In some embodiments, an epigenetic target region set consists of target regions having a type-specific epigenetic variation and/or a copy number variation. In some embodiments, the type-specific epigenetic variations, e.g., differential methylation or a type-specific fragmentation pattern, are likely to differentiate DNA from one or more related cell or tissue types cells from DNA from other cell or tissue types present in a sample or in a subject.

In some embodiments, nucleic acids captured or enriched using a method described herein comprise captured DNA, such as one or more captured sets of DNA. In some embodiments, the captured DNA comprise target regions that are differentially methylated in different immune cell types. In some embodiments, the immune cell types comprise rare or closely related immune cell types, such as activated and naïve lymphocytes or myeloid cells at different stages of differentiation.

In some embodiments, a captured epigenetic target region set captured from a sample or first subsample comprises hypermethylation variable target regions. In some embodiments, the hypermethylation variable target regions are differentially or exclusively hypermethylated in one or more related cell or tissue types. In some embodiments, the hypermethylation variable target regions are differentially or exclusively hypermethylated in one cell type or in one immune cell type, or in one immune cell type within a cluster. In some embodiments, the hypermethylation variable target regions are hypermethylated to an extent that is distinguishably higher or exclusively present in one cell type or one immune cell type or one immune cell type within a cluster. Such hypermethylation variable target regions may be hypermethylated in other cell or tissue types but not to the extent observed in the one or more related cell or tissue types. In some embodiments, the hypermethylation variable target regions show lower methylation in healthy cfDNA than in at least one other tissue type. In some embodiments, the hypermethylation variable target regions show even higher methylation in cfDNA from a diseased cell of the one or more related cell or tissue types. In some embodiments, target regions comprise hypermethylated regions with aberrantly high copy number. In some such embodiments, the target regions are hypermethylated in healthy and diseased colon tissue and have aberrantly high copy number in pre-cancerous or cancerous colon tissue. Examples of such target regions are shown in Table 2 below.

TABLE 2
Hypermethylated target regions with aberrantly high
copy number in colon cancer or pre-cancer

| Chromosomal | |
|---|---|
| region | |
| with copy | Genes comprising DMRs |
| number gain | within the chromosomal region |
| 7p21.3-15.1 | VWDE, TWIST1, DNAH1, |
| | GPNMB, NPY, GSDME, NFE2L3, |
| | HOXA1, HOXA7, EVX1, CREB5 |
| 8q13.3-24.3 | PRDM14, MSC, TRPA1, SBSPON, |
| | CRISPLD1, ZFHX4, RALYL, |
| | MMP16, C8orf88, RUNX1T1, |
| | CDH17, GDF6, SDC2, OSR2, |
| | PABPC1, ZNF706, RIMS2, |
| | ZFPM2, SYBU, CSMD3, TRPS1, |
| | AARD, TNFRSF11B, MAL2, |
| | COL14A1, POU5F1B, ADCY8, |
| | ST3GAL1, TRAPPC9, AGO2, |
| | ADGRB1, LY6E, LY6H, GLI4, |
| | MAFA, TSTA3, PLEC, OPLAH, FOXH1 |
| 13q14.1-33.3 | ELF1, RGCC, TNFSF11, SERP2, |
| | RB1, CNMD, PCDH17, EDNRB, |
| | POU4F1, SLITRK5, GPC6, |
| | SOX21, CLDN10, STK24, ZIC2, |
| | NALCN, FGF14, MYO16, |
| | COL4A1, COL4A2, SOX1, F7 |
| 20q12-13.33 | MAFB, LPIN3, EMILIN3, |
| | PTPRT, TOX2, GDAPIL1, HNF4A, |
| | MMP9, CDH22, PREX1, ZNFX1, |
| | PARD6B, ZFP64, DOK5, |
| | FAM210B, TFAP2C, RBM38, |
| | CTCFL, GNAS, PHACTR3, CDH4, |
| | GATA5, BHLHE23, YTHDF1, |
| | NKAIN4, CHRNA4, KCNQ2, |
| | GMEB2 |

TABLE 3
Exemplary Hypermethylation Target Regions based
on Lung Cancer studies

| Gene Name | Chromosome |
|---|---|
| MARCH11 | chr5 |
| TAC1 | chr7 |
| TCF21 | chr6 |
| SHOX2 | chr3 |
| p16 | chr3 |
| Casp8 | chr2 |
| CDH13 | chr16 |
| MGMT | chr10 |
| MLH1 | chr3 |
| MSH2 | chr2 |
| TSLC1 | chr11 |
| APC | chr5 |
| DKK1 | chr10 |
| DKK3 | chr11 |
| LKB1 | chr11 |
| WIF1 | chr12 |
| RUNX3 | chr1 |
| GATA4 | chr8 |
| GATA5 | chr20 |
| PAX5 | chr9 |
| E-Cadherin | chr16 |
| H-Cadherin | chr16 |

In some embodiments, a captured epigenetic target region set captured from a sample or subsample comprises hypomethylation variable target regions. In some embodiments, the hypomethylation variable target regions are exclusively hypomethylated in one or more related cell or tissue types. In some embodiments, the hypomethylation variable target regions are exclusively hypomethylated in one cell type or in one immune cell type or in one immune cell type within a cluster. In some embodiments, the hypomethylation variable target regions are hypomethylated to an extent that is exclusively present in one cell type or one immune cell type or in one immune cell type within a cluster. Such hypomethylation variable target regions may be hypomethylated in other cell or tissue types but not to the extent observed in the one or more cell or tissue types. In some embodiments, the hypomethylation variable target regions show higher methylation in healthy cfDNA than in at least one other tissue type. Without wishing to be bound by any particular theory, in an individual with cancer, proliferating or activated immune cells and/or dying cancer cells may shed more DNA into the bloodstream than cells (e.g., immune cells) in a healthy individual and/or healthy cells of the same tissue type, respectively. As such, the distribution of cell type and/or tissue of origin of cfDNA may change upon carcinogenesis. Variations in hypermethylation and/or hypomethylation can be an indicator of disease. Thus, the presence and/or levels of cfDNA originating from certain cell or tissue types can be an indicator of disease. For example, an increase in the level of hypermethylation variable target regions and/or hypomethylation variable target regions in a subsample following a partitioning step can be an indicator of the presence (or recurrence, depending on the history of the subject) of cancer.

Exemplary hypermethylation variable target regions and hypomethylation variable target regions useful for distinguishing between various cell types, including but not limited to immune cell types, have been identified by analyzing DNA obtained from various cell types via whole genome bisulfite sequencing, as described, e.g., in Scott, C. A., Duryea, J. D., MacKay, H. et al., “Identification of cell type-specific methylation signals in bulk whole genome bisulfite sequencing data,” Genome Biol 21, 156 (2020) (doi.org/10.1186/s13059-020-02065-5). Whole-genome bisulfite sequencing data is available from the Blueprint consortium, available on the internet at dcc.blueprint-epigenome.eu.

In some embodiments, first and second captured target region sets comprise, respectively, DNA corresponding to a sequence-variable target region set and DNA corresponding to an epigenetic target region set, for example, as described in WO 2020/160414. The first and second captured sets may be combined to provide a combined captured set. In some embodiments, the sequence-variable target region set and epigenetic target region set may have any of the features described for such sets in WO 2020/160414, which is incorporated by reference herein in its entirety. In some embodiments, the epigenetic target region set comprises a hypermethylation variable target region set. In some embodiments, the epigenetic target region set comprises a hypomethylation variable target region set. In some embodiments, the epigenetic target region set comprises CTCF binding regions. In some embodiments, the epigenetic target region set comprises fragmentation variable target regions. In some embodiments, the epigenetic target region set comprises transcriptional start sites. In some embodiments, the epigenetic target region set comprises regions that may show focal amplifications in cancer, e.g., one or more of AR, BRAF, CCND1, CCND2, CCNE1, CDK4, CDK6, EGFR, ERBB2, FGFR1, FGFR2, KIT, KRAS, MET, MYC, PDGFRA, PIK3CA, and RAF1. For example, in some embodiments, the epigenetic target region set comprises at least 2, 3, 4, 5, 6, 7, 8, 9, 10, 11, 12, 13, 14, 15, 16, 17, or 18 of the foregoing targets.

In some embodiments, the sequence-variable target region set comprises a plurality of regions known to undergo somatic mutations in cancer. In some aspects, the sequence-variable target region set targets a plurality of different genes or genomic regions (“panel”) selected such that a determined proportion of subjects having a cancer exhibits a genetic variant or tumor marker in one or more different genes or genomic regions in the panel. The panel may be selected to limit a region for sequencing to a fixed number of base pairs. The panel may be selected to sequence a desired amount of DNA, e.g., by adjusting the affinity and/or amount of the probes as described elsewhere herein. The panel may be further selected to achieve a desired sequence read depth. The panel may be selected to achieve a desired sequence read depth or sequence read coverage for an amount of sequenced base pairs. The panel may be selected to achieve a theoretical sensitivity, a theoretical specificity, and/or a theoretical accuracy for detecting one or more genetic variants in a sample.

Probes for detecting the panel of regions can include those for detecting genomic regions of interest (hotspot regions). Information about chromatin structure can be taken into account in designing probes, and/or probes can be designed to maximize the likelihood that particular sites (e.g., KRAS codons 12 and 13) can be captured, and may be designed to optimize capture based on analysis of cfDNA coverage and fragment size variation impacted by nucleosome binding patterns and GC sequence composition. Regions used herein can also include non-hotspot regions optimized based on nucleosome positions and GC models.

Probes for detecting the panel of regions can include those for detecting genomic regions of interest (hotspot regions). Information about chromatin structure can be taken into account in designing probes, and/or probes can be designed to maximize the likelihood that particular sites (e.g., KRAS codons 12 and 13) can be captured, and may be designed to optimize capture based on analysis of cfDNA coverage and fragment size variation impacted by nucleosome binding patterns and GC sequence composition. Regions used herein can also include non-hotspot regions optimized based on nucleosome positions and GC models.

Examples of listings of genomic locations of interest may be found in Table 3 and Table 4 of WO 2020/160414. In some embodiments, a sequence-variable target region set used in the methods of the present disclosure comprises at least a portion of at least 5, at least 10, at least 15, at least 20, at least 25, at least 30, at least 35, at least 40, at least 45, at least 50, at least 55, at least 60, at least 65, or 70 of the genes of Table 3 of WO 2020/160414. In some embodiments, a sequence-variable target region set used in the methods of the present disclosure comprises at least a portion of at least 5, at least 10, at least 15, at least 20, at least 25, at least 30, at least 35, at least 40, at least 45, at least 50, at least 55, at least 60, at least 65, at least 70, or 73 of the genes of Table 4 of WO 2020/160414. Additionally or alternatively, suitable target region sets are available from the literature. For example, Gale et al., PLOS One 13: e0194630 (2018), which is incorporated herein by reference, describes a panel of 35 cancer-related gene targets that can be used as part or all of a sequence-variable target region set. These 35 targets are AKT1, ALK, BRAF, CCND1, CDK2A, CTNNB1, EGFR, ERBB2, ESR1, FGFR1, FGFR2, FGFR3, FOXL2, GATA3, GNA11, GNAQ, GNAS, HRAS, IDH1, IDH2, KIT, KRAS, MED12, MET, MYC, NFE2L2, NRAS, PDGFRA, PIK3CA, PPP2R1A, PTEN, RET, STK11, TP53, and U2AF1.

In some embodiments, the sequence-variable target region set comprises target regions from at least 10, 20, 30, or 35 cancer-related genes, such as the cancer-related genes listed above and in WO 2020/160414.

In some embodiments, a collection of capture probes is used in methods described herein, e.g., comprising capture probes prepared by any method disclosed herein for doing so. In some embodiments, the collection of capture probes further comprises target-binding probes specific for a sequence-variable target region set and/or target-binding probes specific for a sequence-variable target region set and/or target-binding probes specific an epigenetic target region set. In some embodiments, the capture yield of the capture probes specific for the sequence-variable target region set is higher (e.g., at least 2-fold higher) than the capture yield of the target-binding probes specific for the epigenetic target region set. In some embodiments, the collection of capture probes is configured to have a capture yield specific for the sequence-variable target region set higher (e.g., at least 2-fold higher) than its capture yield specific for the epigenetic target region set.

In some embodiments, the capture yield of the target-binding probes specific for the sequence-variable target region set is at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than the capture yield of the target-binding probes specific for the epigenetic target region set. In some embodiments, the capture yield of the target-binding probes specific for the sequence-variable target region set is 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10 to 11-, 11- to 12-, 13- to 14-, or 14- to 15-fold higher than the capture yield of the target-binding probes specific for the epigenetic target region set.

In some embodiments, the collection of capture probes is configured to have a capture yield specific for the sequence-variable target region set at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than its capture yield for the epigenetic target region set. In some embodiments, the collection of capture probes is configured to have a capture yield specific for the sequence-variable target region set is 1.25 to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10- to 11-, 11- to 12-, 13- to 14-, or 14- to 15-fold higher than its capture yield specific for the epigenetic target region set.

The collection of probes can be configured to provide higher capture yields for the sequence-variable target region set in various ways, including concentration, different lengths and/or chemistries (e.g., that affect affinity), and combinations thereof. Affinity can be modulated by adjusting probe length and/or including nucleotide modifications as discussed below.

In some embodiments, the capture probes specific for the sequence-variable target region set are present at a higher concentration than the capture probes specific for the epigenetic target region set. In some embodiments, concentration of the target-binding probes specific for the sequence-variable target region set is at least 1.25-, 1.5-, 1.75-, 2-, 2.25-, 2.5-, 2.75-, 3-, 3.5-, 4-, 4.5-, 5-, 6-, 7-, 8-, 9-, 10-, 11-, 12-, 13-, 14-, or 15-fold higher than the concentration of the target-binding probes specific for the epigenetic target region set. In some embodiments, the concentration of the target-binding probes specific for the sequence-variable target region set is 1.25- to 1.5-, 1.5- to 1.75-, 1.75- to 2-, 2- to 2.25-, 2.25- to 2.5-, 2.5- to 2.75-, 2.75- to 3-, 3- to 3.5-, 3.5- to 4-, 4- to 4.5-, 4.5- to 5-, 5- to 5.5-, 5.5- to 6-, 6- to 7-, 7- to 8-, 8- to 9-, 9- to 10-, 10 to 11-, 11- to 12-, 13- to 14-, or 14- to 15-fold higher than the concentration of the target-binding probes specific for the epigenetic target region set. In such embodiments, concentration may refer to the average mass per volume concentration of individual probes in each set.

In some embodiments, the capture probes specific for the sequence-variable target region set have a higher affinity for their targets than the capture probes specific for the epigenetic target region set. Affinity can be modulated in any way known to those skilled in the art, including by using different probe chemistries. For example, certain nucleotide modifications, such as cytosine 5-methylation (in certain sequence contexts), modifications that provide a heteroatom at the 2′ sugar position, and LNA nucleotides, can increase stability of double-stranded nucleic acids, indicating that oligonucleotides with such modifications have relatively higher affinity for their complementary sequences. See, e.g., Severin et al., Nucleic Acids Res. 39:8740-8751 (2011); Freier et al., Nucleic Acids Res. 25:4429-4443 (1997); U.S. Pat. No. 9,738,894. Also, longer sequence lengths will generally provide increased affinity. Other nucleotide modifications, such as the substitution of the nucleobase hypoxanthine for guanine, reduce affinity by reducing the amount of hydrogen bonding between the oligonucleotide and its complementary sequence. In some embodiments, the capture probes specific for the sequence-variable target region set have modifications that increase their affinity for their targets. In some embodiments, alternatively or additionally, the capture probes specific for the epigenetic target region set have modifications that decrease their affinity for their targets. In some embodiments, the capture probes specific for the sequence-variable target region set have longer average lengths and/or higher average melting temperatures than the capture probes specific for the epigenetic target region set. These embodiments may be combined with each other and/or with differences in concentration as discussed above to achieve a desired fold difference in capture yield, such as any fold difference or range thereof described above.

In some embodiments, the capture probes comprise a capture moiety. The capture moiety may be any of the capture moieties described herein, e.g., biotin. In some embodiments, the target-specific probes are linked to a solid support, e.g., covalently or non-covalently such as through the interaction of a binding pair of capture moieties. In some embodiments, the solid support is a bead, such as a magnetic bead.

In some embodiments, the capture probes specific for the sequence-variable target region set and/or the capture probes specific for the epigenetic target region set are a capture probe set as discussed above, e.g., probes comprising capture moieties and sequences selected to tile across a panel of regions, such as genes.

In some embodiments, the capture probes are provided in a single composition. The single composition may be a solution (liquid or frozen). Alternatively, it may be a lyophilizate.

Alternatively, the capture probes may be provided as a plurality of compositions, e.g., comprising a first composition comprising probes specific for the epigenetic target region set and a second composition comprising probes specific for the sequence-variable target region set. These probes may be mixed in appropriate proportions to provide a combined probe composition with any of the foregoing fold differences in concentration and/or capture yield. Alternatively, they may be used in separate capture procedures (e.g., with aliquots of a sample or sequentially with the same sample) to provide first and second compositions comprising captured epigenetic target regions and sequence-variable target regions, respectively.

### 1. Collections of Target-Specific Probes

In some embodiments, a collection of target-specific probes is used to capture RNA or DNA (e.g., cDNA or cfDNA), as described herein. In some embodiments, the target-specific probes comprise a capture moiety. The capture moiety may be any of the capture moieties described herein, e.g., biotin. In some embodiments, the target-specific probes are linked to a solid support, e.g., covalently or non-covalently such as through the interaction of a binding pair of capture moieties. In some embodiments, the solid support is a bead, such as a magnetic bead.

In some embodiments, the target-specific probes are provided in a single composition. The single composition may be a solution (liquid or frozen). Alternatively, it may be a lyophilizate.

Alternatively, the target-specific probes may be provided as a composition, e.g., comprising probes specific for a target region set as described herein.

### 2. Probes Specific for a Target Region Set

The probes for a target region set (such as a target region set comprising one or more genes of a target gene list disclosed herein, or comprising one or more genes of a target gene list generated using the methods disclosed herein) may comprise probes specific for a plurality of target regions of interest (such as one or more of the genes of a target gene list disclosed herein, or one or more of the genes of a target gene list generated using the methods disclosed herein). For example, the probes may be specific for any one or more genes of a target gene list described herein. Exemplary target region sets are discussed in detail herein, e.g., in the sections above concerning captured sets. In some embodiments, captured nucleic acids comprise target regions that are differentially expressed in different immune cell types, or in a sample from a subject that has a disease or condition as compared to a sample from a subject that does not have the disease or condition (e.g., a healthy subject).

In some embodiments, the target region probe set has a footprint of at least 0.5 kb, e.g., at least 1 kb, at least 2 kb, at least 5 kb, at least 10 kb, at least 20 kb, at least 30 kb, or at least 40 kb. In some embodiments, the target region probe set has a footprint in the range of 0.5-100 kb, e.g., 0.5-2 kb, 2-10 kb, 10-20 kb, 20-30 kb, 30-40 kb, 40-50 kb, 50-60 kb, 60-70 kb, 70-80 kb, 80-90 kb, and 90-100 kb. In some embodiments, the target region probe set has a footprint of about 10 kb to about 100 kb, such as about 20 kb to about 100 kb, about 30 kb to about 100 kb, about 40 kb to about 100 kb, about 50 kb to about 100 kb, about 60 kb to about 100 kb, or about 70 kb to about 100 kb. In other embodiments, the footprint of the target region probe set is about 10 kb to about 50 kb, about 20 kb to about 50 kb, about 30 kb to about 50 kb, about 40 kb to about 60 kb, or about 25 kb to about 75 kb. In some embodiments, the footprint for the target region probe set is about 10 kb, about 15 kb, about 20 kb, about 25 kb, about 30 kb, about 35 kb, about 40 kb, about 45 kb, about 50 kb, about 55 kb, about 60 kb, about 65 kb, about 70 kb, about 75 kb, about 80 kb, about 85 kb, about 90 kb, about 95 kb, or about 100 kb.

In some embodiments, probes specific for the target region set comprise probes specific for at least a portion of at least 2, at least 3, at least 4, at least 5, at least 6, at least 7, at least 8, at least 9, at least 10, at least 12, at least 13, at least 14, at least 15, at least 16, at least 17, at least 18, at least 19, at least 20, at least 25, at least 30, at least 35, at least 40, at least 45, at least 50, at least 55, or at least 60 of the genes of Table 4.

In some embodiments, the probes specific for the target region set comprise probes specific for at least a portion of one or more (such as 2, 3, 4, 5, 6, 7, 8, 9, 10, 11, 12, 13, 14, 15, 16, 17, 18, 19, 20, or more), or each, of ICA1, CD38, ABCB4, TNFRSF17, RRP12, CHI3L2, MAP3K13, IL4R, IL12RB2, ACHE, LAG3, CD209, GGT5, DEPDC5, UPK3A, GZMH, BPI, ACP5, CD37, MAST1, RASSF4, MS4A6A, PPFIBP1, MAK, IL18RAP, KYNU, FASLG, MYB, CCND2, TRPM6, FLVCR2, CD80, TMEM156, BHLHE41, NFE2, TREM1, TMEM255A, IL1B, PLEKHG3, VPREB3, TEP1, TRPM4, SMPDL3B, LILRB2, CHI3L1, IL2RA, TLR2, BMP2K, TNFRSF11A, PLA2G7, EPHA1, ABCB9, ZFP36L2, HHEX, SKA1, CLIC2, DUSP2, SLAMF8, CDA, ZNF222, ASGR2, PBXIP1, NIPSNAP3B, TSHR, NOD2, CD300A, NAALADL1, ADRB2, SCN9A, CEACAM3, COL8A2, CLEC7A, CTSW, HPSE, CD7, P2RY14, HIC1, P2RY13, GPR19, CCR3, MROH7, CSF1, FAM174B, TNFRSF4, PDCD1, RYR1, HLA-DQA1, NCR3, ZBTB10, APOBEC3G, LILRA2, TNFRSF13B, HLA-DOB, PGLYRP1, HGF, ATP9A, ATP2C2, JMJD6, DHRS9, SLC1A3, CEACAM1, DUSP13, CRISP3, ABLIM1, HSD3B7, OSM, UPB1, BIK, MMP9, SLCO4A1, BMX, KLF5, RETN, GRB10, PRUNE2, ERLIN1, TP5313, IL1R2, EPAS1, LRRC42, GADD45A, PHTF1, RCAN3, ARG1, CYSTM1, DACH1, FKBP9, GOS2, PFKFB2, CDH26, ARMC7, PPPIR3D, ECHDC3, RDH5, ACVR1B, CKAP4, MTHFS, IL10, MFSD13A, GPR84, MYLK3, ZNF787, MYO10, RAB19, OLAH, ANKRD22, RABGEF1, SAMSN1, CACNA1D, RGL4, TFF3, GYG1, ZDHHC19, SLC51A, POC1A, HPGD, FBN1, CLEC4D, SEMA6B, PFKFB3, PAQR8, SH3PXD2B, FOSL1, MSRA, LRRN1, KCNE5, B3GNT5, CD163, CRACR2B, OPLAH, EXOSC4, KCNE1, UPP1, ST6GALNAC3, SEPTIN5, FCAR, SPATC1, MAFG, IL27, ENTPD7, PLIN5, FAM228B, ORM1, STON1, CFH, HS3ST1, DBNDD1, CD22, SLC25A39, KCNG1, TGFBR3, ADD2, COL19A1, CD200, TCL1A, PROCR, CD40, NME4, TSPAN13, RGS9, FAM184A, KHDRBS2, ENPP5, MMP8, SATB2, GPR68, CEACAM8, MYO1B, LARGE1, NT5E, RAPGEF5, ABHD17C, ZNF365, GRTP1, IGFBP3, LCN2, GLB1L2, CNKSR2, PRSS23, RASGRP3, SCN3A, C16orf74, RETREG1, ERG, SNX22, CXCR5, BEND5, SLC1A7, LEXM, CAMK2N1, SPRY1, CDCA7L, SPIB, DLC1, DIPK1B, MTCL1, PARM1, MZB1, SLC23A1, PDGFD, NMUR1, NBEA, OLR1, PTPRM, PAWR, ABCA13, TAFA1, KCNH8, EFNA5, PRSS57, PTCH1, PRTN3, SULT1A1, ZNF667, SHISA4, L1CAM, RASGEFIA, DDR1, GPC2, PATL2, and IGLL5.PDCD1, ZNK222, CCR3, TRPM4, GPR19, LAG3, TMEM156, MAST1, TSHR, TNFRSF17, ASGR2, TRPM6, TNFRSF11A, CRISP3, RYR1, PLEKHG3, CD209, ACP5, OSM, and CSF1.

In some embodiments, the probes specific for the target region set comprise probes specific for at least a portion of target regions from one or more (such as 2, 3, 4, 5, 6, 7, 8, 9, 10, 11, 12, 13, 14, 15, 16, 17, 18, 19, 20, or more), or each, of PGLYRP1, HGF, ATP9A, ATP2C2, JMJD6, DHRS9, SLC1A3, CEACAM1, DUSP13, CRISP3, ABLIM1, HSD3B7, OSM, UPB1, BIK, MMP9, SLCO4A1, BMX, KLF5, RETN, GRB10, PRUNE2, ERLIN1, TP5313, IL1R2, EPAS1, LRRC42, GADD45A, PHTF1, RCAN3, ARG1, CYSTM1, DACH1, FKBP9, GOS2, PFKFB2, CDH26, ARMC7, PPP1R3D, ECHDC3, RDH5, ACVR1B, CKAP4, MTHFS, IL10, MFSD13A, GPR84, MYLK3, ZNF787, MYO10, RAB19, OLAH, ANKRD22, RABGEF1, SAMSN1, CACNA1D, RGL4, TFF3, GYG1, ZDHHC19, SLC51A, POC1A, HPGD, FBN1, CLEC4D, SEMA6B, PFKFB3, PAQR8, SH3PXD2B, FOSL1, MSRA, LRRN1, KCNE5, B3GNT5, CD163, CRACR2B, OPLAH, EXOSC4, KCNE1, UPP1, ST6GALNAC3, SEPTIN5, FCAR, SPATC1, MAFG, IL27, ENTPD7, PLIN5, FAM228B, ORM1, and STON1.

In some embodiments, the probes specific for the target region set comprise probes specific for at least a portion of target regions from one or more (such as 2, 3, 4, 5, 6, 7, 8, 9, 10, 11, 12, 13, 14, 15, 16, 17, 18, 19, 20, or more), or each, of ICA1, CD38, ABCB4, TNFRSF17, RRP12, CHI3L2, MAP3K13, IL4R, IL12RB2, ACHE, LAG3, CD209, GGT5, DEPDC5, UPK3A, GZMH, BPI, ACP5, CD37, MAST1, RASSF4, MS4A6A, PPFIBP1, MAK, IL18RAP, KYNU, FASLG, MYB, CCND2, TRPM6, FLVCR2, CD80, TMEM156, BHLHE41, NFE2, TREM1, TMEM255A, IL1B, PLEKHG3, VPREB3, TEP1, TRPM4, SMPDL3B, LILRB2, CHI3L1, IL2RA, TLR2, BMP2K, TNFRSF11A, PLA2G7, EPHA1, ABCB9, ZFP36L2, HHEX, SKA1, CLIC2, DUSP2, SLAMF8, CDA, ZNF222, ASGR2, PBXIP1, NIPSNAP3B, TSHR, NOD2, CD300A, NAALADL1, ADRB2, SCN9A, CEACAM3, COL8A2, CLEC7A, CTSW, HPSE, CD7, P2RY14, HIC1, P2RY13, GPR19, CCR3, MROH7, CSF1, FAM174B, TNFRSF4, PDCD1, RYR1, HLA-DQA1, NCR3, ZBTB10, APOBEC3G, LILRA2, TNFRSF13B, and HLA-DOB.

In some embodiments, the probes specific for the target region set comprise probes specific for at least a portion of target regions from one or more (such as 2, 3, 4, 5, 6, 7, 8, 9, 10, 11, 12, 13, 14, 15, 16, 17, 18, 19, 20, or more), or each, of CFH, HS3ST1, DBNDD1, CD22, SLC25A39, KCNG1, TGFBR3, ADD2, COL19A1, CD200, TCL1A, PROCR, CD40, NME4, TSPAN13, RGS9, FAM184A, KHDRBS2, ENPP5, MMP8, SATB2, GPR68, CEACAM8, MYO1B, LARGE1, NT5E, RAPGEF5, ABHD17C, ZNF365, GRTP1, IGFBP3, LCN2, GLB1L2, CNKSR2, PRSS23, RASGRP3, SCN3A, C16orf74, RETREG1, ERG, SNX22, CXCR5, BEND5, SLC1A7, LEXM, CAMK2N1, SPRY1, CDCA7L, SPIB, DLC1, DIPK1B, MTCL1, PARM1, MZB1, SLC23A1, PDGFD, NMUR1, NBEA, OLR1, PTPRM, PAWR, ABCA13, TAFA1, KCNH8, EFNA5, PRSS57, PTCH1, PRTN3, SULT1A1, ZNF667, SHISA4, LICAM, RASGEF1A, DDR1, GPC2, PATL2, and IGLL5.

### 3. Probes Specific for Epigenetic Target Regions

The probes for the epigenetic target region set may comprise probes specific for one or more types of target regions likely to differentiate DNA from neoplastic (e.g., tumor or cancer) cells from healthy cells, e.g., non-neoplastic circulating cells. Exemplary types of such regions are discussed in detail herein, e.g., in the sections above concerning captured sets. The probes for the epigenetic target region set may also comprise probes for one or more control regions, e.g., as described herein.

In some embodiments, the probes for the epigenetic target region set have a footprint of at least 100 kbp, e.g., at least 200 kbp, at least 300 kbp, or at least 400 kbp. In some embodiments, the epigenetic target region set has a footprint in the range of 100-20 Mbp, e.g., 100-200 kbp, 200-300 kbp, 300-400 kbp, 400-500 kbp, 500-600 kbp, 600-700 kbp, 700-800 kbp, 800-900 kbp, 900-1,000 kbp, 1-1.5 Mbp, 1.5-2 Mbp, 2-3 Mbp, 3-4 Mbp, 4-5 Mbp, 5-6 Mbp, 6-7 Mbp, 7-8 Mbp, 8-9 Mbp, 9-10 Mbp, or 10-20 Mbp. In some embodiments, the epigenetic target region set has a footprint of at least 20 Mbp.

a. Hypermethylation Variable Target Regions

In some embodiments, the probes for the epigenetic target region set comprise probes specific for one or more hypermethylation variable target regions. Hypermethylation variable target regions may also be referred to herein as hypermethylated DMRs (differentially methylated regions). The hypermethylation variable target regions may be any of those set forth above. For example, in some embodiments, the probes specific for hypermethylation variable target regions comprise probes specific for a plurality of loci listed in Table 2, e.g., at least 10%, 20%, 30%, 40%, 50%, 60%, 70%, 80%, 90%, or 100% of the loci listed in Table 2. In some embodiments, the probes specific for hypermethylation variable target regions comprise probes specific for a plurality of loci listed in Table 3, e.g., at least 10%, 20%, 30%, 40%, 50%, 60%, 70%, 80%, 90%, or 100% of the loci listed in Table 3. In some embodiments, the probes specific for hypermethylation variable target regions comprise probes specific for a plurality of loci listed in Table 2 or Table 3, e.g., at least 10%, 20%, 30%, 40%, 50%, 60%, 70%, 80%, 90%, or 100% of the loci listed in Table 2 or Table 3. In some embodiments, for each locus included as a target region, there may be one or more probes with a hybridization site that binds between the transcription start site and the stop codon (the last stop codon for genes that are alternatively spliced) of the gene. In some embodiments, the one or more probes bind within 300 bp of the listed position, e.g., within 200 or 100 bp. In some embodiments, a probe has a hybridization site overlapping the position listed above. In some embodiments, the probes specific for the hypermethylation target regions include probes specific for one, two, three, four, or five subsets of hypermethylation target regions that collectively show hypermethylation in one, two, three, four, or five of breast, colon, kidney, liver, and lung cancers.

b. Hypomethylation Variable Target Regions

In some embodiments, the probes for the epigenetic target region set comprise probes specific for one or more hypomethylation variable target regions. Hypomethylation variable target regions may also be referred to herein as hypomethylated DMRs (differentially methylated regions). The hypomethylation variable target regions may be any of those set forth above. For example, the probes specific for one or more hypomethylation variable target regions may include probes for regions such as repeated elements, e.g., LINE1 elements, Alu elements, centromeric tandem repeats, pericentromeric tandem repeats, and satellite DNA, and intergenic regions that are ordinarily methylated in healthy cells may show reduced methylation in tumor cells.

In some embodiments, probes specific for hypomethylation variable target regions include probes specific for repeated elements and/or intergenic regions. In some embodiments, probes specific for repeated elements include probes specific for one, two, three, four, or five of LINE1 elements, Alu elements, centromeric tandem repeats, pericentromeric tandem repeats, and/or satellite DNA.

Exemplary probes specific for genomic regions that show cancer-associated hypomethylation include probes specific for nucleotides 8403565-8953708 and/or 151104701-151106035 of human chromosome 1. In some embodiments, the probes specific for hypomethylation variable target regions include probes specific for regions overlapping or comprising nucleotides 8403565-8953708 and/or 151104701-151106035 of human chromosome 1.

c. CTCF Binding Regions

In some embodiments, the probes for the epigenetic target region set include probes specific for CTCF binding regions. In some embodiments, the probes specific for CTCF binding regions comprise probes specific for at least 10, 20, 50, 100, 200, or 500 CTCF binding regions, or 10-20, 20-50, 50-100, 100-200, 200-500, or 500-1000 CTCF binding regions, e.g., such as CTCF binding regions described above or in one or more of CTCFBSDB or the Cuddapah et al., Martin et al., or Rhee et al. articles cited above. In some embodiments, the probes for the epigenetic target region set comprise at least 100 bp, at least 200 bp at least 300 bp, at least 400 bp, at least 500 bp, at least 750 bp, or at least 1000 bp upstream and downstream regions of the CTCF binding sites.

d. Transcription Start Sites

In some embodiments, the probes for the epigenetic target region set include probes specific for transcriptional start sites. In some embodiments, the probes specific for transcriptional start sites comprise probes specific for at least 10, 20, 50, 100, 200, or 500 transcriptional start sites, or 10-20, 20-50, 50-100, 100-200, 200-500, or 500-1000 transcriptional start sites, e.g., such as transcriptional start sites listed in DBTSS. In some embodiments, the probes for the epigenetic target region set comprise probes for sequences at least 100 bp, at least 200 bp, at least 300 bp, at least 400 bp, at least 500 bp, at least 750 bp, or at least 1000 bp upstream and downstream of the transcriptional start sites.

e. Focal Amplifications

As noted above, although focal amplifications are somatic mutations, they can be detected by sequencing based on read frequency in a manner analogous to approaches for detecting certain epigenetic changes such as changes in methylation. As such, regions that may show focal amplifications in cancer can be included in the epigenetic target region set, as discussed above. In some embodiments, the probes specific for the epigenetic target region set include probes specific for focal amplifications. In some embodiments, the probes specific for focal amplifications include probes specific for one or more of AR, BRAF, CCND1, CCND2, CCNE1, CDK4, CDK6, EGFR, ERBB2, FGFR1, FGFR2, KIT, KRAS, MET, MYC, PDGFRA, PIK3CA, and RAF1. For example, in some embodiments, the probes specific for focal amplifications include probes specific for one or more of at least 2, 3, 4, 5, 6, 7, 8, 9, 10, 11, 12, 13, 14, 15, 16, 17, or 18 of the foregoing targets.

f. Control Regions

It can be useful to include control regions to facilitate data validation. In some embodiments, the probes specific for the epigenetic target region set include probes specific for control methylated regions that are expected to be methylated in essentially all samples. In some embodiments, the probes specific for the epigenetic target region set include probes specific for control hypomethylated regions that are expected to be hypomethylated in essentially all samples.

### 4. Probes Specific for Sequence-Variable Target Regions

The probes for the sequence-variable target region set may comprise probes specific for a plurality of regions known to undergo somatic mutations in cancer. The probes may be specific for any sequence-variable target region set described herein. Exemplary sequence-variable target region sets are discussed in detail herein, e.g., in the sections above concerning captured sets.

In some embodiments, the sequence-variable target region probe set has a footprint of at least 0.5 kb, e.g., at least 1 kb, at least 2 kb, at least 5 kb, at least 10 kb, at least 20 kb, at least 30 kb, or at least 40 kb. In some embodiments, the epigenetic target region probe set has a footprint in the range of 0.5-100 kb, e.g., 0.5-2 kb, 2-10 kb, 10-20 kb, 20-30 kb, 30-40 kb, 40-50 kb, 50-60 kb, 60-70 kb, 70-80 kb, 80-90 kb, and 90-100 kb. In some embodiments, the sequence-variable target region probe set has a footprint of at least 50 kbp, e.g., at least 100 kbp, at least 200 kbp, at least 300 kbp, or at least 400 kbp. In some embodiments, the sequence-variable target region probe set has a footprint in the range of 100-2000 kbp, e.g., 100-200 kbp, 200-300 kbp, 300-400 kbp, 400-500 kbp, 500-600 kbp, 600-700 kbp, 700-800 kbp, 800-900 kbp, 900-1,000 kbp, 1-1.5 Mbp or 1.5-2 Mbp. In some embodiments, the sequence-variable target region set has a footprint of at least 2 Mbp.

In some embodiments, probes specific for the sequence-variable target region set comprise probes specific for at least a portion of at least 5, at least 10, at least 15, at least 20, at least 25, at least 30, at least 35, at least 40, at least 45, at least 50, at least 55, at least 60, at least 65, or at 70 of the genes of Table 4. In some embodiments, probes specific for the sequence-variable target region set comprise probes specific for the at least 5, at least 10, at least 15, at least 20, at least 25, at least 30, at least 35, at least 40, at least 45, at least 50, at least 55, at least 60, at least 65, or 70 of the SNVs of Table 4. In some embodiments, probes specific for the sequence-variable target region set comprise probes specific for at least 1, at least 2, at least 3, at least 4, at least 5, or 6 of the fusions of Table 4. In some embodiments, probes specific for the sequence-variable target region set comprise probes specific for at least a portion of at least 1, at least 2, or 3 of the indels of Table 4. In some embodiments, probes specific for the sequence-variable target region set comprise probes specific for at least a portion of at least 5, at least 10, at least 15, at least 20, at least 25, at least 30, at least 35, at least 40, at least 45, at least 50, at least 55, at least 60, at least 65, at least 70, or 73 of the genes of Table 5. In some embodiments, probes specific for the sequence-variable target region set comprise probes specific for at least 5, at least 10, at least 15, at least 20, at least 25, at least 30, at least 35, at least 40, at least 45, at least 50, at least 55, at least 60, at least 65, at least 70, or 73 of the SNVs of Table 5. In some embodiments, probes specific for the sequence-variable target region set comprise probes specific for at least 1, at least 2, at least 3, at least 4, at least 5, or 6 of the fusions of Table 5. In some embodiments, probes specific for the sequence-variable target region set comprise probes specific for at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, or 18 of the indels of Table 5. In some embodiments, probes specific for the sequence-variable target region set comprise probes specific for at least a portion of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 of the genes of Table 6.

In some embodiments, the probes specific for the sequence-variable target region set comprise probes specific for target regions from at least 10, 20, 30, or 35 cancer-related genes, such as AKT1, ALK, BRAF, CCND1, CDK2A, CTNNB1, EGFR, ERBB2, ESR1, FGFR1, FGFR2, FGFR3, FOXL2, GATA3, GNA11, GNAQ, GNAS, HRAS, IDH1, IDH2, KIT, KRAS, MED12, MET, MYC, NFE2L2, NRAS, PDGFRA, PIK3CA, PPP2RIA, PTEN, RET, STK11, TP53, and U2AF1.

TABLE 4

| Point Mutations (SNVs) | | | | | | Fusions |
|---|---|---|---|---|---|---|
| AKT1 | ALK | APC | AR | ARAF | ARID1A | ALK |
| ATM | BRAF | BRCA1 | BRCA2 | CCND1 | CCND2 | FGFR2 |
| CCNE1 | CDH1 | CDK4 | CDK6 | CDKN2A | CDKN2B | FGFR3 |
| CTNNB1 | EGFR | ERBB2 | ESR1 | EZH2 | FBXW7 | NTRK1 |
| FGFR1 | FGFR2 | FGFR3 | GATA3 | GNA11 | GNAQ | RET |
| GNAS | HNF1A | HRAS | IDH1 | IDH2 | JAK2 | ROS1 |
| JAK3 | KIT | KRAS | MAP2K1 | MAP2K2 | MET | |
| MLH1 | MPL | MYC | NF1 | NFE2L2 | NOTCH1 | |
| NPM1 | NRAS | NTRK1 | PDGFRA | PIK3CA | PTEN | |
| PTPN11 | RAF1 | RB1 | RET | RHEB | RHOA | |
| RIT1 | ROS1 | SMAD4 | SMO | SRC | STK11 | |
| TERT | TP53 | TSC1 | VHL | | | |

TABLE 5

| Point Mutations (SNVs) | | | | | | Fusions |
|---|---|---|---|---|---|---|
| AKT1 | ALK | APC | AR | ARAF | ARID1A | ALK |
| ATM | BRAF | BRCA1 | BRCA2 | CCND1 | CCND2 | FGFR2 |
| CCNE1 | CDH1 | CDK4 | CDK6 | CDKN2A | DDR2 | FGFR3 |
| CTNNB1 | EGFR | ERBB2 | ESR1 | EZH2 | FBXW7 | NTRK1 |
| FGFR1 | FGFR2 | FGFR3 | GATA3 | GNA11 | GNAQ | RET |
| GNAS | HNF1A | HRAS | IDH1 | IDH2 | JAK2 | ROS1 |
| JAK3 | KIT | KRAS | MAP2K1 | MAP2K2 | MET | |
| MLH1 | MPL | MYC | NF1 | NFE2L2 | NOTCH1 | |
| NPM1 | NRAS | NTRK1 | PDGFRA | PIK3CA | PTEN | |
| PTPN11 | RAF1 | RB1 | RET | RHEB | RHOA | |
| RIT1 | ROS1 | SMAD4 | SMO | MAPK1 | STK11 | |
| TERT | TP53 | TSC1 | VHL | MAPK3 | MTOR | |
| NTRK3 | | | | | | |

TABLE 6

| | | Start | Stop | Length | Exons | |
|---|---|---|---|---|---|---|
| Gene | Chromosome | Position | Position | (bp) | Covered | Critical Feature |
| ALK | chr2 | 29446405 | 29446655 | 250 | intron 19 | Fusion |
| ALK | chr2 | 29446062 | 29446197 | 135 | intron 20 | Fusion |
| ALK | chr2 | 29446198 | 29446404 | 206 | 20 | Fusion |
| ALK | chr2 | 29447353 | 29447473 | 120 | intron 19 | Fusion |
| ALK | chr2 | 29447614 | 29448316 | 702 | intron 19 | Fusion |
| ALK | chr2 | 29448317 | 29448441 | 124 | 19 | Fusion |
| ALK | chr2 | 29449366 | 29449777 | 411 | intron 18 | Fusion |
| ALK | chr2 | 29449778 | 29449950 | 172 | 18 | Fusion |
| BRAF | chr7 | 140453064 | 140453203 | 139 | 15 | BRAF V600 |
| CTNNB1 | chr3 | 41266007 | 41266254 | 247 | 3 | S37 |
| EGFR | chr7 | 55240528 | 55240827 | 299 | 18 and 19 | G719 and deletions |
| EGFR | chr7 | 55241603 | 55241746 | 143 | 20 | Insertions/T790M |
| EGFR | chr7 | 55242404 | 55242523 | 119 | 21 | L858R |
| ERBB2 | chr17 | 37880952 | 37881174 | 222 | 20 | Insertions |
| ESR1 | chr6 | 152419857 | 152420111 | 254 | 10 | V534, P535, L536, |
| | | | | | | Y537, D538 |
| FGFR2 | chr10 | 123279482 | 123279693 | 211 | 6 | S252 |
| GATA3 | chr10 | 8111426 | 8111571 | 145 | 5 | SS/Indels |
| GATA3 | chr10 | 8115692 | 8116002 | 310 | 6 | SS/Indels |
| GNAS | chr20 | 57484395 | 57484488 | 93 | 8 | R844 |
| IDH1 | chr2 | 209113083 | 209113394 | 311 | 4 | R132 |
| IDH2 | chr15 | 90631809 | 90631989 | 180 | 4 | R140, R172 |
| KIT | chr4 | 55524171 | 55524258 | 87 | 1 | |
| KIT | chr4 | 55561667 | 55561957 | 290 | 2 | |
| KIT | chr4 | 55564439 | 55564741 | 302 | 3 | |
| KIT | chr4 | 55565785 | 55565942 | 157 | 4 | |
| KIT | chr4 | 55569879 | 55570068 | 189 | 5 | |
| KIT | chr4 | 55573253 | 55573463 | 210 | 6 | |
| KIT | chr4 | 55575579 | 55575719 | 140 | 7 | |
| KIT | chr4 | 55589739 | 55589874 | 135 | 8 | |
| KIT | chr4 | 55592012 | 55592226 | 214 | 9 | |
| KIT | chr4 | 55593373 | 55593718 | 345 | 10 and 11 | 557, 559, 560, 576 |
| KIT | chr4 | 55593978 | 55594297 | 319 | 12 and 13 | V654 |
| KIT | chr4 | 55595490 | 55595661 | 171 | 14 | T670, S709 |
| KIT | chr4 | 55597483 | 55597595 | 112 | 15 | D716 |
| KIT | chr4 | 55598026 | 55598174 | 148 | 16 | L783 |
| KIT | chr4 | 55599225 | 55599368 | 143 | 17 | C809, R815, D816, |
| | | | | | | L818, D820, S821F, |
| | | | | | | N822, Y823 |
| KIT | chr4 | 55602653 | 55602785 | 132 | 18 | A829P |
| KIT | chr4 | 55602876 | 55602996 | 120 | 19 | |
| KIT | chr4 | 55603330 | 55603456 | 126 | 20 | |
| KIT | chr4 | 55604584 | 55604733 | 149 | 21 | |
| KRAS | chr12 | 25378537 | 25378717 | 180 | 4 | A146 |
| KRAS | chr12 | 25380157 | 25380356 | 199 | 3 | Q61 |
| KRAS | chr12 | 25398197 | 25398328 | 131 | 2 | G12/G13 |
| MET | chr7 | 116411535 | 116412255 | 720 | 13, 14, | MET exon 14 SS |
| | | | | | intron 13, | |
| | | | | | intron 14 | |
| NRAS | chr1 | 115256410 | 115256609 | 199 | 3 | Q61 |
| NRAS | chr1 | 115258660 | 115258791 | 131 | 2 | G12/G13 |
| PIK3CA | chr3 | 178935987 | 178936132 | 145 | 10 | E545K |
| PIK3CA | chr3 | 178951871 | 178952162 | 291 | 21 | H1047R |
| PTEN | chr10 | 89692759 | 89693018 | 259 | 5 | R130 |
| SMAD4 | chr18 | 48604616 | 48604849 | 233 | 12 | D537 |
| TERT | chr5 | 1294841 | 1295512 | 671 | promoter | chr5:1295228 |
| TP53 | chr17 | 7573916 | 7574043 | 127 | 11 | Q331, R337, R342 |
| TP53 | chr17 | 7577008 | 7577165 | 157 | 8 | R273 |
| TP53 | chr17 | 7577488 | 7577618 | 130 | 7 | R248 |
| TP53 | chr17 | 7578127 | 7578299 | 172 | 6 | R213/Y220 |
| TP53 | chr17 | 7578360 | 7578564 | 204 | 5 | R175/Deletions |
| TP53 | chr17 | 7579301 | 7579600 | 299 | 4 | |
| | | | | 12574 | | |
| | | | | (total | | |
| | | | | target | | |
| | | | | region) | | |
| | | | | 16330 | | |
| | | | | (total | | |
| | | | | probe | | |
| | | | | coverage) | | |

### D. Computer Systems

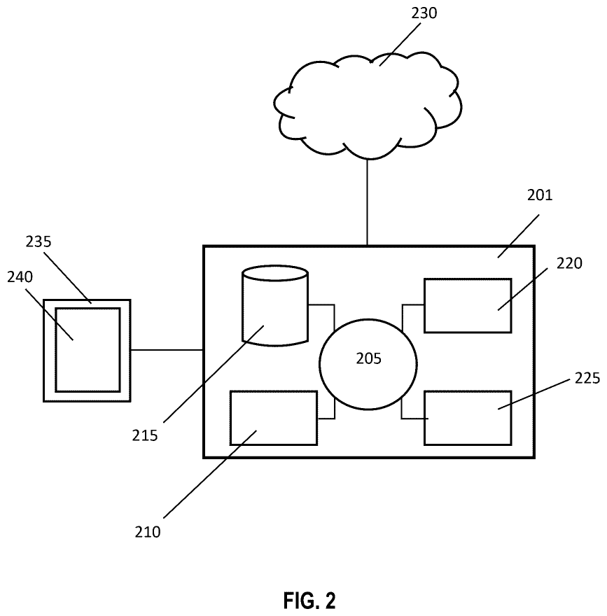

Methods of the present disclosure can be implemented using, or with the aid of, computer systems. FIG. 2 shows a computer system 201 that is programmed or otherwise configured to implement the methods of the present disclosure. The computer system 201 can regulate various aspects sample preparation, sequencing, and/or analysis. In some examples, the computer system 201 is configured to perform sample preparation and sample analysis, including nucleic acid sequencing, e.g., according to any of the methods disclosed herein.

The computer system 201 includes a central processing unit (CPU, also “processor” and “computer processor” herein) 205, which can be a single core or multi core processor, or a plurality of processors for parallel processing. The computer system 201 also includes memory or memory location 210 (e.g., random-access memory, read-only memory, flash memory), electronic storage unit 215 (e.g., hard disk), communication interface 220 (e.g., network adapter) for communicating with one or more other systems, and peripheral devices 225, such as cache, other memory, data storage, and/or electronic display adapters. The memory 210, storage unit 215, interface 220, and peripheral devices 225 are in communication with the CPU 205 through a communication network or bus (solid lines), such as a motherboard. The storage unit 215 can be a data storage unit (or data repository) for storing data. The computer system 201 can be operatively coupled to a computer network 230 with the aid of the communication interface 220. The computer network 230 can be the Internet, an internet and/or extranet, or an intranet and/or extranet that is in communication with the Internet. The computer network 230 in some cases is a telecommunication and/or data network. The computer network 230 can include one or more computer servers, which can enable distributed computing, such as cloud computing. The computer network 230, in some cases with the aid of the computer system 0, can implement a peer-to-peer network, which may enable devices coupled to the computer system 201 to behave as a client or a server.

The CPU 205 can execute a sequence of machine-readable instructions, which can be embodied in a program or software. The instructions may be stored in a memory location, such as the memory 210. Examples of operations performed by the CPU 205 can include fetch, decode, execute, and writeback.

The storage unit 215 can store files, such as drivers, libraries, and saved programs. The storage unit 215 can store programs generated by users and recorded sessions, as well as output(s) associated with the programs. The storage unit 215 can store user data, e.g., user preferences and user programs. The computer system 201 in some cases can include one or more additional data storage units that are external to the computer system 201, such as located on a remote server that is in communication with the computer system 201 through an intranet or the Internet. Data may be transferred from one location to another using, for example, a communication network or physical data transfer (e.g., using a hard drive, thumb drive, or other data storage mechanism).

The computer system 201 can communicate with one or more remote computer systems through the network 230. For embodiment, the computer system 201 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 201 via the network 230.

Methods as described herein can be implemented by way of machine (e.g., computer processor) executable code stored on an electronic storage location of the computer system 201, such as, for example, on the memory 210 or electronic storage unit 215. The machine executable or machine-readable code can be provided in the form of software. During use, the code can be executed by the processor 205. In some cases, the code can be retrieved from the storage unit 215 and stored on the memory 210 for ready access by the processor 205. In some situations, the electronic storage unit 215 can be precluded, and machine-executable instructions are stored on memory 210.

In an aspect, the present disclosure provides a non-transitory computer-readable medium comprising computer-executable instructions which, when executed by at least one electronic processor, perform at least a portion of a method comprising: (a) sequencing RNA (such as RNA isolated from a sample comprising cells or a blood sample) and determining expression levels for a target gene set comprising a plurality of target genes that are differentially expressed in a plurality of immune cell types and/or in samples from subjects with a disease or disorder (such as a cancer, such as advanced adenoma (AA) and/or colorectal cancer (CRC)) relative to samples from healthy subjects; and (b) determining (1) quantities of the immune cell types from which the RNA originated based on the expression levels; and/or (2) expression levels of the plurality of target genes; and (c) determining the presence, absence, or likelihood of the disease or disorder in the subject based on the quantities of the immune cell types and/or the expression levels of the plurality of target genes.

The code can be pre-compiled and configured for use with a machine have a processer adapted to execute the code or can be compiled during runtime. The code can be supplied in a programming language that can be selected to enable the code to execute in a pre-compiled or as-compiled fashion.

Aspects of the systems and methods provided herein, such as the computer system 201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming.

All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as those used across physical interfaces between local devices, through wired and optical landline networks, and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards, paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 201 can include or be in communication with an electronic display that comprises a user interface (UI) for providing, for example, one or more results of sample analysis. Examples of UIs include, without limitation, a graphical user interface (GUI) and web-based user interface.

Additional details relating to computer systems and networks, databases, and computer program products are also provided in, for example, Peterson, Computer Networks: A Systems Approach, Morgan Kaufmann, 5^{th} Ed. (2011), Kurose, Computer Networking: A Top-Down Approach, Pearson, 7^{th} Ed. (2016), Elmasri, Fundamentals of Database Systems, Addison Wesley, 6^{th} Ed. (2010), Coronel, Database Systems: Design, Implementation, & Management, Cengage Learning, 11^{th} Ed. (2014), Tucker, Programming Languages, McGraw-Hill Science/Engineering/Math, 2^{nd} Ed. (2006), and Rhoton, Cloud Computing Architected: Solution Design Handbook, Recursive Press (2011), each of which is hereby incorporated by reference in its entirety.

### E. Applications

### 1. Immune Cell Type Quantification, Cancer, and Other Diseases

The present methods can be used to quantify levels of different immune cell types, including rare immune cell types, such as activated lymphocytes and myeloid cells at particular stages of differentiation. Such quantification can be based on the expression of genes characteristic of a given cell type in a sample.

Sequence information obtained in the present methods may comprise sequence reads of the nucleic acids generated by a nucleic acid sequencer. In some embodiments, the nucleic acid sequencer performs RNA-seq pyrosequencing, single-molecule sequencing, nanopore sequencing, semiconductor sequencing, sequencing-by-synthesis, sequencing-by-ligation or sequencing-by-hybridization on the nucleic acids to generate sequencing reads. In some embodiments, the method further comprises grouping the sequence reads into families of sequence reads, each family comprising sequence reads generated from a nucleic acid in the sample. In some embodiments, the methods comprise determining the likelihood that the subject from which the sample was obtained has cancer, precancer, an infection, transplant rejection, or other diseases or disorder that is related to changes in proportions of types of immune cells or differences in expression of one or more of a plurality of target genes identified using the methods described herein.

Accordingly, the present methods can be used to diagnose the presence of a condition, e.g., cancer or precancer, in a subject, to characterize a condition (such as to determine a cancer stage or heterogeneity of a cancer), to monitor a subject's response to receiving a treatment for a condition (such as a response to a chemotherapeutic or immunotherapeutic), assess prognosis of a subject (such as to predict a survival outcome in a subject having a cancer), to determine a subject's risk of developing a condition, to predict a subsequent course of a condition in a subject, to determine metastasis or recurrence of a cancer in a subject (or a risk of cancer metastasis or recurrence), and/or to monitor a subject's health as part of a preventative health monitoring program (such as to determine whether and/or when a subject is in need of further diagnostic screening). The present disclosure can also be useful in determining the efficacy of a particular treatment option. Successful treatment options may result in changes in levels of different immune cell types (including rare immune cell types), and/or increase the amount of copy number variation, rare mutations, and/or cancer-related epigenetic signatures (such as hypermethylated regions or hypomethylated regions) detected in, e.g., a sample from a subject, such as detected in a subject's blood (such as in DNA isolated from a buffy coat sample or any other sample comprising cells, such as in a blood sample (e.g., a whole blood sample, a leukapheresis sample, or a PBMC sample) from the subject) if the treatment is successful as more cancer cells may die and shed DNA, or, e.g., if a successful treatment results in an increase or decrease in the quantity of a specific immune cell type in the blood and an unsuccessful treatment results in no change. In other examples, this may not occur. These changes may be useful in selecting a therapy.

Additionally, if a cancer is observed to be in remission after treatment, the present methods can be used to monitor the likelihood of residual disease or the likelihood of recurrence of disease.

In some embodiments, the present methods are used for screening for a cancer, such as a metastasis, or in a method for screening cancer, such as in a method of detecting the presence or absence of a metastasis. For example, the sample can be a sample from a subject who has or has not been previously diagnosed with cancer. In some embodiments, a sample is obtained from a subject who was previously diagnosed with the cancer and received one or more previous cancer treatments, optionally wherein the sample is obtained at one or more preselected time points following the one or more previous cancer treatments. In some embodiments, a sample is obtained from a subject who was previously diagnosed with the cancer, and the sample is obtained from the subject before the subject receives a cancer treatment. In some embodiments, one or more, such as 1, 2, 3, 4, 5, 6, 7, 8, 9, 10, or more samples are collected from a subject as described herein, such as before and/or after the subject is diagnosed with a cancer. In some embodiments, the subject may or may not have cancer. In some embodiments, the subject may or may not have an early-stage cancer. In some embodiments, the subject has one or more risk factors for cancer, such as tobacco use (e.g., smoking), being overweight or obese, having a high body mass index (BMI), being of advanced age, poor nutrition, high alcohol consumption, or a family history of cancer.

In some embodiments, the subject has used tobacco, e.g., for at least 1, 5, 10, or 15 years. In some embodiments, the subject has a high BMI, e.g., a BMI of 25 or greater, 26 or greater, 27 or greater, 28 or greater, 29 or greater, or 30 or greater. In some embodiments, the subject is at least 40, 45, 50, 55, 60, 65, 70, 75, or 80 years old. In some embodiments, the subject has poor nutrition, e.g., high consumption of one or more of red meat and/or processed meat, trans fat, saturated fat, and refined sugars, and/or low consumption of fruits and vegetables, complex carbohydrates, and/or unsaturated fats. High and low consumption can be defined, e.g., as exceeding or falling below, respectively, recommendations in Dietary Guidelines for Americans 2020-2025, available at dietaryguidelines.gov/sites/default/files/2021-03/Dietary_Guidelines_for_Americans-2020-2025.pdf. In some embodiments, the subject has high alcohol consumption, e.g., at least three, four, or five drinks per day on average (where a drink is about one ounce or 30 mL of 80-proof hard liquor or the equivalent). In some embodiments, the subject has a family history of cancer, e.g., at least one, two, or three blood relatives were previously diagnosed with cancer. In some embodiments, the relatives are at least third-degree relatives (e.g., great-grandparent, great aunt or uncle, first cousin), at least second-degree relatives (e.g., grandparent, aunt or uncle, or half-sibling), or first-degree relatives (e.g., parent or full sibling).

Typically, the disease under consideration is a type of cancer, such as any referred to herein. The types and number of cancers that may be detected may include blood cancers, brain cancers, eye cancers, oral cancers, head and neck cancers, gallbladder cancers, endometrial cancers, ovarian cancers, uterine cancers, prostate cancers, esophageal cancers, lung cancers, skin cancers, nose cancers, throat cancers, liver cancers, bone cancers, lymphomas, leukemias, pancreatic cancers, skin cancers, gastrointestinal cancers, bowel cancers, colorectal cancers, colon cancers, rectal cancers, thyroid cancers, bladder cancers, kidney cancers, mouth cancers, stomach cancers, breast cancers, solid state tumors, heterogeneous tumors, homogenous tumors and the like. Specific examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic leukemia (CLL), chronic myeloid leukemia (CML), chronic myelomonocytic leukemia (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas, prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma. In some embodiments, the cancer is a hematological cancer. In other embodiments, the cancer is a type of cancer that is not a hematological cancer, e.g., a solid tumor cancer such as a carcinoma, adenocarcinoma, or sarcoma. Type and/or stage of cancer can be detected from genetic variations including mutations, rare mutations, indels, rearrangements, copy number variations, transversions, translocations, recombinations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and abnormal changes in nucleic acid 5-methylcytosine. In some embodiments, the cancer is a type of cancer that is not a hematological cancer, e.g., a solid tumor cancer such as a carcinoma or sarcoma.

The present methods can be used to generate a profile, fingerprint, or set of data that is a summation of information derived from different cells in a heterogeneous disease. This set of data may comprise RNA levels, cell type levels, inferred RNA levels for one or more cell types, and/or additional information obtainable from methods described herein.

Genetic data can also be used for characterizing a specific form of cancer. Cancers are often heterogeneous in both composition and staging. Genetic profile data may allow characterization of specific sub-types of cancer may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The methods of this disclosure may be useful in determining disease progression.

Further, the methods of this disclosure may be used to characterize the heterogeneity of an abnormal condition in a subject. Such methods can include, e.g., generating a gene expression profile using RNA derived from the subject. In some embodiments, an abnormal condition is cancer. In some embodiments, the abnormal condition may be one resulting in a heterogeneous genomic population. In the example of cancer, some tumors are known to comprise tumor cells in different stages of the cancer. In other examples, heterogeneity may comprise multiple foci of disease. Again, in the example of cancer, there may be multiple tumor foci, such as where one or more foci (such as one or more tumor foci) are the result of metastases that have spread from a primary site of a cancer. The tissue(s) of origin can be useful for identifying organs affected by the cancer, including the primary cancer and/or metastatic tumors.

The present methods can be used to diagnose, prognose, monitor or observe cancers, precancers, or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose RNA and other polynucleotides may co-circulate with maternal molecules.

Non-limiting examples of other genetic-based diseases, disorders, or conditions that are optionally evaluated using the methods and systems disclosed herein include achondroplasia, alpha-1 antitrypsin deficiency, antiphospholipid syndrome, autism, autosomal dominant polycystic kidney disease, Charcot-Marie-Tooth (CMT), cri du chat, Crohn's disease, cystic fibrosis, Dercum disease, down syndrome, Duane syndrome, Duchenne muscular dystrophy, Factor V Leiden thrombophilia, familial hypercholesterolemia, familial Mediterranean fever, fragile X syndrome, Gaucher disease, hemochromatosis, hemophilia, holoprosencephaly, Huntington's disease, Klinefelter syndrome, Marfan syndrome, myotonic dystrophy, neurofibromatosis, Noonan syndrome, osteogenesis imperfecta, Parkinson's disease, phenylketonuria, Poland anomaly, porphyria, progeria, retinitis pigmentosa, severe combined immunodeficiency (SCID), sickle cell disease, spinal muscular atrophy, Tay-Sachs, thalassemia, trimethylaminuria, Turner syndrome, velocardio facial syndrome, WAGR syndrome, Wilson disease, or the like.

In some embodiments, a method described herein comprises detecting a presence or absence of, or quantifying, RNA originating or derived from an immune cell at a preselected timepoint following a previous cancer treatment of a subject previously diagnosed with cancer using a set of sequence information obtained as described herein. The method may further comprise determining a cancer recurrence score that is indicative of the presence or absence of, or amount of, the RNA originating or derived from the immune cell for the subject.

Where a cancer recurrence score is determined, it may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

In some embodiments, a cancer recurrence score is compared with a predetermined cancer recurrence threshold, and the subject is classified as a candidate for a subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy.

The present methods can also be used to quantify levels of different cell types, such as immune cell types, including rare immune cell types, such as activated lymphocytes and myeloid cells at particular stages of differentiation. Such quantification can be based on the numbers of molecules corresponding to a given cell type in a sample. In some embodiments, quantities of each of a plurality of cell types, such as immune cell types, are determined based on sequencing and analysis (such as determination of epigenetic and/or genomic signatures) of DNA isolated from at least one sample comprising cells (such as a buffy coat sample or another type of blood sample (e.g., a whole blood sample, a leukapheresis sample, or a PBMC sample) from a subject. The plurality of immune cell types can include, but is not limited to, macrophages (including M1 macrophages and M2 macrophages), activated B cells (including regulatory B cells, memory B cells and plasma cells); T cell subsets, such as central memory T cells, naïve-like T cells, and activated T cells (including cytotoxic T cells, regulatory T cells (Tregs), CD4 effector memory T cells, CD4 central memory T cells, CD8 effector memory T cells, and CD8 central memory T cells); immature myeloid cells (including myeloid-derived suppressor cells (MDSCs), low-density neutrophils, immature neutrophils, and immature granulocytes); and natural killer (NK) cells. As disclosed herein, differences in levels and/or presence of particular genetic signatures (e.g., gene expression levels) in RNA isolated from blood samples from a subject can be used to quantify cell types, such as immune cell types, within the sample.

In some embodiments, the methods disclosed herein comprise determining the likelihood that the subject from which the sample was obtained has cancer, precancer, an infection, transplant rejection, or other diseases or disorder that is related to changes in proportions of types of immune cells. As discussed herein, comparisons of immune cell identities and/or immune cell quantities/proportions between two or more samples collected from a subject at two different time points can allow for monitoring of one or more aspects of a condition in the subject over time, such as a response of the subject to a treatment, the severity of the condition (such as a cancer stage) in the subject, a recurrence of the condition (such as a cancer), and/or the subject's risk of developing the condition (such as a cancer).

The methods discussed above may further comprise any compatible feature or features set forth elsewhere herein, including in the section regarding methods of determining a risk of cancer recurrence in a subject and/or classifying a subject as being a candidate for a subsequent cancer treatment.

2. Methods of determining a risk of cancer recurrence in a subject and/or classifying a subject as being a candidate for a subsequent cancer treatment

In some embodiments, a method provided herein is or comprises a method of determining a risk of cancer recurrence in a subject. In some embodiments, a method provided herein is or comprises a method of detecting the presence of absence of a metastasis in a subject. In some embodiments, a method provided herein is or comprises a method of classifying a subject as being a candidate for a subsequent cancer treatment.

Any of such methods may comprise collecting RNA (e.g., originating or derived from an immune cell or a cancer cell) from the subject diagnosed with the cancer at one or more preselected timepoints following one or more previous cancer treatments to the subject. Similarly, any of such methods may comprise collecting RNA (e.g., originating or derived from an immune cell or a cancer cell) from the subject diagnosed with the cancer at one or more preselected timepoints preceding one or more previous cancer treatments to the subject. The subject may be any of the subjects described herein. The RNA may be RNA from a sample comprising cells or a blood sample (e.g., a buffy coat sample, a whole blood sample, a leukapheresis sample, or a PBMC sample). The RNA may comprise RNA obtained from a tissue sample.

Any of such methods may comprise capturing target regions from RNA (or cDNA prepared from the RNA) from the subject whereby a captured set of nucleic acid molecules is produced. The capturing step may be performed according to any of the embodiments described elsewhere herein.

In any of such methods, the previous cancer treatment may comprise surgery, administration of a therapeutic composition, and/or chemotherapy.

Any of such methods may comprise sequencing the captured nucleic acid molecules, whereby a set of sequence information is produced.

Any of such methods may comprise detecting a presence or absence of, or quantifying, RNA originating or derived from an immune cell or a cancer cell at a preselected timepoint using the set of sequence information. The detection of the presence or absence of, or quantification of RNA originating or derived from an immune cell or a cancer cell may be performed according to any of the embodiments thereof described elsewhere herein.

Methods of determining a risk of cancer recurrence in a subject may comprise determining a cancer recurrence score that is indicative of the presence or absence, or amount, of the RNA originating or derived from an immune cell or a cancer cell for the subject. The cancer recurrence score may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

Methods of classifying a subject as being a candidate for a subsequent cancer treatment may comprise comparing the cancer recurrence score of the subject with a predetermined cancer recurrence threshold, thereby classifying the subject as a candidate for the subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy. In some embodiments, the subsequent cancer treatment comprises chemotherapy or administration of a therapeutic composition.

Any of such methods may comprise determining a disease-free survival (DFS) period for the subject based on the cancer recurrence score; for example, the DFS period may be 1 year, 2 years, 3, years, 4 years, 5 years, or 10 years.

In some embodiments, determining the cancer recurrence score may comprise determining at least a first subscore indicative of the levels of particular immune cell types present based on expression levels of target genes. In some embodiments, determining the cancer recurrence score may comprise determining at least a first subscore indicative of the levels of particular immune cell types present based on whole transcriptome sequencing.

In some embodiments, any of such methods may comprise determining a fraction of tumor or immune cell RNA from the fraction of molecules in the set of sequence information that indicate one or more features indicative of origination from a tumor cell or an immune cell. This may be done for molecules corresponding to some or all of the target genes and/or target regions, e.g., including, e.g., molecules comprising alterations consistent with cancer, such as SNVs, indels, CNVs, and/or fusions. The fraction of tumor or immune cell RNA may be determined based on a combination of molecules corresponding to epigenetic target regions and molecules corresponding to target genes and/or target regions. Determination of a cancer recurrence score may be based at least in part on the fraction of tumor or immune cell RNA, wherein a fraction of tumor or immune cell RNA greater than a threshold in the range of 10⁻¹¹ to 1 or 10⁻¹⁰ to 1 is sufficient for the cancer recurrence score to be classified as positive for cancer recurrence. In some embodiments, a fraction of tumor or immune cell RNA greater than or equal to a threshold in the range of 10⁻¹⁰ to 10⁻⁹, 10⁻⁹ to 10⁻⁸, 10⁻⁸ to 10⁻⁷, 10⁻⁷ to 10⁻⁶, 10⁻⁶ to 10⁻⁵, 10⁻⁵ to 10⁻⁴, 10⁻⁴ to 10⁻³, 10⁻³ to 10⁻², or 10⁻² to 10⁻¹ is sufficient for the cancer recurrence score to be classified as positive for cancer recurrence. In some embodiments, the fraction of tumor or immune cell RNA greater than a threshold of at least 10⁻⁷ is sufficient for the cancer recurrence score to be classified as positive for cancer recurrence. A determination that a fraction of tumor or immune cell RNA is greater than a threshold, such as a threshold corresponding to any of the foregoing embodiments, may be made based on a cumulative probability. For example, the sample was considered positive if the cumulative probability that the tumor fraction was greater than a threshold in any of the foregoing ranges exceeds a probability threshold of at least 0.5, 0.75, 0.9, 0.95, 0.98, 0.99, 0.995, or 0.999. In some embodiments, the probability threshold is at least 0.95, such as 0.99.

In some embodiments, the set of sequence information comprises differential expression data for one or more of a plurality of target genes as described herein, and optionally target region sequences, and determining the cancer recurrence score comprises determining a first subscore indicative of the levels of particular immune cell types, and a second subscore indicative of the amount of SNVs, insertions/deletions, CNVs and/or fusions present in target region sequences, and combining the first and second subscores to provide the cancer recurrence score. Where the subscores are combined, they may be combined by applying a threshold to each subscore independently in target regions, respectively, and greater than a predetermined fraction of abnormal molecules, or training a machine learning classifier to determine status based on a plurality of positive and negative training samples.

In any embodiment where a cancer recurrence score is classified as positive for cancer recurrence, the cancer recurrence status of the subject may be at risk for cancer recurrence and/or the subject may be classified as a candidate for a subsequent cancer treatment.

In some embodiments, the cancer is any one of the types of cancer described elsewhere herein, e.g., colorectal cancer.

### 3. Methods of Monitoring a Cancer in a Subject Over Time; Sample Collection at Two or More Time Points

In some embodiments, the present methods can be used to monitor one or more aspects of a condition in a subject over time, such as a subject's response to receiving a treatment for a condition (such as a response to a chemotherapeutic or immunotherapeutic), the severity of the condition (such as a cancer stage) in the subject, a recurrence of the condition (such as a cancer), and/or the subject's risk of developing the condition (such as a cancer) and/or to monitor a subject's health as part of a preventative health monitoring program (such as to determine whether and/or when a subject is in need of further diagnostic screening), such as based on changes in levels of different immune cell types, including rare immune cell types, in samples collected from a subject over time. In some embodiments, monitoring comprises analysis of at least two samples collected from a subject at at least two different time points as described herein.

The methods according to the present disclosure can also be useful in predicting a subject's response to a particular treatment option. Successful treatment options may result in an increase or decrease in the quantity of a specific immune cell type in the blood, or in the expression of one or more of the plurality of genes of a target gene set, and an unsuccessful treatment may result in no change. In other examples, this may not occur. In another example, certain treatment options may be correlated with genetic profiles of cancers over time. This correlation may be useful in selecting a therapy for a subject.

In some embodiments, quantities of each of a plurality of cell types, such as immune cell types, are determined based on sequencing and analysis of RNA isolated from at least one sample comprising cells (such as a whole blood sample or another type of blood sample (e.g., a buffy coat sample, a leukapheresis sample, or a PBMC sample) from a subject. The plurality of immune cell types can include, but is not limited to, neutrophils, lymphocytes, plasma cells, monocytes, macrophages, dendritic cells, mast cells, and eosinophils, T cells, CD4+ T cells, B cells, NK cells, megakaryocytes, CD8+ central memory cells, CD8+ effector memory cells, CD4+ central memory cells, CD4+ effector memory cells, immature neutrophils, precursor B cells, plasma cells, memory-switched B cells, plasma cells, basophils, naïve B cells, memory B cells, CD8+ T cells, naïve CD4+ T cells, resting CD4+ memory T cells, activated CD4+ memory T cells; follicular helper T cells; regulatory T cells (Tregs); gamma delta T cells; resting NK cells; activated NK cells, M0 macrophages, M1 macrophages, M2 macrophages, resting dendritic cells, activated dendritic cells, resting mast cells, and activated mast cells. As disclosed herein, differences in levels and/or presence of particular genetic signatures in RNA isolated from blood samples from a subject can be used to quantify cell types, such as immune cell types, within the sample. Thus, a comparison of the disclosed genetic signatures in RNA isolated from blood samples collected from a subject at two or more time points can be used to monitor changes in cell type quantities in the subject under different conditions (such as prior to and after a treatment), or over time (e.g., as part of a preventative health monitoring program).

The disclosed methods can include evaluating (such as quantifying) and/or interpreting cell types (such as immune cell types) present in one or more samples comprising cells or a blood sample (e.g., a buffy coat sample, a whole blood sample, a leukapheresis sample, or a PBMC sample), collected from a subject at one or more timepoints in comparison to a selected baseline value or reference standard (or a selected set of baseline values or reference standards). A baseline value or reference standard may be a quantity of cell types (such as immune cell types) measured in one or more samples (such as an average quantity or range of quantities of cell types present in at least two samples) collected from the subject at one or more time points, such as prior to receiving a treatment, prior to diagnosis of a condition (such as a cancer), or as part of a preventative health monitoring program. A baseline value or reference standard may be a quantity of cell types (such as immune cell types) measured in one or more samples (such as an average quantity or range of quantities of cell types present in at least two samples) collected at one or more timepoints from one or more subjects that do not have the condition (such as a healthy subject that does not have a cancer), one or more subjects that responded favorably to the treatment, or one or more subjects that have not received the treatment. In certain embodiments, the baseline value or reference standard utilized is a standard or profile derived from a single reference subject. In other embodiments, the baseline value or reference standard utilized is a standard or profile derived from averaged data from multiple reference subjects. The reference standard, in various embodiments, can be a single value, a mean, an average, a numerical mean or range of numerical means, a numerical pattern, or a graphical pattern created from the cell type quantity data derived from a single reference subject or from multiple reference subjects. Selection of the particular baseline values or reference standards, or selection of the one or more reference subjects, depends upon the use to which the methods described herein are to be put by, for example, a research scientist or a clinician (such as a physician).

In some embodiments, one or more samples comprising cells (such as a sample comprising cells or a blood sample (e.g., a buffy coat sample, a whole blood sample, a leukapheresis sample, or a PBMC sample) may be collected from a subject at two or more timepoints, to assess changes in cell types (such as changes in quantities of cell types, such as immune cell types) between the two or more timepoints. In some embodiments, a sample collected at a first time point is a tissue sample or a blood sample, and a sample collected at a subsequent time point (such as a second time point) is a blood sample. In some embodiments, a sample collected at a first time point is a tissue sample and a sample collected at a subsequent time point (such as a second time point) is a blood sample. By monitoring cell types and identifying differences between cell types in samples collected from a subject at two or more timepoints, the present methods can be used, for example, to determine the presence or absence of a condition (such as a cancer), a response of the subject to a treatment, one or more characteristic of a condition (such as a cancer stage) in the subject, recurrence of a condition (such as a cancer), and/or a subject's risk of developing a condition (such as a cancer). Thus, in some embodiments, methods are provided wherein quantities of cell types present in at least one sample (such as at least one whole blood sample, buffy coat sample, leukapheresis sample, or PBMC sample) collected from a subject at one or more timepoints (such as prior to receiving a treatment) are compared to quantities of cell types present in at least one sample collected from the subject at one or more different time points (such as after receiving the treatment). The disclosed methods can allow for patient-specific monitoring, such that, for example, differences in cell type quantities between samples collected from the subject at different timepoints may indicate changes (such as presence or absence of a condition, response to a treatment, a prognosis, or the like) that are significant with respect to the subject but may yet fall within a normal range of a general healthy population.

As disclosed herein, methods are provided for monitoring one or more aspects of a condition in a subject over time, such as but not limited to, a subject's response to receiving a treatment for a condition (such as a response to a chemotherapeutic or immunotherapeutic). Thus, some embodiments of the disclosed methods further comprise evaluating or monitoring a response to a treatment in the subject. In some embodiments, the evaluating or monitoring the response to the treatment in the subject comprises comparing the expression levels for the target gene set comprising a plurality of target genes that are differentially expressed in a sample from the subject collected at at least a first time point and a sample from the subject collected at at least a second time point. In some embodiments, the evaluating or monitoring the response to the treatment in the subject comprises comparing the quantities of the immune cell types in a sample from the subject collected at at least a first time point and a sample from the subject collected at at least a second time point. In some embodiments, the first time point is a time point prior to administration of the treatment to the subject, and the second time point is a time point after the administration of the treatment to the subject. In some embodiments, the first time point is a time point after administration of the treatment to the subject, and the second time point is a time point after the administration of the treatment to the subject and after the first time point.

In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points prior to the subject receiving the treatment. In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points after the subject has received the treatment. Sample collection from a subject can be ongoing during and/or after treatment to monitor the subject's response to the treatment.

In some embodiments, samples are not collected from a subject prior to diagnosis of a condition (such as a cancer) or prior to receiving a treatment. In such embodiments, wherein the response of a subject to a treatment, or the course or stage of a condition (such as a cancer) in the subject is being monitored over time, cell types are compared between samples taken at at least 2-10, at least 2-5, at least 3-6, or at least 2, such as at least 3, at least 4, at least 5, at least 6, at least 7, at least 8, at least 9, at least 10, at least 15, or at least 20 time points collected after the subject has been diagnosed and/or after the subject has received the treatment. Sample collection from a subject can be ongoing during and/or after treatment to monitor the subject's response to the treatment.

In some embodiments of the disclosed methods, one or more samples comprising cells or a blood sample (such as one or more whole blood, buffy coat, leukapheresis, or PBMC samples) is collected from a subject at least once per year, such as about 1-12 times or about 2-6 times, such as about 1, 2, 3, 4, 5, 6, 7, 8, 9, 10, 11, or 12 times per year. In other embodiments, one or more samples is collected from the subject less than once per year, such as about once every 13, 14, 15, 16, 17, 18, 19, 20, 21, 22, 23, or 24 months. In some embodiments, one or more samples is collected from the subject about once every 1-5 years or about once every 1-2 years, such as about every 1, 1.5, 2, 2.5, 3, 3.5, 4, 4.5, or 5 years.

In other embodiments of the disclosed methods, one or more samples comprising cells or one or more blood samples, e.g., one or more buffy coat samples, whole blood samples, leukapheresis samples, or PBMC samples, are collected from a subject at least once per week, such as on 1-4 days, 1-2 days, or on 1, 2, 3, 4, 5, 6, or 7 days per week. In certain embodiments, one or more samples is collected from the subject at least once per month, such as 1-15 times, 1-10 times, 2-5 times, or 1, 2, 3, 4, 5, 6, 7, 8, 9, 10, 11, 12, 13, 14, or 15 times per month. In other embodiments, one or more samples is collected from the subject every month, every 2 months, every 3 months, every 4 months, every 5 months, every 6 months, every 7 months, every 8 months, every 9 months, every 10 months, every 11 months, or every 12 months. In some embodiments, one or more samples is collected from the subject at least once per day, such as 1, 2, 3, 4, 5, or 6 times per day. Selection of the one or more sample collection timepoints (e.g., the frequency of sample collection), or of the number of samples to be collected at each timepoint, depends upon the use to which the methods described herein are to be put by, for example, a research scientist or a clinician (such as a physician).

### 4. Therapies and Related Administration

In certain embodiments, the methods disclosed herein relate to identifying and administering therapies, such as customized therapies, to patients or subjects. In some embodiments, determination of the levels of particular immune cell types, including rare immune cell types, facilitates selection of appropriate treatment. In some embodiments, the patient or subject has a given disease, disorder or condition, e.g., any of the cancers or other conditions described elsewhere herein. Essentially any cancer therapy (e.g., surgical therapy, radiation therapy, chemotherapy, immunotherapy, and/or the like) may be included as part of these methods. In certain embodiments, the therapy administered to a subject comprises at least one chemotherapy drug. In some embodiments, the chemotherapy drug may comprise alkylating agents (for example, but not limited to, Chlorambucil, Cyclophosphamide, Cisplatin and Carboplatin), nitrosoureas (for example, but not limited to, Carmustine and Lomustine), anti-metabolites (for example, but not limited to, Fluorauracil, Methotrexate and Fludarabine), plant alkaloids and natural products (for example, but not limited to, Vincristine, Paclitaxel and Topotecan), anti-tumor antibiotics (for example, but not limited to, Bleomycin, Doxorubicin and Mitoxantrone), hormonal agents (for example, but not limited to, Prednisone, Dexamethasone, Tamoxifen and Leuprolide) and biological response modifiers (for example, but not limited to, Herceptin and Avastin, Erbitux and Rituxan). In some embodiments, the chemotherapy administered to a subject may comprise FOLFOX or FOLFIRI. In certain embodiments, a therapy may be administered to a subject that comprises at least one PARP inhibitor. In certain embodiments, the PARP inhibitor may include OLAPARIB, TALAZOPARIB, RUCAPARIB, NIRAPARIB (trade name ZEJULA), among others. Typically, therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In some embodiments, therapy is customized based on the status of a nucleic acid variant as being of somatic or germline origin. In some embodiments, essentially any cancer therapy (e.g., surgical therapy, radiation therapy, chemotherapy, immunotherapy, and/or the like) may be included as part of these methods. Customized therapies can include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In some embodiments, the immunotherapy or immunotherapeutic agent targets an immune checkpoint molecule. Certain tumors are able to evade the immune system by co-opting an immune checkpoint pathway. Thus, targeting immune checkpoints has emerged as an effective approach for countering a tumor's ability to evade the immune system and activating anti-tumor immunity against certain cancers. Pardoll, Nature Reviews Cancer, 2012, 12:252-264.

In certain embodiments, the immune checkpoint molecule is an inhibitory molecule that reduces a signal involved in the T cell response to antigen. For example, CTLA4 is expressed on T cells and plays a role in downregulating T cell activation by binding to CD80 (aka B7.1) or CD86 (aka B7.2) on antigen presenting cells. PD-1 is another inhibitory checkpoint molecule that is expressed on T cells. PD-1 limits the activity of T cells in peripheral tissues during an inflammatory response. In addition, the ligand for PD-1 (PD-L1 or PD-L2) is commonly upregulated on the surface of many different tumors, resulting in the downregulation of anti-tumor immune responses in the tumor microenvironment. In certain embodiments, the inhibitory immune checkpoint molecule is CTLA4 or PD-1. In other embodiments, the inhibitory immune checkpoint molecule is a ligand for PD-1, such as PD-L1 or PD-L2. In other embodiments, the inhibitory immune checkpoint molecule is a ligand for CTLA4, such as CD80 or CD86. In other embodiments, the inhibitory immune checkpoint molecule is lymphocyte activation gene 3 (LAG3), killer cell immunoglobulin like receptor (KIR), T cell membrane protein 3 (TIM3), galectin 9 (GAL9), or adenosine A2a receptor (A2aR).

Antagonists that target these immune checkpoint molecules can be used to enhance antigen-specific T cell responses against certain cancers. Accordingly, in certain embodiments, the immunotherapy or immunotherapeutic agent is an antagonist of an inhibitory immune checkpoint molecule. In certain embodiments, the inhibitory immune checkpoint molecule is PD-1. In certain embodiments, the inhibitory immune checkpoint molecule is PD-L1. In certain embodiments, the antagonist of the inhibitory immune checkpoint molecule is an antibody (e.g., a monoclonal antibody). In certain embodiments, the antibody or monoclonal antibody is an anti-CTLA4, anti-PD-1, anti-PD-L1, or anti-PD-L2 antibody. In certain embodiments, the antibody is a monoclonal anti-PD-1 antibody. In some embodiments, the antibody is a monoclonal anti-PD-L1 antibody. In certain embodiments, the monoclonal antibody is a combination of an anti-CTLA4 antibody and an anti-PD-1 antibody, an anti-CTLA4 antibody and an anti-PD-L1 antibody, or an anti-PD-L1 antibody and an anti-PD-1 antibody. In certain embodiments, the anti-PD-1 antibody is one or more of pembrolizumab (Keytruda®) or nivolumab (Opdivo®). In certain embodiments, the anti-CTLA4 antibody is ipilimumab (Yervoy®). In certain embodiments, the anti-PD-L1 antibody is one or more of atezolizumab (Tecentriq®), avelumab (Bavencio®), or durvalumab (Imfinzi®).

In certain embodiments, the immunotherapy or immunotherapeutic agent is an antagonist (e.g., antibody) against CD80, CD86, LAG3, KIR, TIM3, GAL9, or A2aR. In other embodiments, the antagonist is a soluble version of the inhibitory immune checkpoint molecule, such as a soluble fusion protein comprising the extracellular domain of the inhibitory immune checkpoint molecule and an Fc domain of an antibody. In certain embodiments, the soluble fusion protein comprises the extracellular domain of CTLA4, PD-1, PD-L1, or PD-L2. In some embodiments, the soluble fusion protein comprises the extracellular domain of CD80, CD86, LAG3, KIR, TIM3, GAL9, or A2aR. In one embodiment, the soluble fusion protein comprises the extracellular domain of PD-L2 or LAG3.

In certain embodiments, the immune checkpoint molecule is a co-stimulatory molecule that amplifies a signal involved in a T cell response to an antigen. For example, CD28 is a co-stimulatory receptor expressed on T cells. When a T cell binds to antigen through its T cell receptor, CD28 binds to CD80 (aka B7.1) or CD86 (aka B7.2) on antigen-presenting cells to amplify T cell receptor signaling and promote T cell activation. Because CD28 binds to the same ligands (CD80 and CD86) as CTLA4, CTLA4 is able to counteract or regulate the co-stimulatory signaling mediated by CD28. In certain embodiments, the immune checkpoint molecule is a co-stimulatory molecule selected from CD28, inducible T cell co-stimulator (ICOS), CD137, OX40, or CD27. In other embodiments, the immune checkpoint molecule is a ligand of a co-stimulatory molecule, including, for example, CD80, CD86, B7RP1, B7-H3, B7-H4, CD137L, OX40L, or CD70.

Agonists that target these co-stimulatory checkpoint molecules can be used to enhance antigen-specific T cell responses against certain cancers. Accordingly, in certain embodiments, the immunotherapy or immunotherapeutic agent is an agonist of a co-stimulatory checkpoint molecule. In certain embodiments, the agonist of the co-stimulatory checkpoint molecule is an agonist antibody and preferably is a monoclonal antibody. In certain embodiments, the agonist antibody or monoclonal antibody is an anti-CD28 antibody. In other embodiments, the agonist antibody or monoclonal antibody is an anti-ICOS, anti-CD137, anti-OX40, or anti-CD27 antibody. In other embodiments, the agonist antibody or monoclonal antibody is an anti-CD80, anti-CD86, anti-B7RP1, anti-B7-H3, anti-B7-H4, anti-CD137L, anti-OX40L, or anti-CD70 antibody.

In certain embodiments, the status of a nucleic acid variant from a sample from a subject as being of somatic or germline origin may be compared with a database of comparator results from a reference population to identify customized or targeted therapies for that subject. Typically, the reference population includes patients with the same cancer or disease type as the subject and/or patients who are receiving, or who have received, the same therapy as the subject. A customized or targeted therapy (or therapies) may be identified when the nucleic variant and the comparator results satisfy certain classification criteria (e.g., are a substantial or an approximate match).

In certain embodiments, the customized therapies described herein are typically administered parenterally (e.g., intravenously or subcutaneously). Pharmaceutical compositions containing an immunotherapeutic agent are typically administered intravenously. Certain therapeutic agents are administered orally. However, customized therapies (e.g., immunotherapeutic agents, etc.) may also be administered by any method known in the art, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

Therapeutic options for treating specific genetic-based diseases, disorders, or conditions, other than cancer, are generally well-known to those of ordinary skill in the art and will be apparent given the particular disease, disorder, or condition under consideration.

### F. Table of Sequences

The following table shows exemplary sequences provided herein.

| SEQ ID NO | |
|---|---|
| and | |
| description | Sequence |
| SEQ ID NO: 1 | MNSNKDKIKVIKVFEAFAGIGSQFKALKNIARSKNWEIQHSGMVEWFVDAIVSYVAIHSKN |
| A variant of | FNPKIERLDRDILSISNDSKMPISEYGIKKINNTIKASYLNYAKKHFNNLFDIKKVNKDNF |
| M.MpeI | PKNIDIFTYSFPCQDLSVQGLQKGIDKELNTRSGLLWEIERILEEIKNSFSKEEMPKYLLM |
| | ENVKNLLSHKNKKNYNTWLKQLEKFGYKSKTYLLNSKNFDNCQNRERVFCLSIRDDYLEKT |
| | GFKFKELEKVKNPPKKIKDILVDSSNYKYLNLNKYETTTFRETKSNIISRPLKNYTTFNSE |
| | NYVYNINGIGPTLTASGANSRIKIETQQGVRYLTPLECFKYMQFDVNDFKKVQSTNLISEN |
| | KMIYIAGKSIPVKILEAIFNTLEFVNNEELE |
| SEQ ID NO: 2 | MNSNKDKIKVIKVFEAFAGIGSQFKALKNIARSKNWEIQHSGMVEWFVDAIVSYVAIHSKN |
| A variant of | FNPKIERLDRDILSISNDSKMPISEYGIKKINNTIKASYLNYAKKHFNNLFDIKKVNKDNF |
| M.MpeI | PKNIDIFTYSFPCQDLSVQGLQKGIDKELNTRSGLLWEIERILEEIKNSFSKEEMPKYLLM |
| | ENVKNLLSHKNKKNYNTWLKQLEKFGYKSKTYLLNSKNFDNCQNRERVFCLSIRDDYLEKT |
| | GFKFKELEKVKNPPKKIKDILVDSSNYKYLNLNKYETTTFRETKSNIISRPLKNYTTFNSE |
| | NYVYNINGIGPTLTASGANSRIKIETQQGVRYLTPLECFKYMQFDVNDFKKVQSTNLISEN |
| | KMIYIAGRSIPVKILEAIFNTLEFVNNEELE |
| SEQ ID NO: 3 | GGSQSQNGKCEGCNPDKDEAPYYTHLGAGPDVAAIRTLMEERYGEKGKAIRIEKVIYTGKE |
| TETv | GKSSQGCPIAKWVYRRSSEEEKLLCLVRVRPNHTCETAVMVIAIMLWDGIPKLLASELYSE |
| | LTDILGKCGICTNRRCSQNETRNCCCQGENPETCGASFSFGCSWSMYYNGCKFARSKKPRK |
| | FRLHGAEPKEEERLGSHLQNLATVIAPIYKKLAPDAYNNQVEFEHQAPDCCLGLKEGRPES |
| | GVTACLDFSAHSHRDQQNMPNGSTVVVTLNREDNREVGAKPEDEQFHVLPMYIIAPEDEFG |
| | STEGQEKKIRMGSIEVLQSFRRRRVIRIGELPKSCEVSGQDAAAVQEIEYWSDSEHNFQDP |
| | CIGGVAIAPTHGSILIECAKCEVHATTKVNDPDRNHPTRISLVLYRHKNLFLPKHCLALWE |
| | AKMAEKARKEEECGKNGSDHVSQKNHGKQEKREPTGPQEPSYLRFIQSLAENTGSVTTDST |
| | VTTSPYAFTQVTGPYNTFV |
| SEQ ID NO: 4 | QSQNGKCEGCNPDKDEAPYYTHLGAGPDVAAIRTLMEERYGEKGKAIRIEKVIYTGKEGKS |
| TETcd | SQGCPIAKWVYRRSSEEEKLLCLVRVRPNHTCETAVMVIAIMLWDGIPKLLASELYSELTD |
| | ILGKCGICTNRRCSQNETRNCCCQGENPETCGASESFGCSWSMYYNGCKFARSKKPRKFRL |
| | HGAEPKEEERLGSHLQNLATVIAPIYKKLAPDAYNNQVEFEHQAPDCCLGLKEGRPFSGVT |
| | ACLDFSAHSHRDQQNMPNGSTVVVTLNREDNREVGAKPEDEQFHVLPMYIIAPEDEFGSTE |
| | GQEKKIRMGSIEVLQSFRRRRVIRIGELPKSCKKKAEPKKAKTKKAARKRSSLENCSSRTE |
| | KGKSSSHTKLMENASHMKQMTAQPQLSGPVIRQPPTLQRHLQQGQRPQQPQPPQPQPQTTP |
| | QPQPQPQHIMPGNSQSVGSHCSGSTSVYTRQPTPHSPYPSSAHTSDIYGDTNHVNFYPTSS |
| | HASGSYLNPSNYMNPYLGLLNQNNQYAPFPYNGSVPVDNGSPFLGSYSPQAQSRDLHRYPN |
| | QDHLTNQNLPPIHTLHQQTFGDSPSKYLSYGNQNMQRDAFTTNSTLKPNVHHLATFSPYPT |
| | PKMDSHFMGAASRSPYSHPHTDYKTSEHHLPSHTIYSYTAAASGSSSSHAFHNKENDNIAN |
| | GLSRVLPGFNHDRTASAQELLYSLTGSSQEKQPEVSGQDAAAVQEIEYWSDSEHNFQDPCI |
| | GGVAIAPTHGSILIECAKCEVHATTKVNDPDRNHPTRISLVLYRHKNLFLPKHCLALWEAK |
| | MAEKARKEEECGKNGSDHVSQKNHGKQEKREPTGPQEPSYLRFIQSLAENTGSVTTDSTVT |
| | TSPYAFTQVTGPYNTFV |
| SEQ ID NO: 5 | MDYKDDDDKHMGGSDFPSCRCVEQIIEKDEGPFYTHLGAGPNVAAIREIMEERFGQKGKA |
| wild type | IRIERVIYTGKEGKSSQGCPIAKWVVRRSSSEEKLLCLVRERAGHTCEAAVIVILILVWE |
| TET2 | GIPLSLADKLYSELTETLRKYGTLTNRRCALNEERTCACQGLDPETCGASFSFGCSWSMY |
| catalytic | YNGCKFARSKIPRKFKLLGDDPKEEEKLESHLQNLSTLMAPTYKKLAPDAYNNQIEYEHR |
| domain | APECRLGLKEGRPFSGVTACLDFCAHAHRDLHNMQNGSTLVCTLTREDNREFGGKPEDEQ |
| | LHVLPLYKVSDVDEFGSVEAQEEKKRSGAIQVLSSFRRKVRMLAEPVKTCRQRKLEAKKA |
| | AAEKLSGGGGSGGGGSGGGGSDEVWSDSEQSFLDPDIGGVAVAPTHGSILIECAKRELHA |
| | TTPLKNPNRNHPTRISLVFYQHKSMNEPKHGLALWEAKMAEKAREKEEECEKYG |
| SEQ ID NO: 6 | MDYKDDDDKHMGGSDFPSCRCVEQIIEKDEGPFYTHLGAGPNVAAIREIMEERFGQKGKA |
| TET2 V1900A | IRIERVIYTGKEGKSSQGCPIAKWVVRRSSSEEKLLCLVRERAGHTCEAAVIVILILVWE |
| catalytic | GIPLSLADKLYSELTETLRKYGTLTNRRCALNEERTCACQGLDPETCGASFSFGCSWSMY |
| domain | YNGCKFARSKIPRKFKLLGDDPKEEEKLESHLQNLSTLMAPTYKKLAPDAYNNQIEYEHR |
| | APECRLGLKEGRPFSGVTACLDFCAHAHRDLHNMQNGSTLVCTLTREDNREFGGKPEDEQ |
| | LHVLPLYKVSDVDEFGSVEAQEEKKRSGAIQVLSSFRRKVRMLAEPVKTCRQRKLEAKKA |
| | AAEKLSGGGGSGGGGSGGGGSDEVWSDSEQSFLDPDIGGVAVAPTHGSILIECAKRELHA |
| | TTPLKNPNRNHPTRISLAFYQHKSMNEPKHGLALWEAKMAEKAREKEEECEKYG |
| SEQ ID NO: 7 | MDYKDDDDKHMGGSDFPSCRCVEQIIEKDEGPFYTHLGAGPNVAAIREIMEERFGQKGKA |
| TET2 V1900C | IRIERVIYTGKEGKSSQGCPIAKWVVRRSSSEEKLLCLVRERAGHTCEAAVIVILILVWE |
| catalytic | GIPLSLADKLYSELTETLRKYGTLTNRRCALNEERTCACQGLDPETCGASFSFGCSWSMY |
| domain | YNGCKFARSKIPRKFKLLGDDPKEEEKLESHLQNLSTLMAPTYKKLAPDAYNNQIEYEHR |
| | APECRLGLKEGRPFSGVTACLDFCAHAHRDLHNMQNGSTLVCTLTREDNREFGGKPEDEQ |
| | LHVLPLYKVSDVDEFGSVEAQEEKKRSGAIQVLSSFRRKVRMLAEPVKTCRQRKLEAKKA |
| | AAEKLSGGGGSGGGGSGGGGSDEVWSDSEQSFLDPDIGGVAVAPTHGSILIECAKRELHA |
| | TTPLKNPNRNHPTRISLCFYQHKSMNEPKHGLALWEAKMAEKAREKEEECEKYG |
| SEQ ID NO: 8 | MDYKDDDDKHMGGSDFPSCRCVEQIIEKDEGPFYTHLGAGPNVAAIREIMEERFGQKGKA |
| TET2 V1900G | IRIERVIYTGKEGKSSQGCPIAKWVVRRSSSEEKLLCLVRERAGHTCEAAVIVILILVWE |
| catalytic | GIPLSLADKLYSELTETLRKYGTLTNRRCALNEERTCACQGLDPETCGASFSFGCSWSMY |
| domain | YNGCKFARSKIPRKFKLLGDDPKEEEKLESHLQNLSTLMAPTYKKLAPDAYNNQIEYEHR |
| | APECRLGLKEGRPFSGVTACLDFCAHAHRDLHNMQNGSTLVCTLTREDNREFGGKPEDEQ |
| | LHVLPLYKVSDVDEFGSVEAQEEKKRSGAIQVLSSFRRKVRMLAEPVKTCRQRKLEAKKA |
| | AAEKLSGGGGSGGGGSGGGGSDEVWSDSEQSFLDPDIGGVAVAPTHGSILIECAKRELHA |
| | TTPLKNPNRNHPTRISLGFYQHKSMNEPKHGLALWEAKMAEKAREKEEECEKYG |
| SEQ ID NO: 9 | MDYKDDDDKHMGGSDFPSCRCVEQIIEKDEGPFYTHLGAGPNVAAIREIMEERFGQKGKA |
| TET2 V1900I | IRIERVIYTGKEGKSSQGCPIAKWVVRRSSSEEKLLCLVRERAGHTCEAAVIVILILVWE |
| catalytic | GIPLSLADKLYSELTETLRKYGTLTNRRCALNEERTCACQGLDPETCGASFSFGCSWSMY |
| domain | YNGCKFARSKIPRKFKLLGDDPKEEEKLESHLQNLSTLMAPTYKKLAPDAYNNQIEYEHR |
| | APECRLGLKEGRPFSGVTACLDFCAHAHRDLHNMQNGSTLVCTLTREDNREFGGKPEDEQ |
| | LHVLPLYKVSDVDEFGSVEAQEEKKRSGAIQVLSSFRRKVRMLAEPVKTCRQRKLEAKKA |
| | AAEKLSGGGGSGGGGSGGGGSDEVWSDSEQSFLDPDIGGVAVAPTHGSILIECAKRELHA |
| | TTPLKNPNRNHPTRISLIFYQHKSMNEPKHGLALWEAKMAEKAREKEEECEKYG |
| SEQ ID NO: 10 | MDYKDDDDKHMGGSDFPSCRCVEQIIEKDEGPFYTHLGAGPNVAAIREIMEERFGQKGKA |
| TET2 V1900P | IRIERVIYTGKEGKSSQGCPIAKWVVRRSSSEEKLLCLVRERAGHTCEAAVIVILILVWE |
| catalytic | GIPLSLADKLYSELTETLRKYGTLTNRRCALNEERTCACQGLDPETCGASFSFGCSWSMY |
| domain | YNGCKFARSKIPRKFKLLGDDPKEEEKLESHLQNLSTLMAPTYKKLAPDAYNNQIEYEHR |
| | APECRLGLKEGRPFSGVTACLDFCAHAHRDLHNMQNGSTLVCTLTREDNREFGGKPEDEQ |
| | LHVLPLYKVSDVDEFGSVEAQEEKKRSGAIQVLSSFRRKVRMLAEPVKTCRQRKLEAKKA |
| | AAEKLSGGGGSGGGGSGGGGSDEVWSDSEQSFLDPDIGGVAVAPTHGSILIECAKRELHA |
| | TTPLKNPNRNHPTRISLPFYQHKSMNEPKHGLALWEAKMAEKAREKEEECEKYG |
| SEQ ID NO: 11 | MDYKDDDDKHMGGSDFPSCRCVEQIIEKDEGPFYTHLGAGPNVAAIREIMEERFGQ |
| TET2-CS- | KGKAIRIERVIYTGKEGKSSQGCPIAKWVVRRSSSEEKLLCLVRERAGHTCEAAVI |
| T1372S | VILILVWEGIPLSLADKLYSELTETLRKYGTLTNRRCALNEERTCACQGLDPETCG |
| | ASFSFGCSWSMYYNGCKFARSKIPRKFKLLGDDPKEEEKLESHLQNLSTLMAPTYK |
| | KLAPDAYNNQIEYEHRAPECRLGLKEGRPFSGVSACLDFCAHAHRDLHNMQNGSTL |
| | VCTLTREDNREFGGKPEDEQLHVLPLYKVSDVDEFGSVEAQEEKKRSGAIQVLSSF |
| | RRKVRMLAEPVKTCRQRKLEAKKAAAEKLSGGGGSGGGGSGGGGSDEVWSDSEQSF |
| | LDPDIGGVAVAPTHGSILIECAKRELHATTPLKNPNRNHPTRISLVFYQHKSMNEP |
| | KHGLALWEAKMAEKAREKEEECEKYG |
| SEQ ID NO: 12 | MGGSDFPSCRCVEQIIEKDEGPFYTHLGAGPNVAAIREIMEERFGQKGKAIRIERV |
| TET2-CD- | IYTGKEGKSSQGCPIAKWVVRRSSSEEKLLCLVRERAGHTCEAAVIVILILVWEGI |
| T1372S | PLSLADKLYSELTETLRKYGTLTNRRCALNEERTCACQGLDPETCGASFSFGCSWS |
| | MYYNGCKFARSKIPRKFKLLGDDPKEEEKLESHLQNLSTLMAPTYKKLAPDAYNNQ |
| | IEYEHRAPECRLGLKEGRPFSGVSACLDFCAHAHRDLHNMQNGSTLVCTLTREDNR |
| | EFGGKPEDEQLHVLPLYKVSDVDEFGSVEAQEEKKRSGAIQVLSSERRKVRMLAEP |
| | VKTCRQRKLEAKKAAAEKLSSLENSSNKNEKEKSAPSRTKQTENASQAKQLAELLR |
| | LSGPVMQQSQQPQPLQKQPPQPQQQQRPQQQQPHHPQTESVNSYSASGSTNPYMRR |
| | PNPVSPYPNSSHTSDIYGSTSPMNFYSTSSQAAGSYLNSSNPMNPYPGLLNQNTQY |
| | PSYQCNGNLSVDNCSPYLGSYSPQSQPMDLYRYPSQDPLSKLSLPPIHTLYQPRFG |
| | NSQSFTSKYLGYGNQNMQGDGFSSCTIRPNVHHVGKLPPYPTHEMDGHFMGATSRL |
| | PPNLSNPNMDYKNGEHHSPSHIIHNYSAAPGMFNSSLHALHLQNKENDMLSHTANG |
| | LSKMLPALNHDRTACVQGGLHKLSDANGQEKQPLALVQGVASGAEDNDEVWSDSEQ |
| | SFLDPDIGGVAVAPTHGSILIECAKRELHATTPLKNPNRNHPTRISLVFYQHKSMN |
| | EPKHGLALWEAKMAEKAREKEEECEKYGPDYVPQKSHGKKVKREPAEPHETSEPTY |
| | LRFIKSLAERTMSVTTDSTVTTSPYAFTRVTGPYNRYI |
| SEQ ID NO: 13 | FSGVTACLD |
| SEQ ID NO: 14 | FSGVSACLD |
| TET2 T1372S | |

### IV. Kits

Also provided are kits comprising the compositions as described herein. The kits can be useful in performing the methods as described herein. In some embodiments, the kit comprises target-specific probes that specifically bind to target region sets. In some such embodiments, the target-specific probes comprise a capture moiety. In some embodiments, the kit comprises a solid support linked to a binding partner of the capture moiety. In some embodiments, the kit comprises adapters. In some embodiments, the kit comprises PCR primers, wherein the PCR primers anneal to a target region or to an adapter. In some embodiments, the kit comprises additional elements elsewhere herein. In some embodiments, the kit comprises instructions for performing a method described herein.

Kits may further comprise a plurality of oligonucleotide probes that selectively hybridize to least 5, 6, 7, 8, 9, 10, 20, 30, 40 or all genes selected from the group consisting of ICA1, CD38, ABCB4, TNFRSF17, RRP12, CHI3L2, MAP3K13, IL4R, IL12RB2, ACHE, LAG3, CD209, GGT5, DEPDC5, UPK3A, GZMH, BPI, ACP5, CD37, MAST1, RASSF4, MS4A6A, PPFIBP1, MAK, IL18RAP, KYNU, FASLG, MYB, CCND2, TRPM6, FLVCR2, CD80, TMEM156, BHLHE41, NFE2, TREM1, TMEM255A, IL1B, PLEKHG3, VPREB3, TEP1, TRPM4, SMPDL3B, LILRB2, CHI3L1, IL2RA, TLR2, BMP2K, TNFRSF11A, PLA2G7, EPHA1, ABCB9, ZFP36L2, HHEX, SKA1, CLIC2, DUSP2, SLAMF8, CDA, ZNF222, ASGR2, PBXIP1, NIPSNAP3B, TSHR, NOD2, CD300A, NAALADL1, ADRB2, SCN9A, CEACAM3, COL8A2, CLEC7A, CTSW, HPSE, CD7, P2RY14, HIC1, P2RY13, GPR19, CCR3, MROH7, CSF1, FAM174B, TNFRSF4, PDCD1, RYR1, HLA-DQA1, NCR3, ZBTB10, APOBEC3G, LILRA2, TNFRSF13B, HLA-DOB, PGLYRP1, HGF, ATP9A, ATP2C2, JMJD6, DHRS9, SLC1A3, CEACAM1, DUSP13, CRISP3, ABLIM1, HSD3B7, OSM, UPB1, BIK, MMP9, SLCO4A1, BMX, KLF5, RETN, GRB10, PRUNE2, ERLIN1, TP5313, IL1R2, EPAS1, LRRC42, GADD45A, PHTF1, RCAN3, ARG1, CYSTM1, DACH1, FKBP9, GOS2, PFKFB2, CDH26, ARMC7, PPPIR3D, ECHDC3, RDH5, ACVRIB, CKAP4, MTHFS, IL10, MFSD13A, GPR84, MYLK3, ZNF787, MYO10, RAB19, OLAH, ANKRD22, RABGEF1, SAMSN1, CACNA1D, RGL4, TFF3, GYG1, ZDHHC19, SLC51A, POC1A, HPGD, FBN1, CLEC4D, SEMA6B, PFKFB3, PAQR8, SH3PXD2B, FOSL1, MSRA, LRRN1, KCNE5, B3GNT5, CD163, CRACR2B, OPLAH, EXOSC4, KCNE1, UPP1, ST6GALNAC3, SEPTIN5, FCAR, SPATC1, MAFG, IL27, ENTPD7, PLIN5, FAM228B, ORM1, STON1, CFH, HS3ST1, DBNDD1, CD22, SLC25A39, KCNG1, TGFBR3, ADD2, COL19A1, CD200, TCL1A, PROCR, CD40, NME4, TSPAN13, RGS9, FAM184A, KHDRBS2, ENPP5, MMP8, SATB2, GPR68, CEACAM8, MYO1B, LARGE1, NT5E, RAPGEF5, ABHD17C, ZNF365, GRTP1, IGFBP3, LCN2, GLB1L2, CNKSR2, PRSS23, RASGRP3, SCN3A, C16orf74, RETREG1, ERG, SNX22, CXCR5, BEND5, SLC1A7, LEXM, CAMK2N1, SPRY1, CDCA7L, SPIB, DLC1, DIPK1B, MTCL1, PARM1, MZB1, SLC23A1, PDGFD, NMUR1, NBEA, OLR1, PTPRM, PAWR, ABCA13, TAFA1, KCNH8, EFNA5, PRSS57, PTCH1, PRTN3, SULTIA1, ZNF667, SHISA4, L1CAM, RASGEF1A, DDR1, GPC2, PATL2, and IGLL5. The number genes to which the oligonucleotide probes can selectively hybridize can vary. For example, the number of genes can comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, or 54. The kit can include a container that includes the plurality of oligonucleotide probes and instructions for performing any of the methods described herein.

The oligonucleotide probes can selectively hybridize to exon regions of the genes, e.g., of the at least 5 genes. In some cases, the oligonucleotide probes can selectively hybridize to at least 30 exons of the genes, e.g., of the at least 5 genes. In some cases, the multiple probes can selectively hybridize to each of the at least 30 exons. The probes that hybridize to each exon can have sequences that overlap with at least 1 other probe. In some embodiments, the oligoprobes can selectively hybridize to non-coding regions of genes disclosed herein, for example, intronic regions of the genes. The oligoprobes can also selectively hybridize to regions of genes comprising both exonic and intronic regions of the genes disclosed herein.

Any number of exons can be targeted by the oligonucleotide probes. For example, at least 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 400, 500, 600, 700, 800, 900, 1,000, or more, exons can be targeted.

The kit can comprise at least 4, 5, 6, 7, or 8 different library adapters having distinct molecular barcodes and identical sample barcodes. The library adapters may not be sequencing adapters. For example, the library adapters do not include flow cell sequences or sequences that permit the formation of hairpin loops for sequencing. The different variations and combinations of molecular barcodes and sample barcodes are described throughout, and are applicable to the kit. Further, in some cases, the adapters are not sequencing adapters. Additionally, the adapters provided with the kit can also comprise sequencing adapters. A sequencing adapter can comprise a sequence hybridizing to one or more sequencing primers. A sequencing adapter can further comprise a sequence hybridizing to a solid support, e.g., a flow cell sequence. For example, a sequencing adapter can be a flow cell adapter. The sequencing adapters can be attached to one or both ends of a polynucleotide fragment. In some cases, the kit can comprise at least 8 different library adapters having distinct molecular barcodes and identical sample barcodes. The library adapters may not be sequencing adapters. The kit can further include a sequencing adapter having a first sequence that selectively hybridizes to the library adapters and a second sequence that selectively hybridizes to a flow cell sequence. In another example, a sequencing adapter can be hairpin shaped. For example, the hairpin shaped adapter can comprise a complementary double stranded portion and a loop portion, where the double stranded portion can be attached (e.g., ligated) to a double-stranded polynucleotide. Hairpin shaped sequencing adapters can be attached to both ends of a polynucleotide fragment to generate a circular molecule, which can be sequenced multiple times. A sequencing adapter can be up to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more bases from end to end. The sequencing adapter can comprise 20-30, 20-40, 30-50, 30-60, 40-60, 40-70, 50-60, 50-70, bases from end to end. In a particular example, the sequencing adapter can comprise 20-30 bases from end to end. In another example, the sequencing adapter can comprise 50-60 bases from end to end. A sequencing adapter can comprise one or more barcodes. For example, a sequencing adapter can comprise a sample barcode. The sample barcode can comprise a pre-determined sequence. The sample barcodes can be used to identify the source of the polynucleotides. The sample barcode can be at least 1, 2, 3, 4, 5, 6, 7, 8, 9, 10, 11, 12, 13, 14, 15, 16, 17, 18, 19, 20, 21, 22, 23, 24, 25, or more (or any length as described throughout) nucleic acid bases, e.g., at least 8 bases. The barcode can be contiguous or non-contiguous sequences, as described above.

The library adapters can be blunt ended and Y-shaped and can be less than or equal to 40 nucleic acid bases in length. Other variations of the can be found throughout and are applicable to the kit.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the disclosure described herein may be employed in practicing the invention. It is therefore contemplated that the disclosure shall also cover any such alternatives, modifications, variations or equivalents. It is intended that the following claims define the scope of the invention and that methods and structures within the scope of these claims and their equivalents be covered thereby.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, computer readable media, and/or component features, steps, elements, or other aspects thereof can be used in various combinations.

All patents, patent applications, websites, other publications or documents, accession numbers and the like cited herein are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number, if applicable. Likewise, if different versions of a publication, website or the like are published at different times, the version most recently published at the effective filing date of the application is meant, unless otherwise indicated.

### EXAMPLES

### Example 1: RNA Isolation and Sequencing

Sequencing and quantifying of nucleic acids isolated from patient samples (such as whole blood samples from one or more cohorts comprising healthy subjects and/or subjects that have advanced adenoma (AA) or colorectal cancer (CRC)) is achieved by performing RNA sequencing (RNA-seq) analysis. RNA-seq enables determinations of gene expression and the systematic identification of cell populations (e.g., quantification of immune cell types) in a sample. Gene expression can be compared across samples (such as between samples from healthy subjects and samples from subjects having AA or CRC) to identify genes differentially expressed in different samples. Similarly, quantification of cell populations allows comparisons of such populations between samples (e.g., comparisons of immune cell populations between samples from healthy subjects and samples from subjects having AA or CRC).

RNA-seq involves isolation of RNA (such as total RNA) from tissues or cells of interest followed by construction of cDNA libraries and sequencing of these libraries (such as using a next-generation sequencing instrument). In this Example, RNA was extracted from whole blood samples collected from healthy subjects or from subjects having AA or CRC using the PAXgene® Blood RNA Kit (PreAnalytix GmbH) essentially according to the manufacturer's protocol.

Briefly, whole blood samples were lysed and then stored in tubes containing cationic detergent and frozen for later analysis. After thawing, the sample was centrifuged for 10 minutes at 4000 x g to produce a pellet. The supernatant was removed and the pellet was resuspended and washed (such as in RNase-free water), and centrifuged again for 10 min at 4000 x g. The supernatant was removed and the pellet was incubated with buffers containing proteinase K to digest proteins. Residual cellular debris was removed from the sample (such as via centrifugation through a PAXgene Shredder spin column). Ethanol was added to the sample, and the lysate was applied to a column (such as the PAXgene RNA spin column). During a brief centrifugation, RNA was selectively bound to the silica membrane of the column as contaminants passed through. Remaining contaminants were removed in several wash steps. While the RNA was bound to the silica, the column membrane was treated with DNase I to remove bound DNA. After additional wash steps, RNA was eluted and used in downstream applications. The RNA was stored at or below-20° C. until use.

The isolated RNA (approximately 200-600 ng total RNA) was depleted of ribosomal RNA (rRNA) and globin RNA using a commercially available kit (e.g., the Watchmaker Genomics RNA Library Prep Kit with Polaris Depletion). RNA spike-in controls (i.e., ERCC spike-in controls) were added as a standard. RNA was fragmented to produce fragments of approximately 150 bp. RNA was converted to cDNA using random priming, followed by second strand synthesis, end repair, and optional A-tailing. Adapters comprising barcodes were ligated to the cDNA, which was then amplified. Library amplification was performed using PCR with indexed oligonucleotide primers that included sample index tags and sequencing primer binding sites. Amplified libraries were then sequenced to a depth of approximately 50 million reads per sample and analyzed (with deduplication).

### Example 2: Analysis of Gene Expression Data to Determine the Presence, Absence, or Likelihood of AA and/or CRC: Model Training and Testing

RNA-seq was performed essentially as described above, using sets of samples from healthy subjects and subjects with advanced adenoma (AA) or colorectal cancer (CRC) to identify genes differentially expressed in healthy subjects versus those with AA or CRC, including genes associated with certain immune cell types (Table 2). A logistic regression model was then developed to determine whether the presence, absence, or likelihood of AA and/or CRC could be determined based on the quantities (e.g., proportions) of the immune cell types and/or the expression levels of the identified genes.

TABLE 2
Training and testing data (cohorts). Duplicates, sex mismatched
samples, and those with RNA integrity number (RIN) <4 were removed.

| | | Cancer | No. | |
|---|---|---|---|---|
| Batch | Cohort | Status | Subjects | Utility |
| 0 | A, B | CRC | 41 | |
| | | Healthy | 41 | Training CRC models; |
| | | | | Test set for AA models |
| 1*, 2* | C | AA | 21 | Training AA models; |
| | | Healthy | 54 | Test set for CRC models |
| | | NAA | 2 | |
| | B | AA | 11 | Test set for AA and CRC |
| | | Healthy | 23 | models |
| | | NAA | 7 | |
| | | CRC | 51 | |
| 3 | D | AA | 40 | Test set for AA models |
| | | CRC | 6 | |
| | | Healthy | 11 | |
| | | NAA | 13 | |

*Cohorts B and C were randomized across two batches (batches 1 and 2).

Cell type deconvolution using the RNA-seq data returned the cell type composition of each sample across more than 20 cell types, which could be grouped into broader cell types including naïve B cells, memory B cells, plasma cells, CD8+ T cells, CD4+ T cells, Treg cells, NK cells, monocytes, macrophages, and dendritic cells. Based on these cell type proportions, meta-features were also defined and were calculated from cell-type abundances (quantities): (1) normalized lymphocyte proportions (T, B, and NK cells normalized by total lymphocytes); (2) neutrophil to lymphocyte ratio (NLR); and (3) neutrophil to T, B, and NK cell ratios. The cell type proportions (excluding meta features) sum to 1 across all cell types. Therefore, if one cell type is higher in abundance in a subset of samples, the other cell types will be lower in proportion. Cell type proportions are biologically meaningful and genes used in cell type proportion calculations can be included as features for a model.

On the other hand, using normalized gene expression values (independently of determining cell type proportions) allows for identification of genes differentially expressed in AA or CRC samples versus healthy samples that are not related to changes to cell type proportions. Upregulation of one gene can lead to reduced expression for all remaining genes because RNA is competitive; however, this is less likely to impact gene expression measurements because there are many genes. In other words, competitiveness can be buffered across a large number of genes. Given that more than 100 samples were used in generating the model, and there are thousands of genes, the model was under-specified. Regularization helped minimize this issue, as did reducing the number of genes using biological (and other) constraints.

Models from the following gene sets were used for training (see Table 3 below): (1) a cell type deconvolution (CTD) gene panel (approximately 400 genes); (2) the top 100 differentially expressed genes for CRC; (3) the top 100 differentially expressed genes for AA (for two sets of cohorts) and (4) the top 500 variable genes based on variance.

Expression data were obtained using Salmon (available on the Internet: github.com/COMBINE-lab/salmon). The expression counts were de-duplicated, restricted to protein coding genes only, and finally transcripts per million (TPM)-normalized. The TPM-normalized values were further mean-centered and scaled to unit variance for each gene. Performing this standardization allows for scaling of all features in a similar way based on the L2 penalty. L1 regularization penalizes the sum of absolute values of the weights, whereas L2 regularization penalizes the sum of squares of the weights.

Model training was performed using the sklearn (scikit-learn) package in python. Three folds of cross validation were randomly generated. For each test fold, the area under the curve (AUC) was calculated. The L2 and L1 penalty corresponding with the best average AUC across all five test folds was taken, and a final refit was performed using the entire training dataset with the optimal penalty.

Class imbalances can reduce model performance and result in overfitting. For example, if there are many more samples from healthy subjects than from subjects with AA or CRC, then the decision surface between healthy and AA or CRC samples will be predominantly determined by the healthy samples. To address this, healthy samples can be down-weighted and AA or CRC samples can be up-weighted so that the decision surface is equally influenced by both healthy samples and AA or CRC samples. Additionally, sample-specific weights can be assigned based on any other covariate that may be over-influencing the decision surface.

A sex-imbalance was observed in the AA samples, particularly in cohort C. Of the 54 healthy samples in cohort C, 43 were female. Since the healthy subject group was mostly females, a model could potentially learn genes associated with sex differences rather than genes that are associated with AA. Thus, female healthy samples can be down-weighted in this instance, and male AA samples, female AA samples, and male healthy samples can each be up-weighted.

Table 3 below summarizes the gene expression models evaluated in this Example:

TABLE 3
Gene expression models

| Features | No. genes |
|---|---|
| CTD gene panel | 400 |
| Top variable genes | 500 |
| Top CRC differentially expressed genes | 100 |
| Top 100 cohort C AA differentially | 100 |
| expressed genes | |
| Top 100 cohort C + cohort B AA | 100 |
| differentially expressed genes | |

For each feature type, L1 or L2 regularization can be performed, sex may or may not be regressed out, and hyperparameter optimization, class weighting, and sample sex weighting may or may not be performed. As a result of these five parameters, for each training dataset, there are 5*2*2*2*2*2=160 models. Training both a CRC model and an AA model doubles this number of models to 320, and each model would be evaluated using three independent test sets. Rather than training and testing such a large number of models, each option in Table 3 above was evaluated while keeping the other options fixed. For example, evaluating how L2 optimization versus default penalty performs across the five feature sets can be done without hyperparameter optimization, class weighting, sample sex weighting, or regressing out sex. In this way, only 10 models need be compared at a time (five features, optimized or not). This method can be repeated for the other binary options (e.g., five features, class weighted or not). A binary decision can thus be made for each option to identify the best performing “greedy” configuration.

While the top differentially expressed (variable) genes can depend on the training cohort, the other feature sets are fixed. For each model, performance was evaluated using receiver operating characteristic (ROC) curves, AUC, and sensitivity at 90% precision. Evaluating the models showed that input features have the biggest influence over model performance across both the AA and CRC cohorts. The cell type deconvolution genes showed the best AA test performance. The Top 100 CRC differentially expressed genes showed the best CRC test performance, although this was expected given that the top 100 differentially expressed genes were derived from the same test set. Further, L1 regularization generally performed better than L2 regularization for a similar range of penalty values. The biggest discrepancy observed was in the top 100 CRC differentially expressed genes model, likely due to L1 doing a superior job of eliminating individual genes from this highly correlated gene set. The ROC curves for four of the five models listed in Table 3 are shown in FIGS. 1A-1D.

As discussed above, class weights and sample weights provide a way to account for imbalances in the training data. Class weights in this Example account for an imbalance of AA samples versus healthy samples in cohort C. Given the relatively larger number of healthy samples, AA samples were up-weighted and healthy samples are down-weighted. This was effectively similar to re-sampling the data to an equal proportion of AA samples and healthy samples. Class weighting (versus refraining from class weighting) did not appear to substantially impact model performance.

Also as discussed above, the sex imbalance in the testing and training data could potentially reduce model performance. Plotting the model scores showed that the average score was higher for male samples. Although there were more male AA samples than female AA samples in cohort D, plotting the model scores showed that the average score was higher for male samples than for female samples. Considering that healthy samples were mostly female in the training data, it was plausible that the model was predicting male and AA status.

To attempt to mitigate this possible confounding of sex covariates in model training, the effect of sex on gene expression was regressed out, using healthy samples only to learn the sex coefficient to remove. In the CTD gene panel model, cohort D AA performance went down from 0.4 to 0.25 after regressing out sex from the training data, whereas cohort B CRC and cohort B AA model sensitivity increased. However, this trend was not consistent across models. For example, in the top variable genes model, the performance of cohort D AA increased, while that of cohort B CRC decreased.

Given the above, it appeared evident that the CTD gene panel model was identifying genes that differed between sex. After sex-regression, model scores for female AA and healthy samples were scored higher. The model specificity decreased in general, likely because power was lost due to confounded genes that contain both a sex effect and an AA or healthy effect. An alternative approach to regressing out sex was to simply remove genes that showed a significant association with sex. This approach avoided potential artifacts introduced by regressing out sex. DESeq2 was used to perform differential expression between male and female healthy samples. The differentially expressed genes having a false discovery rate (FDR)<10% were considered sex associated and were removed from the expression matrix. After removing such genes, female samples scored slightly higher, but not as high as in the sex regression approach.

Exemplary genes identified as being associated with cell type (“CTD”), differentially expressed in AA versus healthy samples (“AA DEG”), or differentially expressed in CRC versus healthy samples (“CRC DEG”) are shown in Table 4 below.

TABLE 4
Exemplary genes.

| Gene | Association | Gene | Association | Gene | Association |
|---|---|---|---|---|---|
| CFH | AA DEG | TMEM156 | CTD | DLC1 | AA DEG |
| HS3ST1 | AA DEG | FKBP9 | CRC DEG | NIPSNAP3B | CTD |
| ICA1 | CTD | BHLHE41 | CTD | TSHR | CTD |
| DBNDD1 | AA DEG | NFE2 | CTD | DIPK1B | AA DEG |
| CD38 | CTD | G0S2 | CRC DEG | FBN1 | CRC DEG |
| ABCB4 | CTD | PFKFB2 | CRC DEG | CLEC4D | CRC DEG |
| PGLYRP1 | CRC DEG | CDH26 | CRC DEG | NOD2 | CTD |
| CD22 | AA DEG | CEACAM8 | AA DEG | SEMA6B | CRC DEG |
| SLC25A39 | AA DEG | TREM1 | CTD | CD300A | CTD |
| HGF | CRC DEG | TMEM255A | CTD | NAALADL1 | CTD |
| KCNG1 | AA DEG | ARMC7 | CRC DEG | MTCL1 | AA DEG |
| TNFRSF17 | CTD | IL1B | CTD | PARM1 | AA DEG |
| RRP12 | CTD | PLEKHG3 | CTD | ADRB2 | CTD |
| ATP9A | CRC DEG | VPREB3 | CTD | SCN9A | CTD |
| ATP2C2 | CRC DEG | MYO1B | AA DEG | MZB1 | AA DEG |
| CHI3L2 | CTD | TEP1 | CTD | SLC23A1 | AA DEG |
| TGFBR3 | AA DEG | TRPM4 | CTD | PFKFB3 | CRC DEG |
| JMJD6 | CRC DEG | SMPDL3B | CTD | PAQR8 | CRC DEG |
| DHRS9 | CRC DEG | LILRB2 | CTD | CEACAM3 | CTD |
| MAP3K13 | CTD | PPP1R3D | CRC DEG | PDGFD | AA DEG |
| ADD2 | AA DEG | CHI3L1 | CTD | NMUR1 | AA DEG |
| IL4R | CTD | LARGE1 | AA DEG | COL8A2 | CTD |
| SLC1A3 | CRC DEG | IL2RA | CTD | CLEC7A | CTD |
| CEACAM1 | CRC DEG | ECHDC3 | CRC DEG | CTSW | CTD |
| DUSP13 | CRC DEG | NT5E | AA DEG | NBEA | AA DEG |
| IL12RB2 | CTD | RDH5 | CRC DEG | HPSE | CTD |
| COL19A1 | AA DEG | ACVR1B | CRC DEG | OLR1 | AA DEG |
| ACHE | CTD | CKAP4 | CRC DEG | PTPRM | AA DEG |
| LAG3 | CTD | RAPGEF5 | AA DEG | CD7 | CTD |
| CD209 | CTD | MTHFS | CRC DEG | SH3PXD2B | CRC DEG |
| CD200 | AA DEG | ABHD17C | AA DEG | P2RY14 | CTD |
| CRISP3 | CRC DEG | IL10 | CRC DEG | FOSL1 | CRC DEG |
| ABLIM1 | CRC DEG | TLR2 | CTD | MSRA | CRC DEG |
| HSD3B7 | CRC DEG | MFSD13A | CRC DEG | LRRN1 | CRC DEG |
| OSM | CRC DEG | ZNF365 | AA DEG | KCNE5 | CRC DEG |
| GGT5 | CTD | BMP2K | CTD | B3GNT5 | CRC DEG |
| UPB1 | CRC DEG | GPR84 | CRC DEG | HIC1 | CTD |
| DEPDC5 | CTD | GRTP1 | AA DEG | PAWR | AA DEG |
| BIK | CRC DEG | MYLK3 | CRC DEG | CD163 | CRC DEG |
| UPK3A | CTD | TNFRSF11A | CTD | CRACR2B | CRC DEG |
| GZMH | CTD | ZNF787 | CRC DEG | OPLAH | CRC DEG |
| TCL1A | AA DEG | MYO10 | CRC DEG | EXOSC4 | CRC DEG |
| MMP9 | CRC DEG | PLA2G7 | CTD | ABCA13 | AA DEG |
| PROCR | AA DEG | IGFBP3 | AA DEG | KCNE1 | CRC DEG |
| CD40 | AA DEG | EPHA1 | CTD | P2RY13 | CTD |
| SLCO4A1 | CRC DEG | RAB19 | CRC DEG | GPR19 | CTD |
| BPI | CTD | LCN2 | AA DEG | CCR3 | CTD |
| BMX | CRC DEG | GLB1L2 | AA DEG | TAFA1 | AA DEG |
| KLF5 | CRC DEG | CNKSR2 | AA DEG | UPP1 | CRC DEG |
| ACP5 | CTD | PRSS23 | AA DEG | KCNH8 | AA DEG |
| NME4 | AA DEG | ABCB9 | CTD | ST6GALNAC3 | CRC DEG |
| CD37 | CTD | OLAH | CRC DEG | MROH7 | CTD |
| RETN | CRC DEG | ZFP36L2 | CTD | EFNA5 | AA DEG |
| MAST1 | CTD | RASGRP3 | AA DEG | CSF1 | CTD |
| GRB10 | CRC DEG | ANKRD22 | CRC DEG | SEPTIN5 | CRC DEG |
| TSPAN13 | AA DEG | HHEX | CTD | PRSS57 | AA DEG |
| PRUNE2 | CRC DEG | SCN3A | AA DEG | FAM174B | CTD |
| RASSF4 | CTD | C16orf74 | AA DEG | PTCH1 | AA DEG |
| ERLIN1 | CRC DEG | RETREG1 | AA DEG | FCAR | CRC DEG |
| RGS9 | AA DEG | RABGEF1 | CRC DEG | SPATC1 | CRC DEG |
| MS4A6A | CTD | SKA1 | CTD | TNFRSF4 | CTD |
| PPFIBP1 | CTD | SAMSN1 | CRC DEG | PDCD1 | CTD |
| MAK | CTD | CLIC2 | CTD | RYR1 | CTD |
| FAM184A | AA DEG | CACNA1D | CRC DEG | PRTN3 | AA DEG |
| KHDRBS2 | AA DEG | ERG | AA DEG | SULT1A1 | AA DEG |
| ENPP5 | AA DEG | SNX22 | AA DEG | HLA-DQA1 | CTD |
| TP5313 | CRC DEG | DUSP2 | CTD | MAFG | CRC DEG |
| IL1R2 | CRC DEG | SLAMF8 | CTD | IL27 | CRC DEG |
| IL18RAP | CTD | CDA | CTD | ENTPD7 | CRC DEG |
| KYNU | CTD | RGL4 | CRC DEG | ZNF667 | AA DEG |
| EPAS1 | CRC DEG | ZNF222 | CTD | SHISA4 | AA DEG |
| LRRC42 | CRC DEG | TFF3 | CRC DEG | L1CAM | AA DEG |
| GADD45A | CRC DEG | CXCR5 | AA DEG | RASGEF1A | AA DEG |
| PHTF1 | CRC DEG | ASGR2 | CTD | NCR3 | CTD |
| FASLG | CTD | BEND5 | AA DEG | DDR1 | AA DEG |
| RCAN3 | CRC DEG | SLC1A7 | AA DEG | ZBTB10 | CTD |
| MMP8 | AA DEG | LEXM | AA DEG | GPC2 | AA DEG |
| MYB | CTD | CAMK2N1 | AA DEG | PLIN5 | CRC DEG |
| ARG1 | CRC DEG | PBXIP1 | CTD | FAM228B | CRC DEG |
| CCND2 | CTD | GYG1 | CRC DEG | ORM1 | CRC DEG |
| SATB2 | AA DEG | ZDHHC19 | CRC DEG | PATL2 | AA DEG |
| TRPM6 | CTD | SLC51A | CRC DEG | APOBEC3G | CTD |
| FLVCR2 | CTD | SPRY1 | AA DEG | LILRA2 | CTD |
| GPR68 | AA DEG | POC1A | CRC DEG | TNFRSF13B | CTD |
| CYSTM1 | CRC DEG | HPGD | CRC DEG | HLA-DOB | CTD |
| CD80 | CTD | CDCA7L | AA DEG | STON1 | CRC DEG |
| DACH1 | CRC DEG | SPIB | AA DEG | IGLL5 | AA DEG |

### Example 3: Analysis of Cell Type Proportion Estimates to Determine the Presence, Absence, or Likelihood of AA: Model Training and Testing

RNA-seq was performed essentially as described above, using sets of samples from healthy subjects and subjects with advanced adenoma (AA) to identify cell types present in different proportions in healthy subjects versus those with AA (Table 5). Cell type deconvolution using the RNA-seq data was used to estimate the relative proportions of each cell type within each sample. A logistic regression model was then developed to determine whether the presence, absence, or likelihood of AA could be determined based on the quantities (e.g., proportions) of the immune cell types.

TABLE 5
Training and testing data (cohorts). Duplicates, sex mismatched
samples, and those with RNA integrity number (RIN) <4 were removed.

| | Cancer | | Number | |
|---|---|---|---|---|
| Cohort | Status | Sex | samples | Utility |
| A | AA | M | 56 | Training set for AA model |
| | | F | 44 | |
| | Healthy | M | 44 | |
| | | F | 67 | |
| B | AA | M | 16 | Test set for AA model |
| | | F | 10 | |
| | Healthy | M | 34 | |
| | | F | 87 | |

Expression data were obtained using transcripts per million (TPM)-normalized counts processed by Salmon (available on the Internet: github.com/COMBINE-lab/salmon), as described in Example 2. Table 1 lists genes observed to be differentially expressed in particular cell types (specifically, CD8+ T cells, NK cells, B cells, monocytes and macrophages, neutrophils, and CD4+ T cells), inferred as a component of bulk expression. Cell type deconvolution was performed using iSort Fractions (isort.cibermed.com), using as input the Salmon-processed RNA-seq data. This returned the cell type composition of each sample across more than 20 cell types, which could be grouped into broader cell types including naïve B cells, memory B cells, plasma cells, CD8+ T cells, CD4+ T cells, Treg cells, NK cells, monocytes, macrophages, and dendritic cells. Based on the proportions of these cell types, meta-features were also defined and were calculated from the abundances (quantities) of the cell types: (1) normalized lymphocyte proportions (T, B, and NK cells normalized by total lymphocytes); (2) neutrophil to lymphocyte ratio (NLR); and (3) neutrophil to T, B, and NK cell ratios. The cell type proportions (excluding meta features) sum to 1 across all cell types. Therefore, if one cell type is higher in abundance in a subset of samples, the other cell types will be lower in proportion compared to that cell type. Cell type proportions are biologically meaningful, and genes used in cell type proportion calculations can also be included as features for a model.

Modeling features included the estimated proportions of the cell types described above. Since some cell types are difficult to quantify precisely at low values, we added a small pseudocount to each feature before logit-transforming the features, to minimize the effects of noise. We used a pseudocount in the range of 0.0006-0.002 (as a proportion out of 1) because this range corresponds roughly to our limit of detection for most cell types. A pseudocount value in this range achieved superior model performance in training data, and produced a more balanced overall distribution of output model probabilities than a model trained using an overly small pseudocount, such as 0.00001.

Model training was performed using the sklearn (scikit-learn) package in python with L2 regularization, with a penalty value set as described in Example 2. A final fit was performed using the entire training dataset with the optimal L2 penalty coefficient.

Class imbalances can reduce model performance and result in overfitting. For example, if there are many more samples from healthy subjects than from subjects with AA or CRC, then the decision surface between healthy and AA or CRC samples will be predominantly determined by the healthy samples. To address this, healthy samples can be down-weighted, and AA or CRC samples can be up-weighted, so that the decision surface is equally influenced by both healthy samples and AA or CRC samples. Additionally, sample-specific weights can be assigned based on any other covariate that may be over-influencing the decision surface.

A sex-imbalance was observed in the AA vs. healthy samples. In Cohort A, 44% of the AA samples and 60% of the healthy samples were female. Similarly in Cohort B, 38% of the AA samples and 72% of the healthy samples were female. To attempt to mitigate the possible confounding of sex covariates in model training, the effect of sex on cell type proportions was regressed out, using healthy samples only to learn the sex coefficient to remove.

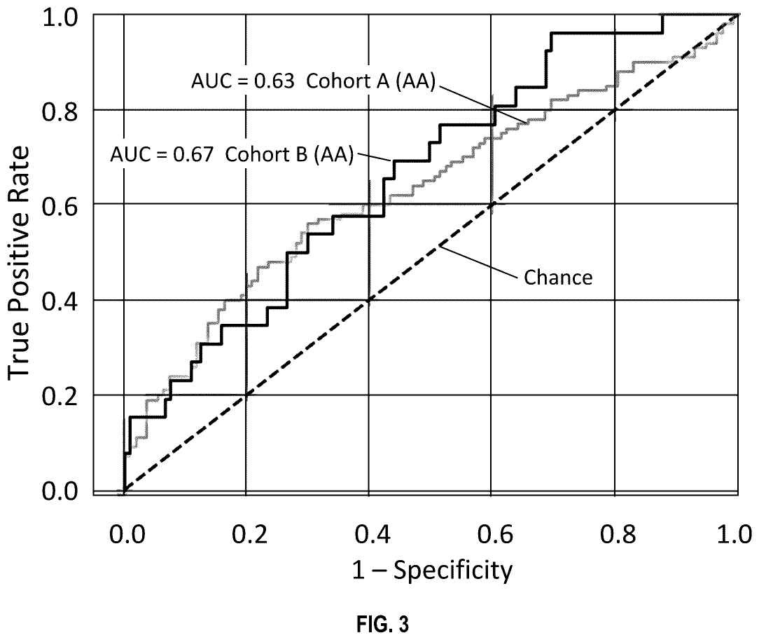

Model performance was evaluated using receiver operating characteristic (ROC) curves, AUC, and sensitivity at 90% precision. ROC curves for this model are shown in FIG. 3. The ROC curve for Cohort A (training data) uses scores generated by leave-one-out cross validation, to reduce potential for overfitting. The ROC curve for Cohort B (test data) shows data that were excluded from training.

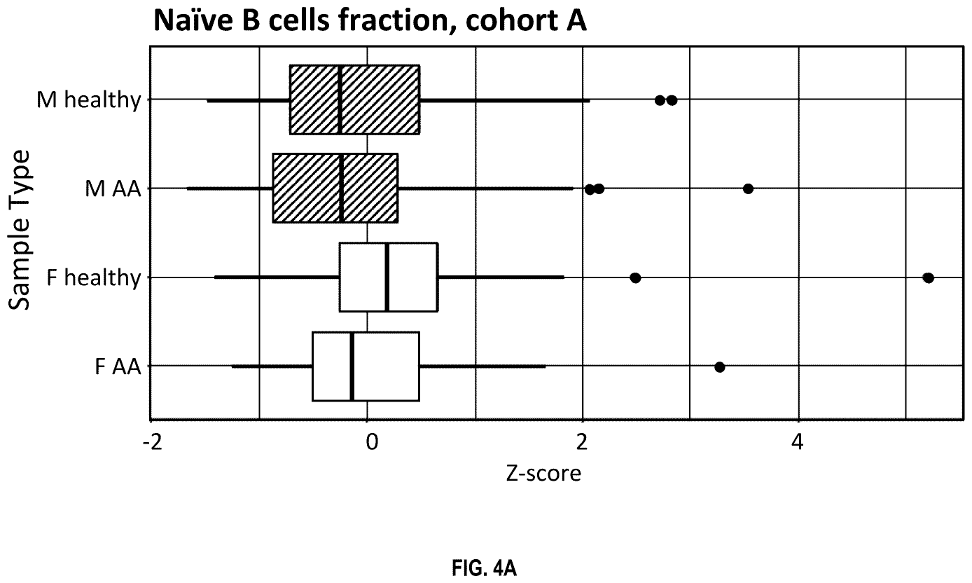

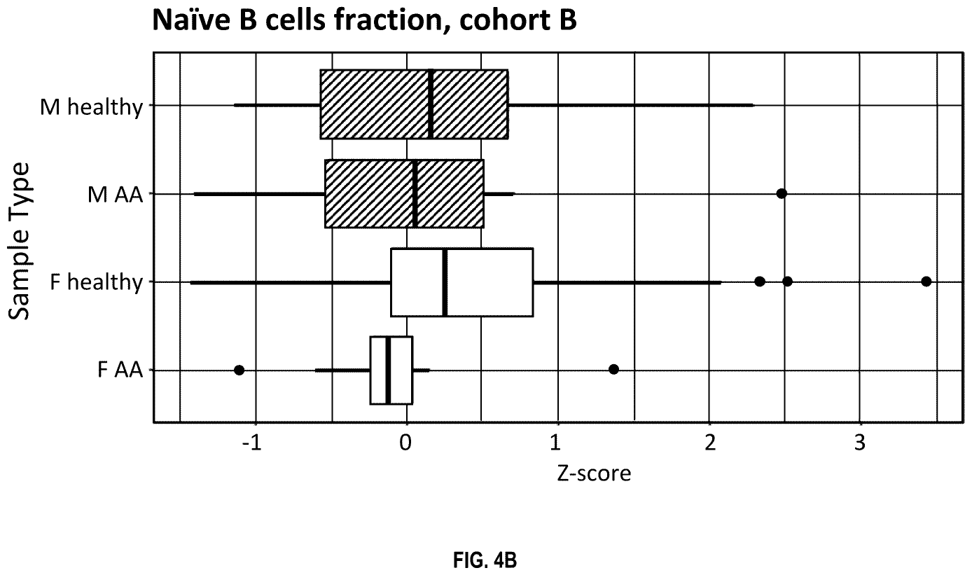

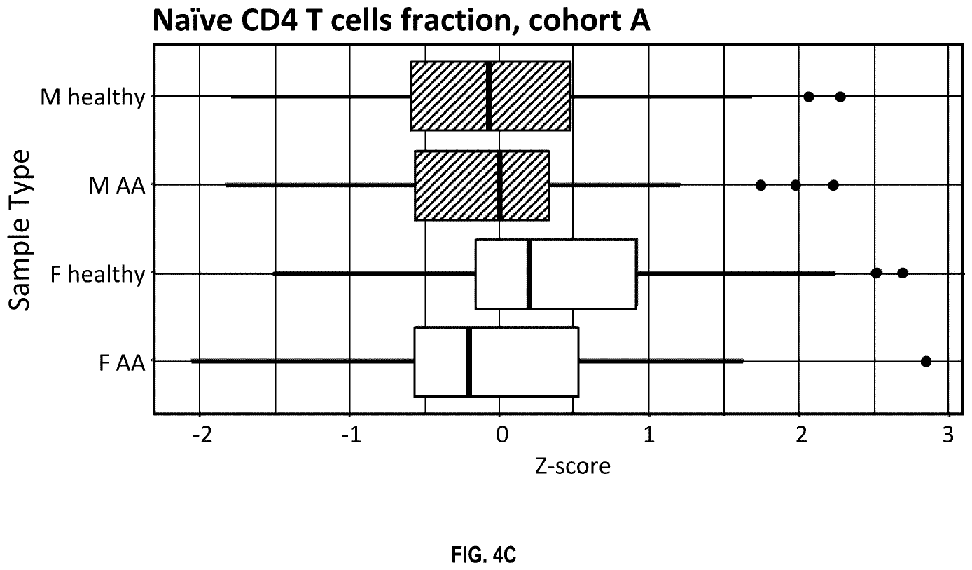

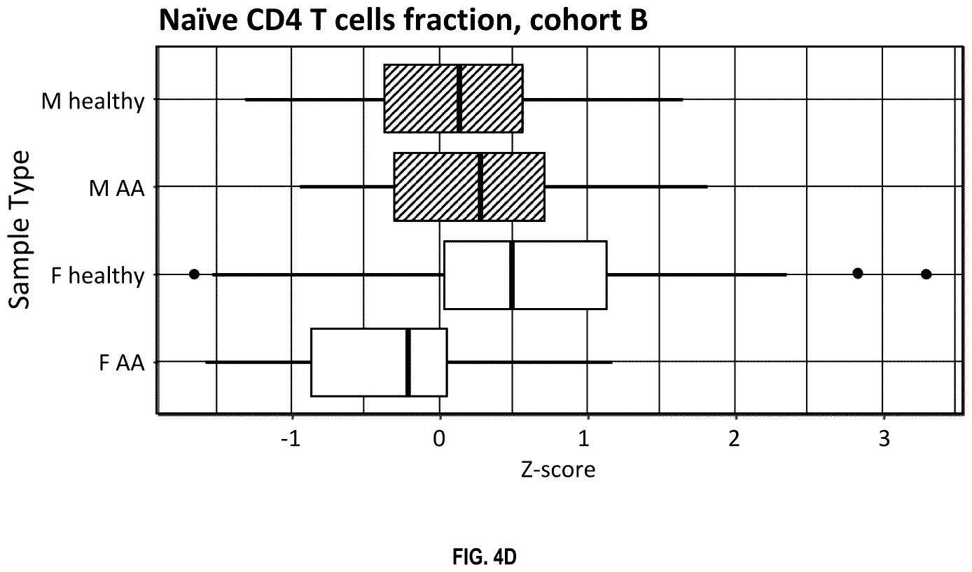

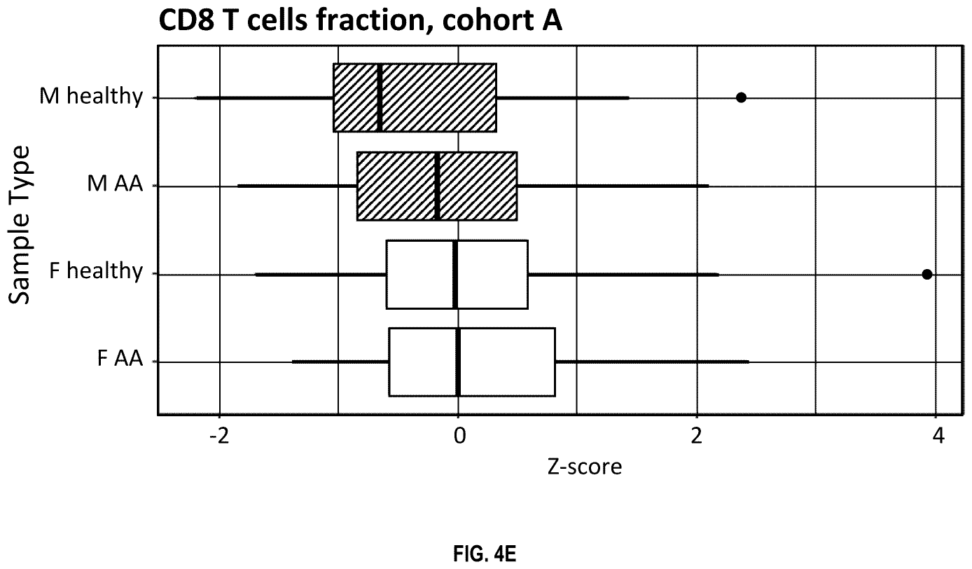

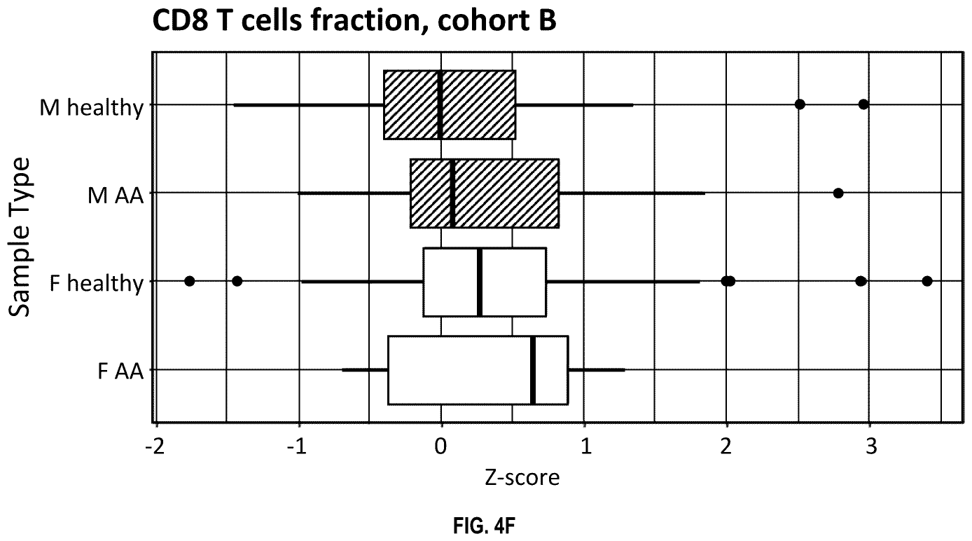

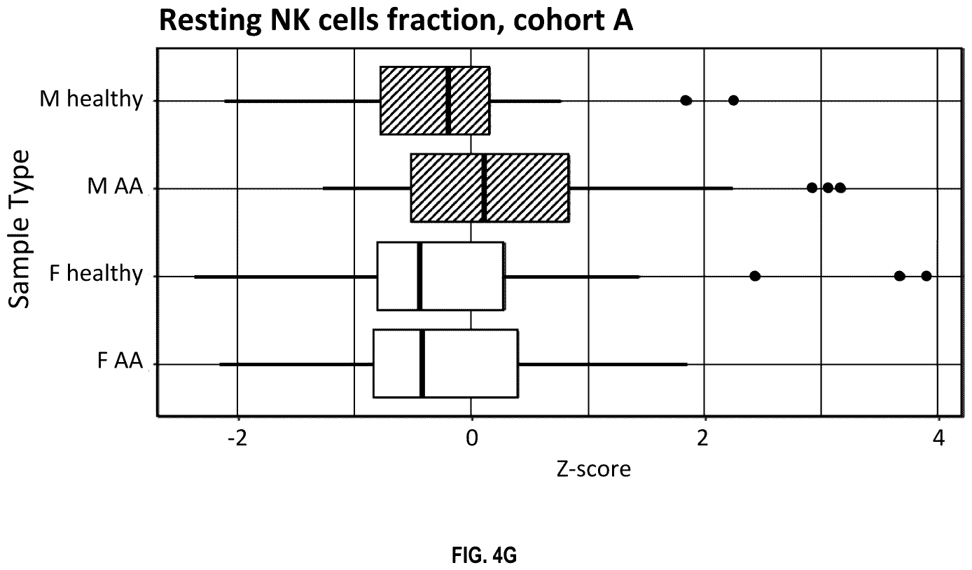

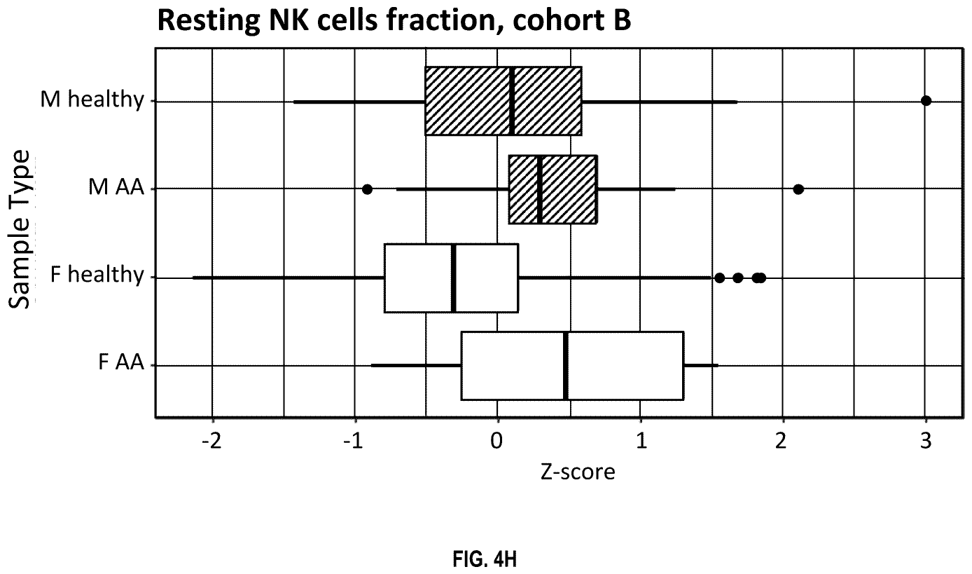

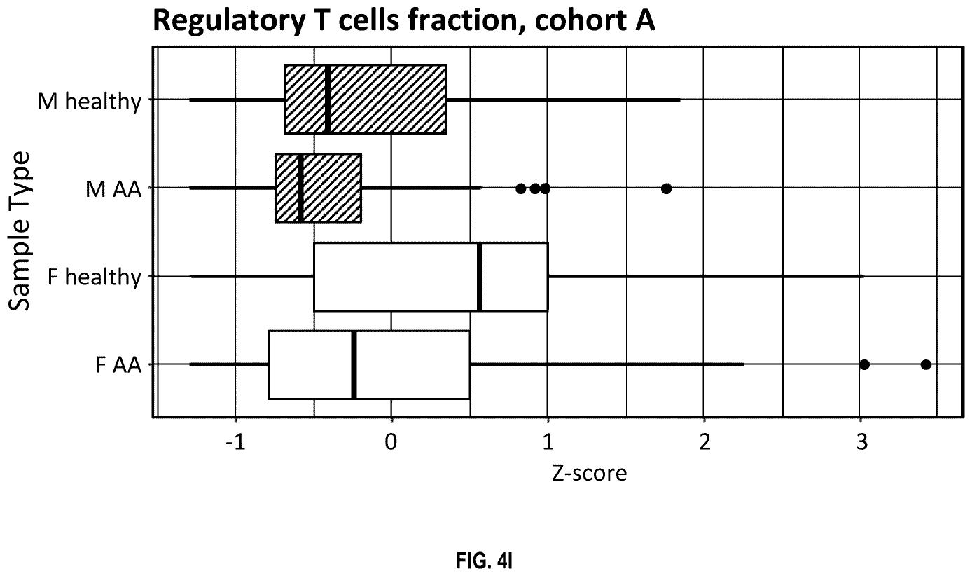

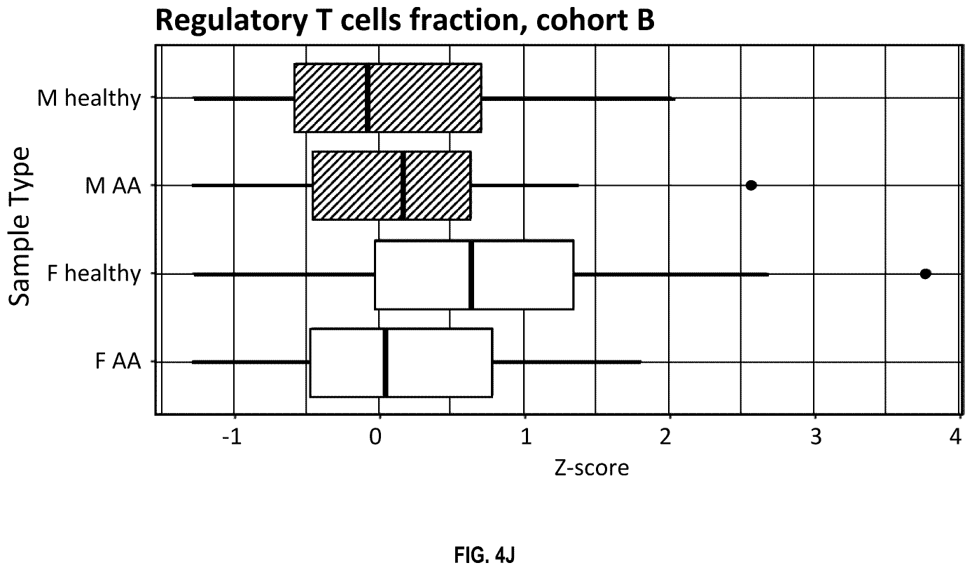

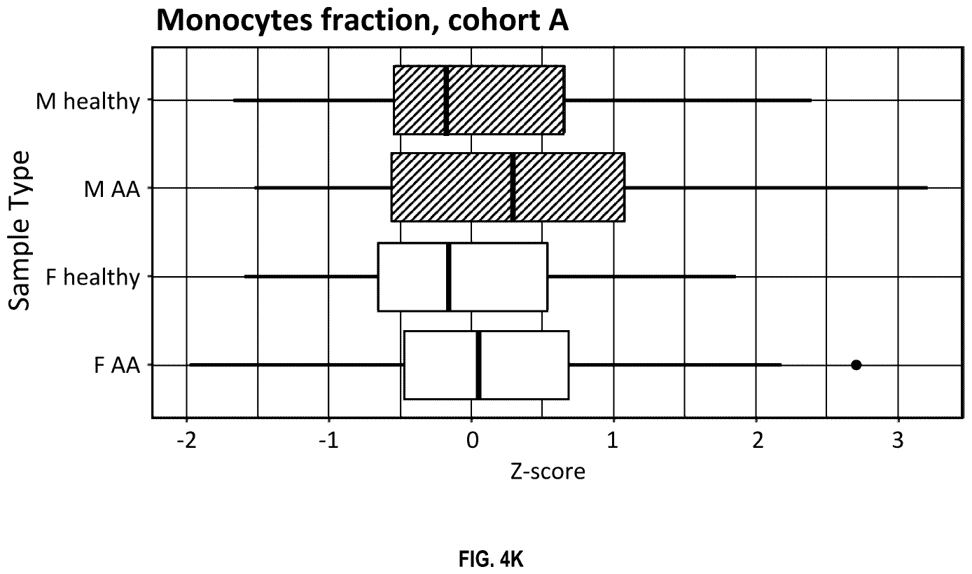

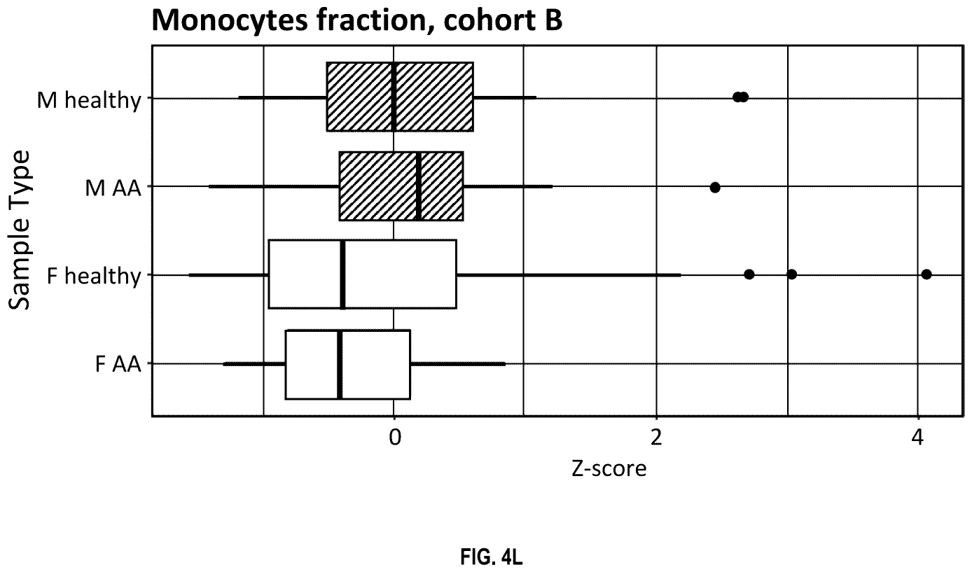

The model learned significant weights for several cell types, including CD8 T cells, resting NK cells, naïve B cells, naïve CD4 T cells, monocytes, and regulatory T cells. Despite observed differences in baseline cell proportions between healthy males and healthy females, these cell types generally showed consistent within-sex differences between AA and healthy samples, in both cohorts (FIG. 4A-4L; cell proportions were mean-centered and scaled to obtain Z-scores for plotting). For CD8 T cells, an increase was observed in cohort B in both male and female AA samples relative to male and female healthy samples, respectively, while in cohort A an increase was observed in male AA samples relative to male healthy samples, and no change observed between female AA and female healthy samples. Similarly, for resting NK cells, an increase was observed in cohort B, in both male and female AA samples relative to male and female healthy samples, respectively, while in cohort A an increase was observed in male AA samples relative to male healthy samples, and no change observed between female AA and female healthy samples. For naïve B cells, in both cohorts A and B, a decrease was observed in female AA samples relative to female healthy samples, while for males, no substantial difference was observed between AA and healthy samples in either cohort. For naïve CD4 T cells, in both cohorts A and B, a decrease was likewise observed in female AA samples relative to female healthy samples, while for males, no substantial difference was observed between AA and healthy samples in either cohort. For monocytes, an increase was observed in cohort A in both male and female AA samples relative to male and female healthy samples, respectively, while in cohort B a slight increase was observed in male AA samples relative to male healthy samples, and no change observed between female AA and female healthy samples. Finally, for Tregs, a decrease was observed in cohort A in both male and female AA samples relative to male and female healthy samples, respectively, while in cohort B a decrease increase was similarly observed in female AA samples relative to female healthy samples, while a slight increase was observed in male AA samples relative to male healthy samples.

1. A method of analyzing RNA in a sample from a subject, the method comprising:
a) sequencing the RNA and determining expression levels for a target gene set comprising a plurality of target genes that are differentially expressed in a plurality of immune cell types and/or in samples from subjects with a disease or disorder relative to samples from healthy subjects; and
b) determining
(1) quantities of the immune cell types from which the RNA originated based on the expression levels; and/or
(2) expression levels of the plurality of target genes; and
c) determining the presence, absence, or likelihood of the disease or disorder in the subject based on the quantities of the immune cell types and/or the expression levels of the plurality of target genes.

2. The method of claim 1, wherein the plurality of immune cell types comprises one or more, or each, of neutrophils, lymphocytes, plasma cells, monocytes, macrophages, dendritic cells, mast cells, eosinophils, T cells, CD4+ T cells, B cells, NK cells, megakaryocytes, CD8+ central memory cells, CD8+ effector memory cells, CD4+ central memory cells, CD4+ effector memory cells, immature neutrophils, precursor B cells, plasma cells, memory-switched B cells, plasma cells, basophils, naïve B cells, memory B cells, CD8+ T cells, naïve CD4+ T cells, resting CD4+ memory T cells, activated CD4+ memory T cells, follicular helper T cells; regulatory T cells (Tregs), gamma delta T cells, resting NK cells, activated NK cells, M0 macrophages, M1 macrophages, M2 macrophages, resting dendritic cells, activated dendritic cells, resting mast cells, and activated mast cells, optionally wherein the plurality of immune cell types comprises one or more, two or more, or three or more of CD8+ T cells, resting CD4+ memory T cells, Tregs, and naïve B cells.

3. (canceled)

4. The method of claim 1, wherein the plurality of immune cell types comprises:
a) CD8+ T cells, resting CD4+ memory T cells, Tregs, and naïve B cells;
b) CD8+ T cells, resting CD4+ memory T cells, and T regs;
c) CD8+ T cells, Tregs, and naïve B cells;
d) CD8+ T cells, resting CD4+ memory T cells, and naïve B cells;
e) resting CD4+ memory T cells, Tregs, and naïve B cells;
f) CD8+ T cells;
g) resting CD4+ memory T cells;
h) Tregs;
i) naïve B cells; or
j) neutrophils.

5. (canceled)

6. (canceled)

7. (canceled)

8. (canceled)

9. (canceled)

10. (canceled)

11. (canceled)

12. (canceled)

13. (canceled)

14. The method of claim 1, wherein the plurality of immune cell types comprises:
a) T cells, B cells, and NK cells;
b) neutrophils and lymphocytes;
c) neutrophils, T cells, B cells, and NK cells;
d) granulocytes and lymphocytes; or
e) granulocytes, T cells, B cells, and NK cells.

15. The method of claim 1, wherein determining the presence, absence, or likelihood of the disease or disorder in the subject based on the quantities of the immune cell types comprises determining:
a) the levels of one or more, or each, of the plurality of immune cell types relative to total blood cells;
b) the levels of one or more, or each, of the plurality of immune cell types relative to total white blood cells;
c) the levels of one or more, or each, of T, B, and NK cells relative to all lymphocytes;
d) relative proportions of neutrophils and lymphocytes; or
e) relative proportions of neutrophils and one or more, or each, of T cells, B cells, and NK cells.

16. (canceled)

17. (canceled)

18. (canceled)

19. (canceled)

20. The method of claim 1, wherein the plurality of target genes comprises:
a) genes differentially expressed in an activated cell type relative to the same cell type that is not activated;
b) genes differentially expressed in at least (i) a first cell type that is activated relative to the same first cell type that is not activated, and (ii) a second cell type that is activated relative to the same second cell type that is not activated;
c) genes differentially expressed in neutrophils relative to a non-neutrophil cell type; or
d) genes differentially expressed in lymphocytes relative to a non-lymphocyte cell type.

21. (canceled)

22. The method of claim 20, wherein a) the activated cell type is neutrophils, lymphocytes, plasma cells, monocytes, macrophages, dendritic cells, mast cells, or eosinophils, optionally wherein the non-neutrophil cell type is one or more, or each, of a non-immune cell type, a non-granulocyte cell type, a myeloid non-granulocyte cell type, a lymphoid cell type, lymphocytes, T cells, B cells, and NK cells, and/or b) the genes having above-average expression variance comprise genes having an expression variance in the top 25^{th} top 20^{th}, top 15^{th}, top 10^{th}, top 9^{th}, top 8^{th}, top 7^{th}, top 6^{th}, top 5^{th}, top 4^{th}, top 3^{rd}, top 2^{nd}, or top 1^{st} percentile of the genes of the training set, optionally wherein the genes having above-average expression variance are genes with an expression variance ranking in the top 1000, top 750, top 500, top 250, top 200, top 150, top 100, top 90, top 80, top 70, top 60, top 50, top 40, top 30, top 25, top 20, top 15, top 10, or top 5 genes in the training set.

23. (canceled)

24. (canceled)

25. (canceled)

26. The method of claim 1, wherein the plurality of target genes comprises genes differentially expressed in a first cell type relative to a second cell type different from the first cell type, and the first or second cell type is neutrophils, lymphocytes, plasma cells, monocytes, macrophages, dendritic cells, mast cells, eosinophils, T cells, CD4+ T cells, B cells, NK cells, megakaryocytes, CD8+ central memory cells, CD8+ effector memory cells, CD4+ central memory cells, CD4+ effector memory cells, immature neutrophils, precursor B cells, plasma cells, memory-switched B cells, plasma cells, basophils, naïve B cells, memory B cells, CD8+ T cells, naïve CD4+ T cells, resting CD4+ memory T cells, activated CD4+ memory T cells; follicular helper T cells; regulatory T cells (Tregs); gamma delta T cells; resting NK cells; activated NK cells, M0 macrophages, M1 macrophages, M2 macrophages, resting dendritic cells, activated dendritic cells, resting mast cells, or activated mast cells.

27. (canceled)

28. The method of claim 1, wherein the plurality of target genes comprises:
a) genes differentially expressed in a first cell type relative to a second cell type different from the first cell type, and the first or second cell type is B cells, T cells, or NK cells;
b) genes differentially expressed when the disease or disorder is present relative to when the disease or disorder is not present;
c) genes differentially expressed in the disease or disorder cells relative to healthy cells of the same cell type as the disease or disorder cells;
d) genes differentially expressed in the disease or disorder cells relative to healthy colon epithelial cells;
e) genes differentially expressed in the disease or disorder cells relative to a myeloid cell type or an erythroid cell type;
f) genes differentially expressed in colon epithelial cells relative to a myeloid cell type or an erythroid cell type; or
g) genes having above-average expression variance in a training set comprising gene expression data from samples from healthy subjects and from subjects with the disease or disorder.

29. (canceled)

30. (canceled)

31. (canceled)

32. (canceled)

33. (canceled)

34. (canceled)

35. (canceled)

36. (canceled)

37. The method of claim 1, wherein a) one or more, a majority, at least 75%, at least 80%, at least 85%, at least 90%, at least 95%, at least 96%, at least 97%, at least 98%, at least 99%, or all, of the target genes are protein-coding genes and/or b) the expression levels of the plurality of target genes are: (i) transcripts per million (TPM)-normalized, reads per kilobase million (RPKM)-normalized, or fragments per kilobase million (FPKM)-normalized; or (ii) mean-centered and/or scaled to unit variance.

38. (canceled)

39. (canceled)

40. (canceled)

41. The method of claim 1, wherein determining the presence, absence, or likelihood of the disease or disorder comprises:
a) compensating for effects of sex on gene expression;
b) compensating for effects of sex on cell type quantity or cell type proportion;
c) compensating for effects of age on gene expression; or
d) compensating for effects of age on cell type abundance or cell type proportion.

42. The method of claim 41, wherein
a) compensating for effects of sex on gene expression comprises regressing out the effects of sex on gene expression;
b) compensating for effects of sex on cell type quantity or cell type proportion comprises regressing out the effects of sex on cell type quantity or cell type proportion;
c) compensating for effects of age on gene expression comprises regressing out the effects of age on gene expression; or
d) compensating for effects of age on cell type abundance or cell type proportion comprises regressing out the effects of age on cell type abundance or cell type proportion.

43. (canceled)

44. (canceled)

45. The method of claim 1, wherein the target genes comprise genes that are not differentially expressed according to sex or age.

46. The method of claim 45, wherein
a) at least 50%, at least 60%, at least 70%, at least 75%, at least 80%, at least 85%, at least 90%, at least 95%, at least 96%, at least 97%, at least 98%, at least 99%, or 100% of the target genes are not differentially expressed according to sex or age.

47. The method of claim 1, wherein the target genes were identified using a training set comprising:
a) samples from individuals of the same sex as the subject; or
b) samples from individuals that, at the time of sample collection from each individual, were of an age that is within 1 year, within 2 years, within 3 years, within 4 years, within 5 years, within 6 years, within 7 years, within 8 years, within 9 years, within 10 years, within 11 years, within 12 years, within 13 years, within 14 years, or within 15 years of the age of the subject at the time of sample collection from the subject.

48. (canceled)

49. (canceled)

50. (canceled)

51. (canceled)

52. (canceled)

53. (canceled)

54. (canceled)

55. (canceled)

56. (canceled)

57. The method of claim 1, wherein the target genes comprise:
a) one or more, or each, of GZMH, PATL2, FCRL6, ZNF600, IL10RA, DTHD1, PYHIN1, HDAC11, XCL1, GZMA, RAB37, ID2, SLC9A3R1, MOSPD3, TES, EML4, CD99, ACSL6, YPEL1, H2AC17, FGFBP2, TBX21, F2R, PTGDS, DDX3Y, SYNGR1, ZNF683, SERPIN86, PRSS23, ID2, SRSF2, PRR5, CST3, SHISA4, PLAC8, BRD2, BMF, NIPAL2, UTS2, RARS2, ZNF320, RBL2, CREB3L2, RNF38, CCDC88A, MEX3C, SLC38A11, COL19A1, PNPLA7, AFF3, STEAP1B, CELSR1, GRAPL, CBX5, AKAP6, EVL, CRISP3, BPI, MMP8, OLFM4, MPO, MROH6, KDM5D, IDH2, ABI3, S100A4, H2AC17, GIMAP4, PPP1CB, XAF1, RPL37, PTPRO, CD177, RPS13, NAIP, RPS12, ID2, SH3BP1, RAB37, PLEKHG3, AFDN, SLC9A3R1, FLNA, SSBP3, RHOB, LIPA, ISCA1, PAG1, DDX3X, KDM5C, PIM2, IL4R, ZFX, FCGR2B, KDM6A, SARAF, TTC39C, NCOR2, ATP5MD, ARHGD1A, TRAF5, SCAMP2, RPS26, GSTM4, MICAL3, TOMM20, SSBP3, RALGDS, ITGB1, DYRK1B, ARRB1, TRIM11, LFNG, PAXX, ACCS, and SLC2A6, optionally wherein the target genes comprise one or more, or each, of GZMH, PATL2, FCRL6, ZNF600, IL10RA, DTHD1, PYHIN1, HDAC11, XCL1, GZMA, RAB37, ID2, SLC9A3R1, MOSPD3, TES, EML4, CD99, ACSL6, YPEL1, H2AC17, FGFBP2, TBX21, F2R, PTGDS, DDX3Y, SYNGR1, ZNF683, SERPIN86, PRSS23, ID2, SRSF2, PRR5, CST3, SHISA4, PLAC8, BRD2, BMF, NIPAL2, UTS2, RARS2, ZNF320, RBL2, CREB3L2, RNF38, CCDC88A, MEX3C, SLC38A11, COL19A1, PNPLA7, AFF3, STEAP1B, CELSR1, GRAPL, CBX5, AKAP6, EVL, CRISP3, BPI, MMP8, OLFM4, MPO, MROH6, KDM5D, IDH2, ABI3, S100A4, H2AC17, GIMAP4, PPP1CB, XAF1, RPL37, PTPRO, CD177, RPS13, NAIP, RPS12, ID2, SH3BP1, RAB37, PLEKHG3, AFDN, SLC9A3R1, FLNA, SSBP3, RHOB, LIPA, ISCA1, PAG1, DDX3X, KDM5C, PIM2, IL4R, ZFX, FCGR2B, KDM6A, SARAF, TTC39C, NCOR2, ATP5MD, ARHGD1A, TRAF5, SCAMP2, RPS26, GSTM4, MICAL3, TOMM20, SSBP3, RALGDS, ITGB1, DYRK1B, ARRB1, TRIM11, LFNG, PAXX, ACCS, and SLC2A6;
b) one or more, or each, of ICA1, CD38, ABCB4, TNFRSF17, RRP12, CHI3L2, MAP3K13, IL4R, IL12RB2, ACHE, LAG3, CD209, GGT5, DEPDC5, UPK3A, GZMH, BPI, ACP5, CD37, MAST1, RASSF4, MS4A6A, PPFIBP1, MAK, IL18RAP, KYNU, FASLG, MYB, CCND2, TRPM6, FLVCR2, CD80, TMEM156, BHLHE41, NFE2, TREM1, TMEM255A, IL1B, PLEKHG3, VPREB3, TEP1, TRPM4, SMPDL3B, LILRB2, CHI3L1, IL2RA, TLR2, BMP2K, TNFRSF11A, PLA2G7, EPHA1, ABCB9, ZFP36L2, HHEX, SKA1, CLIC2, DUSP2, SLAMF8, CDA, ZNF222, ASGR2, PBXIP1, NIPSNAP3B, TSHR, NOD2, CD300A, NAALADL1, ADRB2, SCN9A, CEACAM3, COL8A2, CLEC7A, CTSW, HPSE, CD7, P2RY14, HIC1, P2RY13, GPR19, CCR3, MROH7, CSF1, FAM174B, TNFRSF4, PDCD1, RYR1, HLA-DQA1, NCR3, ZBTB10, APOBEC3G, LILRA2, TNFRSF13B, HLA-DOB, PGLYRP1, HGF, ATP9A, ATP2C2, JMJD6, DHRS9, SLC1A3, CEACAM1, DUSP13, CRISP3, ABLIM1, HSD3B7, OSM, UPB1, BIK, MMP9, SLCO4A1, BMX, KLF5, RETN, GRB10, PRUNE2, ERLIN1, TP5313, ILIR2, EPAS1, LRRC42, GADD45A, PHTF1, RCAN3, ARG1, CYSTM1, DACH1, FKBP9, GOS2, PFKFB2, CDH26, ARMC7, PPP1R3D, ECHDC3, RDH5, ACVRIB, CKAP4, MTHFS, IL10, MFSD13A, GPR84, MYLK3, ZNF787, MY010, RAB19, OLAH, ANKRD22, RABGEF1, SAMSN1, CACNA1D, RGL4, TFF3, GYG1, ZDHHC19, SLC51A, POC1A, HPGD, FBN1, CLEC4D, SEMA6B, PFKFB3, PAQR8, SH3PXD2B, FOSL1, MSRA, LRRN1, KCNE5, B3GNT5, CD163, CRACR2B, OPLAH, EXOSC4, KCNE1, UPP1, ST6GALNAC3, SEPTIN5, FCAR, SPATC1, MAFG, IL27, ENTPD7, PLIN5, FAM228B, ORM1, STON1, CFH, HS3ST1, DBNDD1, CD22, SLC25A39, KCNG1, TGFBR3, ADD2, COL19A1, CD200, TCL1A, PROCR, CD40, NME4, TSPAN13, RGS9, FAM184A, KHDRBS2, ENPP5, MMP8, SATB2, GPR68, CEACAM8, MYO1B, LARGE1, NT5E, RAPGEF5, ABHD17C, ZNF365, GRTP1, IGFBP3, LCN2, GLB1L2, CNKSR2, PRSS23, RASGRP3, SCN3A, C16orf74, RETREG1, ERG, SNX22, CXCR5, BEND5, SLC1A7, LEXM, CAMK2N1, SPRY1, CDCA7L, SPIB, DLC1, DIPK1B, MTCL1, PARM1, MZB1, SLC23A1, PDGFD, NMUR1, NBEA, OLR1, PTPRM, PAWR, ABCA13, TAFA1, KCNH8, EFNA5, PRSS57, PTCH1, PRIN3, SULT1A1, ZNF667, SHISA4, L1CAM, RASGEFIA, DDR1, GPC2, PATL2, and IGLL5, optionally wherein the target genes comprise 2, 3, 4, 5, 6, 7, 8, 9, 10, 11, 12, 13, 14, 15, 16, 17 18, 19, or 20 of ICA1, CD38, ABCB4, TNFRSF17, RRP12, CHI3L2, MAP3K13, IL4R, IL12RB2, ACHE, LAG3, CD209, GGT5, DEPDC5, UPK3A, GZMH, BPI, ACP5, CD37, MAST1, RASSF4, MS4A6A, PPFIBP1, MAK, IL18RAP, KYNU, FASLG, MYB, CCND2, TRPM6, FLVCR2, CD80, TMEM156, BHLHE41, NFE2, TREM1, TMEM255A, IL1B, PLEKHG3, VPREB3, TEP1, TRPM4, SMPDL3B, LILRB2, CHI3L1, IL2RA, TLR2, BMP2K, TNFRSF11A, PLA2G7, EPHA1, ABCB9, ZFP36L2, HHEX, SKA1, CLIC2, DUSP2, SLAMF8, CDA, ZNF222, ASGR2, PBXIP1, NIPSNAP3B, TSHR, NOD2, CD300A, NAALADL1, ADRB2, SCN9A, CEACAM3, COL8A2, CLEC7A, CTSW, HPSE, CD7, P2RY14, HIC1, P2RY13, GPR19, CCR3, MROH7, CSF1, FAM174B, TNFRSF4, PDCD1, RYR1, HLA-DQA1, NCR3, ZBTB10, APOBEC3G, LILRA2, TNFRSF13B, HLA-DOB, PGLYRP1, HGF, ATP9A, ATP2C2, JMJD6, DHRS9, SLC1A3, CEACAM1, DUSP13, CRISP3, ABLIM1, HSD3B7, OSM, UPB1, BIK, MMP9, SLCO4A1, BMX, KLF5, RETN, GRB10, PRUNE2, ERLIN1, TP5313, IL1R2, EPAS1, LRRC42, GADD45A, PHTF1, RCAN3, ARG1, CYSTM1, DACH1, FKBP9, GOS2, PFKFB2, CDH26, ARMC7, PPPIR3D, ECHDC3, RDH5, ACVR1B, CKAP4, MTHFS, IL10, MFSD13A, GPR84, MYLK3, ZNF787, MYO10, RAB19, OLAH, ANKRD22, RABGEF1, SAMSN1, CACNA1D, RGL4, TFF3, GYG1, ZDHHC19, SLC51A, POC1A, HPGD, FBN1, CLEC4D, SEMA6B, PFKFB3, PAQR8, SH3PXD2B, FOSL1, MSRA, LRRN1, KCNE5, B3GNT5, CD163, CRACR2B, OPLAH, EXOSC4, KCNE1, UPP1, ST6GALNAC3, SEPTIN5, FCAR, SPATC1, MAFG, IL27, ENTPD7, PLIN5, FAM228B, ORM1, STON1, CFH, HS3ST1, DBNDD1, CD22, SLC25A39, KCNG1, TGFBR3, ADD2, COL19A1, CD200, TCLIA, PROCR, CD40, NME4, TSPAN13, RGS9, FAM184A, KHDRBS2, ENPP5, MMP8, SATB2, GPR68, CEACAM8, MYO1B, LARGE1, NT5E, RAPGEF5, ABHD17C, ZNF365, GRTP1, IGFBP3, LCN2, GLBIL2, CNKSR2, PRSS23, RASGRP3, SCN3A, C16orf74, RETREG1, ERG, SNX22, CXCR5, BEND5, SLC1A7, LEXM, CAMK2N1, SPRY1, CDCA7L, SPIB, DLC1, DIPK1B, MTCL1, PARM1, MZB1, SLC23A1, PDGFD, NMUR1, NBEA, OLR1, PTPRM, PAWR, ABCA13, TAFA1, KCNH8, EFNA5, PRSS57, PTCH1, PRTN3, SULT1A1, ZNF667, SHISA4, L1CAM, RASGEFIA, DDR1, GPC2, PATL2, and IGLL5;
c) one or more, or each, of PGLYRP1, HGF, ATP9A, ATP2C2, JMJD6, DHRS9, SLC1A3, CEACAM1, DUSP13, CRISP3, ABLIM1, HSD3B7, OSM, UPB1, BIK, MMP9, SLCO4A1, BMX, KLF5, RETN, GRB10, PRUNE2, ERLIN1, TP5313, ILIR2, EPAS1, LRRC42, GADD45A, PHTF1, RCAN3, ARG1, CYSTM1, DACH1, FKBP9, GOS2, PFKFB2, CDH26, ARMC7, PPPIR3D, ECHDC3, RDH5, ACVR1B, CKAP4, MTHFS, IL10, MFSD13A, GPR84, MYLK3, ZNF787, MYO10, RAB19, OLAH, ANKRD22, RABGEF1, SAMSN1, CACNA1D, RGL4, TFF3, GYG1, ZDHHC19, SLC51A, POC1A, HPGD, FBN1, CLEC4D, SEMA6B, PFKFB3, PAQR8, SH3PXD2B, FOSL1, MSRA, LRRN1, KCNE5, B3GNT5, CD163, CRACR2B, OPLAH, EXOSC4, KCNE1, UPP1, ST6GALNAC3, SEPTIN5, FCAR, SPATC1, MAFG, IL27, ENTPD7, PLIN5, FAM228B, ORM1, and STON1, optionally wherein the target genes comprise 2, 3, 4, 5, 6, 7, 8, 9, 10, 11, 12, 13, 14, 15, 16, 17, 18, 19, or 20 of PGLYRP1, HGF, ATP9A, ATP2C2, JMJD6, DHRS9, SLC1A3, CEACAM1, DUSP13, CRISP3, ABLIM1, HSD3B7, OSM, UPB1, BIK, MMP9, SLCO4A1, BMX, KLF5, RETN, GRB10, PRUNE2, ERLIN1, TP5313, ILIR2, EPAS1, LRRC42, GADD45A, PHTF1, RCAN3, ARG1, CYSTM1, DACH1, FKBP9, GOS2, PFKFB2, CDH26, ARMC7, PPPIR3D, ECHDC3, RDH5, ACVRIB, CKAP4, MTHFS, IL10, MFSD13A, GPR84, MYLK3, ZNF787, MY010, RAB19, OLAH, ANKRD22, RABGEF1, SAMSN1, CACNA1D, RGL4, TFF3, GYG1, ZDHHC19, SLC51A, POCIA, HPGD, FBN1, CLEC4D, SEMA6B, PFKFB3, PAQR8, SH3PXD2B, FOSL1, MSRA, LRRN1, KCNE5, B3GNT5, CD163, CRACR2B, OPLAH, EXOSC4, KCNE1, UPP1, ST6GALNAC3, SEPTIN5, FCAR, SPATC1, MAFG, IL27, ENTPD7, PLIN5, FAM228B, ORM1, and STON1;
d) one or more, or each, of ICA1, CD38, ABCB4, TNFRSF17, RRP12, CHI3L2, MAP3K13, IL4R, IL12RB2, ACHE, LAG3, CD209, GGT5, DEPDC5, UPK3A, GZMH, BPI, ACP5, CD37, MAST1, RASSF4, MS4A6A, PPFIBP1, MAK, IL18RAP, KYNU, FASLG, MYB, CCND2, TRPM6, FLVCR2, CD80, TMEM156, BHLHE41, NFE2, TREM1, TMEM255A, IL1B, PLEKHG3, VPREB3, TEP1, TRPM4, SMPDL3B, LILRB2, CHI3L1, IL2RA, TLR2, BMP2K, TNFRSF11A, PLA2G7, EPHA1, ABCB9, ZFP36L2, HHEX, SKA1, CLIC2, DUSP2, SLAMF8, CDA, ZNF222, ASGR2, PBXIP1, NIPSNAP3B, TSHR, NOD2, CD300A, NAALADL1, ADRB2, SCN9A, CEACAM3, COL8A2, CLEC7A, CTSW, HPSE, CD7, P2RY14, HIC1, P2RY13, GPR19, CCR3, MROH7, CSF1, FAM174B, TNFRSF4, PDCD1, RYR1, HLA-DQA1, NCR3, ZBTB10, APOBEC3G, LILRA2, TNFRSF13B, and HLA-DOB, optionally wherein the target genes comprise 2, 3, 4, 5, 6, 7, 8, 9, 10, 11, 12, 13, 14, 15, 16, 17, 18, 19, or 20 of ICA1, CD38, ABCB4, TNFRSF17, RRP12, CHI3L2, MAP3K13, IL4R, IL12RB2, ACHE, LAG3, CD209, GGT5, DEPDC5, UPK3A, GZMH, BPI, ACP5, CD37, MAST1, RASSF4, MS4A6A, PPFIBP1, MAK, IL18RAP, KYNU, FASLG, MYB, CCND2, TRPM6, FLVCR2, CD80, TMEM156, BHLHE41, NFE2, TREM1, TMEM255A, IL1B, PLEKHG3, VPREB3, TEP1, TRPM4, SMPDL3B, LILRB2, CHI3L1, IL2RA, TLR2, BMP2K, TNFRSF11A, PLA2G7, EPHA1, ABCB9, ZFP36L2, HHEX, SKA1, CLIC2, DUSP2, SLAMF8, CDA, ZNF222, ASGR2, PBXIP1, NIPSNAP3B, TSHR, NOD2, CD300A, NAALADL1, ADRB2, SCN9A, CEACAM3, COL8A2, CLEC7A, CTSW, HPSE, CD7, P2RY14, HIC1, P2RY13, GPR19, CCR3, MROH7, CSF1, FAM174B, TNFRSF4, PDCD1, RYR1, HLA-DQA1, NCR3, ZBTB10, APOBEC3G, LILRA2, TNFRSF13B, and HLA-DOB; or
e) one or more, or each, of CFH, HS3ST1, DBNDD1, CD22, SLC25A39, KCNG1, TGFBR3, ADD2, COL19A1, CD200, TCL1A, PROCR, CD40, NME4, TSPAN13, RGS9, FAM184A, KHDRBS2, ENPP5, MMP8, SATB2, GPR68, CEACAM8, MYO1B, LARGE1, NT5E, RAPGEF5, ABHD17C, ZNF365, GRTP1, IGFBP3, LCN2, GLB1L2, CNKSR2, PRSS23, RASGRP3, SCN3A, C16orf74, RETREG1, ERG, SNX22, CXCR5, BEND5, SLCIA7, LEXM, CAMK2N1, SPRY1, CDCA7L, SPIB, DLC1, DIPK1B, MTCL1, PARM1, MZB1, SLC23A1, PDGFD, NMUR1, NBEA, OLR1, PTPRM, PAWR, ABCA13, TAFA1, KCNH8, EFNA5, PRSS57, PTCH1, PRTN3, SULT1A1, ZNF667, SHISA4, L1CAM, RASGEFIA, DDR1, GPC2, PATL2, and IGLL5, optionally wherein the target genes comprise 2, 3, 4, 5, 6, 7, 8, 9, 10, 11, 12, 13, 14, 15, 16, 17, 18, 19, or 20 of CFH, HS3ST1, DBNDD1, CD22, SLC25A39, KCNG1, TGFBR3, ADD2, COL19A1, CD200, TCL1A, PROCR, CD40, NME4, TSPAN13, RGS9, FAM184A, KHDRBS2, ENPP5, MMP8, SATB2, GPR68, CEACAM8, MYO1B, LARGE1, NT5E, RAPGEF5, ABHD17C, ZNF365, GRTP1, IGFBP3, LCN2, GLBIL2, CNKSR2, PRSS23, RASGRP3, SCN3A, C16orf74, RETREG1, ERG, SNX22, CXCR5, BEND5, SLC1A7, LEXM, CAMK2N1, SPRY1, CDCA7L, SPIB, DLC1, DIPK1B, MTCL1, PARM1, MZB1, SLC23A1, PDGFD, NMUR1, NBEA, OLR1, PTPRM, PAWR, ABCA13, TAFA1, KCNH8, EFNA5, PRSS57, PTCH1, PRTN3, SULT1A1, ZNF667, SHISA4, L1CAM, RASGEFIA, DDR1, GPC2, PATL2, and IGLL5.

58. (canceled)

59. (canceled)

60. (canceled)

61. (canceled)

62. (canceled)

63. (canceled)

64. (canceled)

65. (canceled)

66. The method of claim 1, wherein the method comprises preparing cDNA from the RNA and ligating adapters to the cDNA, thereby producing adapter-ligated cDNA, optionally wherein the adapter-ligated cDNA is amplified prior to the sequencing and/or the adapters comprise barcodes.

67. (canceled)

68. (canceled)

69. The method of claim 1, further comprising enriching for at least one target region set from the RNA, cDNA prepared from the RNA, or a subsample thereof, comprising contacting the RNA, cDNA prepared from the RNA, or a subsample thereof with target-specific probes specific for the at least one target region set.

70. The method of claim 1, further comprising a step of ribosomal RNA depletion, globin mRNA depletion, selection of polyadenylated RNA (poly (A)) transcripts, or RNA fragmentation prior to the sequencing, optionally wherein the fragmenting provides RNA fragments of 25-400, 25-300, 25-200, 50-400, 50-300, 50-250, 50-200, 100-400, 100-300, 100-200, 125-400, 125-300, 125-200, 125-175, 150-400, 150-300, 200-400, 250-400, 300-400, 200-350, 200-300, 225-375, 250-350, or 275-325 base pairs in length.

71. (canceled)

72. (canceled)

73. (canceled)

74. (canceled)

75. The method of claim 1, wherein a) the sample is a whole blood sample; b) the RNA comprises RNA isolated from intact cells originally present in the sample; and/or c) the RNA comprises one or more of mRNA, lncRNA, or miRNA.

76. (canceled)

77. The method of claim 1, wherein the determining quantities of each of the plurality of immune cell types or sequencing comprises generating a plurality of sequencing reads, and wherein the method further comprises mapping the plurality of sequence reads to one or more reference sequences to generate mapped sequence reads, and processing the mapped sequence reads to determine the presence or absence of the disease or disorder in the subject, or the likelihood that the subject has the disease or disorder, optionally wherein the disease or disorder is a cancer or precancer comprising advanced adenoma (AA) and/or colorectal cancer (CRC),
further optionally wherein the method comprises:
a) determining the presence or absence of AA;
b) determining the likelihood of AA;
c) determining the presence or absence of CRC;
d) determining the likelihood of CRC;
e) determining the presence or absence of AA and CRC; or
f) determining the likelihood of AA and CRC,
further optionally wherein the sample is obtained from: (i) a subject who was previously diagnosed with the cancer and received one or more previous cancer treatments, further optionally wherein the sample is obtained at one or more preselected time points following the one or more previous cancer treatments; or (ii) a subject who was previously diagnosed with the cancer, and the sample is obtained from the subject before the subject receives a cancer treatment.

78. (canceled)

79. (canceled)

80. (canceled)

81. (canceled)

82. (canceled)

83. The method of claim 80, further comprising:
a) determining a cancer recurrence score, optionally wherein a cancer recurrence status of the subject is determined to be at risk for cancer recurrence when the cancer recurrence score is determined to be at or above a predetermined threshold or the cancer recurrence status of the subject is determined to be at lower risk for cancer recurrence when the cancer recurrence score is below the predetermined threshold;
b) comparing the cancer recurrence score of the subject with a predetermined cancer recurrence threshold, wherein the subject is classified as a candidate for a subsequent cancer treatment when the cancer recurrence score is above the cancer recurrence threshold or not a candidate for a subsequent cancer treatment when the cancer recurrence score is below the cancer recurrence threshold; and/or
c) evaluating or monitoring a response to a treatment in the subject, optionally wherein the evaluating or monitoring the response to the treatment in the subject comprises: (i) comparing the expression levels for the target gene set comprising a plurality of target genes that are differentially expressed in a sample from the subject collected at at least a first time point and a sample from the subject collected at at least a second time point; or (ii) comparing the quantities of the immune cell types in a sample from the subject collected at at least a first time point and a sample from the subject collected at at least a second time point, optionally wherein the quantities of the immune cell types are cell type abundances or cell type proportions, further optionally wherein (i) the first time point is a time point prior to administration of the treatment to the subject, and the second time point is a time point after the administration of the treatment to the subject; or (ii) the first time point is a time point after administration of the treatment to the subject, and the second time point is a time point after the administration of the treatment to the subject and after the first time point.

84. (canceled)

85. (canceled)

86. (canceled)

87. (canceled)

88. (canceled)

89. (canceled)

90. (canceled)


SEQUENCE LISTING (14 sequences)

SEQ ID NO: 1 - protein, 397 aa, synthetic construct
MNSNKDKIKVIKVFEAFAGIGSQFKALKNIARSKNWEIQHSGMVEWFVDAIVSYVAIHSKNFNPKIERLDRDILSISNDSKMPISEYGIKKINNTIKASYLNYAKKHFNNLFDIKKVNKDNFPKNIDIFTYSFPCQDLSVQGLQKGIDKELNTRSGLLWEIERILEEIKNSFSKEEMPKYLLMENVKNLLSHKNKKNYNTWLKQLEKFGYKSKTYLLNSKNFDNCQNRERVFCLSIRDDYLEKTGFKFKELEKVKNPPKKIKDILVDSSNYKYLNLNKYETTTFRETKSNIISRPLKNYTTFNSENYVYNINGIGPTLTASGANSRIKIETQQGVRYLTPLECFKYMQFDVNDFKKVQSTNLISENKMIYIAGKSIPVKILEAIFNTLEFVNNEELE

SEQ ID NO: 2 - protein, 397 aa, synthetic construct
MNSNKDKIKVIKVFEAFAGIGSQFKALKNIARSKNWEIQHSGMVEWFVDAIVSYVAIHSKNFNPKIERLDRDILSISNDSKMPISEYGIKKINNTIKASYLNYAKKHFNNLFDIKKVNKDNFPKNIDIFTYSFPCQDLSVQGLQKGIDKELNTRSGLLWEIERILEEIKNSFSKEEMPKYLLMENVKNLLSHKNKKNYNTWLKQLEKFGYKSKTYLLNSKNFDNCQNRERVFCLSIRDDYLEKTGFKFKELEKVKNPPKKIKDILVDSSNYKYLNLNKYETTTFRETKSNIISRPLKNYTTFNSENYVYNINGIGPTLTASGANSRIKIETQQGVRYLTPLECFKYMQFDVNDFKKVQSTNLISENKMIYIAGRSIPVKILEAIFNTLEFVNNEELE

SEQ ID NO: 3 - protein, 507 aa, Homo sapiens
GGSQSQNGKCEGCNPDKDEAPYYTHLGAGPDVAAIRTLMEERYGEKGKAIRIEKVIYTGKEGKSSQGCPIAKWVYRRSSEEEKLLCLVRVRPNHTCETAVMVIAIMLWDGIPKLLASELYSELTDILGKCGICTNRRCSQNETRNCCCQGENPETCGASFSFGCSWSMYYNGCKFARSKKPRKFRLHGAEPKEEERLGSHLQNLATVIAPIYKKLAPDAYNNQVEFEHQAPDCCLGLKEGRPFSGVTACLDFSAHSHRDQQNMPNGSTVVVTLNREDNREVGAKPEDEQFHVLPMYIIAPEDEFGSTEGQEKKIRMGSIEVLQSFRRRRVIRIGELPKSCEVSGQDAAAVQEIEYWSDSEHNFQDPCIGGVAIAPTHGSILIECAKCEVHATTKVNDPDRNHPTRISLVLYRHKNLFLPKHCLALWEAKMAEKARKEEECGKNGSDHVSQKNHGKQEKREPTGPQEPSYLRFIQSLAENTGSVTTDSTVTTSPYAFTQVTGPYNTFV

SEQ ID NO: 4 - protein, 871 aa, synthetic construct
QSQNGKCEGCNPDKDEAPYYTHLGAGPDVAAIRTLMEERYGEKGKAIRIEKVIYTGKEGKSSQGCPIAKWVYRRSSEEEKLLCLVRVRPNHTCETAVMVIAIMLWDGIPKLLASELYSELTDILGKCGICTNRRCSQNETRNCCCQGENPETCGASFSFGCSWSMYYNGCKFARSKKPRKFRLHGAEPKEEERLGSHLQNLATVIAPIYKKLAPDAYNNQVEFEHQAPDCCLGLKEGRPFSGVTACLDFSAHSHRDQQNMPNGSTVVVTLNREDNREVGAKPEDEQFHVLPMYIIAPEDEFGSTEGQEKKIRMGSIEVLQSFRRRRVIRIGELPKSCKKKAEPKKAKTKKAARKRSSLENCSSRTEKGKSSSHTKLMENASHMKQMTAQPQLSGPVIRQPPTLQRHLQQGQRPQQPQPPQPQPQTTPQPQPQPQHIMPGNSQSVGSHCSGSTSVYTRQPTPHSPYPSSAHTSDIYGDTNHVNFYPTSSHASGSYLNPSNYMNPYLGLLNQNNQYAPFPYNGSVPVDNGSPFLGSYSPQAQSRDLHRYPNQDHLTNQNLPPIHTLHQQTFGDSPSKYLSYGNQNMQRDAFTTNSTLKPNVHHLATFSPYPTPKMDSHFMGAASRSPYSHPHTDYKTSEHHLPSHTIYSYTAAASGSSSSHAFHNKENDNIANGLSRVLPGFNHDRTASAQELLYSLTGSSQEKQPEVSGQDAAAVQEIEYWSDSEHNFQDPCIGGVAIAPTHGSILIECAKCEVHATTKVNDPDRNHPTRISLVLYRHKNLFLPKHCLALWEAKMAEKARKEEECGKNGSDHVSQKNHGKQEKREPTGPQEPSYLRFIQSLAENTGSVTTDSTVTTSPYAFTQVTGPYNTFV

SEQ ID NO: 5 - protein, 474 aa, Homo sapiens
MDYKDDDDKHMGGSDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSGGGGSGGGGSGGGGSDEVWSDSEQSFLDPDIGGVAVAPTHGSILIECAKRELHATTPLKNPNRNHPTRISLVFYQHKSMNEPKHGLALWEAKMAEKAREKEEECEKYG

SEQ ID NO: 6 - protein, 474 aa, synthetic construct
MDYKDDDDKHMGGSDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSGGGGSGGGGSGGGGSDEVWSDSEQSFLDPDIGGVAVAPTHGSILIECAKRELHATTPLKNPNRNHPTRISLAFYQHKSMNEPKHGLALWEAKMAEKAREKEEECEKYG

SEQ ID NO: 7 - protein, 474 aa, synthetic construct
MDYKDDDDKHMGGSDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSGGGGSGGGGSGGGGSDEVWSDSEQSFLDPDIGGVAVAPTHGSILIECAKRELHATTPLKNPNRNHPTRISLCFYQHKSMNEPKHGLALWEAKMAEKAREKEEECEKYG

SEQ ID NO: 8 - protein, 474 aa, synthetic construct
MDYKDDDDKHMGGSDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSGGGGSGGGGSGGGGSDEVWSDSEQSFLDPDIGGVAVAPTHGSILIECAKRELHATTPLKNPNRNHPTRISLGFYQHKSMNEPKHGLALWEAKMAEKAREKEEECEKYG

SEQ ID NO: 9 - protein, 474 aa, synthetic construct
MDYKDDDDKHMGGSDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSGGGGSGGGGSGGGGSDEVWSDSEQSFLDPDIGGVAVAPTHGSILIECAKRELHATTPLKNPNRNHPTRISLIFYQHKSMNEPKHGLALWEAKMAEKAREKEEECEKYG

SEQ ID NO: 10 - protein, 474 aa, synthetic construct
MDYKDDDDKHMGGSDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVTACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSGGGGSGGGGSGGGGSDEVWSDSEQSFLDPDIGGVAVAPTHGSILIECAKRELHATTPLKNPNRNHPTRISLPFYQHKSMNEPKHGLALWEAKMAEKAREKEEECEKYG

SEQ ID NO: 11 - protein, 474 aa, synthetic construct
MDYKDDDDKHMGGSDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVSACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSGGGGSGGGGSGGGGSDEVWSDSEQSFLDPDIGGVAVAPTHGSILIECAKRELHATTPLKNPNRNHPTRISLVFYQHKSMNEPKHGLALWEAKMAEKAREKEEECEKYG

SEQ ID NO: 12 - protein, 878 aa, synthetic construct
MGGSDFPSCRCVEQIIEKDEGPFYTHLGAGPNVAAIREIMEERFGQKGKAIRIERVIYTGKEGKSSQGCPIAKWVVRRSSSEEKLLCLVRERAGHTCEAAVIVILILVWEGIPLSLADKLYSELTETLRKYGTLTNRRCALNEERTCACQGLDPETCGASFSFGCSWSMYYNGCKFARSKIPRKFKLLGDDPKEEEKLESHLQNLSTLMAPTYKKLAPDAYNNQIEYEHRAPECRLGLKEGRPFSGVSACLDFCAHAHRDLHNMQNGSTLVCTLTREDNREFGGKPEDEQLHVLPLYKVSDVDEFGSVEAQEEKKRSGAIQVLSSFRRKVRMLAEPVKTCRQRKLEAKKAAAEKLSSLENSSNKNEKEKSAPSRTKQTENASQAKQLAELLRLSGPVMQQSQQPQPLQKQPPQPQQQQRPQQQQPHHPQTESVNSYSASGSTNPYMRRPNPVSPYPNSSHTSDIYGSTSPMNFYSTSSQAAGSYLNSSNPMNPYPGLLNQNTQYPSYQCNGNLSVDNCSPYLGSYSPQSQPMDLYRYPSQDPLSKLSLPPIHTLYQPRFGNSQSFTSKYLGYGNQNMQGDGFSSCTIRPNVHHVGKLPPYPTHEMDGHFMGATSRLPPNLSNPNMDYKNGEHHSPSHIIHNYSAAPGMFNSSLHALHLQNKENDMLSHTANGLSKMLPALNHDRTACVQGGLHKLSDANGQEKQPLALVQGVASGAEDNDEVWSDSEQSFLDPDIGGVAVAPTHGSILIECAKRELHATTPLKNPNRNHPTRISLVFYQHKSMNEPKHGLALWEAKMAEKAREKEEECEKYGPDYVPQKSHGKKVKREPAEPHETSEPTYLRFIKSLAERTMSVTTDSTVTTSPYAFTRVTGPYNRYI

SEQ ID NO: 13 - protein, 9 aa, synthetic construct
FSGVTACLD

SEQ ID NO: 14 - protein, 9 aa, synthetic construct
FSGVSACLD